SEQUENCE LISTING (777 sequences)

SEQ ID NO: 1 - protein, 32 aa, Amblyomma variegatum
SDQGDVAEPKMHKTAPPFDFEAIPEEYLDDES

SEQ ID NO: 2 - protein, 10 aa, Amblyomma variegatum
SDQGDVAEPK

SEQ ID NO: 3 - protein, 22 aa, Amblyomma variegatum
MHKTAPPFDFEAIPEEYLDDES

SEQ ID NO: 4 - protein, 32 aa, Artificial Sequence
  variegin E31H
SDQGDVAEPKMHKTAPPFDFEAIPEEYLDDHS

SEQ ID NO: 5 - protein, 22 aa, Artificial Sequence
  variegin MH22A22E
MHKTAPPFDFEEIPEEYLDDES

SEQ ID NO: 6 - protein, 25 aa, Amblyomma variegatum
EPKMHKTAPPFDFEAIPEEYLDDES

SEQ ID NO: 7 - protein, 25 aa, Artificial Sequence
  variegin EP25A22E
EPKMHKTAPPFDFEEIPEEYLDDES

SEQ ID NO: 8 - protein, 21 aa, Amblyomma variegatum
EPKMHKTAPPFDFEAIPEEYL

SEQ ID NO: 9 - protein, 18 aa, Amblyomma variegatum
MHKTAPPFDFEAIPEEYL

SEQ ID NO: 10 - protein, 24 aa, Amblyomma variegatum
DVAEPKMHKTAPPFDFEAIPEEYL

SEQ ID NO: 11 - protein, 24 aa, Artificial Sequence
  variegin DV24K10R
DVAEPRMHKTAPPFDFEAIPEEYL

SEQ ID NO: 12 - protein, 23 aa, Artificial Sequence
  variegin DV23K10R
DVAEPRMHKTAPFDFEAIPEEYL

SEQ ID NO: 13 - protein, 23 aa, Amblyomma variegatum
DVAEPKMHKTAPFDFEAIPEEYL

SEQ ID NO: 14 - protein, 20 aa, Artificial Sequence
  Hirulog
  MISC_FEATURE (1)..(1): F=D-phenylalanine
FPRPGGGGNGDFEEIPEEYL

SEQ ID NO: 15 - protein, 22 aa, Artificial Sequence
  variegin MH22A22E
MHKTAPPFDFEEIPEEYLDDES

SEQ ID NO: 16 - protein, 24 aa, Artificial Sequence
  variegin DV24K10RYsulf
  SULFATION (23)..(23)
DVAEPRMHKTAPPFDFEAIPEEYL

SEQ ID NO: 17 - protein, 62 aa, Hirudinaria manillensis
VSYTDCTSGQNYCLCGGNFCGDGKHCEMDGSENKCVDGEGTPKRQTSGPSDFEEFSLDDIEQ

SEQ ID NO: 18 - protein, 65 aa, Hirudo medicinalis
VVYTDCTESGQNLCLCEGSNVCGQGNKCILGSDGEKNQCVTGEGTPKPQSHNDGDFEEIPEEYLQ

SEQ ID NO: 19 - protein, 72 aa, Hirudo medicinalis
AICVSQAITYTDCTESGQNLCLCEGSNVCGKGNKCILGSNGKGNQCVTGEGTPNPESHNNGDFEEIPEEYLQ

SEQ ID NO: 20 - protein, 65 aa, Hirudo medicinalis
VVYTDCTESGQNLCLCQGSNVCGQGNKCILGSNGEKNQCVTGEGTPKPQSHNDGDFEEIPEEYLQ

SEQ ID NO: 21 - protein, 65 aa, Hirudo medicinalis
VVYTDCTESGQNLCLCQDSNVCGQGNKCILGSNGEKNQCVTGEGTPKPQSHNDGDFEEIPEEYLQ

SEQ ID NO: 22 - protein, 65 aa, Hirudo medicinalis
VVYTDCTESGEDLCLCEGSNVCGEGNKCILGSDGEKNECVTGEGTPKPQSHNDGDFEEIPEEYLQ

SEQ ID NO: 23 - protein, 65 aa, Hirudo medicinalis
VVYTDCTESGQNLCLCEDSNVCGEGNKCILGSNGEKNQCVTGEGTPKPQSHNDGDFEEIPEEYLQ

SEQ ID NO: 24 - protein, 63 aa, Hirudo medicinalis
VVYTDCTESGQNLCLCEGSNVCGQGNKCILGSDGEKNQCVTGEGTPGPQSHNDGDFEEPEEYL

SEQ ID NO: 25 - protein, 84 aa, Hirudo medicinalis
MFSLKLFVVFLAVCICVSQAVSYTDCTESGQNYCLCVGSNVCGEGKNCQLSSSGNQCVHGEGTPKPKSQTEGDFEEIPDEDILN

SEQ ID NO: 26 - protein, 84 aa, Hirudo medicinalis
MFSLKLFVVFLAVCICVSQAVSYTDCTESGQNYCLCVGGNLCGGGKHCEMDGSGNKCVDGEGTPKPKSQTEGDFEEIPDEDILN

SEQ ID NO: 27 - protein, 65 aa, Hirudo medicinalis
VVYTDCTESGQNLCLCEDSNVCGQGNKCILGSNGEKNQCVTGEGTPKPQSHNDGDFEEIPEEYLQ

SEQ ID NO: 28 - protein, 65 aa, Hirudo medicinalis
ITYTDCTESGQNLCLCEGSNVCGKGNKCILGSNGEENQCVTGEGTPKPQSHNDGDFEEIPEEYLQ

SEQ ID NO: 29 - protein, 65 aa, Hirudo medicinalis
ITYTDCTESGQDLCLCEGSDVCGKGNKCILGSNGEENQCVTGEGTPKPQSHNDGDFEEIPEEYLQ

SEQ ID NO: 30 - protein, 65 aa, Hirudo medicinalis
ITYTDCTESGQDLCLCEGSNVCGKGNKCILGSNGEENQCVTGEGTPKPQSHNDGDFEEIPEEYLQ

SEQ ID NO: 31 - protein, 65 aa, Hirudo medicinalis
ITYTDCTESGQNLCLCEGSNVCGNGNKCKLGSDGEENQCVTGEGTPKPQSHNDGDFEEIPEEYLQ

SEQ ID NO: 32 - protein, 63 aa, Hirudo medicinalis
MRYTACTESGQNQCICEGNDVCGQGRNCQFDSSGKKCVEGEGTRKPQNEGQHDFDPIPEEYLS

SEQ ID NO: 33 - protein, 66 aa, Hirudo medicinalis
ITYTDCTESGQNLCLCEGSNVCGKGNKCILGSQGKDNQCVTGEGTPKPQSHNQGDFEPIPEDAYDE

SEQ ID NO: 34 - protein, 12 aa, Hirudo medicinalis
NGDFEEIPEEYL

SEQ ID NO: 35 - protein, 11 aa, Homo sapiens
VEYEALYPEDD

SEQ ID NO: 36 - protein, 12 aa, Homo sapiens
EVEYEALYPEDD

SEQ ID NO: 37 - protein, 11 aa, Homo sapiens
AEVEYEALYPE

SEQ ID NO: 38 - protein, 12 aa, Homo sapiens
AEVEYEALYPED

SEQ ID NO: 39 - protein, 13 aa, Homo sapiens
AEVEYEALYPEDD

SEQ ID NO: 40 - protein, 9 aa, Homo sapiens
EALYPEDDL

SEQ ID NO: 41 - protein, 12 aa, Homo sapiens
VSVEHEVDVEYP

SEQ ID NO: 42 - protein, 12 aa, Bos taurus
VRVEHHVEIEYD

SEQ ID NO: 43 - protein, 13 aa, Hirudo medicinalis
NGDFEEIPEEYLQ

SEQ ID NO: 44 - protein, 7 aa, Aspergillus flavus
FPRFPRP

SEQ ID NO: 45 - protein, 6 aa, Homo sapiens
QSHNDG

SEQ ID NO: 46 - protein, 21 aa, Homo sapiens [sequence omitted]

SEQ ID NO: 47 - protein, 9 aa, Homo sapiens
PEHPAETEY

SEQ ID NO: 48 - protein, 17 aa, Homo sapiens
EHPAETEYESLYPEDDL

SEQ ID NO: 49 - protein, 17 aa, Homo sapiens
EHPAETEFESLYPEDDL

SEQ ID NO: 50 - protein, 14 aa, Homo sapiens
AETEYESLYPEDDL

SEQ ID NO: 51 - protein, 20 aa, Homo sapiens
VRPEHPAEVEYEALYPEDDL

SEQ ID NO: 52 - protein, 9 aa, Homo sapiens
PEHPAEVEY

SEQ ID NO: 53 - protein, 17 aa, Homo sapiens
EHPAEVEYEALYPEDDL

SEQ ID NO: 54 - protein, 14 aa, Homo sapiens
AEVEYEALYPEDDL

SEQ ID NO: 55 - protein, 11 aa, Homo sapiens
EYEALYPEDDL

SEQ ID NO: 56 - protein, 4 aa, Homo sapiens
AGDV

SEQ ID NO: 57 - protein, 20 aa, Homo sapiens
VRPEHPAETEYESLYPEDDL

SEQ ID NO: 58 - protein, 174 aa, Ixodes scapularis
MHQEGDFKMGHCSDLKVSALEIPYKGNKMSMVILLPEDVEGLSDLEEHLTAPKLLALLGGMYVTSDVNLHFPKFKLEQSMGLKDVLMAMGVKDFFTFLADLSGISATGNLCASDVIHKAFVEVNEEGTEAAAATAILMDCIPQVVNFFVDHPFMFLICSHDPDAVLFMGSIREL

SEQ ID NO: 59 - protein, 178 aa, Ixodes scapularis
MHQKGDFKMGHCSDLKVTALEIPYKGNKMSMIILLPEDVEGLSVLEEHLTAPKLSALLGGMYVTPDVNLRLPKFKLEQSIGLKDVLMAMGVKDFFTSLADLSGISAAGNLCASDVIHKAFVEVNEEGTEAAAATAIPMMLMCARFPQVVNFFVDHPFMFLIHSHDPDVVLFMGSIREL

SEQ ID NO: 60 - protein, 374 aa, Ixodes scapularis
MASDFSNSLISFSVDLYKKLKSESDGASNFICSPFSIAAALSMTLAGAKHDTAKQISNALHMQDTTVHENFAYFFSKLPGYAPDVILHVANRLYAEETYNTLDEFTHLLEKSYSTTVEKVDFKRNAEKTRLQVNTWVEEVTQSKIKDLLAEGTIDDFTSLIIINAVYFKGLWHDQFDPKRTSQQEFHLTADRTKMVDMMHHKQRFRMCRHPNFKVSALEIPYKGQKMSMVILLPEEIDGLADLEETLTSSKIREIIQELSYQGDIELSLPRFKLEHTVGLKNVLAAMGIEDMFDALKCDLSGISPDNALVVSDVVHKAFIEVNEEGTEAAAATAMVMLCCMSFPTRFTVDHPFLFLIRCHDPDVILFIGSVAQI

SEQ ID NO: 61 - protein, 382 aa, Echinococcus multilocularis
MFAKTSFIPFTQALYAQLQPSEGRSNFFMSPLSVYSALSLALAGSESETREELVSVLGLAPGKDIDTIVKSLGENLQAVADGDAKKTLVEANGVFIQAGSQIRETYTSAVSKHLKADMKQLDFGGDSEGSRISINRWIAEKTREKVKDLLAQGSITPMTHVVLANAVYFKGVWKCKFEKSKTDRSGVFHSLDSGDVRVSMMTQKASYPMADFVDLEVRALKVPFETHEMLIVLPEKNDGLPNLLKQLSANAKHLEEMLTSDQYFDTEVVLKLPKFSLGGHNMKLKEPLHKMGLKSAFDAERADFSGITNDRSLAVSDVYHQAVIDVDEEGAEAAAATAMPMMVRCMPAPPVDFFVDHPFIFFIVTKTGIPVFMGHVVHPESK

SEQ ID NO: 62 - protein, 309 aa, Stomoxys calcitrans
MKYFVFIGIIALSAVSQAQNGRWQGDLHGLSGHRGSGLPGLSGHRGSGQPGLSGHRGSEQSGEAGAPSYDYFSQPGLSGSRRHGQRDLEVEDSRPARSLDPLNSVPEWNENDEDDHEFGPYRKPQDNQRDRRRPQRSFDSFYSVPEWNEDNEADQGYRKRQDNQQNRHRAQPQRPLHPHYSHPEWEEEDEGDQQFGRYRKPHDNQQDRRRGKAQPQMRLHPFNSHPEWEDEDEGDQQFGPYRRPQDNQQNRRGESRPIRSNGDELSSDDQLASFFGFPAGDVPEDLKNLVRLFSGPNNDFGGNNQFEED

SEQ ID NO: 63 - protein, 465 aa, Bos taurus
MISNGIGTVTAGKRSICLLPLLLIGLWGCVTCHRSPVEDVCTAKPRDIPVNPMCIYRSSEKKATEGQGSEQKIPGATNRRVWELSKANSHFATAFYQHLADSKNNNDNIFLSPLSISTAFAMTKLGACNNTLKQLMEVFKFDTISEKTSDQIHFFFAKLNCRLYRKANKSSELVSANRLFGGKSITFNETYQDISEVVYGAKLQPLDFKGNAEQSRLTINQWISNKTEGRITDVIPPQAINEFTVLVLVNTIYFKGLWKSKFSPENTRKELFYKADGESCSVLMMYQESKFRYRRVAESTQVLELPFKGDDITMVLILPKLEKTLAKVEQELTPDMLQEWLDELTETLLVVHMPRFRIEDSFSVKEQLQDMGLEDLFSPEKSRLPGIVAEGRSDLYVSDAFHKAFLEVNEEGSEAAASTVISIAGRSLNSDRVTFKANRPILVLIREVALNTIIFMGRVANPCVD

SEQ ID NO: 64 - protein, 413 aa, Spermophilus tridecemlineatus
MPSSISWGLLLLAGLSCLVAGSLAEDAQETGASKHDQEHPASHRIAPNLAEFALSLYRVLAHESNTTNIFFSPVSIAMALASLSLGTKADTHTQIMEGLGFNLTETAESDIHQGFQHLLQTLNKPNSQLQLTTGNGLFIDHNLKLLDKFLQDVKNLYHSEAFSTDFTNTEEAKKQINTYVEKGTQGKIVDLVKDLNRDSVLALVNYIFFKGKWEKPFEVDHTKEEDFHVDQVTTVRVPMMNRMGMFEVHYCSTLASWVLQMDYLGNATAIFLLPDEGKLQHLEDTITKEILAKFLKNRESSSVNLHFPKLNISGTMDLKPVLTRLGITNVFSYKADLSGITEDDPLRVSQALHKAVLTIDERGTEAAGATFLEMMPMSLPPEVKFDKPFLVVIIEHSTKSPLFVGKVVNPTLH

SEQ ID NO: 65 - protein, 346 aa, Homo sapiens
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 66 - protein, 410 aa, Mus musculus
MTYLELLALLALQSVVTGATFPDETITEWSVNMYNHLRGTGEDENILFSPLSIALAMGMMELGAQGSTRKEIRHSMGYEGLKGGEEFSFLRDFSNMASAEENQYVMKLANSLFVQNGFHVNEEFLQMLKMYFNAEVNHVDFSQNVAVANSINKWVENYTNSLLKDLVSPEDFDGVTNLALINAVYFKGNWKSQFRPENTRTFSFTKDDESEVQIPMMYQQGEFYYGEFSDGSNEAGGIYQVLEIPYEGDEISMMLALSRQEVPLATLEPLLKAQLIEEWANSVKKQKVEVYLPRFTVEQEIDLKDILKALGVTEIFIKDANLTAMSDKKELFLSKAVHKSCIEVNEEGSEAAAASGMIAISRMAVLYPQVIVDHPFLYLIRNRKSGIILFMGRVMNPETMNTSGHDFEEL

SEQ ID NO: 67 - protein, 398 aa, Homo sapiens
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITTGSENLHVSHILQKAKIEVSEDGTKASAATTAILIARSSPPWFIVDRPFLFFIRHNPTGAVLFMGQINKP

SEQ ID NO: 68 - protein, 397 aa, Mus musculus
MNWHFPFFILTTVTLYSVHSQFNSLSLEELGSNTGIQVFNQIIKSRPHENVVVSPHGIASILGMLQLGADGKTKKQLSTVMRYNVNGVGKVLKKINKAIVSKKNKDIVTVANAVFLRNGFKMEVPFAVRNKDVFQCEVQNVNFQDPASASESINFWVKNETRGMIDNLLSPNLIDGALTRLVLVNAVYFKGLWKSRFQPESTKKRTFVAGDGKSYQVPMLAQLSVFRSGSTRTPNGLWYNFIELPYHGESISMLIALPTESSTPLSAIIPHITTKTIDSWMNTMVPKRMQLVLPKFTAVAQTDLKEPLKALGITEMFEPSKANFTKITRSESLHVSHILQKAKIEVSEDGTKASAATTAILIARSSPPWFIVDRPFLFSIRHNPTGAILFLGQVNKP

SEQ ID NO: 69 - protein, 410 aa, Rattus norvegicus
MAYLGLLSLVALQSLVTGAAFPDETIAEWSVNVYNHLRATGEDENILFSPLSIALAMGVMELGAQGSTLKEIRHSMGYESLKSGEEFSFLRDFSSMVSAEEGQYVMKIANSLFVQNGFHINEEFLQMMKMYFNAEVNHVDFSENVAVANYINKWVENYTNSLLKDLVSPGDFDAVTHLALINAVYFKGNWKSQFRPENTRTFSFTKDDESEVQIPMMYQQGEFYYGEFSDGSNEAGGIYQVLEIPYEGDEISMMLVLSRQEVPLATLEPLLKPQLIEEWANSVKKQKVEVYLPRFTVEQEIDLKDILKALGVTEIFIKDANLTAMSDKKELFLSKAVHKSFIEVNEEGSEAAVASGMIAISRMAVLFPQVIVDHPFLFLIKNRKTGTILFMGRVMHPETMNTSGHDFEEL

SEQ ID NO: 70 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 71 - protein, 484 aa, Xenopus laevis
MKLLHLATIFLLIHATLGGVKDLQEHFEDTSTGINPRGSQTQAVENLLDDTVTNDLSTEGEDEEDYLDFDKIFGEDEDYIDIIDAAPEIKNSETQQGNIFELFHGKTRVQRLNIINANFGFNLYRAIKNNTDASENILLAPVGISTAMATISLGTKGQTLEQVLLTLGFKDFLNASSKYEILTLHNVFRKLTHRLFRRNFGYTLRSVNDIYVKRDFLIREPFKNNLKNYYFAEAQTVDFGYKDFLTKANKRIQQLTKGLIEEALTNVDPALLMLLVNCIYFKGTWENKFPVEYTQNMNFRLNEKELVKVPMMKTKGNFLVAADPELDCAVLQLPYVGNISMLIVLPHKLSGMKLLEKQISPQVVERWQNIMTNRTREVFLPRFKLEKSYDLQKVLSNMGATDLFTHGDFSGVSDKDINIGLFQHQGTITVNEEGTEAAAVTVVGFMPLSTQARFVADRPFLFLIYEHRTNCLVFMGRVANPTKS

SEQ ID NO: 72 - protein, 473 aa, Gallus gallus
GTFCGIKDFSDHFESLKDAHTHENGTYNMPDLPLEFHRENTITNDLIPEEEEEEDYLDLDKILGEDDYSDIIDAAPHIVSEIQQGNILELFQGKTRIQRLNILNANFGFNLYRSVADKANSSDNILMAPVGISTAMAMISLGLKGQTQQEVLSVLGFEDFINASAKYELMTVHNLFRKLTHRLFRRNFGYTLRSVNDLYIRKDFSILNDFRNNMKTYYFADAQPADFSDPNFITKTNERILKLTKGLIKEALVNVNPTTLMMILNCLYFKGTWENKFPVEMTTKRSFRLNEKQTIKVPMMQTKGNFLAAADPELDCGVIQLPFVGNISMLIVLPHKLSGMKALEKQITPQVVEKWQKSMTNRTREVVLPKFKLEKNYNLIGFLRSMGIEELFSEKGNYCGVSEEKVSIDRFNHQGTITVNEEGTEAGAITNVGFMPLSTQIRFIVDRPFLFLIYEHRTNCLLFMGRVVNPAKP

SEQ ID NO: 73 - protein, 480 aa, Oryctolagus cuniculus
MQHRPHLLLISLTIMSVCGGSNGLTDQLNNKNLTMPLLPIEFHKENTVTNDWIPEGEEDDDYLDLEKLLSEDDDYIDIIDAVSPTDSEASAGNILQLFQGKSRIQRLNILNAKFAFSLYRALKDQANAFDNIFIAPVGISTAMGMISLGLKGETHEQVHSVLHFRDFVNASSKYEILTIHNLFRKLTHRLFRRNFGYTLRSVNDLYVQKQFPIREDFKAKVREYYFAEAQAADFSDPAFISKANNHILKVTKGLIKEALENVDPATQMMILNCIYFKGTWVNKFPVEMTHNHNFRLNEREVVKVSMMQTKGNFLAANDQELACDVLQLEYVGGISMLIVVPHKLSGMKTLEAQLTPQVVERWQKSMTNRTREVLLPKFKLEKNYNLVEALKSMGVTELFDKNGNMSGISDQGITMDLFKHQGTITVNEEGTQAAAVTTVGFMPLSTQVRFTVDRPFLFLVYEHRTSCLLFMGKVANPVRS

SEQ ID NO: 74 - protein, 478 aa, Mus musculus
MKHPLCTLLSLITFMCIGSKGLAEQLTNENLTTSFLPANFHKENTVTNDWIPEGEEDEDYLDLEKLLGEDDDYIYIIDAVSPTDSESSAGNILQLFQGKSRIQRLNILNAKFAFNLYRVLKDQATTSDNLFIAPVGISTAMGMISLGLRGETHEEVHSVLHFRDFVNASSKYEVTTIHNLFRKLTHRLFRRNFGYTLRSVNGLYIQKQFPIREDFKAAMREFYFAEAQEANFPDPAFISKANNHILKLTKGLIKEALENIDPATQMLILNCIYFKGTWVNKFPVEMTHNHNFRLNEREVVKVSMMQTKGNFLAANDQELDCDILQLEYVGGISMLIVVPRKLSGMKTLEAQLTPQVVERWQKSMTNRTREVLLPKFKLEKNYNLVEVLKSMGITKLFNKNGNMSGISDQRIAIDLFKHQSTITVNEEGTQAAAVTTVGFMPLSTQVRFTVDRPFLFLVYEHRTSCLLFMGKVTNPAKS

SEQ ID NO: 75 - protein, 479 aa, Rattus norvegicus
MKHPAYTLLLSLIMSMCAGSKGLAEQLTKENLTVSLLPPNFHKENTVTNDWIPEGEEDDDYLDLEKLLSEDDDYIYVVDAVSPTDSESSAGNILQLFQGKSRIQRLNILNAKFAFNLYRVLKDQATSSDNIFIAPVGISTAMGMISLGLRGETHEEVHSVLHFKDFVNASSKYEVTTIHNLFRKLTHRLFRRNFGYTLQSVNDLYIQKQFPIREDFKAAMREFYFAEAQEADFSDPAFISKANSHILKLTKGLIKEALENTDSATQMMILNCIYFKGAWMNKFPVEMTHNHNFRLNEREVVKVSMMQTKGNFLAANDQELDCDILQLEYVGGISMLIVIPRKLSGMKTLEAQLTPQVVERWQKSMTNRTREVLLPKFKLEKNYNLVEVLKSMGITKLFNKNGNMSGISDQRIIIDLFKHQSTITVNEEGTQAAAVTTVGFMPLSTQVRFTVDRPFLFLVYEHRTSCLLFMGRVANPAKS

SEQ ID NO: 76 - protein, 507 aa, Danio rerio
MWLVPVIVVACLLNSPALAGVKDLSSHFSTLEKEKTVDARGLSPGGENTDMESIPLDFHRENTVTNDLPEGQDDEDYVDFDKILGEDDYSEGDHIDEISTPAPDLDLFYEPSDPKIRRARLLRLFHGQTRLQRINVVNARFGFRLYRKLRNRLNQTDNILLAPVGISIAMGMMGLGVGPNTQEQLFQTVGFAEFVNASNHYDNSTVHKLFRKLTHRLFRRNFGYTLRSVNDLYVKRNVQIQDSFRADAKTYYFAEPQSVDFADPAFLVKANQRIQKITKGLIKEPLKSVDPNMAVMLLNYLYFKGTWEQKFPKELTHHRQFRVNEKKQVRVLMMQNKGSYLAAADHELNCDILQLPYAGNISMLIAVPQKLSGMRSLEQEISPTLVNKWLSNMTNRTREVVFPRFKLEQNYDLIEHLKEMGMTDIFTEKGDFSPMTSEKVIINWFKHQGSITVNEEGTEAAAMTHIGFMPLSTQTRFIVDRPFLFLIYEHRTGCVVFMGRVVDPSQT

SEQ ID NO: 77 - protein, 507 aa, Danio rerio
MWLVPVIVVACLLNSPALAGVKDLSSHFSTLEKEKTVDARGLSPGGENTDMESIPLDFHRENTVTNDLPEGQDDEDYVDFDKILGEDDYSEGDHIDEISTPAPDLDLFYEPSDPKIRRARLLRLFHGQTRLQRINVVNARFGFRLYRKLRNRLNQTDNILLAPVGISIAMGMMGLGVGPNTQEQLFQTVGFAEFVNASNHYDNSTVHKLFRKLTHRLFRRNFGYTLRSVNDLYVKRNVQIQDSFRADAKTYYFAEPQSVDFADPAFLVKANQRIQKITKGLIKEPLKSVDPNMAVMLLNYLYFKGTWEQKFPKELTHHRQFRVNEKKQVRVLMMQNKGSYLAAADHELNCDILQLPYAGNISMLIAVPQKLSGMRSLEQEISPTLVNKWLSNMTNRTREVVFPRFKLEQNYDLIEHLKEMGMTDIFTEKGDFSPMTSEKVIINWFKHQGSITVNEEGTEAAAMTHIGFMPLSTQTRFIVDRPFLFLIYEHRTGCVVFMGRVVDPSQS

SEQ ID NO: 78 - protein, 527 aa, Artificial Sequence [sequence omitted]

SEQ ID NO: 79 - protein, 507 aa, Danio rerio
MWLVPVIVVACLLNSPALAGVKDLSSHFSTLEKEKTVDARGLSPGGENTDMESIPLDFHRENTVTNDLPEGQDDEDYVDFDKILGEDDYSEGDHIDEISTPAPDLDLFYEPSDPKIRRARLLRLFHGQTRLQRINVVNARFGFRLYRKLRNRLNQTDNILLAPVGISIAMGMMGLGVGPNTQEQLFQTVGFAEFVNASNHYDNSTVHKLFRKLTHRLFRRNFGYTLRSVNDLYVKRNVQIQDSFRADAKTYYFAEPQSVDFADPAFLVKANQRIQKITKGLIKEPLKSVDPNMAVMLLNYLYFKGTWEQKFPKELTHHRQFRVNEKKQVRVLMMQNKGSYLAAADHELNCDILQLPYAGNISMLIAVPQKLSGMRSLEQEISPTLVNKWLSNMTNRTREVVFPRFKLEQNYDLIEHLKEMGMTDIFTEKGDFSPMTSEKVIINWFKHQGSITVNEEGTEAAAMTHIGFMPLSTQTRFIVDRPFPFLIYEHRTGCVVFMGRVVDPSQT

SEQ ID NO: 80 - protein, 376 aa, Homo sapiens
MDVLAEANGTFALNLLKTLGKDNSKNVFFSPMSMSCALAMVYMGAKGNTAAQMAQILSFNKSGGGGDIHQGFQSLLTEVNKTGTQYLLRMANRLFGEKSCDFLSSFRDSCQKFYQAEMEELDFISAVEKSRKHINTWVAEKTEGKIAELLSPGSVDPLTRLVLVNAVYFRGNWDEQFDKENTEERLFKVSKNEEKPVQMMFKQSTFKKTYIGEIFTQILVLPYVGKELNMIIMLPDETTDLRTVEKELTYEKFVEWTRLDMMDEEEVEVSLPRFKLEESYDMESVLRNLGMTDAFELGKADFSGMSQTDLSLSKVVHKSFVEVNEEGTEAAAATAAIMMMRCARFVPRFCADHPFLFFIQHSKTNGILFCGRFSSP

SEQ ID NO: 81 - protein, 376 aa, Homo sapiens
MDVLAEANGTFALNLLKTLGKDNSKNVFFSPMSMSCALAMVYMGAKGNTAAQMAQILSFNKSGGGGDIHQGFQSLLTEVNKTGTQYLLRVANRLFGEKSCDFLSSFRDSCQKFYQAEMEELDFISAVEKSRKHINTWVAEKTEGKIAELLSPGSVDPLTRLVLVNAVYFRGNWDGQFDKENTEERLFKVSKNEEKPVQMMFKQSTFKKTYIGEIFTQILVLPYVGKELNMIIMLPDETTDLRTVEKELTYEKFVEWTRLDMMDEEEVEVSLPRFKLEESYDMESVLRNLGMTDAFELGKADFSGMSQTDLSLSKVVHKSFVEVNEEGTEAAAATAAIMMMRCARFVPRFCADHPFLFFIQHRKTNGILFCGRFSSP

SEQ ID NO: 82 - protein, 376 aa, Homo sapiens
MDVLAEANGTFALNLLKTLGKDNSKNVFFSPMSMSCALAMVYMGAKGNTAAQMAQVLSFNKSGGGGDIHQGFQSLLTEVNKTGTQYLLRTANRLFGEKSCDFLSSFRDSCQKFYQAEMEELDFISAVEKSRKHINSWVAEKTEGKIAELLSPGSVDPLTRLVLVNAVYFKGNWNEQFDKENTEERRFKVSKNEEKPVQMMFMQSTFRKTYIGEIFTQILVLPYVGKELNMIIMLPDETTDLRTVEKELTYEKFVEWTRLDMMDEEKVEVSLPRFKLEESYDMESVLCSLGMTDAFELGKADFSGMSKADLCLSKVVHKSFVEVNEEGTEAAAATAAIMMMRCARFVPRFCADHPFLFFIQHSKTNGVLFCGRFSSP

SEQ ID NO: 83 - protein, 381 aa, Canis familiaris
MDTLSEANGTFAISLLKKLGEDGSKNVFFSPMSISSALSMVFMGAKGNTAAQMSQTLSLSKSGGGGDVHQGFQALLNEVNSAEARYLLRTANRLFGEKTCGFLSSFKDSCRTFYQAEMEELDFLSACEQSREHITAGVTEGRKVKTRGKIVDLLSPGSVDPGTNLILVNAIYFKGNWDKQFNKEQTTERPFKVSKNEKKPVQMMFKKSTFQMTYIGEIFTKILVLPYVGRELNMIIMLPDENVSLETVETELTYEKFTEWTRPDMLDEEEVEVFLPRFKLEEEYDMKAVLCSLGMTDAFEQSKADFSGMSSRGDLYLSKVVHKSFVEVNEEGTEAAAASAAVMMLRCARIVPRFCADRPFLFFIQHSKSRSVLFCGRFSSP

SEQ ID NO: 84 - protein, 345 aa, Canis familiaris
MDTLSEANGTFAISLLKKLGEDGSKNVFFSPMSISSALSMVFMGAKGNTAAQMSQTLSLSKSGGGGDVHQGFQALLNEVNSAEARYLLRTANRLFGEKTCGFLSVSPLSPARKIVDLLSPGSVDPGTNLILVNAIYFKGNWDKQFNKEQTTERPFKVSKNEKKPVQMMFKKSTFQMTYIGEIFTKILVLPYVGRELNMIIMLPDENVSLETVETELTYEKFTEWTRPDMLDEEEVEVFLPRFKLEEEYDMKAVLCSLGMTDAFEQSKADFSGMSSRGDLYLSKVVHKSFVEVNEEGTEAAAASAAVMMLRCARIVPRFCADRPFLFFIQHSKSRSVLFCGRFSSP

SEQ ID NO: 85 - protein, 378 aa, Equus caballus
MDTLSEANGTFALNLLKKLGEDNSKNVFFSPMSISSALAMVFMGAKGNTAAQMSQVLSLSKSGGEVGDVHQGFQSLLSEINRPGTQYLLRTANRLFGEKSYDFLSSFKDSCHKFYQAEMEQLDFISATEESRKHINTWVAKKTEGKITELLSSDSVDLLTKLILVNAIYFKGNWDDQFDKQQTKERPFKVSKNEEKPVQMMFKKSTFKRTYIGEIFTQILMLPYVGEELNMIIMLPDENTDLKTVEKELTYEKFVEWTRPDMMDETEMEVFLPRFKLEEDYDMEAVLRSLGMTDAFEQARADFSGMSSRADLFLSKVVHKSFVEVNEEGTEAAAATAAVMMMRCVRIIPRFCADHPFLFFIQHSKTNSILFCGRFSSP

SEQ ID NO: 86 - protein, 378 aa, Mus musculus
MDPLQEANGTFALNLLKILGEDSSKNVFLSPMSISSALAMVFMGAKGTTASQMAQALALDKCSGNGGGDVHQGFQSLLTEVNKTGTQYLLRTANRLFGDKTCDLLASFKDSCLKFYEAELEELDFQGATEESRQHINTWVAKKTEDKIKEVLSPGTVNSDTSLVLVNAIYFKGNWEKQFNKEHTREMPFKVSKNEEKPVQMMFKKSTFKMTYIGEIFTKILLLPYVSSELNMIIMLPDEHVELSTVEKEVTYEKFIEWTRLDKMDEEEVEVFLPKFKLEENYNMNDALYKLGMTDAFGGRADFSGMSSKQGLFLSKVVHKAFVEVNEEGTEAAAATAGMMTVRCMRFTPRFCADHPFLFFIHHVKTNGILFCGRFSSP

SEQ ID NO: 87 - protein, 260 aa, Bos taurus
MEELDFLSATEESRKHINTWVAEKTEGKIRDLLPANSVNAMTRLVLVNAIYFKGNWDTQFNKEHTKERPFRVSKNMEKPVQMMFKKSTFKSTYIGEISTQILVLPYVGKELNMVILLPSESTDLNTVEKALTYEKFVTWTKLDVMDEDEVEVFLPRFTLEESYDMECVLRDLGVTDAFEAAQADFSGMSCQQDLHLSKIVHKSFVEVTEEGTEAAAATVARITPRILRIVPRFCADHPFLFFIQHSRTGAILFCGRFCSP

SEQ ID NO: 88 - protein, 324 aa, Bos taurus
QTLSLNKSSGGGEDVHQGFQNLLSEVNRRDTQYLLRTANRLFGEKTYDFLSSFKDSCHKFYQAEMEELDFVSATEQSRKHINTWVAEKTEGKIRDLLPANSVNPMTRLVLVNAIYFKGNWDTQFNKEHTEERPFRVSKNVEKPVQMMFKKSTCKITYIGEISTQILVLPYVGQELNMVILLPSESTDLNTVEKALTYEKFIAWTKPDVMDEEEVEVFLPRFTLEESYDMEEFLQELGMTDAFEETRADFSGMSSGRGLHLSKVMHKSFVEVTEEGTEAAAATGAVVMMRCLMVVPRFNANHPFLFFIQHSKTGAILFCGRFCSP

SEQ ID NO: 89 - protein, 402 aa, Monodelphis domestica
MVMNFPITFEPNILLSGSQVYKRWCVRMPALSEANNTFALNFFKKIGEEESEENVFYSPLSLYYALTMVLEGATGETAEQIQQVLSLSKNTDVHQSFQSFLAEVNKTGAPPLLRVANALFGEKTCGFLSPFKESCQKFYFSNVEELDFAHMPEAARKHINDWVEEKTEGKISELLANDSVDVMTNLVLVNAIYFNGKWEKPFDKAKTAEKMFNISEKKQKPVQMMYQRSTFSMTFIEDIPTQILVLPYAGGHMDMVILLPVENKHLKMLKKRLTSENLVDWINPEMMNEIEVEVFLPKFILAEHLDVEVILQKLGMLDAFDKTKADFSKMSARNDLCLSKVIHKAYVEVNEEGTVAVSSTAAVMMTRSERMSLEFKADHPFIFYLIEKQTNKIVFIGEVTSP

SEQ ID NO: 90 - protein, 407 aa, Rattus norvegicus
METLNVRRQILTRSSPDDFGMLSIKITSGEMLKDLESLRLAFWDSKKVSILLSVSDEIVQLTIMDPLLKANGNFAIKLFKVLGEDISKNVFFSLPSISSALSMILMGANGTTASQICQAMSLDKCNSIGGGDVHQHFLSLLTKVNKTDTRCMLRKANSVFIEDSFEILASFKDACHKLYEAEIEELDFKGAPEQSRQHINTWVAKKTEDIIRELLPPCTVNSNTCLFLVNVIYFKGSLEKPFNKADTREMPFKVSMVTTVHLLNERKSVYMFYKISTFKMTYVEEVSTKILLLLYVSIELTMIMLFSPRGWKHEIVGGCANCLDARDRNKAQIEEWACALRHLPAASGVKWDSACPEVEIMDSFHMKFSSELHFGLDFSLLCGWTESFVVLSKRYASEKQVSKPLFS

SEQ ID NO: 91 - protein, 462 aa, Macaca mulatta
MAAGKASGESEEASPSLTAEEREALGGLDRWRQDEGPTGQGKAAASRSADTVSPAGAALAPGPAAGSALIVAADDGRGASGKLFPASALPPPTVNGRVSGAGPFLEPGQHSVSLEAPETRRWGPELVKAKASGQGGVDPRVPPRLSQCSGPGTPPASPRVFEVRGGSGVSTRRSTRSPPWVGKAGGASVCGGRGSGAGQRAAGPGTRVGQRRAGTREPREWGVAARGCDPRRPSRAGARASVRLGFPSGASQLRRTATRPVGRAWPRARPVTPLTRGPRAHRPSAHGRGGRRGMGAAQSLPGHRSAIMDVLAEANGTFALNLLKTLGKDNSKNVFFSPMSMSCALAMVYMGAKGNTAAQMAQVLSFNKSGGGGDIHQGFQSLLTEVNKTGTQYLLRTANRLFGEKSCDFLSSFRDSCQKFYQAEMEELDFISAVEKSRKHINSWVAEKTEGENVLLSTRNSI

SEQ ID NO: 92 - protein, 269 aa, Macaca mulatta
MAKAHYRFLTENSQAVAVFTRIEIGRFAHIRKSRGLRDPPRPPAQAPAGLTVMDALSEGNGTFALNLLKKLGENNSPNLKILFGNWQGPNKEKPVQMMFKKSTFQMTYAKEILNKILVLSYVGKELNMLPDENTDLKMLMSVEKELSYERLIEWTKPDNMHEREMEVFLPRFKLEETYNMEDVLRSMDMVDALEQDRADLKDLYLSKVMHKSFVEVNEEGTEAAAATTEEIVLCCASYSLRFCADHPFLFFIQHNKTNGILFCCRFSSP

SEQ ID NO: 93 - protein, 377 aa, Bos taurus
MDALSEANGTFALTLLKKLGEGNSKNVLIAPLSISSALAMVLLGARGNTAAQMCQTLSLNKSSGGGEDVHQGFQNLLCEVNRTDTRYLLRTANRLFGEKTYNFLSSFKDSCRKFYQAEMEELDFVCATEESRKHINTWVAEKTEGKIRDLLSANSVYPMTCLVLVNAIYFKGNWDKQFYKVHTKERPFQVSKVNKTVQMMFRKSTFKMTYIAEICTQILVLPYVGQELNMVILLPRERTDLNTVEKALTYEKFVVWTKPDMLAEEEVEVFLPRFTLEESYDMECVLRDLGMTDGFNMARADFIGLSCQPGLHLSKVVHKPFVEVTEEGTEAVAASEARIRGLSLRTVPRFCANRPFLFFIQHSSTGAILFCGRFCSP

SEQ ID NO: 94 - protein, 377 aa, Bos taurus
MDALSEANGTFALTLLKKLGEGSSKNVLIAPLSISSALAMVLLGARGNTAAQMCQTLSLNKSSGGGEDVHQDFQNLLSEVNRTDTQYLLRTANRLFGEKPYDFLSSFKNACHIFYQAEMEELDFVSATEEPTKHINTWVAEKTEGKIRDLLPANSVNPMTRLVLVSAVYFKGNWAKPFLKGRTMEGIFNVCKNVQKRALMIYNWSTFKTACIAEICSQILVLPYVGQELNMVILLPFESTDLITVEKALTYEKFVTWTKPDVLAEVEVEVFLPCFTLEESYDMECVLRDLGMTDAFNAARADFSGMSCQPGLHLSKVMHKSFLEVTEEGTEAVAASEARIRRSLGVVHHFYANRPFLFFIQHSRTGAILFCGRFCSP

SEQ ID NO: 95 - protein, 413 aa, Mus musculus
MTPSISWGLLLLAGLCCMVPSFLAEDVQETDTSQKDQSPASHEIATNLGDFAISLYRELVHQSNTSNIFFSPVSIATAFAMLSLGSKGDTHTQILEGLQFNLTQTSEADIHKSFQHLLQTLNRPDSELQLSTGNGLFVNNDLKLVEKFLEEAKNHYQAEVFSVNFAESEEAKKVINDFVEKGTQGKIVEAVKELDQDTVFALANYILFKGKWKKPFDPENTEEAEFHVDKSTTVKVPMMMLSGMLDVHHCSILSSWVLLMDYAGNASAVFLLPEDGKMQHLEQTLNKELISKILLNRRRRLVQIHIPRLSISGDYNLKTLMSPLGITRIFNNGADLSGITEENAPLKLSKAVHKAVLTIDETGTEAAAATVFEAVPMSMPPILRFDHPFLFIIFEEHTQSPIFVGKVVDPTHK

SEQ ID NO: 96 - protein, 413 aa, Mus musculus
MTPSISWGLLLLAGLCCLVPSFLAEDVQETDTSQKDQSPASHEIATNLGDFAISLYRELVHQSNTSNIFFSPVSIATAFAMLSLGSKGDTHTQILEGLQFNLTQTSEADIHKSFQHLLQTLNRPDSELQLSTGNGLFVNNDLKLVEKFLEEAKNHYQAEVFSVNFAESEEAKKVINDFVEKGTQGKIAEAVKKLDQDTVFALANYILFKGKWKKPFDPENTEEAEFHVDESTTVKVPMMTLSGMLHVHHCSTLSSWVLLMDYAGNATAVFLLPDDGKMQHLEQTLSKELISKFLLNRRRRLAQIHFPRLSISGEYNLKTLMSPLGITRIFNNGADLSGITEENAPLKLSQAVHKAVLTIDETGTEAAAVTVLQMVPMSMPPILRFDHPFLFIIFEEHTQSPIFLGKVVDPTHK

SEQ ID NO: 97 - protein, 412 aa, Mus caroli
MTPSISWGLLLLAGLFCLVPSFLAEDVQETDTSRRDSVPASHDTPYNLELSISLYRELGHKSTTSNIFFSQVSIATAFAMLSLGEKGDTHTQILEGLQFNLTQTSEADIHKAFQHLLQTLNRPDSELQLSTGNGSLLNNDLKLVEKFLEEAKNNYHSEVFSVNFAESEEAKKVINDFVEKGTQGKIAEAVKDPDEDTVFALANYILFKGKWKKPFDPKHTEEAEFHVDTVTTVKVPMMTLTGMLDVHHCSTLSSWVLLMDYLGNRTAVFLLPDDGKMQHLEQTLNKELISKFLLNRHRRLAQVHLPRLSLSGNYTLNTLMSHLGITRIFNNGADLSGITEENAPLKLSKAADKAVLTMDETGTEAAAATVLQAVPMSMPPILNFNKPFIFIIVEEHTQSPLFVGKVVDPTRK

SEQ ID NO: 98 - protein, 413 aa, Mus saxicola
MTPSISWRLLLLAGLCCLVPSYLAEDVQETDTSQKDQSPASHEMATNLGDFAFSLYRELVHQSNTSNIFFSPVSIATAFALLSLGSKGDTQTQILEGLQFNLTQTSEADIHKVFQHLLQTLNRPDSELQLSTGNGLFVNNDLKLVEKFLEEAKNHYQSEVFSVNFAKSEEARKMINDFVEKGTQGKIVDAVKDLDEDTVFALANYIFFQGKWKTPFDPEHTTEADFHVNESTTVRVPMMNLMRMLDVHYCSTLSSWVLMMDYLGNATAVFLLPDDGKMQHLEQTLNKELISKFLLNRHRSLAEIHFPRLSISGSYNLKALMAPLGITRVFNNGADLSGITEENAPLRLSKAVHKAVLTIDERGTEAAATTIVEAVFMSLPPILHFNHPFVFTIVETHTQTPLFVGKVVDPTRK

SEQ ID NO: 99 - protein, 411 aa, Rattus norvegicus
MAPSISRGLLLLAALCCLAPSFLAEDAQETDTSQQDQSPTYRKISSNLADFAFSLYRELVHQSNTSNIFFSPMSITTAFAMLSLGSKGDTRKQILEGLEFNLTQIPEADIHKAFHHLLQTLNRPDSELQLNTGNGLFVNKNLKLVEKFLEEVKNNYHSEAFSVNFADSEEAKKVINDYVEKGTQGKIVDLMKQLDEDTVFALVNYIFFKGKWKRPFNPEHTRDADFHVDKSTTVKVPMMNRLGMFDMHYCSTLSSWVLMMDYLGNATAIFLLPDDGKMQHLEQTLTKDLISRFLLNRQTRSAILYFPKLSISGTYNLKTLLSSLGITRVFNNDADLSGITEDAPLKLSQAVHKAVLTLDERGTEAAGATVVEAVPMSLPPQVKFDHPFIFMIVESETQSPLFVGKVIDPTR

SEQ ID NO: 100 - protein, 413 aa, Mesocricetus auratus
MKPSISWGILLLAGLCCLVPSFLAEDAQETDASKQDQEHQACCKIAPNLADFSFNLYRELVHQSNTTNIFFSPVSIATAFAMLSLGTKGVTHTQILEGLGFNLTEIAEAEVHKGFHNLLQTFNRPDNELQLTTGNGLFIHNNLKLVDKFLEEVKNDYHSEAFSVNFTDSEEAKKVINGFVEKGTQGKIVDLVKDLDKDTVLALVNYIFFKGKWKKPFDADNTEEADFHVDKTTTVKVPMMSRLGMFDVHYVSTLSSWVLLMDYLGNATAIFILPDDGKMQHLEQTLNKEIIGKFLKDRHTRSANVHFPKLSISGTYNLKTALDPLGITQVFSNGADLSGITEDVPLKLGKAVHKAVLTIDERGTEAAGATFMEIIPMSVPPEVNFNSPFIAIIYDRQTAKSPLFVGKVVDPTR

SEQ ID NO: 101 - protein, 396 aa, Cercopithecus aethiops
HVEDPQGDAAQKTDTSHHDQEHSTFNKITPSLAEFAFSLYRQLAHQSNSTNIFFSPVSIATAFAMLSLGTKADTHSEILEGLNFNLTEIPEAQIHEGFQELLHTLNKPDSQLQLTTGNGLFLNKSVKVVDKFLEDVKKLYHSEAFSVNFEDTEEAKKQINNYVEKGTQGKIVDLVKELDRDTVFALVNYIFFKGKWERPFEVEATKEEDFHVDQATTVKVPMMRRLGMFNIYHCEKLSSWVLLMKYLGNATAIFFLPDEGKLQHLENELTHDIITKFLENENRRSANLHLPKLAITGTYDLKTVLGHLGITKVFSNGADLSGVTEDAPLKLSKAVHKAVLTIDEKGTEAAGAMFLEAIPMSIPPEVKFNKPFVFLMIEQNTKSPLFMGKVVNPTQK

SEQ ID NO: 102 - protein, 409 aa, Papio anubis
LLLAGLCCLLPGSLAEDPQGDAAQKTDTPPHDQNHPTLNKITPSLAEFAFSLYRQLAHQSNSTNIFFSPVSIATAFAMLSLGTKADTHSEILEGLNFNLTEIPEAQVHEGFQELLRTLNKPDSQLQLTTGNGLFLNKSLKVVDKFLEDVKNLYHSEAFSVNFEDTEEAKKQINNYVEKGTQGKVVDLVKELDRDTVFALVNYIFFKGKWERPFEVEATEEEDFHVDQATTVKVPMMRRLGMFNIYHCEKLSSWVLLMKYLGNATAIFFLPDEGKLQHLENELTHDIITKFLENENRRSANLHLPKLAITGTYDLKTVLGHLGITKVFSNGADLSGVTEDAPLKLSKAVHKAVLTIDEKGTEAAGAMFLEAIPMSIPPEVKFNKPFVFLMIEQNTKSPLFIGKVVNPTQK

SEQ ID NO: 103 - protein, 418 aa, Pongo abelii
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHSEILEGLHFNLTEIPEAQVHEGFQELLRTLNQPDSQLQLTTGNGLFLNESLKLVDKFLEDVKKLYHSDAFTVNFGDTEEAKKQINDYVEKGTQGKIVDLVKELDRDTVFALVNYIFFKGKWERPFEVKDTKEEDFHVDEVTTVKVPMMRRLGMFNIHYCEKLSSWVLLMKYLGNATAIFFLPDEGKLQHLENELTHDIITKFLENENRRSASLHLPKLSITGTYDLKRVLGQLGITKVFSNGADLSGVTEEAPLKLSKAVHKAVLTIDEKGTEAAGAMFLEAIPMSIPPEVKFNKPFVFLMIEQNTKSPLFVGKVVNPTQK

SEQ ID NO: 104 - protein, 416 aa, Bos taurus
MALSITRGLLLLAALCCLAPISLAGVLQGHAVQETDDTSHQEAACHKIAPNLANFAFSIYHHLAHQSNTSNIFFSPVSIASAFAMLSLGAKGNTHTEILKGLGFNLTELAEAEIHKGFQHLLHTLNQPNHQLQLTTGNGLFINESAKLVDTFLEDVKNLYHSEAFSINFRDAEEAKKKINDYVEKGSHGKIVELVKVLDPNTVFALVNYISFKGKWEKPFEMKHTTERDFHVDEQTTVKVPMMNRLGMFDLHYCDKLASWVLLLDYVGNVTACFILPDLGKLQQLEDKLNNELLAKFLEKKYASSANLHLPKLSISETYDLKSVLGDVGITEVFSDRADLSGITKEQPLKVSKALHKAALTIDEKGTEAVGSTFLEAIPMSLPPDVEFNRPFLCILYDRNTKSPLFVGKVVNPTQA

SEQ ID NO: 105 - protein, 421 aa, Sus scrofa
MASSSTWGLLLLAGLCCLVPISLAEGLQGHAVQETDVPRHDHEQHQEAACHRIAPNLADFAFSLYRQVARQSNTSNIFLSPVTIARAFAMLSLGTKGATHAEILEGLQFNLTEKAEAEIHEGFQHLLHTLNQPDNQLQLTTGNGLFIDEKAKLVPKFLEDVKNLYHSEAFSINFRDTEEAKKCINDYVEKGSQGKIVDLVDELDKDTVFALVNYIFFKGKWEKPFEVEQTTEEDFHVDEETTVKVPMMNRLGMFDLHHCDKLSSWVLLMDYVATATAFFILPDQGKLHQLEDMLTKEIRAKFLEKRYPSSANLHLPKLTISGTYDLKSLLGNLGITKVFSDEADLSGVTEEQPLKLSKALHRAVLTIDEKGTEATGATILEAIPMSIPPNVKFNKPFLFLIYDTKTKAVLFMGKVMNPTQK

SEQ ID NO: 106 - protein, 413 aa, Oryctolagus cuniculus
MPPSVSRALLLLAGLGCLLPGFLADEAQETAVSSHEQDHPACHRIAPSLAEFALSLYREVAHESNTTNIFFSPVSIALAFAMLSLGAKGDTHTQVLEGLKFNLTETAEAQIHDGFRHLLHTVNRPDSELQLAARNALVVHENLKLQHKFLEDAKNLYQSEAFLVDFRDPEQAKTKINSHVEKGTRGKIVDLVQELDARTLLALVNYVFFKGKWEKPFEPENTKEEDFHVNATTTVRVPMMSRLGRYDLFHCSTLASTVLRMDYKGNATALFLLPDEGKLQHLEDTLTTELITKFLAKSSLRSVTVHFPKLSISGTYDLKPLLGKLGITQVFSDNADLSGITEQEPLKASQALHKAVLTIDERGTEAAGATYMEIIPMSLPDSITLDRPFLFVIYSHEIKSPLFVGKVVDPTQH

SEQ ID NO: 107 - protein, 410 aa, Didelphis virginiana
MMPSTLSLCLMLAGLCSLVTSHLTEEIQASNDTENEYSSTRRISPYMTDFSIDFYRLLVSKSNTTNIFFSPISIYTAFTLLALGAKSATRDQILTGLRFNRTEISEEHIFEGFQQLLNTFNLPENELQLTTSNGLFIDKNLKLVAKFLEDSKRLYASDTFSTNFEDNMAAKKQINDYVEKETQGKIVDLIQNLDSNVVFVLVNCIFFKGKWEKPFMTELTTECPFHVDSKTTVPVQTMRRLGMFNVFYDQDLSCWVLKMKYMGNATALFILPDTGKIEKVENALNKMLFHKWTRNLKRRAISLYFPKVSISGNYDLKILRELGITDVFGSNADLSGITEETNLKLSQAVHKAVVNIDEKGTEASGATFAEGIPMSIPPTVEFLRPFIFIILEENTKSVLFMGKVMNPTGN

SEQ ID NO: 108 - protein, 406 aa, Homo sapiens
MQLFLLLCLVLLSPQGASLHRHHPREMKKRVEDLHVGATVAPSSRRDFTFDLYRALASAAPSQSIFFSPVSISMSLAMLSLGAGSSTKMQILEGLGLNLQKSSEKELHRGFQQLLQELNQPRDGFQLSLGNALFTDLVVDLQDTFVSAMKTLYLADTFPTNFRDSAGAMKQINDYVAKQTKGKIVDLLKNLDSNAVVIMVNYIFFKAKWETSFNHKGTQEQDFYVTSETVVRVPMMSREDQYHYLLDRNLSCRVVGVPYQGNATALFILPSEGKMQQVENGLSEKTLRKWLKMFKKRQLELYLPKFSIEGSYQLEKVLPSLGISNVFTSHADLSGISNHSNIQVSEMVHKAVVEVDESGTRAAAATGTIFTFRSARLNSQRLVFNRPFLMFIVDNNILFLGKVNRP

SEQ ID NO: 109 - protein, 405 aa, Mus musculus
MRFFPILCLVLFISHGVASRRHSHSKKKKAKESSVGAVGPPSSKDFAFRLYRALVSESPGQNVFFSPLSVSMSLGMLSLGAGLKTKTQILDGLGLSLQQGQEDKLHKGFQQLLQRFRQPSDGLQLSLGSALFKDPAVHIRDDFLSAMKTLYMSDTFSTNFGNPEIAKKQINNYVAKQTKGKIVDFIKDLDSTHVMIVVNYIFFRAKWQTAFSETNTHKMDFHVTPKRTTQVPMMNREDGYSYYLDQNISCTVVGIPYQGNAIALFILPSEGKMKQVEDGLDERTLRNWLKMFTKRRLDLYLPKFSIEATYKLENVLPKLGIQDVFTTHADLSGITDHTNIKLSEMVHKSMMEVEESGTTAAAITGAIFTFRSARPSSLKIEFTRPFLLTLMEDSHILFVGKVTRP

SEQ ID NO: 110 - protein, 465 aa, Rattus norvegicus
MYSPGIGSAVAGERKLCLLSLLLIGALGCAVCHGNPVDDICIAKPRDIPVNPMCIYRSPAKKATEEDVLEQKVPEATNRRVWELSKANSRFATNFYQHLADSKNDNDNIFLSPLSISTAFAMTKLGACNNTLKQLMEVFKFDTISEKTSDQIHFFFAKLNCRLYRKANKSSNLVSANRLFGDKSLTFNESYQDVSEIVYGAKLQPLDFKENPEQSRVTINNWVANKTEGRIKDVIPQGAIDELTALVLVNTIYFKGLWKSKFSPENTRKEPFHKVDGQSCLVPMMYQEGKFKYRRVGEGTQVLEMPFKGDDITMVLILPKPEKSLAKVEQELTPELLQEWLDELSEVMLVVHVPRFRIEDSFSLKEQLQDMGLVDLFSPEKSQLPGIIAEGRDDLFVSDAFHKAFLEVNEEGSEAAASTSVVITGRSLNPSRVTFKANRPFLVLIREVALNTIIFMGRVSNPCVN

SEQ ID NO: 111 - protein, 465 aa, Mus musculus
MYSPGAGSGAAGERKLCLLSLLLIGALGCAICHGNPVDDICIAKPRDIPVNPLCIYRSPGKKATEEDGSEQKVPEATNRRVWELSKANSRFATNFYQHLADSKNDNDNIFLSPLSISTAFAMTKLGACNDTLKQLMEVFKFDTISEKTSDQIHFFFAKLNCRLYRKANKSSDLVSANRLFGDKSLTFNESYQDVSEVVYGAKLQPLDFKENPEQSRVTINNWVANKTEGRIKDVIPQGAINELTALVLVNTIYFKGLWKSKFSPENTRKEPFYKVDGQSCPVPMMYQEGKFKYRRVAEGTQVLELPFKGDDITMVLILPKPEKSLAKVEQELTPELLQEWLDELSETMLVVHMPRFRTEDGFSLKEQLQDMGLIDLFSPEKSQLPGIVAGGRDDLYVSDAFHKAFLEVNEEGSEAAASTSVVITGRSLNPNRVTFKANRPFLVLIREVALNTIIFMGRVANPCVN

SEQ ID NO: 112 - protein, 464 aa, Pongo abelii
MYSNVIGTITSGKRKVYLLSLLLIGFWDCVTCHGSPVDICIAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPPEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 113 - protein, 465 aa, Ovis aries
MISNGIGTVTTGKRSMCLFPLLLIGLWGCVTCHRSPVEDICTAKPRDIPVNPMCIYRSPEKKATEGEGSEQKIPGATNRRVWELSKANSHFATAFYQHLADSKNNNDNIFLSPLSISTAFAMTKLGACNNTLKQLMEVFKFDTISEKTSDQIHFFFAKLNCRLYRKANKSSELVSANRLFGDKSITFNETYQDISEVVYGAKLQPLDFKGNAEQSRLTINQWISNKTEGRITDVIPPQAIDEFTVLVLVNTIYFKGLWKSKFSPENTKKELFYKADGESCSVPMMYQEGKFRYRRVAEGTQVLELPFKGDDITMVLILPKLEKPLAKVERELTPDMLQEWLDELTETLLVVHMPHFRIEDSFSVKEQLQDMGLEDLFSPEKSRLPGIVAEGRNDLYVSDAFHKAFLEVNEEGSEAAASTVISIAGRSLNLNRVTFQANRPFLVLIREVALNTIIFMGRVANPCVN

SEQ ID NO: 114 - protein, 222 aa, Triatoma infestans
LEENDCACPRVLHRVCGSDGNTYSNPCTLDCAKHEGKPDLVQVHEGPCDPNDHDFEDPCECDNKFEPVCGTDHITYSNLCHLECAAFTTSPGVEVKYEGECHAEIMEQHQILKSCICTKMYKPVCGTDGHTYPNLCVLKCRISSKPGLKLAHVGKCGIGLLAVETKEVRNPCACFRNYVPVCGSDGKTYGNPCMLNCAAQTKVPGLKLVHEGRCQRSNVEQF

SEQ ID NO: 115 - protein, 471 aa, Triatoma brasiliensis
  misc_feature (223)..(223): Xaa can be any naturally occurring amino acid
MRYLLLLGLAAFSAVSAEEKDPPCACPLIWKPVCGNDGQTYPNECLLNCIKYVLKKDIEVAYQGICKHFVHAAAEAEELVESKPPCACPLIWKPVCGNDGQTYPNECMLNCMKYILKKDIEVAYQGMCKHFVHAAAEAEELVESKNPCECPRALHRVCGSDGNTYSNPCTLNCAKHEGKPDLVQVHEGPCSPDEHDFEDPCECDNKFDPVCGTDKVTYRNLCXLECAMFTTSPGVEVDYEGECLAETVLLEENHCACPRVLHRVCGSDGNTYSNPCTLDCAKHEGKPDLVQVHEGPCDPNDHDFEDPCECDNKFEPVCGTDHITYSNLCHLECAAFTTSPGVEVKYEGECHAEIMEQHQILKSCICTKIYSPVCGTDGHTYPNLCILECHISFNPGLKLAHVGKCGTDLQDIETKQVRNPCACFRNYLPVCGSDGKTYGNPCMLNCAAHTKVPGLKLAHKGRCQRSDVEQF

SEQ ID NO: 116 - protein, 351 aa, Dipetalogaster maximus
LIKELVNMVIQHAEEEEVKELKNPCECPRALHRVCGSDGNTYSNPCMLNCAKHEGNPDLVQVHKGPCDEHDHDFEDPCKCDNKFEPVCGDDQITYLNLCHLECATFTTSPGVEVAYEGECHAETTNAMEVLFQGNPCECPRALHRVCGSDGNTYSNPCMLTCAKHEGNPDLVQVHEGPCDEHDHDFEDTCQCDDTFQPVCGDDEITYRNLCHLECATFTTSPGVEVKHEGECHPETKVNQLILKSCMCPKIYKPVCGTDGRTYPNICVLKCHISSNPGLGLAHLGECKVAVLAKETGEVRNPCNCFRNFNPVCGTDGKTYGNLCMLGCAAETKVPGLKLLHNGRCLPKEQL

SEQ ID NO: 117 - protein, 368 aa, Rhodnius prolixus
RLLLLLGLAALVAAEGGEPCACPHALHRVCGSDGETYSNPCTLNCAKFNGKPELVKVHDGPCEPDEDEDVCQECDGDEYKPVCGSDDITYDNNCRLECASISSSPGVELKHEGPCRTEEKKILKRSDEFEMYRCACPKIYYPVCGTDGETYPNLCVLECHMRMNPGLQLHHYGHCQHHHHHHPPPHHHHHHHPHHTTEKPVEPCACPHALHRVCGSDGETYSNPCTLNCAKHNGKPGLVKVHDGPCEPDEDEDVCQECDDVDYEPVCGTDDKTYDNNCRLECASISSSPGVELKHEGICRKEEKKLPKRSVGLEHTCVCPYNYFPVCGTDGETYPNLCALQCRMREVPGLELKHTGKCLPHLDFPDPV

SEQ ID NO: 118 - protein, 186 aa, Rhodnius prolixus
MKRLLLLLGLAALVAAEGGEPCACPHALHRVCGSDGETYSNPCTLNCAKFNGKPELVKVHDGPCEPDEDEDVCQECDGDEYKPVCGSDGITYDNNCRLECASISSSPGVELKHEGICRKEEKKLPKRSVGLEHTCVCPYNYFPVCGTDGETYPNLCALQCRMREVPGLELKHTGKCLPHLDFPDPV

SEQ ID NO: 119 - protein, 141 aa, Haemaphysalis longicornis
MKLFVFLALFGAAFAQRNGFCRLPAEPGICRAFMPRYYFDVEKGQCEQFIYGGCKGNENNFETLKECQDACGEPERASDFEKADFETGCKAAPETGLCKASFERWFFNAASGECEEFIYGGCGGNDNNYENKEECEFACKY

SEQ ID NO: 120 - protein, 142 aa, Boophilus microplus
MKCIILLAVLGTAFAQRNGFCRLPADEGICKALIPRFYFNTETGKCTMFSYGGCGGNENNFETIEECQKACGAPERVNDFESADFKTGCEPAADSGSCAGQLERWFYNVQSGECETFVYGGCGGNDNNYESEEECELVCKNM

SEQ ID NO: 121 - protein, 142 aa, Boophilus microplus
MKYLILLAVLGTAFAQRNGFCRLPADEGICKALIPRFYFNTETGKCTMFSYGGCGGNENNFETIEDCQKACGAPERVSDFEGADFKTGCEPAADSGSCAGQLERWFYNVRSGECETFVYGGCGGNDNNYESEEECELVCKNM

SEQ ID NO: 122 - protein, 119 aa, Ornithodoros moubata
LNVLCNNPHTADCNNDAQVDRYFREGTTCLMSPACTSEGYASQHECQQACFVGGEDHSSEMHSSCLGDPPTSCAEGTDITYYDSDSKTCKVLAASCPSGENTFESEVECQVACGAPIEG

SEQ ID NO: 123 - protein, 91 aa, Ornithodoros moubata
LNVLSNNPHTADCNNDAQVDRYFREGTTCLMSPACTSEGYASQHECLRPALLAGKTTAVKCTAHALVTRPLPARKARTSPTTILIAKHVRY

SEQ ID NO: 124 - protein, 134 aa, Ornithodoros savignyi
MLFYVVITLVAGTVSGLNVRCNNPHTANCENGAKLESYFREGETCVGSPACPGEGYATKEDCQKACFPGGGDHSTNVDSSCFGQPPTSCETGAEVTYYDSGSRTCKVLQHGCPSSENAFDSEIECQVACGVSME

SEQ ID NO: 125 - protein, 166 aa, Amblyomma hebraeum
MGFLVASAVLVCVTSQRVPGYCKKKPAVGPCKALIEKWYFDYSTQSCKTFYYGGCGGNGNKFSSRKKCREACLPKRPSVPVCKQMPDPGFCRAYMPHWFFNSKSGYCEGFVYGGCQGNDNRFKSCWQCMKKCRTAREANRLCWKLTKEFNKKFLRNVPTAKPLPPK

SEQ ID NO: 126 - protein, 183 aa, Amblyomma americanum
MRPQAFIGAFVFTLVLRQAAGIKWSRCFRPKAVGNCQNKVPAWYYDFWSFRCKGFLYSGCGGNSNRFPTEEECQKSCLRKSKRKEVCSLKPKTGKCKAAIPLWYYDPELDECRGLIYGGCKGNANRFETCLKCMKRCSGNNNARKICKKQTKKFLEENNLGSNRHHKKPSWPQLSIRIPFIEK

SEQ ID NO: 127 - protein, 174 aa, Ixodes scapularis; identical to SEQ ID NO: 58

SEQ ID NO: 128 - protein, 178 aa, Ixodes scapularis; identical to SEQ ID NO: 59

SEQ ID NO: 129 - protein, 374 aa, Ixodes scapularis; identical to SEQ ID NO: 60

SEQ ID NO: 130 - protein, 53 aa, Glossina morsitans morsitans
MKFFTVLFFLLSIIYLIVAAPGEPGAPIDYDEYGDSSEEVGGTPLHEIPGIRL

SEQ ID NO: 131 - protein, 80 aa, Haemaphysalis longicornis
MKHFVILILAVVASAVVMAYPERDSAKDGNQEKERALLVKVQERYQGNQGDYDEYDQDETTPPPDPTAQTARPRLRQNQD

SEQ ID NO: 132 - protein, 79 aa, Haemaphysalis longicornis
MKHFAILILAVVASAVVMAYPERDSAKEGNQEQERALHVKVQKRTDGDADYDEYEEDGTTPTPDPTAPTAKPRLRGNKP

SEQ ID NO: 133 - protein, 105 aa, Gallus gallus
MHLFIGVSLRPLGHGIPAPYAVEDICTAKPRDIPVNPICIYRNPEKKPQERRGAGAGEGQDPGVHKPPASGSCPGPTRAFGRRSFLQAPGPTPRTMRRTSSCRPS

SEQ ID NO: 134 - protein, 166 aa, Amblyomma hebraeum; identical to SEQ ID NO: 125

SEQ ID NO: 135 - protein, 183 aa, Amblyomma hebraeum; identical to SEQ ID NO: 126

SEQ ID NO: 136 - protein, 20 aa, Glossina morsitans
GEPGAPIDYDEYGDSSEEIG

SEQ ID NO: 137 - protein, 126 aa, Amblyomma americanum
MEIKRCVHVLLTLATVYGTSVSISSICSLPQVKGNCRGIFEMWHYNSTKDTCSLFTYGGCGGNENRFENCTQCMESCSTNENRTEICQLLEKEADEDYYSGWDDNTGGDGYTAPPREAYDEDYDEE

SEQ ID NO: 138 - protein, 67 aa, Theromyzon tessulatum
ECENTECPRACPGEYEFDEDGCNTCVCKGCDDAQCRCSSDANGCESFCTCNTRCSAADECNPRCTCK

SEQ ID NO: 139 - protein, 144 aa, Argas monolakensis
MRLLALFAFAVAVVSAQRNQMCQQPRTQGSCDASNQITKFFYTGSGCTSAPVCSDTDGGYGTEDECIQACTVQGGHHNEGAGEEGCSGDPPRGDCGGQVEERYYFDSTTRTCQTFEYRGCSSGNPDNSYETEIECEIACPSASS

SEQ ID NO: 140 - protein, 135 aa, Hydra magnipapillata
MCLRCRPCEPDLCPPRPLCNGGYVKGICGCCDTCAKVDGEECGGLWNMYGKCDVGFVCAQVKRHTKGVCVSVKKKSNNKENTCALKPETGPCRAAIKAWYYDYKTDTCKRFVYGGCGGNSNRFREKKMCQKTCYA

SEQ ID NO: 141 - protein, 15 aa, Bothrops jararaca
  misc_feature (14)..(14): Xaa can be any naturally occurring amino acid
EKFPAVNQKPQAAXL

SEQ ID NO: 142 - protein, 141 aa, Bos taurus
  misc_feature (114)..(114): Xaa can be any naturally occurring amino acid
MDALSEANGTFALTLLKKLGEGSSKNVFISPLSISSALAMVLMGARGNTAAQMCQTLSLNKSSGGGEDVHQGFQNLLSEVNRTDPRLLAQNRQRGFSGDKTYDFLSSFKDSCRXILPSRDGRAGLYQRYGAVQEAHKHLGS

SEQ ID NO: 143 - protein, 56 aa, Helianthus annuus
MATTMAKLITLVVLAILAFVEVSVSGYKTSISTITIEDNGRCTKSIPPICFPDGRP

SEQ ID NO: 144 - protein, 187 aa, Canis familiaris
MPVDLGKWSGPLSLQEVEERPQHALHVKYTGTEVDELGKVLTPTQVKNRPTSIAWDGLDPGKLYTLVLTDPDAPSRKDPKYREWHHFLVVNMKGNDISSGTVLSDYVGSGPPKGTGLHRYVWLVYEQSGPLKCDEPILSNRSGDHRGKFKVASFRKKYELGPPVAGTCYQAEWDDYVPKLCEQLSGK

SEQ ID NO: 145 - protein, 16 aa, Bothrops jararaca
  misc_feature (15)..(15): Xaa can be any naturally occurring amino acid
DCPSDWSPYGQGCYXF

SEQ ID NO: 146 - protein, 179 aa, Haematobia irritans
NINIMKHFVVIGILALSAVCQAQNVLSGRRQHGAQGLSGYSGDNDWGYYGEAGAPGSDYSGSSGQWAPLDFDYNSLPGLSGYNHEQQDYEEDSYRHVRSAGPITLQLDDDDDDDSGIPIFEMDDEDEDSNDNQKFPLSFERFPENEKNQVGLRARFNKFMAKFTSLFGRRRGVNVPNAA

SEQ ID NO: 147 - protein, 39 aa, Haemadipsa sylvestris
CDCGEKICLYGQSCNDGQCSGDPKPSSEFEEFEIDEEEK

SEQ ID NO: 148 - protein, 103 aa, Anopheles gambiae
MASKLFVLAFLCLALVVVVQSAPQYARGDVPTYDEEDFDEESLKPHSSSSSDDGEEEFDPSLLEEHADAPTARDPGRNPEFLRNSNTDEQASAPAASSSESDE

SEQ ID NO: 149 - protein, 102 aa, Anopheles funestus
MATKLIVIAFLCAALIAVVQSAPQYAQGEEPTYDEDDDEPVKPHSSADPDASYEEFDPSQLTEYANTAQDPGRRPHFLEQANSNNGDQLPSQSDSSSESTEH

SEQ ID NO: 150 - protein, 101 aa, Anopheles stephensi
MASKVIVIALLCIALAAFVQGAPQYTHGEEPEYDEDDGADEPVQPHSSSNHADTEDDFDLSLLDKPYANAPENADPGRRPEFLKQHNNENQSDSSSGSTEN

SEQ ID NO: 151 - protein, 81 aa, Anopheles darlingi
MANKLFLISLLCVALVAKIAQAAPQYAPGEEPSYDEDTDDKLIENDTSITDEDYAEIEASLSQAFGTAADPGRRLGEGKKP

SEQ ID NO: 152 - protein, 83 aa, Anopheles albimanus
MANKLVLISLLCVVLVAKITQAAPQYAPGDEPSYDEDTDDSDKLVENDTSITDEDYAAIEASLSETFNTAADPGRRLGEGSKP

SEQ ID NO: 153 - protein, 64 aa, Hirudo medicinalis
  SULFATION (62)..(62)
VYTDCTESGQNLCLCEGSNVCGQGNKCILGSDGEKNQCVTGEGTPKPQSHNDGDFEEIPEEYLQ

SEQ ID NO: 154 - protein, 9 aa, Artificial Sequence
  synthetic peptide
FEEIPEEYL

SEQ ID NO: 155 - protein, 8 aa, Artificial Sequence
  synthetic peptide
YEPIPEEA

SEQ ID NO: 156 - protein, 12 aa, Artificial Sequence
  synthetic peptide
NGDFEEIPEEYL

SEQ ID NO: 157 - protein, 14 aa, Artificial Sequence
  synthetic peptide
APPFDFEAIPEEYL

SEQ ID NO: 158 - protein, 21 aa, Artificial Sequence
  modified hirulog
FPRMHKGGGNGDFEEIPEEYL

SEQ ID NO: 159 - protein, 20 aa, Artificial Sequence
  modified hirulog
FPRMHKGGNGDFEEIPEEYL

SEQ ID NO: 160 - protein, 22 aa, Artificial Sequence
  modified hirulog
FPRMHKGGGGNGDFEEIPEEYL

SEQ ID NO: 161 - protein, 21 aa, Artificial Sequence
  modified hirulog
FPRMHKTGGNGDFEEIPEEYL

SEQ ID NO: 162 - protein, 20 aa, Artificial Sequence
  modified hirulog
FPRMHKTGNGDFEEIPEEYL

SEQ ID NO: 163 - protein, 22 aa, Artificial Sequence
  modified hirulog
FPRMHKTGGGNGDFEEIPEEYL

SEQ ID NO: 164 - protein, 21 aa, Artificial Sequence
  modified hirulog
FPRMHKTAGNGDFEEIPEEYL

SEQ ID NO: 165 - protein, 20 aa, Artificial Sequence
  modified hirulog
FPRMHKTANGDFEEIPEEYL

SEQ ID NO: 166 - protein, 22 aa, Artificial Sequence
  modified hirulog
FPRMHKTAGGNGDFEEIPEEYL

SEQ ID NO: 167 - protein, 21 aa, Artificial Sequence
  modified hirulog
  MISC_FEATURE (1)..(1): F=D-phenylalanine
FPRMHKGGGNGDFEEIPEEYL

SEQ ID NO: 168 - protein, 20 aa, Artificial Sequence
  modified hirulog
  MISC_FEATURE (1)..(1): F=D-phenylalanine
FPRMHKGGNGDFEEIPEEYL

SEQ ID NO: 169 - protein, 22 aa, Artificial Sequence
  modified hirulog
  MISC_FEATURE (1)..(1): F=D-phenylalanine
FPRMHKGGGGNGDFEEIPEEYL

SEQ ID NO: 170 - protein, 21 aa, Artificial Sequence
  modified hirulog
  MISC_FEATURE (1)..(1): F=D-phenylalanine
FPRMHKTGGNGDFEEIPEEYL

SEQ ID NO: 171 - protein, 20 aa, Artificial Sequence
  modified hirulog
  MISC_FEATURE (1)..(1): F=D-phenylalanine
FPRMHKTGNGDFEEIPEEYL

SEQ ID NO: 172 - protein, 22 aa, Artificial Sequence
  modified hirulog
  MISC_FEATURE (1)..(1): F=D-phenylalanine
FPRMHKTGGGNGDFEEIPEEYL

SEQ ID NO: 173 - protein, 21 aa, Artificial Sequence
  modified hirulog
  MISC_FEATURE (1)..(1): F=D-phenylalanine
FPRMHKTAGNGDFEEIPEEYL

SEQ ID NO: 174 - protein, 20 aa, Artificial Sequence
  modified hirulog
  MISC_FEATURE (1)..(1): F=D-phenylalanine
FPRMHKTANGDFEEIPEEYL

SEQ ID NO: 175 - protein, 22 aa, Artificial Sequence
  modified hirulog
  MISC_FEATURE (1)..(1): F=D-phenylalanine
FPRMHKTAGGNGDFEEIPEEYL

SEQ ID NO: 176 - protein, 21 aa, Artificial Sequence
  modified hirulog
  misc_feature (4)..(4): Xaa can be any naturally occurring amino acid
FPRXHKGGGNGDFEEIPEEYL

SEQ ID NO: 177 - protein, 20 aa, Artificial Sequence
  modified hirulog
  misc_feature (4)..(4): Xaa can be any naturally occurring amino acid
FPRXHKGGNGDFEEIPEEYL

SEQ ID NO: 178 - protein, 22 aa, Artificial Sequence
  modified hirulog
  misc_feature (4)..(4): Xaa can be any naturally occurring amino acid
FPRXHKGGGGNGDFEEIPEEYL

SEQ ID NO: 179 - protein, 21 aa, Artificial Sequence
  modified hirulog
  misc_feature (4)..(4): Xaa can be any naturally occurring amino acid
FPRXHKTGGNGDFEEIPEEYL

SEQ ID NO: 180 - protein, 20 aa, Artificial Sequence
  modified hirulog
  misc_feature (4)..(4): Xaa can be any naturally occurring amino acid
FPRXHKTGNGDFEEIPEEYL

SEQ ID NO: 181 - protein, 22 aa, Artificial Sequence
  modified hirulog
  misc_feature (4)..(4): Xaa can be any naturally occurring amino acid
FPRXHKTGGGNGDFEEIPEEYL

SEQ ID NO: 182 - protein, 21 aa, Artificial Sequence
  modified hirulog
  misc_feature (4)..(4): Xaa can be any naturally occurring amino acid
FPRXHKTAGNGDFEEIPEEYL

SEQ ID NO: 183 - protein, 20 aa, Artificial Sequence
  modified hirulog
  misc_feature (4)..(4): Xaa can be any naturally occurring amino acid
FPRXHKTANGDFEEIPEEYL

SEQ ID NO: 184 - protein, 22 aa, Artificial Sequence
  modified hirulog
  misc_feature (4)..(4): Xaa can be any naturally occurring amino acid
FPRXHKTAGGNGDFEEIPEEYL

SEQ ID NO: 185 - protein, 21 aa, Artificial Sequence
  modified hirulog
  MISC_FEATURE (1)..(1): F=D-phenylalanine
  misc_feature (4)..(4): Xaa can be any naturally occurring amino acid
FPRXHKGGGNGDFEEIPEEYL

SEQ ID NO: 186 - protein, 20 aa, Artificial Sequence
  modified hirulog
  MISC_FEATURE (1)..(1): F=D-phenylalanine
  misc_feature (4)..(4): Xaa can be any naturally occurring amino acid
FPRXHKGGNGDFEEIPEEYL

SEQ ID NO: 187 - protein, 22 aa, Artificial Sequence
  modified hirulog
  MISC_FEATURE (1)..(1): F=D-phenylalanine
  misc_feature (4)..(4): Xaa can be any naturally occurring amino acid
FPRXHKGGGGNGDFEEIPEEYL

SEQ ID NO: 188 - protein, 21 aa, Artificial Sequence
  modified hirulog
  MISC_FEATURE (1)..(1): F=D-phenylalanine
  misc_feature (4)..(4): Xaa can be any naturally occurring amino acid
FPRXHKTGGNGDFEEIPEEYL

SEQ ID NO: 189 - protein, 20 aa, Artificial Sequence
  modified hirulog
  MISC_FEATURE (1)..(1): F=D-phenylalanine
  misc_feature (4)..(4): Xaa can be any naturally occurring amino acid
FPRXHKTGNGDFEEIPEEYL

SEQ ID NO: 190 - protein, 22 aa, Artificial Sequence
  modified hirulog
  MISC_FEATURE (1)..(1): F=D-phenylalanine
  misc_feature (4)..(4): Xaa can be any naturally occurring amino acid
FPRXHKTGGGNGDFEEIPEEYL

SEQ ID NO: 191 - protein, 21 aa, Artificial Sequence
  modified hirulog
  MISC_FEATURE (1)..(1): F=D-phenylalanine
  misc_feature (4)..(4): Xaa can be any naturally occurring amino acid
FPRXHKTAGNGDFEEIPEEYL

SEQ ID NO: 192 - protein, 20 aa, Artificial Sequence
  modified hirulog
  MISC_FEATURE (1)..(1): F=D-phenylalanine
  misc_feature (4)..(4): Xaa can be any naturally occurring amino acid
FPRXHKTANGDFEEIPEEYL

SEQ ID NO: 193 - protein, 22 aa, Artificial Sequence
  modified hirulog
  MISC_FEATURE (1)..(1): F=D-phenylalanine
  misc_feature (4)..(4): Xaa can be any naturally occurring amino acid
FPRXHKTAGGNGDFEEIPEEYL

SEQ ID NO: 194 - protein, 21 aa, Artificial Sequence
  modified hirulog
  misc_feature (6)..(6): Xaa can be any naturally occurring amino acid
FPRMHXGGGNGDFEEIPEEYL

SEQ ID NO: 195 - protein, 20 aa, Artificial Sequence
  modified hirulog
  misc_feature (6)..(6): Xaa can be any naturally occurring amino acid
FPRMHXGGNGDFEEIPEEYL

SEQ ID NO: 196 - protein, 22 aa, Artificial Sequence
  modified hirulog
  misc_feature (6)..(6): Xaa can be any naturally occurring amino acid
FPRMHXGGGGNGDFEEIPEEYL

SEQ ID NO: 197 - protein, 21 aa, Artificial Sequence
  modified hirulog
  misc_feature (6)..(6): Xaa can be any naturally occurring amino acid
FPRMHXTGGNGDFEEIPEEYL

SEQ ID NO: 198 - protein, 20 aa, Artificial Sequence
  modified hirulog
  misc_feature (6)..(6): Xaa can be any naturally occurring amino acid
FPRMHXTGNGDFEEIPEEYL

SEQ ID NO: 199 - protein, 22 aa, Artificial Sequence
  modified hirulog
  misc_feature (6)..(6): Xaa can be any naturally occurring amino acid
FPRMHXTGGGNGDFEEIPEEYL

SEQ ID NO: 200 - protein, 21 aa, Artificial Sequence
  modified hirulog
  misc_feature (6)..(6): Xaa can be any naturally occurring amino acid
FPRMHXTAGNGDFEEIPEEYL

SEQ ID NO: 201 - protein, 20 aa, Artificial Sequence
  modified hirulog
  misc_feature (6)..(6): Xaa can be any naturally occurring amino acid
FPRMHXTANGDFEEIPEEYL

SEQ ID NO: 202 - protein, 22 aa, Artificial Sequence
  modified hirulog
  misc_feature (6)..(6): Xaa can be any naturally occurring amino acid
FPRMHXTAGGNGDFEEIPEEYL

SEQ ID NO: 203 - protein, 21 aa, Artificial Sequence
  modified hirulog
  MISC_FEATURE (1)..(1): F=D-phenylalanine
  misc_feature (6)..(6): Xaa can be any naturally occurring amino acid
FPRMHXGGGNGDFEEIPEEYL

SEQ ID NO: 204 - protein, 20 aa, Artificial Sequence
  modified hirulog
  MISC_FEATURE (1)..(1): F=D-phenylalanine
  misc_feature (6)..(6): Xaa can be any naturally occurring amino acid
FPRMHXGGNGDFEEIPEEYL

SEQ ID NO: 205 - protein, 22 aa, Artificial Sequence
  modified hirulog
  MISC_FEATURE (1)..(1): F=D-phenylalanine
  misc_feature (6)..(6): Xaa can be any naturally occurring amino acid
FPRMHXGGGGNGDFEEIPEEYL

SEQ ID NO: 206 - protein, 21 aa, Artificial Sequence
  modified hirulog
  MISC_FEATURE (1)..(1)
  misc_feature (6)..(6): Xaa can be any naturally occurring amino acid
FPRMHXTGGNGDFEEIPEEYL

SEQ ID NO: 207 - protein, 20 aa, Artificial Sequence
  modified hirulog
  MISC_FEATURE (1)..(1): F=D-phenylalanine
  misc_feature (6)..(6): Xaa can be any naturally occurring amino acid
FPRMHXTGNGDFEEIPEEYL

SEQ ID NO: 208 - protein, 22 aa, Artificial Sequence
  modified hirulog
  MISC_FEATURE (1)..(1): F=D-phenylalanine
  misc_feature (6)..(6): Xaa can be any naturally occurring amino acid
FPRMHXTGGGNGDFEEIPEEYL

SEQ ID NO: 209 - protein, 21 aa, Artificial Sequence
  modified hirulog
  MISC_FEATURE (1)..(1): F=D-phenylalanine
  misc_feature (6)..(6): Xaa can be any naturally occurring amino acid
FPRMHXTAGNGDFEEIPEEYL

SEQ ID NO: 210 - protein, 20 aa, Artificial Sequence
  modified hirulog
  MISC_FEATURE (1)..(1): F=D-phenylalanine
  misc_feature (6)..(6): Xaa can be any naturally occurring amino acid
FPRMHXTANGDFEEIPEEYL

SEQ ID NO: 211 - protein, 22 aa, Artificial Sequence
  modified hirulog
  MISC_FEATURE (1)..(1): F=D-phenylalanine
  misc_feature (6)..(6): Xaa can be any naturally occurring amino acid
FPRMHXTAGGNGDFEEIPEEYL

SEQ ID NO: 212 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGNGDFEEIPEEYL

SEQ ID NO: 213 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGNGDFEEIPEEYL

SEQ ID NO: 214 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGGNGDFEEIPEEYL

SEQ ID NO: 215 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGNGDFEEIPEEYL

SEQ ID NO: 216 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGNGDFEEIPEEYL

SEQ ID NO: 217 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGGNGDFEEIPEEYL

SEQ ID NO: 218 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGNGDFEEIPEEYL

SEQ ID NO: 219 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTANGDFEEIPEEYL

SEQ ID NO: 220 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGGNGDFEEIPEEYL

SEQ ID NO: 221 - protein, 21 aa, Artificial Sequence
  modified hirulog
FPRMHRGGGNGDFEEIPEEYL

SEQ ID NO: 222 - protein, 20 aa, Artificial Sequence
  modified hirulog
FPRMHRGGNGDFEEIPEEYL

SEQ ID NO: 223 - protein, 22 aa, Artificial Sequence
  modified hirulog
FPRMHRGGGGNGDFEEIPEEYL

SEQ ID NO: 224 - protein, 21 aa, Artificial Sequence
  modified hirulog
FPRMHRTGGNGDFEEIPEEYL

SEQ ID NO: 225 - protein, 20 aa, Artificial Sequence
  modified hirulog
FPRMHRTGNGDFEEIPEEYL

SEQ ID NO: 226 - protein, 22 aa, Artificial Sequence
  modified hirulog
FPRMHRTGGGNGDFEEIPEEYL

SEQ ID NO: 227 - protein, 21 aa, Artificial Sequence
  modified hirulog
FPRMHRTAGNGDFEEIPEEYL

SEQ ID NO: 228 - protein, 20 aa, Artificial Sequence
  modified hirulog
FPRMHRTANGDFEEIPEEYL

SEQ ID NO: 229 - protein, 22 aa, Artificial Sequence
  modified hirulog
FPRMHRTAGGNGDFEEIPEEYL

SEQ ID NO: 230 - protein, 21 aa, Artificial Sequence
  modified hirulog
  MISC_FEATURE (1)..(1): F=D-phenylalanine
FPRMHRGGGNGDFEEIPEEYL

SEQ ID NO: 231 - protein, 20 aa, Artificial Sequence
  modified hirulog
  MISC_FEATURE (1)..(1): F=D-phenylalanine
FPRMHRGGNGDFEEIPEEYL

SEQ ID NO: 232 - protein, 22 aa, Artificial Sequence
  modified hirulog
  MISC_FEATURE (1)..(1): F=D-phenylalanine
FPRMHRGGGGNGDFEEIPEEYL

SEQ ID NO: 233 - protein, 21 aa, Artificial Sequence
  modified hirulog
  MISC_FEATURE (1)..(1): F=D-phenylalanine
FPRMHRTGGNGDFEEIPEEYL

SEQ ID NO: 234 - protein, 20 aa, Artificial Sequence
  modified hirulog
  MISC_FEATURE (1)..(1): F=D-phenylalanine
FPRMHRTGNGDFEEIPEEYL

SEQ ID NO: 235 - protein, 22 aa, Artificial Sequence
  modified hirulog
  MISC_FEATURE (1)..(1): F=D-phenylalanine
FPRMHRTGGGNGDFEEIPEEYL

SEQ ID NO: 236 - protein, 21 aa, Artificial Sequence
  modified hirulog
  MISC_FEATURE (1)..(1): F=D-phenylalanine
FPRMHRTAGNGDFEEIPEEYL

SEQ ID NO: 237 - protein, 20 aa, Artificial Sequence
  modified hirulog
  MISC_FEATURE (1)..(1): F=D-phenylalanine
FPRMHRTANGDFEEIPEEYL

SEQ ID NO: 238 - protein, 23 aa, Artificial Sequence
  modified hirulog
FDPRMHRTAGGNGDFEEIPEEYL

SEQ ID NO: 239 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGNGDFEEIPEEYL

SEQ ID NO: 240 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGNGDFEEIPEEYL

SEQ ID NO: 241 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGGNGDFEEIPEEYL

SEQ ID NO: 242 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGNGDFEEIPEEYL

SEQ ID NO: 243 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGNGDFEEIPEEYL

SEQ ID NO: 244 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGGNGDFEEIPEEYL

SEQ ID NO: 245 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGNGDFEEIPEEYL

SEQ ID NO: 246 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTANGDFEEIPEEYL

SEQ ID NO: 247 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGGNGDFEEIPEEYL

SEQ ID NO: 248 - protein, 21 aa, Artificial Sequence
  modified hirulog
FPKMHKGGGNGDFEEIPEEYL

SEQ ID NO: 249 - protein, 20 aa, Artificial Sequence
  modified hirulog
FPKMHKGGNGDFEEIPEEYL

SEQ ID NO: 250 - protein, 22 aa, Artificial Sequence
  modified hirulog
FPKMHKGGGGNGDFEEIPEEYL

SEQ ID NO: 251 - skipped

SEQ ID NO: 252 - protein, 21 aa, Artificial Sequence
  modified hirulog
FPKMHKTGGNGDFEEIPEEYL

SEQ ID NO: 253 - protein, 20 aa, Artificial Sequence
  modified hirulog
FPKMHKTGNGDFEEIPEEYL

SEQ ID NO: 254 - protein, 22 aa, Artificial Sequence
  modified hirulog
FPKMHKTGGGNGDFEEIPEEYL

SEQ ID NO: 255 - protein, 21 aa, Artificial Sequence
  modified hirulog
FPKMHKTAGNGDFEEIPEEYL

SEQ ID NO: 256 - protein, 20 aa, Artificial Sequence
  modified hirulog
FPKMHKTANGDFEEIPEEYL

SEQ ID NO: 257 - protein, 22 aa, Artificial Sequence
  modified hirulog
FPKMHKTAGGNGDFEEIPEEYL

SEQ ID NO: 258 - protein, 21 aa, Artificial Sequence
  modified hirulog
  MISC_FEATURE (1)..(1): F=D-phenylalanine
FPKMHKGGGNGDFEEIPEEYL

SEQ ID NO: 259 - protein, 20 aa, Artificial Sequence
  modified hirulog
  MISC_FEATURE (1)..(1): F=D-phenylalanine
FPKMHKGGNGDFEEIPEEYL

SEQ ID NO: 260 - protein, 22 aa, Artificial Sequence
  modified hirulog
  MISC_FEATURE (1)..(1): F=D-phenylalanine
FPKMHKGGGGNGDFEEIPEEYL

SEQ ID NO: 261 - protein, 21 aa, Artificial Sequence
  modified hirulog
  MISC_FEATURE (1)..(1): F=D-phenylalanine
FPKMHKTGGNGDFEEIPEEYL

SEQ ID NO: 262 - protein, 20 aa, Artificial Sequence
  modified hirulog
  MISC_FEATURE (1)..(1): F=D-phenylalanine
FPKMHKTGNGDFEEIPEEYL

SEQ ID NO: 263 - protein, 22 aa, Artificial Sequence
  modified hirulog
  MISC_FEATURE (1)..(1): F=D-phenylalanine
FPKMHKTGGGNGDFEEIPEEYL

SEQ ID NO: 264 - protein, 21 aa, Artificial Sequence
  modified hirulog
  MISC_FEATURE (1)..(1): F=D-phenylalanine
FPKMHKTAGNGDFEEIPEEYL

SEQ ID NO: 265 - protein, 20 aa, Artificial Sequence
  modified hirulog
  MISC_FEATURE (1)..(1): F=D-phenylalanine
FPKMHKTANGDFEEIPEEYL

SEQ ID NO: 266 - protein, 22 aa, Artificial Sequence
  modified hirulog
  MISC_FEATURE (1)..(1): F=D-phenylalanine
FPKMHKTAGGNGDFEEIPEEYL

SEQ ID NO: 267 - protein, 21 aa, Artificial Sequence
  modified hirulog
  MISC_FEATURE (1)..(1): F=D-phenylalanine
  misc_feature (4)..(4): Xaa can be any naturally occurring amino acid
FPKXHKGGGNGDFEEIPEEYL

SEQ ID NO: 268 - protein, 20 aa, Artificial Sequence
  modified hirulog
  MISC_FEATURE (1)..(1): F=D-phenylalanine
  misc_feature (4)..(4): Xaa can be any naturally occurring amino acid
FPKXHKGGNGDFEEIPEEYL

SEQ ID NO: 269 - protein, 22 aa, Artificial Sequence
  modified hirulog
  MISC_FEATURE (1)..(1): F=D-phenylalanine
  misc_feature (4)..(4): Xaa can be any naturally occurring amino acid
FPKXHKGGGGNGDFEEIPEEYL

SEQ ID NO: 270 - protein, 21 aa, Artificial Sequence
  modified hirulog
  MISC_FEATURE (1)..(1): F=D-phenylalanine
  misc_feature (4)..(4): Xaa can be any naturally occurring amino acid
FPKXHKTGGNGDFEEIPEEYL

SEQ ID NO: 271 - protein, 20 aa, Artificial Sequence
  modified hirulog
  MISC_FEATURE (1)..(1): F=D-phenylalanine
  misc_feature (4)..(4): Xaa can be any naturally occurring amino acid
FPKXHKTGNGDFEEIPEEYL

SEQ ID NO: 272 - protein, 22 aa, Artificial Sequence
  modified hirulog
  MISC_FEATURE (1)..(1): F=D-phenylalanine
  misc_feature (4)..(4): Xaa can be any naturally occurring amino acid
FPKXHKTGGGNGDFEEIPEEYL

SEQ ID NO: 273 - protein, 21 aa, Artificial Sequence
  modified hirulog
  MISC_FEATURE (1)..(1): F=D-phenylalanine
  misc_feature (4)..(4): Xaa can be any naturally occurring amino acid
FPKXHKTAGNGDFEEIPEEYL

SEQ ID NO: 274 - protein, 20 aa, Artificial Sequence
  modified hirulog
  MISC_FEATURE (1)..(1): F=D-phenylalanine
  misc_feature (4)..(4): Xaa can be any naturally occurring amino acid
FPKXHKTANGDFEEIPEEYL

SEQ ID NO: 275 - protein, 22 aa, Artificial Sequence
  modified hirulog
  MISC_FEATURE (1)..(1): F=D-phenylalanine
  misc_feature (4)..(4): Xaa can be any naturally occurring amino acid
FPKXHKTAGGNGDFEEIPEEYL

SEQ ID NO: 276 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGNGDFEEIPEEYL

SEQ ID NO: 277 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGNGDFEEIPEEYL

SEQ ID NO: 278 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGGNGDFEEIPEEYL

SEQ ID NO: 279 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGNGDFEEIPEEYL

SEQ ID NO: 280 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGNGDFEEIPEEYL

SEQ ID NO: 281 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGGNGDFEEIPEEYL

SEQ ID NO: 282 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGNGDFEEIPEEYL

SEQ ID NO: 283 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTANGDFEEIPEEYL

SEQ ID NO: 284 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGGNGDFEEIPEEYL

SEQ ID NO: 285 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGNGDFEEIPEEYL

SEQ ID NO: 286 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGNGDFEEIPEEYL

SEQ ID NO: 287 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGGNGDFEEIPEEYL

SEQ ID NO: 288 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGNGDFEEIPEEYL

SEQ ID NO: 289 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGNGDFEEIPEEYL

SEQ ID NO: 290 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGGNGDFEEIPEEYL

SEQ ID NO: 291 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGNGDFEEIPEEYL

SEQ ID NO: 292 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTANGDFEEIPEEYL

SEQ ID NO: 293 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGGNGDFEEIPEEYL

SEQ ID NO: 294 - protein, 20 aa, Artificial Sequence
  modified hirulog
PRMHKGGGNGDFEEIPEEYL

SEQ ID NO: 295 - protein, 19 aa, Artificial Sequence
  modified hirulog
PRMHKGGNGDFEEIPEEYL

SEQ ID NO: 296 - protein, 21 aa, Artificial Sequence
  modified hirulog
PRMHKGGGGNGDFEEIPEEYL

SEQ ID NO: 297 - protein, 20 aa, Artificial Sequence
  modified hirulog
PRMHKTGGNGDFEEIPEEYL

SEQ ID NO: 298 - protein, 19 aa, Artificial Sequence
  modified hirulog
PRMHKTGNGDFEEIPEEYL

SEQ ID NO: 299 - protein, 21 aa, Artificial Sequence
  modified hirulog
PRMHKTGGGNGDFEEIPEEYL

SEQ ID NO: 300 - protein, 20 aa, Artificial Sequence
  modified hirulog
PRMHKTAGNGDFEEIPEEYL

SEQ ID NO: 301 - protein, 19 aa, Artificial Sequence
  modified hirulog
PRMHKTANGDFEEIPEEYL

SEQ ID NO: 302 - protein, 21 aa, Artificial Sequence
  modified hirulog
PRMHKTAGGNGDFEEIPEEYL

SEQ ID NO: 303 - protein, 20 aa, Artificial Sequence
  modified hirulog
  misc_feature (3)..(3): Xaa can be any naturally occurring amino acid
PRXHKGGGNGDFEEIPEEYL

SEQ ID NO: 304 - protein, 19 aa, Artificial Sequence
  modified hirulog
  misc_feature (3)..(3): Xaa can be any naturally occurring amino acid
PRXHKGGNGDFEEIPEEYL

SEQ ID NO: 305 - protein, 21 aa, Artificial Sequence
  modified hirulog
  misc_feature (3)..(3): Xaa can be any naturally occurring amino acid
PRXHKGGGGNGDFEEIPEEYL

SEQ ID NO: 306 - protein, 20 aa, Artificial Sequence
  modified hirulog
  misc_feature (3)..(3): Xaa can be any naturally occurring amino acid
PRXHKTGGNGDFEEIPEEYL

SEQ ID NO: 307 - protein, 19 aa, Artificial Sequence
  modified hirulog
  misc_feature (3)..(3): Xaa can be any naturally occurring amino acid
PRXHKTGNGDFEEIPEEYL

SEQ ID NO: 308 - protein, 21 aa, Artificial Sequence
  modified hirulog
  misc_feature (3)..(3): Xaa can be any naturally occurring amino acid
PRXHKTGGGNGDFEEIPEEYL

SEQ ID NO: 309 - protein, 20 aa, Artificial Sequence
  modified hirulog
  misc_feature (3)..(3): Xaa can be any naturally occurring amino acid
PRXHKTAGNGDFEEIPEEYL

SEQ ID NO: 310 - protein, 19 aa, Artificial Sequence
  modified hirulog
  misc_feature (3)..(3): Xaa can be any naturally occurring amino acid
PRXHKTANGDFEEIPEEYL

SEQ ID NO: 311 - protein, 21 aa, Artificial Sequence
  modified hirulog
  misc_feature (3)..(3): Xaa can be any naturally occurring amino acid
PRXHKTAGGNGDFEEIPEEYL

SEQ ID NO: 312 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PRXHXGGGNGDFEEIPEEYL

SEQ ID NO: 313 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PRXHXGGNGDFEEIPEEYL

SEQ ID NO: 314 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PRXHXGGGGNGDFEEIPEEYL

SEQ ID NO: 315 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PRXHXTGGNGDFEEIPEEYL

SEQ ID NO: 316 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PRXHXTGNGDFEEIPEEYL

SEQ ID NO: 317 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PRXHXTGGGNGDFEEIPEEYL

SEQ ID NO: 318 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PRXHXTAGNGDFEEIPEEYL

SEQ ID NO: 319 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PRXHXTANGDFEEIPEEYL

SEQ ID NO: 320 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PRXHXTAGGNGDFEEIPEEYL

SEQ ID NO: 321 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PRXHXGGGNGDFEEIPEEYL

SEQ ID NO: 322 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PRXHXGGNGDFEEIPEEYL

SEQ ID NO: 323 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PRXHXGGGGNGDFEEIPEEYL

SEQ ID NO: 324 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PRXHXTGGNGDFEEIPEEYL

SEQ ID NO: 325 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PRXHXTGNGDFEEIPEEYL

SEQ ID NO: 326 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PRXHXTGGGNGDFEEIPEEYL

SEQ ID NO: 327 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PRXHXTAGNGDFEEIPEEYL

SEQ ID NO: 328 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PRXHXTANGDFEEIPEEYL

SEQ ID NO: 329 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PRXHXTAGGNGDFEEIPEEYL

SEQ ID NO: 330 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGNGDFEEIPEEYL

SEQ ID NO: 331 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGNGDFEEIPEEYL

SEQ ID NO: 332 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGGNGDFEEIPEEYL

SEQ ID NO: 333 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGNGDFEEIPEEYL

SEQ ID NO: 334 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGNGDFEEIPEEYL

SEQ ID NO: 335 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGGNGDFEEIPEEYL

SEQ ID NO: 336 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGNGDFEEIPEEYL

SEQ ID NO: 337 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXTANGDFEEIPEEYL

SEQ ID NO: 338 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGGNGDFEEIPEEYL

SEQ ID NO: 339 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGNGDFEEIPEEYL

SEQ ID NO: 340 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGNGDFEEIPEEYL

SEQ ID NO: 341 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGGNGDFEEIPEEYL

SEQ ID NO: 342 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGNGDFEEIPEEYL

SEQ ID NO: 343 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGNGDFEEIPEEYL

SEQ ID NO: 344 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGGNGDFEEIPEEYL

SEQ ID NO: 345 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGNGDFEEIPEEYL

SEQ ID NO: 346 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXTANGDFEEIPEEYL

SEQ ID NO: 347 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGGNGDFEEIPEEYL

SEQ ID NO: 348 - protein, 19 aa, Artificial Sequence
  modified hirulog
RMHKGGGNGDFEEIPEEYL

SEQ ID NO: 349 - protein, 18 aa, Artificial Sequence
  modified hirulog
RMHKGGNGDFEEIPEEYL

SEQ ID NO: 350 - protein, 20 aa, Artificial Sequence
  modified hirulog
RMHKGGGGNGDFEEIPEEYL

SEQ ID NO: 351 - protein, 19 aa, Artificial Sequence
  modified hirulog
RMHKTGGNGDFEEIPEEYL

SEQ ID NO: 352 - protein, 18 aa, Artificial Sequence
  modified hirulog
RMHKTGNGDFEEIPEEYL

SEQ ID NO: 353 - protein, 20 aa, Artificial Sequence
  modified hirulog
RMHKTGGGNGDFEEIPEEYL

SEQ ID NO: 354 - protein, 19 aa, Artificial Sequence
  modified hirulog
RMHKTAGNGDFEEIPEEYL

SEQ ID NO: 355 - protein, 18 aa, Artificial Sequence
  modified hirulog
RMHKTANGDFEEIPEEYL

SEQ ID NO: 356 - protein, 20 aa, Artificial Sequence
  modified hirulog
RMHKTAGGNGDFEEIPEEYL

SEQ ID NO: 357 - protein, 19 aa, Artificial Sequence
  modified hirulog
  misc_feature (2)..(2): Xaa can be any naturally occurring amino acid
RXHKGGGNGDFEEIPEEYL

SEQ ID NO: 358 - protein, 18 aa, Artificial Sequence
  modified hirulog
  misc_feature (2)..(2): Xaa can be any naturally occurring amino acid
RXHKGGNGDFEEIPEEYL

SEQ ID NO: 359 - protein, 20 aa, Artificial Sequence
  modified hirulog
  misc_feature (2)..(2): Xaa can be any naturally occurring amino acid
RXHKGGGGNGDFEEIPEEYL

SEQ ID NO: 360 - protein, 19 aa, Artificial Sequence
  modified hirulog
  misc_feature (2)..(2): Xaa can be any naturally occurring amino acid
RXHKTGGNGDFEEIPEEYL

SEQ ID NO: 361 - protein, 18 aa, Artificial Sequence
  modified hirulog
  misc_feature (2)..(2): Xaa can be any naturally occurring amino acid
RXHKTGNGDFEEIPEEYL

SEQ ID NO: 362 - protein, 20 aa, Artificial Sequence
  modified hirulog
  misc_feature (2)..(2): Xaa can be any naturally occurring amino acid
RXHKTGGGNGDFEEIPEEYL

SEQ ID NO: 363 - protein, 19 aa, Artificial Sequence
  modified hirulog
  misc_feature (2)..(2): Xaa can be any naturally occurring amino acid
RXHKTAGNGDFEEIPEEYL

SEQ ID NO: 364 - protein, 18 aa, Artificial Sequence
  modified hirulog
  misc_feature (2)..(2): Xaa can be any naturally occurring amino acid
RXHKTANGDFEEIPEEYL

SEQ ID NO: 365 - protein, 20 aa, Artificial Sequence
  modified hirulog
  misc_feature (2)..(2): Xaa can be any naturally occurring amino acid
RXHKTAGGNGDFEEIPEEYL

SEQ ID NO: 366 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
RXHXGGGNGDFEEIPEEYL

SEQ ID NO: 367 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
RXHXGGNGDFEEIPEEYL

SEQ ID NO: 368 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
RXHXGGGGNGDFEEIPEEYL

SEQ ID NO: 369 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
RXHXTGGNGDFEEIPEEYL

SEQ ID NO: 370 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
RXHXTGNGDFEEIPEEYL

SEQ ID NO: 371 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
RXHXTGGGNGDFEEIPEEYL

SEQ ID NO: 372 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
RXHXTAGNGDFEEIPEEYL

SEQ ID NO: 373 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
RXHXTANGDFEEIPEEYL

SEQ ID NO: 374 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
RXHXTAGGNGDFEEIPEEYL

SEQ ID NO: 375 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
RXHXGGGNGDFEEIPEEYL

SEQ ID NO: 376 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
RXHXGGNGDFEEIPEEYL

SEQ ID NO: 377 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
RXHXGGGGNGDFEEIPEEYL

SEQ ID NO: 378 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
RXHXTGGNGDFEEIPEEYL

SEQ ID NO: 379 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
RXHXTGNGDFEEIPEEYL

SEQ ID NO: 380 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
RXHXTGGGNGDFEEIPEEYL

SEQ ID NO: 381 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
RXHXTAGNGDFEEIPEEYL

SEQ ID NO: 382 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
RXHXTANGDFEEIPEEYL

SEQ ID NO: 383 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
RXHXTAGGNGDFEEIPEEYL

SEQ ID NO: 384 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGNGDFEEIPEEYL

SEQ ID NO: 385 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXGGNGDFEEIPEEYL

SEQ ID NO: 386 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGGNGDFEEIPEEYL

SEQ ID NO: 387 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGNGDFEEIPEEYL

SEQ ID NO: 388 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXTGNGDFEEIPEEYL

SEQ ID NO: 389 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGGNGDFEEIPEEYL

SEQ ID NO: 390 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGNGDFEEIPEEYL

SEQ ID NO: 391 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXTANGDFEEIPEEYL

SEQ ID NO: 392 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGGNGDFEEIPEEYL

SEQ ID NO: 393 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGNGDFEEIPEEYL

SEQ ID NO: 394 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXGGNGDFEEIPEEYL

SEQ ID NO: 395 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGGNGDFEEIPEEYL

SEQ ID NO: 396 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGNGDFEEIPEEYL

SEQ ID NO: 397 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXTGNGDFEEIPEEYL

SEQ ID NO: 398 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGGNGDFEEIPEEYL

SEQ ID NO: 399 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGNGDFEEIPEEYL

SEQ ID NO: 400 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXTANGDFEEIPEEYL

SEQ ID NO: 401 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGGNGDFEEIPEEYL

SEQ ID NO: 402 - protein, 18 aa, Artificial Sequence
  modified hirulog
MHKGGGNGDFEEIPEEYL

SEQ ID NO: 403 - protein, 17 aa, Artificial Sequence
  modified hirulog
MHKGGNGDFEEIPEEYL

SEQ ID NO: 404 - protein, 19 aa, Artificial Sequence
  modified hirulog
MHKGGGGNGDFEEIPEEYL

SEQ ID NO: 405 - protein, 18 aa, Artificial Sequence
  modified hirulog
MHKTGGNGDFEEIPEEYL

SEQ ID NO: 406 - protein, 17 aa, Artificial Sequence
  modified hirulog
MHKTGNGDFEEIPEEYL

SEQ ID NO: 407 - protein, 19 aa, Artificial Sequence
  modified hirulog
MHKTGGGNGDFEEIPEEYL

SEQ ID NO: 408 - protein, 18 aa, Artificial Sequence
  modified hirulog
MHKTAGNGDFEEIPEEYL

SEQ ID NO: 409 - protein, 17 aa, Artificial Sequence
  modified hirulog
MHKTANGDFEEIPEEYL

SEQ ID NO: 410 - protein, 19 aa, Artificial Sequence
  modified hirulog
MHKTAGGNGDFEEIPEEYL

SEQ ID NO: 411 - protein, 18 aa, Artificial Sequence
  modified hirulog
  misc_feature (1)..(1): Xaa can be any naturally occurring amino acid
XHKGGGNGDFEEIPEEYL

SEQ ID NO: 412 - protein, 17 aa, Artificial Sequence
  modified hirulog
  misc_feature (1)..(1): Xaa can be any naturally occurring amino acid
XHKGGNGDFEEIPEEYL

SEQ ID NO: 413 - protein, 19 aa, Artificial Sequence
  modified hirulog
  misc_feature (1)..(1): Xaa can be any naturally occurring amino acid
XHKGGGGNGDFEEIPEEYL

SEQ ID NO: 414 - protein, 18 aa, Artificial Sequence
  modified hirulog
  misc_feature (1)..(1): Xaa can be any naturally occurring amino acid
XHKTGGNGDFEEIPEEYL

SEQ ID NO: 415 - protein, 17 aa, Artificial Sequence
  modified hirulog
  misc_feature (1)..(1): Xaa can be any naturally occurring amino acid
XHKTGNGDFEEIPEEYL

SEQ ID NO: 416 - protein, 19 aa, Artificial Sequence
  modified hirulog
  misc_feature (1)..(1): Xaa can be any naturally occurring amino acid
XHKTGGGNGDFEEIPEEYL

SEQ ID NO: 417 - protein, 18 aa, Artificial Sequence
  modified hirulog
  misc_feature (1)..(1): Xaa can be any naturally occurring amino acid
XHKTAGNGDFEEIPEEYL

SEQ ID NO: 418 - protein, 17 aa, Artificial Sequence
  modified hirulog
  misc_feature (1)..(1): Xaa can be any naturally occurring amino acid
XHKTANGDFEEIPEEYL

SEQ ID NO: 419 - protein, 19 aa, Artificial Sequence
  modified hirulog
  misc_feature (1)..(1): Xaa can be any naturally occurring amino acid
XHKTAGGNGDFEEIPEEYL

SEQ ID NO: 420 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXGGGNGDFEEIPEEYL

SEQ ID NO: 421 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXGGNGDFEEIPEEYL

SEQ ID NO: 422 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXGGGGNGDFEEIPEEYL

SEQ ID NO: 423 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXTGGNGDFEEIPEEYL

SEQ ID NO: 424 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXTGNGDFEEIPEEYL

SEQ ID NO: 425 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXTGGGNGDFEEIPEEYL

SEQ ID NO: 426 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXTAGNGDFEEIPEEYL

SEQ ID NO: 427 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXTANGDFEEIPEEYL

SEQ ID NO: 428 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXTAGGNGDFEEIPEEYL

SEQ ID NO: 429 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXGGGNGDFEEIPEEYL

SEQ ID NO: 430 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXGGNGDFEEIPEEYL

SEQ ID NO: 431 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXGGGGNGDFEEIPEEYL

SEQ ID NO: 432 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXTGGNGDFEEIPEEYL

SEQ ID NO: 433 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXTGNGDFEEIPEEYL

SEQ ID NO: 434 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXTGGGNGDFEEIPEEYL

SEQ ID NO: 435 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXTAGNGDFEEIPEEYL

SEQ ID NO: 436 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXTANGDFEEIPEEYL

SEQ ID NO: 437 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXTAGGNGDFEEIPEEYL

SEQ ID NO: 438 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXGGGNGDFEEIPEEYL

SEQ ID NO: 439 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXGGNGDFEEIPEEYL

SEQ ID NO: 440 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXGGGGNGDFEEIPEEYL

SEQ ID NO: 441 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXTGGNGDFEEIPEEYL

SEQ ID NO: 442 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXTGNGDFEEIPEEYL

SEQ ID NO: 443 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXTGGGNGDFEEIPEEYL

SEQ ID NO: 444 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXTAGNGDFEEIPEEYL

SEQ ID NO: 445 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXTANGDFEEIPEEYL

SEQ ID NO: 446 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXTAGGNGDFEEIPEEYL

SEQ ID NO: 447 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXGGGNGDFEEIPEEYL

SEQ ID NO: 448 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXGGNGDFEEIPEEYL

SEQ ID NO: 449 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXGGGGNGDFEEIPEEYL

SEQ ID NO: 450 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXTGGNGDFEEIPEEYL

SEQ ID NO: 451 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXTGNGDFEEIPEEYL

SEQ ID NO: 452 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXTGGGNGDFEEIPEEYL

SEQ ID NO: 453 - protein, 18 aa, Artificial Sequence
  modified hirulog
  misc_feature (1)..(1): Xaa can be any naturally occurring amino acid
  misc_feature (3)..(3): Xaa can be any naturally occurring amino acid
XHXTAGNGDFEEIPEEYL

SEQ ID NO: 454 - protein, 17 aa, Artificial Sequence
  modified hirulog
  misc_feature (1)..(1): Xaa can be any naturally occurring amino acid
  misc_feature (3)..(3): Xaa can be any naturally occurring amino acid
XHXTANGDFEEIPEEYL

SEQ ID NO: 455 - protein, 19 aa, Artificial Sequence
  modified hirulog
  misc_feature (1)..(1): Xaa can be any naturally occurring amino acid
  misc_feature (3)..(3): Xaa can be any naturally occurring amino acid
XHXTAGGNGDFEEIPEEYL

SEQ ID NO: 456 - protein, 20 aa, Artificial Sequence
  modified hirulog
FPRMHKGGGNGDYEPIPEEA

SEQ ID NO: 457 - protein, 19 aa, Artificial Sequence
  modified hirulog
FPRMHKGGNGDYEPIPEEA

SEQ ID NO: 458 - protein, 21 aa, Artificial Sequence
  modified hirulog
FPRMHKGGGGNGDYEPIPEEA

SEQ ID NO: 459 - protein, 20 aa, Artificial Sequence
  modified hirulog
FPRMHKTGGNGDYEPIPEEA

SEQ ID NO: 460 - protein, 19 aa, Artificial Sequence
  modified hirulog
FPRMHKTGNGDYEPIPEEA

SEQ ID NO: 461 - protein, 21 aa, Artificial Sequence
  modified hirulog
FPRMHKTGGGNGDYEPIPEEA

SEQ ID NO: 462 - protein, 20 aa, Artificial Sequence
  modified hirulog
FPRMHKTAGNGDYEPIPEEA

SEQ ID NO: 463 - protein, 19 aa, Artificial Sequence
  modified hirulog
FPRMHKTANGDYEPIPEEA

SEQ ID NO: 464 - protein, 21 aa, Artificial Sequence
  modified hirulog
FPRMHKTAGGNGDYEPIPEEA

SEQ ID NO: 465 - protein, 20 aa, Artificial Sequence
  modified hirulog
  MISC_FEATURE (1)..(1): F=D-phenylalanine
FPRMHKGGGNGDYEPIPEEA

SEQ ID NO: 466 - protein, 19 aa, Artificial Sequence
  modified hirulog
  MISC_FEATURE (1)..(1): F=D-phenylalanine
FPRMHKGGNGDYEPIPEEA

SEQ ID NO: 467 - protein, 21 aa, Artificial Sequence
  modified hirulog
  MISC_FEATURE (1)..(1): F=D-phenylalanine
FPRMHKGGGGNGDYEPIPEEA

SEQ ID NO: 468 - protein, 20 aa, Artificial Sequence
  modified hirulog
  MISC_FEATURE (1)..(1): F=D-phenylalanine
FPRMHKTGGNGDYEPIPEEA

SEQ ID NO: 469 - protein, 19 aa, Artificial Sequence
  modified hirulog
  MISC_FEATURE (1)..(1): F=D-phenylalanine
FPRMHKTGNGDYEPIPEEA

SEQ ID NO: 470 - protein, 21 aa, Artificial Sequence
  modified hirulog
  MISC_FEATURE (1)..(1): F=D-phenylalanine
FPRMHKTGGGNGDYEPIPEEA

SEQ ID NO: 471 - protein, 20 aa, Artificial Sequence
  modified hirulog
  MISC_FEATURE (1)..(1): F=D-phenylalanine
FPRMHKTAGNGDYEPIPEEA

SEQ ID NO: 472 - protein, 19 aa, Artificial Sequence
  modified hirulog
  MISC_FEATURE (1)..(1): F=D-phenylalanine
FPRMHKTANGDYEPIPEEA

SEQ ID NO: 473 - protein, 21 aa, Artificial Sequence
  modified hirulog
  MISC_FEATURE (1)..(1): F=D-phenylalanine
FPRMHKTAGGNGDYEPIPEEA

SEQ ID NO: 474 - protein, 20 aa, Artificial Sequence
  modified hirulog
  MISC_FEATURE (1)..(1): F=D-phenylalanine
  misc_feature (4)..(4): Xaa can be any naturally occurring amino acid
FPRXHKGGGNGDYEPIPEEA

SEQ ID NO: 475 - protein, 18 aa, Artificial Sequence
  modified hirulog
  MISC_FEATURE (1)..(1): F=D-phenylalanine
  misc_feature (4)..(4): Xaa can be any naturally occurring amino acid
FPRXHKGGNGDYEPIPEE

SEQ ID NO: 476 - protein, 21 aa, Artificial Sequence
  modified hirulog
  MISC_FEATURE (1)..(1): F=D-phenylalanine
  misc_feature (4)..(4): Xaa can be any naturally occurring amino acid
FPRXHKGGGGNGDYEPIPEEA

SEQ ID NO: 477 - protein, 20 aa, Artificial Sequence
  modified hirulog
  MISC_FEATURE (1)..(1): F=D-phenylalanine
  misc_feature (4)..(4): Xaa can be any naturally occurring amino acid
FPRXHKTGGNGDYEPIPEEA

SEQ ID NO: 478 - protein, 19 aa, Artificial Sequence
  modified hirulog
  MISC_FEATURE (1)..(1): F=D-phenylalanine
  misc_feature (4)..(4): Xaa can be any naturally occurring amino acid
FPRXHKTGNGDYEPIPEEA

SEQ ID NO: 479 - protein, 22 aa, Artificial Sequence
  modified hirulog
  MISC_FEATURE (1)..(1): F=D-phenylalanine
  misc_feature (4)..(4): Xaa can be any naturally occurring amino acid
FPRXHKTGGGNGDFYEPIPEEA

SEQ ID NO: 480 - protein, 20 aa, Artificial Sequence
  modified hirulog
  MISC_FEATURE (1)..(1): F=D-phenylalanine
  misc_feature (4)..(4): Xaa can be any naturally occurring amino acid
FPRXHKTAGNGDYEPIPEEA

SEQ ID NO: 481 - protein, 19 aa, Artificial Sequence
  modified hirulog
  MISC_FEATURE (1)..(1): F=D-phenylalanine
  misc_feature (4)..(4): Xaa can be any naturally occurring amino acid
FPRXHKTANGDYEPIPEEA

SEQ ID NO: 482 - protein, 21 aa, Artificial Sequence
  modified hirulog
  MISC_FEATURE (1)..(1): F=D-phenylalanine
  misc_feature (4)..(4): Xaa can be any naturally occurring amino acid
FPRXHKTAGGNGDYEPIPEEA

SEQ ID NO: 483 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGNGDYEPIPEEA

SEQ ID NO: 484 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGNGDYEPIPEEA

SEQ ID NO: 485 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGGNGDYEPIPEEA

SEQ ID NO: 486 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGNGDYEPIPEEA

SEQ ID NO: 487 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGNGDYEPIPEEA

SEQ ID NO: 488 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGGNGDYEPIPEEA

SEQ ID NO: 489 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGNGDYEPIPEEA

SEQ ID NO: 490 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTANGDYEPIPEEA

SEQ ID NO: 491 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGGNGDYEPIPEEA

SEQ ID NO: 492 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGNGDYEPIPEEA

SEQ ID NO: 493 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGNGDYEPIPEEA

SEQ ID NO: 494 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGGNGDYEPIPEEA

SEQ ID NO: 495 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGNGDYEPIPEEA

SEQ ID NO: 496 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGNGDYEPIPEEA

SEQ ID NO: 497 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGGNGDYEPIPEEA

SEQ ID NO: 498 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGNGDYEPIPEEA

SEQ ID NO: 499 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTANGDYEPIPEEA

SEQ ID NO: 500 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGGNGDYEPIPEEA

SEQ ID NO: 501 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGNGDYEPIPEEA

SEQ ID NO: 502 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGNGDYEPIPEEA

SEQ ID NO: 503 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGGNGDYEPIPEEA

SEQ ID NO: 504 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGNGDYEPIPEEA

SEQ ID NO: 505 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGNGDYEPIPEEA

SEQ ID NO: 506 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGGNGDYEPIPEEA

SEQ ID NO: 507 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGNGDYEPIPEEA

SEQ ID NO: 508 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTANGDYEPIPEEA

SEQ ID NO: 509 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGGNGDYEPIPEEA

SEQ ID NO: 510 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGNGDYEPIPEEA

SEQ ID NO: 511 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGNGDYEPIPEEA

SEQ ID NO: 512 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGGNGDYEPIPEEA

SEQ ID NO: 513 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGNGDYEPIPEEA

SEQ ID NO: 514 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGNGDYEPIPEEA

SEQ ID NO: 515 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGGNGDYEPIPEEA

SEQ ID NO: 516 - protein, 20 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGNGDYEPIPEEA

SEQ ID NO: 517 - protein, 19 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTANGDYEPIPEEA

SEQ ID NO: 518 - protein, 21 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGGNGDYEPIPEEA

SEQ ID NO: 519 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGNGDYEPIPEEA

SEQ ID NO: 520 - protein, 18 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGNGDYEPIPEEA

SEQ ID NO: 521 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGGNGDYEPIPEEA

SEQ ID NO: 522 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGNGDYEPIPEEA

SEQ ID NO: 523 - protein, 18 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGNGDYEPIPEEA

SEQ ID NO: 524 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGGNGDYEPIPEEA

SEQ ID NO: 525 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGNGDYEPIPEEA

SEQ ID NO: 526 - protein, 18 aa, Artificial Sequence
  modified hirulog
  misc_feature (3)..(3): Xaa can be any naturally occurring amino acid
  misc_feature (5)..(5): Xaa can be any naturally occurring amino acid
PKXHXTANGDYEPIPEEA

SEQ ID NO: 527 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGGNGDYEPIPEEA

SEQ ID NO: 528 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGNGDYEPIPEEA

SEQ ID NO: 529 - protein, 18 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGNGDYEPIPEEA

SEQ ID NO: 530 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGGNGDYEPIPEEA

SEQ ID NO: 531 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGNGDYEPIPEEA

SEQ ID NO: 532 - protein, 18 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGNGDYEPIPEEA

SEQ ID NO: 533 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGGNGDYEPIPEEA

SEQ ID NO: 534 - protein, 19 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGNGDYEPIPEEA

SEQ ID NO: 535 - skipped

SEQ ID NO: 536 - protein, 18 aa, Artificial Sequence
  modified hirulog
  misc_feature (3)..(3): Xaa can be any naturally occurring amino acid
  misc_feature (5)..(5): Xaa can be any naturally occurring amino acid
PKXHXTANGDYEPIPEEA

SEQ ID NO: 537 - protein, 20 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGGNGDYEPIPEEA

SEQ ID NO: 538 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGNGDYEPIPEEA

SEQ ID NO: 539 - protein, 17 aa, Artificial Sequence
  modified hirulog
  misc_feature (2)..(2): Xaa can be any naturally occurring amino acid
  misc_feature (4)..(4): Xaa can be any naturally occurring amino acid
KXHXGGNGDYEPIPEEA

SEQ ID NO: 540 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGGNGDYEPIPEEA

SEQ ID NO: 541 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGNGDYEPIPEEA

SEQ ID NO: 542 - protein, 17 aa, Artificial Sequence
  modified hirulog
  misc_feature (2)..(2): Xaa can be any naturally occurring amino acid
  misc_feature (4)..(4): Xaa can be any naturally occurring amino acid
KXHXTGNGDYEPIPEEA

SEQ ID NO: 543 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGGNGDYEPIPEEA

SEQ ID NO: 544 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGNGDYEPIPEEA

SEQ ID NO: 545 - protein, 17 aa, Artificial Sequence
  modified hirulog
  misc_feature (2)..(2): Xaa can be any naturally occurring amino acid
  misc_feature (4)..(4): Xaa can be any naturally occurring amino acid
KXHXTANGDYEPIPEEA

SEQ ID NO: 546 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGGNGDYEPIPEEA

SEQ ID NO: 547 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGNGDYEPIPEEA

SEQ ID NO: 548 - protein, 17 aa, Artificial Sequence
  modified hirulog
  misc_feature (2)..(2): Xaa can be any naturally occurring amino acid
  misc_feature (4)..(4): Xaa can be any naturally occurring amino acid
KXHXGGNGDYEPIPEEA

SEQ ID NO: 549 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGGNGDYEPIPEEA

SEQ ID NO: 550 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGNGDYEPIPEEA

SEQ ID NO: 551 - protein, 17 aa, Artificial Sequence
  modified hirulog
  misc_feature (2)..(2): Xaa can be any naturally occurring amino acid
  misc_feature (4)..(4): Xaa can be any naturally occurring amino acid
KXHXTGNGDYEPIPEEA

SEQ ID NO: 552 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGGNGDYEPIPEEA

SEQ ID NO: 553 - protein, 18 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGNGDYEPIPEEA

SEQ ID NO: 554 - protein, 17 aa, Artificial Sequence
  modified hirulog
  misc_feature (2)..(2): Xaa can be any naturally occurring amino acid
  misc_feature (4)..(4): Xaa can be any naturally occurring amino acid
KXHXTANGDYEPIPEEA

SEQ ID NO: 555 - protein, 19 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGGNGDYEPIPEEA

SEQ ID NO: 556 - protein, 23 aa, Artificial Sequence
  modified hirulog
FPRMHKGGGAPPFDFEAIPEEYL

SEQ ID NO: 557 - protein, 22 aa, Artificial Sequence
  modified hirulog
FPRMHKGGAPPFDFEAIPEEYL

SEQ ID NO: 558 - protein, 24 aa, Artificial Sequence
  modified hirulog
FPRMHKGGGGAPPFDFEAIPEEYL

SEQ ID NO: 559 - protein, 23 aa, Artificial Sequence
  modified hirulog
FPRMHKTGGAPPFDFEAIPEEYL

SEQ ID NO: 560 - protein, 22 aa, Artificial Sequence
  modified hirulog
FPRMHKTGAPPFDFEAIPEEYL

SEQ ID NO: 561 - protein, 24 aa, Artificial Sequence
  modified hirulog
FPRMHKTGGGAPPFDFEAIPEEYL

SEQ ID NO: 562 - protein, 23 aa, Artificial Sequence
  modified hirulog
FPRMHKTAGAPPFDFEAIPEEYL

SEQ ID NO: 563 - protein, 22 aa, Artificial Sequence
  modified hirulog
FPRMHKTAAPPFDFEAIPEEYL

SEQ ID NO: 564 - protein, 24 aa, Artificial Sequence
  modified hirulog
FPRMHKTAGGAPPFDFEAIPEEYL

SEQ ID NO: 565 - protein, 23 aa, Artificial Sequence
  modified hirulog
  MISC_FEATURE (1)..(1): F=D-phenylalanine
FPRMHKGGGAPPFDFEAIPEEYL

SEQ ID NO: 566 - protein, 22 aa, Artificial Sequence
  modified hirulog
  MISC_FEATURE (1)..(1): F=D-phenylalanine
FPRMHKGGAPPFDFEAIPEEYL

SEQ ID NO: 567 - protein, 24 aa, Artificial Sequence
  modified hirulog
  MISC_FEATURE (1)..(1): F=D-phenylalanine
FPRMHKGGGGAPPFDFEAIPEEYL

SEQ ID NO: 568 - protein, 23 aa, Artificial Sequence
  modified hirulog
  MISC_FEATURE (1)..(1): F=D-phenylalanine
FPRMHKTGGAPPFDFEAIPEEYL

SEQ ID NO: 569 - protein, 22 aa, Artificial Sequence
  modified hirulog
  MISC_FEATURE (1)..(1): F=D-phenylalanine
FPRMHKTGAPPFDFEAIPEEYL

SEQ ID NO: 570 - protein, 24 aa, Artificial Sequence
  modified hirulog
  MISC_FEATURE (1)..(1): F=D-phenylalanine
FPRMHKTGGGAPPFDFEAIPEEYL

SEQ ID NO: 571 - protein, 23 aa, Artificial Sequence
  modified hirulog
  MISC_FEATURE (1)..(1): F=D-phenylalanine
FPRMHKTAGAPPFDFEAIPEEYL

SEQ ID NO: 572 - protein, 22 aa, Artificial Sequence
  modified hirulog
  MISC_FEATURE (1)..(1): F=D-phenylalanine
FPRMHKTAAPPFDFEAIPEEYL

SEQ ID NO: 573 - protein, 24 aa, Artificial Sequence
  modified hirulog
  MISC_FEATURE (1)..(1): F=D-phenylalanine
FPRMHKTAGGAPPFDFEAIPEEYL

SEQ ID NO: 574 - protein, 23 aa, Artificial Sequence
  modified hirulog
  MISC_FEATURE (1)..(1): F=D-phenylalanine
  misc_feature (4)..(4): Xaa can be any naturally occurring amino acid
FPRXHKGGGAPPFDFEAIPEEYL

SEQ ID NO: 575 - protein, 22 aa, Artificial Sequence
  modified hirulog
  MISC_FEATURE (1)..(1): F=D-phenylalanine
  misc_feature (4)..(4): Xaa can be any naturally occurring amino acid
FPRXHKGGAPPFDFEAIPEEYL

SEQ ID NO: 576 - protein, 24 aa, Artificial Sequence
  modified hirulog
  MISC_FEATURE (1)..(1): F=D-phenylalanine
  misc_feature (4)..(4): Xaa can be any naturally occurring amino acid
FPRXHKGGGGAPPFDFEAIPEEYL

SEQ ID NO: 577 - protein, 23 aa, Artificial Sequence
  modified hirulog
  MISC_FEATURE (1)..(1): F=D-phenylalanine
  misc_feature (4)..(4): Xaa can be any naturally occurring amino acid
FPRXHKTGGAPPFDFEAIPEEYL

SEQ ID NO: 578 - protein, 22 aa, Artificial Sequence
  modified hirulog
  MISC_FEATURE (1)..(1): F=D-phenylalanine
  misc_feature (4)..(4): Xaa can be any naturally occurring amino acid
FPRXHKTGAPPFDFEAIPEEYL

SEQ ID NO: 579 - protein, 24 aa, Artificial Sequence
  modified hirulog
  MISC_FEATURE (1)..(1): F=D-phenylalanine
  misc_feature (4)..(4): Xaa can be any naturally occurring amino acid
FPRXHKTGGGAPPFDFEAIPEEYL

SEQ ID NO: 580 - protein, 23 aa, Artificial Sequence
  modified hirulog
  MISC_FEATURE (1)..(1): F=D-phenylalanine
  misc_feature (4)..(4): Xaa can be any naturally occurring amino acid
FPRXHKTAGAPPFDFEAIPEEYL

SEQ ID NO: 581 - protein, 22 aa, Artificial Sequence
  modified hirulog
  MISC_FEATURE (1)..(1): F=D-phenylalanine
  misc_feature (4)..(4): Xaa can be any naturally occurring amino acid
FPRXHKTAAPPFDFEAIPEEYL

SEQ ID NO: 582 - protein, 23 aa, Artificial Sequence
  modified hirulog
  MISC_FEATURE (1)..(1): F=D-phenylalanine
  misc_feature (4)..(4): Xaa can be any naturally occurring amino acid
FPRXHKTAGAPPFDFEAIPEEYL

SEQ ID NO: 583 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGAPPFDFEAIPEEYLL

SEQ ID NO: 584 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGAPPFDFEAIPEEYL

SEQ ID NO: 585 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGGAPPFDFEAIPEEYL

SEQ ID NO: 586 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGAPPFDFEAIPEEYL

SEQ ID NO: 587 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGAPPFDFEAIPEEYL

SEQ ID NO: 588 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGGAPPFDFEAIPEEYL

SEQ ID NO: 589 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGAPPFDFEAIPEEYL

SEQ ID NO: 590 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAAPPFDFEAIPEEYL

SEQ ID NO: 591 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGGAPPFDFEAIPEEYL

SEQ ID NO: 592 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGAPPFDFEAIPEEYL

SEQ ID NO: 593 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXGGGGAPPFDFEAIPEEYL

SEQ ID NO: 594 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGAPPFDFEAIPEEYL

SEQ ID NO: 595 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGAPPFDFEAIPEEYL

SEQ ID NO: 596 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTGGGAPPFDFEAIPEEYL

SEQ ID NO: 597 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGAPPFDFEAIPEEYL

SEQ ID NO: 598 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAAPPFDFEAIPEEYL

SEQ ID NO: 599 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPRXHXTAGGAPPFDFEAIPEEYL

SEQ ID NO: 600 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGAPPFDFEAIPEEYL

SEQ ID NO: 601 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGAPPFDFEAIPEEYL

SEQ ID NO: 602 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGGAPPFDFEAIPEEYL

SEQ ID NO: 603 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGAPPFDFEAIPEEYL

SEQ ID NO: 604 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGAPPFDFEAIPEEYL

SEQ ID NO: 605 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGGAPPFDFEAIPEEYL

SEQ ID NO: 606 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGAPPFDFEAIPEEYL

SEQ ID NO: 607 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAAPPFDFEAIPEEYL

SEQ ID NO: 608 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGGAPPFDFEAIPEEYL

SEQ ID NO: 609 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGAPPFDFEAIPEEYL

SEQ ID NO: 610 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGAPPFDFEAIPEEYL

SEQ ID NO: 611 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXGGGGAPPFDFEAIPEEYL

SEQ ID NO: 612 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGAPPFDFEAIPEEYL

SEQ ID NO: 613 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGAPPFDFEAIPEEYL

SEQ ID NO: 614 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTGGGAPPFDFEAIPEEYL

SEQ ID NO: 615 - protein, 23 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGAPPFDFEAIPEEYL

SEQ ID NO: 616 - protein, 22 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAAPPFDFEAIPEEYL

SEQ ID NO: 617 - protein, 24 aa, Artificial Sequence
  modified hirulog
  MISC_FEATURE (1)..(1): F=D-phenylalanine
  misc_feature (4)..(4): Xaa can be any naturally occurring amino acid
  misc_feature (6)..(6): Xaa can be any naturally occurring amino acid
FPKXHXTAGGAPPFDFEAIPEEYL

SEQ ID NO: 618 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGAPPFDFEAIPEEYL

SEQ ID NO: 619 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGAPPFDFEAIPEEYL

SEQ ID NO: 620 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGGAPPFDFEAIPEEYL

SEQ ID NO: 621 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGAPPFDFEAIPEEYL

SEQ ID NO: 622 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGAPPFDFEAIPEEYL

SEQ ID NO: 623 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGGAPPFDFEAIPEEYL

SEQ ID NO: 624 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGAPPFDFEAIPEEYL

SEQ ID NO: 625 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAAPPFDFEAIPEEYL

SEQ ID NO: 626 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGGAPPFDFEAIPEEYL

SEQ ID NO: 627 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGAPPFDFEAIPEEYL

SEQ ID NO: 628 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGAPPFDFEAIPEEYL

SEQ ID NO: 629 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGGAPPFDFEAIPEEYL

SEQ ID NO: 630 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGAPPFDFEAIPEEYL

SEQ ID NO: 631 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGAPPFDFEAIPEEYL

SEQ ID NO: 632 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGGAPPFDFEAIPEEYL

SEQ ID NO: 633 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGAPPFDFEAIPEEYL

SEQ ID NO: 634 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAAPPFDFEAIPEEYL

SEQ ID NO: 635 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGGAPPFDFEAIPEEYL

SEQ ID NO: 636 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGAPPFDFEAIPEEYL

SEQ ID NO: 637 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGAPPFDFEAIPEEYL

SEQ ID NO: 638 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGGAPPFDFEAIPEEYL

SEQ ID NO: 639 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGAPPFDFEAIPEEYL

SEQ ID NO: 640 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGAPPFDFEAIPEEYL

SEQ ID NO: 641 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGGAPPFDFEAIPEEYL

SEQ ID NO: 642 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGAPPFDFEAIPEEYL

SEQ ID NO: 643 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAAPPFDFEAIPEEYL

SEQ ID NO: 644 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGGAPPFDFEAIPEEYL

SEQ ID NO: 645 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGAPPFDFEAIPEEYL

SEQ ID NO: 646 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGAPPFDFEAIPEEYL

SEQ ID NO: 647 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXGGGGAPPFDFEAIPEEYL

SEQ ID NO: 648 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGAPPFDFEAIPEEYL

SEQ ID NO: 649 - protein, 21 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGAPPFDFEAIPEEYL

SEQ ID NO: 650 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXTGGGAPPFDFEAIPEEYL

SEQ ID NO: 651 - protein, 22 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGAPPFDFEAIPEEYL

SEQ ID NO: 652 - protein, 21 aa, Artificial Sequence
  modified hirulog3
  misc_feature (3)..(3): Xaa can be any naturally occurring amino acid
  misc_feature (5)..(5): Xaa can be any naturally occurring amino acid
PKXHXTAAPPFDFEAIPEEYL

SEQ ID NO: 653 - protein, 23 aa, Artificial Sequence
  modified hirulog
  misc_feature (3)..(3): Xaa can be any naturally occurring amino acid
  misc_feature (5)..(5): Xaa can be any naturally occurring amino acid
PKXHXTAGGAPPFDFEAIPEEYL

SEQ ID NO: 654 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGAPPFDFEAIPEEYL

SEQ ID NO: 655 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXGGAPPFDFEAIPEEYL

SEQ ID NO: 656 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGGAPPFDFEAIPEEYL

SEQ ID NO: 657 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGAPPFDFEAIPEEYL

SEQ ID NO: 658 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTGAPPFDFEAIPEEYL

SEQ ID NO: 659 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGGAPPFDFEAIPEEYL

SEQ ID NO: 660 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGAPPFDFEAIPEEYL

SEQ ID NO: 661 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTAAPPFDFEAIPEEYL

SEQ ID NO: 662 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGGAPPFDFEAIPEEYL

SEQ ID NO: 663 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGAPPFDFEAIPEEYL

SEQ ID NO: 664 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXGGAPPFDFEAIPEEYL

SEQ ID NO: 665 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGGAPPFDFEAIPEEYL

SEQ ID NO: 666 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGAPPFDFEAIPEEYL

SEQ ID NO: 667 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTGAPPFDFEAIPEEYL

SEQ ID NO: 668 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGGAPPFDFEAIPEEYL

SEQ ID NO: 669 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGAPPFDFEAIPEEYL

SEQ ID NO: 670 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTAAPPFDFEAIPEEYL

SEQ ID NO: 671 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGGAPPFDFEAIPEEYL

SEQ ID NO: 672 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGAPPFDFEAIPEEYL

SEQ ID NO: 673 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXGGAPPFDFEAIPEEYL

SEQ ID NO: 674 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGGAPPFDFEAIPEEYL

SEQ ID NO: 675 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGAPPFDFEAIPEEYL

SEQ ID NO: 676 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTGAPPFDFEAIPEEYL

SEQ ID NO: 677 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGGAPPFDFEAIPEEYL

SEQ ID NO: 678 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGAPPFDFEAIPEEYL

SEQ ID NO: 679 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTAAPPFDFEAIPEEYL

SEQ ID NO: 680 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGGAPPFDFEAIPEEYL

SEQ ID NO: 681 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGAPPFDFEAIPEEYL

SEQ ID NO: 682 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXGGAPPFDFEAIPEEYL

SEQ ID NO: 683 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXGGGGAPPFDFEAIPEEYL

SEQ ID NO: 684 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGAPPFDFEAIPEEYL

SEQ ID NO: 685 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTGAPPFDFEAIPEEYL

SEQ ID NO: 686 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXTGGGAPPFDFEAIPEEYL

SEQ ID NO: 687 - protein, 21 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGAPPFDFEAIPEEYL

SEQ ID NO: 688 - protein, 20 aa, Artificial Sequence
  modified hirulog
  misc_feature (2)..(2): Xaa can be any naturally occurring amino acid
  misc_feature (4)..(4): Xaa can be any naturally occurring amino acid
KXHXTAAPPFDFEAIPEEYL

SEQ ID NO: 689 - protein, 22 aa, Artificial Sequence
  modified hirulog
  misc_feature (2)..(2): Xaa can be any naturally occurring amino acid
  misc_feature (4)..(4): Xaa can be any naturally occurring amino acid
KXHXTAGGAPPFDFEAIPEEYL

SEQ ID NO: 690 - protein, 21 aa, Artificial Sequence
  modified hirudin
FPRMHKGNGDFEEIPEEDILN

SEQ ID NO: 691 - protein, 22 aa, Artificial Sequence
  modified hirudin
FPRMHKGGNGDFEEIPEEDILN

SEQ ID NO: 692 - protein, 23 aa, Artificial Sequence
  modified hirudin
FPRMHKGGGNGDFEEIPEEDILN

SEQ ID NO: 693 - protein, 21 aa, Artificial Sequence
  modified hirudin
FPRMHKGNGDFEEIPDEDILN

SEQ ID NO: 694 - protein, 22 aa, Artificial Sequence
  modified hirudin
FPRMHKGGNGDFEEIPDEDILN

SEQ ID NO: 695 - protein, 23 aa, Artificial Sequence
  modified hirudin
FPRMHKGGGNGDFEEIPDEDILN

SEQ ID NO: 696 - protein, 20 aa, Artificial Sequence
  modified hirudin
FPRMHKGNGDFEEIPEAYDE

SEQ ID NO: 697 - protein, 21 aa, Artificial Sequence
  modified hirudin
FPRMHKGGNGDFEEIPEAYDE

SEQ ID NO: 698 - protein, 22 aa, Artificial Sequence
  modified hirudin
FPRMHKGGGNGDFEEIPEAYDE

SEQ ID NO: 699 - protein, 20 aa, Artificial Sequence
  modified hirudin
FPRMHKGNGDFEAIPEAYDE

SEQ ID NO: 700 - protein, 21 aa, Artificial Sequence
  modified hirudin
FPRMHKGGNGDFEAIPEAYDE

SEQ ID NO: 701 - protein, 22 aa, Artificial Sequence
  modified hirudin
FPRMHKGGGNGDFEAIPEAYDE

SEQ ID NO: 702 - protein, 20 aa, Artificial Sequence
  modified hirudin
FPRMHKGNGDFEPIPEAYDE

SEQ ID NO: 703 - protein, 21 aa, Artificial Sequence
  modified hirudin
FPRMHKGGNGDFEPIPEAYDE

SEQ ID NO: 704 - protein, 22 aa, Artificial Sequence
  modified hirudin
FPRMHKGGGNGDFEPIPEAYDE

SEQ ID NO: 705 - protein, 20 aa, Artificial Sequence
  modified hirudin
FPRMHKGNGDFEEFPEAYDE

SEQ ID NO: 706 - protein, 21 aa, Artificial Sequence
  modified hirudin
FPRMHKGGNGDFEEFPEAYDE

SEQ ID NO: 707 - protein, 22 aa, Artificial Sequence
  modified hirudin
FPRMHKGGGNGDFEEFPEAYDE

SEQ ID NO: 708 - protein, 20 aa, Artificial Sequence
  modified hirudin
FPRMHKGNGDFEAFPEAYDE

SEQ ID NO: 709 - protein, 21 aa, Artificial Sequence
  modified hirudin
FPRMHKGGNGDFEAFPEAYDE

SEQ ID NO: 710 - protein, 22 aa, Artificial Sequence
  modified hirudin
FPRMHKGGGNGDFEAFPEAYDE

SEQ ID NO: 711 - protein, 20 aa, Artificial Sequence
  modified hirudin
FPRMHKGNGDFEPFPEAYDE

SEQ ID NO: 712 - protein, 21 aa, Artificial Sequence
  modified hirudin
FPRMHKGGNGDFEPFPEAYDE

SEQ ID NO: 713 - protein, 22 aa, Artificial Sequence
  modified hirudin
FPRMHKGGGNGDFEPFPEAYDE

SEQ ID NO: 714 - protein, 20 aa, Artificial Sequence
  modified hirudin
FPRMHKGNGDFEEIPDAYDE

SEQ ID NO: 715 - protein, 21 aa, Artificial Sequence
  modified hirudin
FPRMHKGGNGDFEEIPDAYDE

SEQ ID NO: 716 - protein, 22 aa, Artificial Sequence
  modified hirudin
FPRMHKGGGNGDFEEIPDAYDE

SEQ ID NO: 717 - protein, 20 aa, Artificial Sequence
  modified hirudin
FPRMHKGNGDFEAIPDAYDE

SEQ ID NO: 718 - protein, 21 aa, Artificial Sequence
  modified hirudin
FPRMHKGGNGDFEAIPDAYDE

SEQ ID NO: 719 - protein, 22 aa, Artificial Sequence
  modified hirudin
FPRMHKGGGNGDFEAIPDAYDE

SEQ ID NO: 720 - protein, 20 aa, Artificial Sequence
  modified hirudin
FPRMHKGNGDFEPIPDAYDE

SEQ ID NO: 721 - protein, 21 aa, Artificial Sequence
  modified hirudin
FPRMHKGGNGDFEPIPDAYDE

SEQ ID NO: 722 - protein, 22 aa, Artificial Sequence
  modified hirudin
FPRMHKGGGNGDFEPIPDAYDE

SEQ ID NO: 723 - protein, 20 aa, Artificial Sequence
  modified hirudin
FPRMHKGNGDFEEFPDAYDE

SEQ ID NO: 724 - protein, 21 aa, Artificial Sequence
  modified hirudin
FPRMHKGGNGDFEEFPDAYDE

SEQ ID NO: 725 - protein, 22 aa, Artificial Sequence
  modified hirudin
FPRMHKGGGNGDFEEFPDAYDE

SEQ ID NO: 726 - protein, 21 aa, Artificial Sequence
  modified hirudin
FPRMHKGGNGDFEAFPDAYDE

SEQ ID NO: 727 - protein, 20 aa, Artificial Sequence
  modified hirudin
FPRMHKGNGDFEAFPDAYDE

SEQ ID NO: 728 - protein, 22 aa, Artificial Sequence
  modified hirudin
FPRMHKGGGNGDFEAFPDAYDE

SEQ ID NO: 729 - protein, 20 aa, Artificial Sequence
  modified hirudin
FPRMHKGNGDFEPFPDAYDE

SEQ ID NO: 730 - protein, 21 aa, Artificial Sequence
  modified hirudin
FPRMHKGGNGDFEPFPDAYDE

SEQ ID NO: 731 - protein, 22 aa, Artificial Sequence
  modified hirudin
FPRMHKGGGNGDFEPFPDAYDE

SEQ ID NO: 732 - protein, 19 aa, Artificial Sequence
  modified haemadin
FPRMHKGNGDFEEIEIDEE

SEQ ID NO: 733 - protein, 20 aa, Artificial Sequence
  modified haemadin
FPRMHKGGNGDFEEIEIDEE

SEQ ID NO: 734 - protein, 21 aa, Artificial Sequence
  modified haemadin
FPRMHKGGGNGDFEEIEIDEE

SEQ ID NO: 735 - protein, 19 aa, Artificial Sequence
  modified haemadin
FPRMHKGNGDFEEFEIDEE

SEQ ID NO: 736 - protein, 20 aa, Artificial Sequence
  modified haemadin
FPRMHKGGNGDFEEFEIDEE

SEQ ID NO: 737 - protein, 21 aa, Artificial Sequence
  modified haemadin
FPRMHKGGGNGDFEEFEIDEE

SEQ ID NO: 738 - protein, 19 aa, Artificial Sequence
  modified boophilin
FPRMHKGVNDFEEIPEEYL

SEQ ID NO: 739 - protein, 20 aa, Artificial Sequence
  modified boophilin
FPRMHKGGVNDFEEIPEEYL

SEQ ID NO: 740 - protein, 21 aa, Artificial Sequence
  modified boophilin
FPRMHKGGGVNDFEEIPEEYL

SEQ ID NO: 741 - protein, 19 aa, Artificial Sequence
  modified boophilin
FPRMHKGVNDFEEIEIDEE

SEQ ID NO: 742 - protein, 20 aa, Artificial Sequence
  modified boophilin
FPRMHKGGVNDFEEIEIDEE

SEQ ID NO: 743 - protein, 21 aa, Artificial Sequence
  modified boophilin
FPRMHKGGGVNDFEEIEIDEE

SEQ ID NO: 744 - protein, 19 aa, Artificial Sequence
  modified boophilin
FPRMHKGVNDFEEFEIDEE

SEQ ID NO: 745 - protein, 20 aa, Artificial Sequence
  modified boophilin
FPRMHKGGVNDFEEFEIDEE

SEQ ID NO: 746 - protein, 21 aa, Artificial Sequence
  modified boophilin
FPRMHKGGGVNDFEEFEIDEE

SEQ ID NO: 747 - protein, 19 aa, Artificial Sequence
  modified heparin co-factor Ii
FPRMHKGNGDFDYLDLEYL

SEQ ID NO: 748 - protein, 20 aa, Artificial Sequence
  modified heparin co-factor Ii
FPRMHKGGNGDFDYLDLEYL

SEQ ID NO: 749 - protein, 21 aa, Artificial Sequence
  modified heparin co-factor Ii
FPRMHKGGGNGDFDYLDLEYL

SEQ ID NO: 750 - protein, 19 aa, Artificial Sequence
  modified heparin co-factor Ii
FPRMHKGNGDFDYVDLEYL

SEQ ID NO: 751 - protein, 20 aa, Artificial Sequence
  modified heparin co-factor Ii
FPRMHKGGNGDFDYVDLEYL

SEQ ID NO: 752 - protein, 21 aa, Artificial Sequence
  modified heparin co-factor Ii
FPRMHKGGGNGDFDYVDLEYL

SEQ ID NO: 753 - protein, 19 aa, Artificial Sequence
  modified heparin co-factor Ii
FPRMHKGNGDFDYLDFEYL

SEQ ID NO: 754 - protein, 20 aa, Artificial Sequence
  modified heparin co-factor Ii
FPRMHKGGNGDFDYLDFEYL

SEQ ID NO: 755 - protein, 21 aa, Artificial Sequence
  modified heparin co-factor Ii
FPRMHKGGGNGDFDYLDFEYL

SEQ ID NO: 756 - protein, 19 aa, Artificial Sequence
  modified heparin co-factor Ii
FPRMHKGNGDFDYVDFEYL

SEQ ID NO: 757 - protein, 20 aa, Artificial Sequence
  modified heparin co-factor Ii
FPRMHKGGNGDFDYVDFEYL

SEQ ID NO: 758 - protein, 21 aa, Artificial Sequence
  modified heparin co-factor Ii
FPRMHKGGGNGDFDYVDFEYL

SEQ ID NO: 759 - protein, 19 aa, Artificial Sequence
  modified heparin co-factor Ii
FPRMHKGNGDFDYLDLDYL

SEQ ID NO: 760 - protein, 20 aa, Artificial Sequence
  modified heparin co-factor Ii
FPRMHKGGNGDFDYLDLDYL

SEQ ID NO: 761 - protein, 21 aa, Artificial Sequence
  modified heparin co-factor Ii
FPRMHKGGGNGDFDYLDLDYL

SEQ ID NO: 762 - protein, 19 aa, Artificial Sequence
  modified heparin co-factor Ii
FPRMHKGNGDFDYVDLDYL

SEQ ID NO: 763 - protein, 20 aa, Artificial Sequence
  modified heparin co-factor Ii
FPRMHKGGNGDFDYVDLDYL

SEQ ID NO: 764 - protein, 21 aa, Artificial Sequence
  modified heparin co-factor Ii
FPRMHKGGGNGDFDYVDLDYL

SEQ ID NO: 765 - protein, 19 aa, Artificial Sequence
  modified heparin co-factor Ii
FPRMHKGNGDFDYLDFDYL

SEQ ID NO: 766 - protein, 20 aa, Artificial Sequence
  modified heparin co-factor Ii
FPRMHKGGNGDFDYLDFDYL

SEQ ID NO: 767 - protein, 21 aa, Artificial Sequence
  modified heparin co-factor Ii
FPRMHKGGGNGDFDYLDFDYL

SEQ ID NO: 768 - protein, 19 aa, Artificial Sequence
  modified heparin co-factor Ii
FPRMHKGNGDFDYVDFDYL

SEQ ID NO: 769 - protein, 20 aa, Artificial Sequence
  modified heparin co-factor Ii
FPRMHKGGNGDFDYVDFDYL

SEQ ID NO: 770 - protein, 21 aa, Artificial Sequence
  modified heparin co-factor Ii
FPRMHKGGGNGDFDYVDFDYL

SEQ ID NO: 771 - skipped

SEQ ID NO: 772 - protein, 9 aa, Artificial Sequence
  synthetic peptide
FEEIPEEYL

SEQ ID NO: 773 - protein, 8 aa, Artificial Sequence
  synthetic peptide
YEPIPEEA

SEQ ID NO: 774 - protein, 12 aa, Artificial Sequence
  synthetic peptide
NGDFEEIPEEYL

SEQ ID NO: 775 - protein, 14 aa, Artificial Sequence
  synthetic peptide
APPFDFEAIPEEYL

SEQ ID NO: 776 - protein, 100 aa, Bos taurus
MKMSRLCLSVALLVLLGTLAASTPGCDTSNQAKAQRPDFCLEPPYTGPCKARIIRYFYNAKAGLCQTFVYGGCRAKRNDFKSAEDCMRTCGGAIGPWENL

SEQ ID NO: 777 - protein, 24 aa, Artificial Sequence
  modified hirulog
  MISC_FEATURE (1)..(1): F=D-phenylalanine
  misc_feature (5)..(5): Xaa can be any naturally occurring amino acid
  misc_feature (7)..(7): Xaa can be any naturally occurring amino acid
FDPRXHXGGGAPPFDFEAIPEEYL

(19) United States
(12) Patent Application Publication
(10) Pub. No.: US 2012/0135931 A1
(43) Pub. Date: May 31, 2012

(54) METHOD OF MODIFYING SERINE PROTEASE INHIBITORS

(75) Inventors: R. Manjunatha Kini, Singapore (SG); Cho Yeow Koh, Singapore (SG); Kunchithapadam Swaminathan, Singapore (SG); Kumar Sundramurthy, Singapore (SG)

(73) Assignee: NATURAL ENVIRONMENT RESEARCH COUNCIL, Swindon (GB)

(21) Appl. No.: 13/319,051

(22) PCT Filed: May 5, 2010
(86) PCT No.: PCT/GB10/00889

§ 371 Date: Feb. 21, 2012

Related U.S. Application Data
(60) Provisional application No. 61/250,412, filed on Oct. 9, 2009.

(30) Foreign Application Priority Data
May 5, 2009 (GB) 0907698.5

(51) Int. Cl. A61K 38/58; C07K 14/815; C07K 7/06; C07K 14/81; C12N 1/21; C12N 1/19; C12N 5/10; C12N 15/15; C12N 15/113; C12N 15/63; A61K 38/57; A61P 7/00; A61K 38/08; A61K 38/10; C07K 1/107; C07K 7/08

(52) U.S. Cl. 514/13.7; 530/326; 530/324; 530/327; 530/350; 530/393; 530/381; 530/392; 435/252.33; 435/254.2; 435/325; 536/23.5; 536/24.5; 435/320.1; 514/20.3; 530/409; 530/402

(57) ABSTRACT

The present invention relates to a method of modifying serine protease inhibitors in order to acquire or enhance any one of a variety of desired properties, including extent of inhibition, maintenance of inhibition following cleavage of the serine protease inhibitor by the target serine protease, speed of binding to the serine protease, neutralisation, and binding affinity. The present invention also relates to the products of such modifications and the uses of such products, in particular, their use in therapy.

### FIELD OF THE INVENTION

The present invention relates to methods of modifying serine protease inhibitors in order to acquire or enhance any one of a variety of desired properties. The present invention also relates to the products of such modifications and the uses of such products, in particular, their use in therapy.

### BACKGROUND TO THE INVENTION

Serine proteases, also known as serine endopeptidases, are protein digesting enzymes containing a serine residue at the active site. These enzymes are widespread in nature, and play a part in a wide range of biological functions including digestion, blood clotting, the immune system and inflammation.

Due to the widespread distribution and function of serine proteases, inhibitors for these enzymes are common. Many proteinaceous serine protease inhibitors can be found in nature, and many synthetic, chemical serine protease inhibitors have been developed for use in research and therapy.

Thrombin is a member of the serine protease family which plays a central role in blood coagulation; the process by which circulating zymogens of serine proteases are sequentially activated by limited proteolysis to produce fibrin clots in response to vascular injury. Thrombin interacts with most of the zymogens and their cofactors, playing multiple procoagulant and anticoagulant roles in blood coagulation (Huntington (2005), and Di Cera (2003)). As a procoagulant protease, the first traces of thrombin generated in the initiation phase activate factor V (FV) and factor VIII (FVIII) to provide positive feedback leading to thrombin burst. Thrombin can also activate factor XI, triggering the intrinsic pathway. Thrombin cleaves fibrinogen to fibrin, forming insoluble clots. Fibrin polymers are further strengthened and stabilized through covalent cross-linking driven by thrombin activated factor XIII. Thrombin also contributes to the generation of a platelet plug, possibly through two mechanisms: (a) it activates platelets by interacting with protease-activated receptors (PARs) and glycoprotein V; and (b) it prevents destabilization of the platelet plug, by inactivating ADAMTS13, a disintegrin and metalloprotease with a thrombospondin type 1 motif, that cleaves von Willebrand factor (VWF). As an anticoagulant protease, thrombin activates protein C (APC) in the presence of the cofactor thrombomodulin. APC inactivates factor Va (FVa) and factor VIIIa (FVIIIa), down-regulating the generation of thrombin (Huntington (2005), Di Cera (2003), Davie et al. (1991), Davie (2003), and Lane et al. (2005)).

Due to its central role, thrombin is a prime target for inhibition in order to control the coagulation cascade, and many thrombin inhibitors have been used in therapy and research for many years. Heparin is the archetypal thrombin inhibitor, and functions as an indirect inhibitor of thrombin, meaning that it acts via an anti-thrombin complex and does not interact directly with the active site of thrombin. Indirect thrombin inhibitors can only interact with soluble thrombin and are therefore unable to inhibit thrombin once a clot has formed.

More recently, a number of direct thrombin inhibitors including hirudin, bivalirudin, argatroban and dabigatran etexilate have been isolated and/or developed. These have the therapeutic advantage of being able to inhibit thrombin in both its soluble and fibrin-bound form. However, such direct inhibitors have certain properties which are far from optimal. For example, hirudin causes risk of bleeding, pharmacokinetics that depends on renal function, lack of antidote, immunogenicity and rebound hypercoagulability. Bivalirudin, which is eliminated by a combination of proteolysis and renal routes, has negligible immunogenic potential, but still has sub-optimal therapeutic properties.

In view of the therapeutic importance of serine protease inhibitors, there is a need to identify additional serine protease inhibitors which display improved properties. In particular, there is a need to identify direct thrombin inhibitors with the ability to inhibit thrombin in both its soluble and fibrin-bound form but without the disadvantages associated with currently available direct thrombin inhibitors.

### SUMMARY OF THE INVENTION

The present invention provides modified serine protease inhibitors, methods of producing modified serine protease inhibitors, and methods of using modified serine protease inhibitors, e.g., for inhibiting a target serine protease in a subject.

Accordingly, in a first aspect, the invention provides a method of producing a modified serine protease inhibitor (SPI) displaying enhanced inhibition of a target serine protease (SP), comprising modifying the SPI such that binding of the SPI to its target SP displaces one or more of the amino acid residues in the catalytic triad of the target SP, or one or more atoms of said amino acid residues.

In one embodiment of this aspect, the method comprises the introduction of one or more amino acid residues into the SPI which are capable of displacing one or more of the amino acid residues of the catalytic triad of the target SP, or one or more atoms of said amino acid residues. In another embodiment, method produces a modified SPI which displays a prolonged duration of inhibition. In one embodiment, said one or more introduced amino acid residues are introduced by substitution or insertion.

In another embodiment of this aspect, said one or more amino acid residues capable of displacing one or more of the residues of the catalytic triad of the target SP, or one or more atoms thereof comprises a histidine residue. In one embodiment, said one or more introduced amino acids comprises a methionine-histidine sequence. In a further embodiment, said one or more introduced amino acids comprises a methionine-histidine-lysine sequence. In another embodiment, said one or more introduced amino acids comprises a methionine-histidine-lysine-threonine sequence. In one embodiment, the one or more residues in the catalytic triad of the target serine protease which is displaced comprises the catalytic serine residue.

In another embodiment of this aspect, the method further contains a step of modifying the SPI so that it is capable of being neutralised, comprising the introduction of an area of ionic charge into the SPI, wherein the area of ionic charge is capable of interacting with an area of opposite ionic charge on a neutralising agent. In one embodiment, said introduced area of ionic charge is introduced towards the carboxy-terminus of the SPI. In another embodiment, said introduced area of ionic charge is an area of anionic charge. In one embodiment, said introduced area of ionic charge comprises one or more acidic residues. In another embodiment, said one or more acidic residues comprises one or more glutamine residues. In a further embodiment, said neutralising agent is protamine sulphate.

In an exemplary embodiment of the foregoing methods, the SPI is a thrombin inhibitor. In another exemplary embodiment, the SPI is selected from the group consisting of any one of SEQ ID NOs: 14 and 17-153.

In another aspect, the invention provides a modified SPI obtainable or obtained by any of the foregoing methods, or a fragment or functional equivalent thereof. In one embodiment, said modified SPI is a thrombin inhibitor. In an exemplary embodiment, the modified SPI contains the following consensus sequence: N-terminal peptide) —X₁—H—X₂-(G)ₙ- (exosite I binding peptide) (SEQ ID NO: 771).

In another aspect, the invention provides a modified SPI which displays enhanced inhibition of a target SP, wherein the binding of the SPI to its target SP displaces one or more of the amino acid residues in the catalytic triad of the target SP, or one or more atoms of said amino acid residues. In one embodiment of this aspect, the modified SPI comprises one or more amino acid residues which are capable of displacing one or more of the amino acid residues of the catalytic triad of the target SP, or one or more atoms of said amino acid residues. In another embodiment, the modified SPI displays a prolonged duration of inhibition.

In another embodiment of this aspect, the one or more amino acid residues capable of displacing one or more of the residues of the catalytic triad of the target SP, or one or more atoms thereof comprises a histidine residue. In another embodiment, the one or more amino acid residues capable of displacing one or more of the residues of the catalytic triad of the target SP comprises a methionine-histidine sequence. In another embodiment, the one or more amino acid residues capable of displacing one or more of the residues of the catalytic triad of the target SP comprises a methionine-histidine-lysine sequence. In a further embodiment, the one or more amino acid residues capable of displacing one or more of the residues of the catalytic triad of the target SP comprises a methionine-histidine-lysine-threonine sequence. In another embodiment, the one or more amino acid residues in the catalytic triad of the target serine protease which is displaced comprises the catalytic serine residue.

In another embodiment of this aspect, the modified SPI further comprises an area of ionic charge, wherein the area of ionic charge is capable of interacting with an area of opposite ionic charge on a neutralising agent. In one embodiment, the area of ionic charge is positioned towards the carboxy-terminus of the SPI. In another embodiment, the area of ionic charge is an area of anionic charge. In one embodiment, the area of ionic charge comprises one or more acidic residues. In another embodiment, the one or more acidic residues comprise one or more glutamine residues. In an exemplary embodiment, the neutralising agent is protamine sulphate. In another exemplary embodiment, the foregoing modified SPIs are thrombin inhibitors. In one embodiment, the modified SPIs contain the following consensus sequence: N-terminal peptide) —X₁—H—X₂-(G)ₙ- (exosite I binding peptide) (SEQ ID NO: 771).

In another aspect, the invention provides a modified SPI comprising a sequence selected from any one of SEQ ID NOs: 158-770, or a fragment or functional equivalent thereof. In a further aspect, the invention provides a modified SPI consisting of a sequence selected from any one of SEQ ID NOs: 158-770, or a fragment or functional equivalent thereof.

In another aspect, the invention provides a nucleic acid molecule encoding a modified SPI described herein. In another aspect, the invention provides an anti-sense nucleic acid molecule which hybridises under high stringency hybridisation conditions to nucleic acid molecule encoding a modified SPI described herein.

In one embodiment, the invention comprises a vector containing a nucleic acid sequence encoding a modified SPI described herein, or an anti-sense nucleic acid molecule which hybridizes under high stringency hybridisation conditions to nucleic acid molecule encoding a modified SPI described herein. In another embodiment, the invention provides a host cell containing the foregoing vector, and/or the foregoing nucleic acid molecule.

In another aspect, the invention provides a method of inhibiting a target SP comprising administering a modified SPI described herein. In another aspect, the invention provides a method of treating a subject suffering from a coagulopathy or preventing a subject from developing a coagulopathy comprising administering a modified SPI, e.g., a thrombin inhibitor, described herein. In another embodiment, the invention provides a method of neutralising thrombin inhibition in a subject comprising administering a modified thrombin inhibitor described herein, and subsequently administering to the subject an amount of protamine sulphate sufficient to result in neutralisation of the thrombin inhibition.

Other features and advantages of the invention will be apparent from the following detailed description, and from the claims.

### DESCRIPTION OF THE INVENTION

The present invention provides a method of producing a modified serine protease inhibitor (SPI) displaying enhanced inhibition of a target serine protease (SP) comprising modifying the SPI such that binding of the SPI to its target SP displaces one or more of the amino acid residues in the catalytic triad of the target SP, or one or more atoms of said amino acid residues.

As discussed above, serine proteases are peptide cleaving enzymes. It is accepted in the art that these enzymes act via a catalytic triad, present in the active site of the enzyme, and comprising a serine residue, a histidine residue and an aspartate residue. The function of the histidine and aspartate residues is to activate the serine residue through a charge relay system, making it nucleophilic and capable of cleaving the scissile bond of the substrate. The interaction between the residues of the catalytic triad in a typical serine protease is shown in FIG. 1.

The inventors have surprisingly established that in addition to sterically blocking the active site in the manner of a conventional competitive inhibitor, variegin, a direct inhibitor of the serine protease thrombin, also acts by disrupting the interaction between the residues of the catalytic triad of thrombin, thereby inhibiting its catalytic activity. Variegin is a protein having the amino acid sequence shown in SEQ ID NO: 1. It is a tick-derived protein first described in WO03/091284. The ability of variegin to bind thrombin is described in WO08/155,658. However, neither document suggests that variegin acts to disrupt interactions between amino acids in the catalytic triad of thrombin. The contents of WO03/091284 and WO08/155,658 are incorporated herein by reference in their entirety.

FIG. 9A depicts the positioning of the residues of the catalytic triad of thrombin and the interaction between these residues which functions to activate the catalytic serine residue. FIG. 9B depicts the residues of variegin which interact with the catalytic triad, and the effect of this interaction on the positioning of the residues of the catalytic triad. This Figure diagrammatically shows the unexpected finding that the histidine residue of variegin functions to displace the γO of serine by 1.1 Å, disrupting the interaction between the serine and histidine residues of the catalytic triad, and dramatically reducing the activity of thrombin. As far as the inventors are aware, variegin is the first SPI that has been found to act by displacing one or more of the amino acid residues in the catalytic triad of the target SP, or one or more atoms of said amino acid residues.

The realisation by the inventors that the potent anti-thrombin activity of variegin is at least partly due to the disruption of the catalytic triad in the active site of thrombin and the mechanism by which this is achieved can be applied to other serine protease inhibitors including thrombin inhibitors. In particular, the properties of known serine protease inhibitors can be improved by modification so that they disrupt interactions between residues of the catalytic triad of the target serine protease. Such modifications function to improve the properties of the serine protease inhibitor, and overcome many of the disadvantages of existing serine protease inhibitors, in particular known direct thrombin inhibitors.

The term “target serine protease”, or “target SP” relates to the serine protease which is normally inhibited by a given serine protease inhibitor. One example of a target SP is thrombin. Further examples of target SPs according to the invention include the coagulation factors FXa, FVIIa, FXIIa, FXIa, and FIXa.

The serine protease inhibitor or SPI which is modified by the method of the invention may be a direct SPI or an indirect SPI. The term “direct SPI” means that the SPI interacts with its target SP at the active site of the SP without being present as part of an anti-SP complex or acting through an intermediate. The term “indirect SPI” means that the SPI does not interact directly with the active site of the target SP. An indirect SPI may interact with a site on the target SP which is distinct from the active site, or the indirect SPI may interact with the active site or another site on the target SP through an anti-SP complex comprising the indirect SPI.

Examples of SPIs that may be modified by the method of the invention include hirulog (SEQ ID NO: 14), Kunitz/BPTI-type inhibitors (e.g. bovine pancreatic trypsin inhibitor, shown in SEQ ID NO: 776), hirudin-related thrombin inhibitors, serpins, heparin cofactors, α1-antitrypsin-like serpins, kazal type direct inhibitors, and kunitz type/STI (sybean trypsin inhibitor) inhibitors. Further examples of SPIs which may be modified by the method of the invention are given in SEQ ID NOs: 17-153. By “displaced” is meant that the amino acid residue in the target SP or one or more atoms within the amino acid residue occupy a conformation in space which is different from that which it would naturally adopt in the absence of any outside influences. It should be appreciated that such displacement may be in any direction.

In one aspect, the displacement may be such that the interaction between the amino acid residues of the catalytic triad of the target SP is disrupted. Such disruption may be complete, i.e. the residues of the catalytic triad no longer interact, or it may be partial, i.e. the interaction between the residues is only 90%, 80%, 70%, 60%, 50%, 40%, 30%, 20%, 10%, or less as strong as it would have been if one or more of the residues of the catalytic triad was not displaced. The presence of an interaction between the amino acid residues of the catalytic triad may be measured by any method known in the art, e.g crystallography or NMR, computational methods including but not limited to molecular mechanics, molecular dynamics and docking, hydrogen/deuterium exchange and mass spectroscopy.

The displacement of one or more residues of the catalytic triad of the target SP, or one or more atoms of said amino acid residues may disrupt the charge replay system of the catalytic triad of the target SP.

In one aspect of the invention, the displacement of one or more of the residues of the catalytic triad of the target SP may comprise the displacement of the serine residue of the catalytic triad. In one aspect, the γO atom of the serine residue of the catalytic triad may be displaced. In another aspect the βC of the serine residue of the catalytic triad may be displaced. In another aspect the γC of the serine residue of the catalytic triad may be displaced.

In one aspect, the atom of the serine residue of the catalytic triad may be displaced by 0.1 Å. In further aspects, the atom of the serine residue of the catalytic triad may be displaced by 0.2 Å, 0.3 Å, 0.4 Å, 0.5 Å, 0.6 Å, 0.7 Å, 0.8 Å, 0.9 Å, 1.0 Å, 1.1 Å, 1.2 Å, 1.3 Å, 1.4 Å, 1.5 Å, 1.6 Å, 1.7 Å, 1.8 Å, 1.9 Å, 2.0 Å, 2.5 Å, 3.0 Å, or more.

In one aspect of the invention, the displacement of one or more of the residues of the catalytic triad may comprise the displacement of the histidine residue of the catalytic triad. In one aspect, the γC atom of the histidine residue of the catalytic triad may be displaced. In another aspect the δC atom of the histidine residue of the catalytic triad may be displaced. In another aspect the εN₂ atom of the histidine residue of the catalytic triad may be displaced. In another aspect the atom of the histidine residue of the catalytic triad may be displaced. In another aspect the δN₁ atom of the histidine residue of the catalytic triad may be displaced. In another aspect the βC atom of the histidine residue of the catalytic triad may be displaced. In another aspect the αC atom of the histidine residue of the catalytic triad may be displaced.

In one aspect, the atom of the histidine residue of the catalytic triad may be displaced by 0.1 Å. In further aspects, the atom of the histidine residue of the catalytic triad may be displaced by 0.2 Å, 0.3 Å, 0.4 Å, 0.5 Å, 0.6 Å, 0.7 Å, 0.8 Å, 0.9 Å, 1.0 Å, 1.1 Å, 1.2 Å, 1.3 Å, 1.4 Å, 1.5 Å, 1.6 Å, 1.7 Å, 1.8 Å, 1.9 Å, 2.0 Å, 2.5 Å, 3.0 Å, or more.

In one aspect of the invention, the displacement of one or more of the residues of the catalytic triad may comprise the displacement of the aspartate residue of the catalytic triad. In one aspect the γO atom of the aspartate residue of the catalytic triad may be displaced. In another aspect the βC atom of the aspartate residue of the catalytic triad may be displaced. In another aspect the αC atom of the aspartate residue of the catalytic triad may be displaced. In another aspect the γC atom of the aspartate residue of the catalytic triad may be displaced. In another aspect the δO₁ atom of the aspartate residue of the catalytic triad may be displaced. In another aspect the δO₂ atom of the serine residue of the catalytic triad may be displaced.

In one aspect, the atom of the aspartate residue of the catalytic triad may be displaced by 0.1 Å. In further aspects, the atom of the aspartate residue of the catalytic triad may be displaced by 0.2 Å, 0.3 Å, 0.4 Å, 0.5 Å, 0.6 Å, 0.7 Å, 0.8 Å, 0.9 Å, 1.0 Å, 1.1 Å, 1.2 Å, 1.3 Å, 1.4 Å, 1.5 Å, 1.6 Å, 1.7 Å, 1.8 Å, 1.9 Å, 2.0 Å, 2.5 Å, 3.0 Å, or more.

The displacement of one or more amino acid residues of the target SP, or one or more atom of said amino acid residues may be measured by any method known in the art, e.g crystallography or NMR, computational methods including but not limited to molecular mechanics, molecular dynamics and docking, hydrogen/deuterium exchange and mass spectroscopy.

In one aspect of the invention, the SPI is a protein and the modification comprises the introduction of one or more amino acid residues into the SPI which are capable of displacing one or more of the amino acid residues in the catalytic triad of the target SP, or one or more atoms of said amino acid residues. These amino acid residues may displace the amino acid residues in the catalytic triad by interacting with them. The introduced amino acid residues may comprise a histidine residue. Such a histidine residue may be present as part of any other sequence which may be introduced into the SPI in addition to the histidine residue. In one embodiment, the introduced amino acids may comprise a methionine-histidine (MH) sequence. In another embodiment the introduced amino acids may comprise a methionine-histidine-lysine (MHK) sequence. In another embodiment the introduced amino acid may comprise a methionine-histidine-arginine (MHR) sequence. In a further embodiment, the introduced amino acids may comprise a methionine-histidine-lysine-threonine (MHKT) sequence. In another embodiment the introduced amino acids may comprise a methionine-histidine-arginine-threonine (MHRT) sequence. In another embodiment the introduced amino acids may comprise a methionine-histidine-lysine-threonine-alanine (MHKTA) sequence. In another embodiment the introduced amino acids may comprise a methionine-histidine-arginine-threonine-alanine (MHRTA) sequence.

Alternative amino acid residues may also be introduced provided they are capable of displacing one or more residues of the catalytic triad of the target SP, or one or more atoms thereof. When considering the MHKT sequence, for example, leucine, isoleucine, valine or alanine may be used in place of methionine and/or lysine, arginine or tyrosine may be used in place of histidine, and/or serine or alanine may be used in place of threonine.

In another aspect the introduced one or more amino acid residues may comprise a linker region. In another aspect the linker region may comprise one or more amino acids e.g. glycine or alanine. In a further aspect the linker region may comprise one, two, three, four, or five glycine residues. In another aspect, the linker region may consist of one, two, three, four, or five glycine residues.

In aspects of the invention where the target SP is thrombin, the method of producing a modified SPI may involve the introduction or maintenance of a peptide sequence which is capable of interacting with exosite I of thrombin. By maintenance of such a peptide sequence is meant that the peptide sequence is already present in the SPI sequence prior to modification, and that this sequence is not disrupted or removed by the modification.

In one aspect, the peptide sequence which is capable of interacting with exosite I of thrombin may comprise one of the following sequences:

FEEIPEEYL;
YEPIPEEA;
NGDFEEIPEEYL;
or
APPFDFEAIPEEYL.

The modified SPI produced by any of the methods of the invention displays enhanced inhibition of its target SP compared to the unmodified SPI.

It will be apparent to a person skilled in the art that any one of a variety of assays may be used to determine the extent of SP inhibition, and to confirm that the modification enhances inhibition of a target SP. By way of example, where the SP is thrombin such an assay may be an amidolytic assay, wherein the formation of p-nitroaniline following incubation of thrombin with the modified thrombin inhibitor in the presence of S2238 is detected.

The modified SPIs of the invention may have an IC₅₀ of less than 30 nM, less than 25 nM, less than 20 nM, less than 15 nM, less than 14 nM, less than 13 nM, less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM. SPIs produced according to the method of the invention may have a Ki of less than less than 15 nM, less than 10 nM, less than 5 nM, less than 1 nM, less than 750 pM, less than 500 pM, less than 400 pM, less than 300 pM, less than 250 pM, less than 200 pM, less than 150 pM, less than 100 pM, less than 50 pM, less than 30 pM, less than 25 pM, less than 20 pM, less than 15 pM, less than 10 pM, less than 5 pM, less than 1 pM, or less than 100 pM.

It has been established that conventional direct SPIs act by binding to at least the active site of the target SP, where they may be cleaved by the target SP, therefore competing with the substrate of the target SP for binding, and competitively inhibiting the target SP. An example of a SPI which acts in this manner is hirulog-1. Although such competitive inhibition can be an effective inhibitory mechanism, it has certain drawbacks, in particular in relation to the transient nature of the inhibition, and the rapid depletion of the SPI.

As described in J. Biol. Chem., 2007, 282(40) 29101-29113 (Cho Yeow Koh, Maria Kazimirova, Adama Trimnell, Peter Takac, Milan Labuda, Patricia A. Nuttall, and R. Manjunatha Kini), variegin functions as a competitive inhibitor in the same manner as other direct SPIs. However, upon cleavage of variegin by thrombin a fragment of variegin known as MH22, shown as SEQ ID NO: 3, remains bound to thrombin, and functions as a non-competitive inhibitor of thrombin. This increases the inhibitory potential of variegin, and overcomes some of the disadvantages of other direct SPIs. Upon analysing the crystal structure of variegin bound to thrombin, the inventors have surprisingly discovered that MH22 binds to the active site of thrombin. This is unusual since non-competitive inhibitors generally bind at a site distinct from the enzyme active site. Furthermore, the crystal structure revealed that the histidine residue of variegin which is responsible for displacing one or more of the residues of the catalytic triad of thrombin is part of the MH22 sequence, and that this variegin fragment therefore disrupts the catalytic triad of thrombin, following cleavage of variegin, resulting in an increased duration of inhibition.

The method of the invention may thus result in a modified SPI that remains bound to the target SP following cleavage of the modified SPI by the target SP. Such modified SPIs display an increased duration of inhibition.

By the term “prolonged duration of action” is meant that the duration of inhibition of the target SP is increased relative to the duration of inhibition using a non-modified SPI. In one aspect the duration of action may be increased at least two-fold. In another aspect the duration of action may be increased at least three-fold, at least four-fold, at least five-fold, at least six-fold, at least seven-fold, at least eight-fold, at least nine-fold, or more relative to the duration of inhibition using a non-modified SPI. The duration of inhibition by the modified SPI may be greater than 5 minutes, great than 10 minutes, greater than 15 minutes, greater than 20 minutes, greater than 25 minutes, greater than 30 minutes, greater than 1 hour, greater than 2 hours, greater than 3 hours, greater than 4 hours, greater than 5 hours, greater than 6 hours, greater than 12 hours, greater than 1 day, greater than 2 days, greater than 3 days or more. Methods for determination of the extent of inhibition of the target SP have been described above. In certain aspects of the invention, the one or more introduced amino acid residues described above may be positioned towards the amino-terminus of the portion of the modified SPI retained in the active site following cleavage by the target SP.

By “towards the amino-terminus” is intended to mean that the one or more introduced residues are within five amino acids of the amino-terminus of the retained portion of the SPI following cleavage by the target SP. In certain aspects, the one or more introduced residues may be within one residue, within two residues, within three residues, within four residues or within five residues of the amino-terminus of the portion of the modified direct SPI retained in the active site following cleavage by the target SP.

It will be apparent to the skilled person that in order for the one or more introduced residues to be “towards the amino-terminus” of the portion of the modified direct SPI retained in the active site following cleavage by the target SP, the one or more introduced residues must be within five residues of the cleavage site of the modified direct SPI.

In one aspect, the method of the invention may comprise the additional or alternative step of modifying an SPI to make it capable of being neutralised, comprising introducing an area of ionic charge into the SPI, wherein the area of ionic charge is capable of interacting with an area of opposite ionic charge on a neutralising agent such that the resulting ionic interaction between the modified SPI and the neutralising agent neutralises the inhibitory activity of the modified SPI, such that the modified SPI no longer displaces one or more of the amino acid residues in the catalytic triad of the target SP, or one or more atoms of said amino acid residues.

Based on the sequence of variegin and data obtained from the crystal structure of variegin bound to thrombin, the inventors have surprisingly discovered that the inhibitory activity of variegin can be neutralised. This neutralisation mechanism is based on the finding of an ionic interaction between an area of ionic charge on the carboxy-terminus of variegin, and an area of opposite ionic charge on a neutralisation agent. The ionic interaction between variegin and the neutralising agent appears to neutralise the inhibitory activity of variegin by disrupting an ionic interaction between an area of ionic charge on variegin and an area of opposite ionic charge on thrombin. From analysis of the structure of variegin bound to thrombin, it is thought that the area of ionic charge on thrombin is within exosite-I.

This information allows other SPIs to be modified so they are capable of being neutralised. Given the therapeutic uses of SPIs, which are discussed above, modified SPIs that are capable of being neutralised will have considerable therapeutic benefits. By “capable of being neutralised” is meant that the activity of the SPI is able to be wholly or partially undone by the addition of a neutralising agent, i.e. the activity of the SP is able to be restored upon addition of a neutralising agent. Within this definition, 10%, 20%, 30%, 40%, 50%, 60%, 70%, 80%, 90%, 95%, 99%, or 100% of the SP activity may be restored. Taken another way, 90%, 80%, 70%, 60%, 50%, 40%, 30%, 20%, 10%, 5%, 1% of the inhibitory activity of the SPI may remain following disruption of the ionic interaction between the modified SPI and the target SP.

It will be apparent to a person skilled in the art that the action of most inhibitors which act by binding to their target, including but not limited to competitive inhibitors, will be neutralisable to some extent due to the inherent equilibrium which is set up between bound and unbound inhibitor. This equilibrium position is altered upon the addition of a certain substance; described herein as a “neutralising agent”, which acts to bias the equilibrium in favour of unbound inhibitor, and therefore undo the inhibitory effects of the inhibitor. However, in the case of a “non-neutralisable” inhibitor, this equilibrium is heavily biased towards bound inhibitor, and the addition of the neutralising agent does not upset the equilibrium balance. FIG. 8 shows the equilibrium scheme for the binding of variegin to thrombin. This scheme is provided by way of example only.

In the context of the invention, the term “neutralisation” is intended to relate only to neutralisation brought about by the addition of a neutralising agent, which disrupts the equilibrium balance, and not to inherent neutralisation which is a by-product of such an inherent equilibrium.

The neutralising agent may function to neutralise the inhibitory activity of the modified SPI by possessing an area of ionic charge opposite to the area of ionic charge introduced onto the modified SPI. The formation of an ionic interaction between the modified SPI and the neutralising agent may result in the disruption of an ionic interaction between the area of ionic charge on the modified SPI and an area of opposite ionic charge on the target SP. The area of ionic charge on the target SP may be within one of the exosites. In another aspect, the area of ionic charge may be within exosite-L In one aspect of the invention, the area of ionic charge on the neutralising agent may be an area of cationic charge. In this aspect of the invention, the area of ionic charge introduced into the SPI by the method of the invention may therefore be an area of anionic charge. In another aspect of the invention, the area of ionic charge on the target SP may be an area of cationic charge.

The area of ionic charge introduced into the SPI may be introduced towards the carboxy-terminus of the SPI. By “towards the carboxy-terminus” is intended to mean that the introduced area of ionic charge is located within ten amino acids of the carboxy-terminus of the modified SPI. The introduced area of ionic charge may be within one residue, within two residues, within three residues, within four residues, within five residues, within six residues, within seven residues, within eight residues, within nine residues or within ten residues of the carboxy-terminus of the modified SPI. The neutralising agent may be a cationic substance. Such a cationic substance may compete with the SP for binding to the area of anionic charge on the target SPI, resulting in a displacement of the modified SPI, and a loss of inhibition of the target SP. The neutralising agent may be a cationic peptide, such as protamine sulphate.

The area of ionic charge which is introduced into the SPI may comprise one or more acidic residues. The one or more acidic residues may comprise one, two, three, four, five or more acidic residues. The term “acidic residue” may comprise aspartate and glutamate. The one or more acidic residues may comprise a glutamine residue and/or an aspartate residue.

A specific example of an area of ionic charge that may be introduced comprises two glutamate amino acid residues and two aspartate amino acid residues. In a further specific example an area of ionic charge that may be introduced comprises the sequence glu-glu-X-X-asp-asp, where X is any amino acid residue. In a still further example, a region of ionic charge that may be introduced comprises the sequence glu-glu-tyr-lys-asp-asp.

### General

Certain general aspects of the invention will now be described. The features and methods included in this section are applicable to any of the methods of the invention described above.

As described in the preceding sections, the methods of the invention may comprise the introduction of one or more residues into the SPI. In one aspect, such introduced residues may be introduced by insertion. In another aspect, residues may be introduced by substitution.

Methods of substitution or insertion will be apparent to a person skilled in the art. By way of example, but not limitation, these may include site-directed mutagenesis, PCR mutagenesis, transposon mutagenesis, directed mutagenesis, insertional mutagenesis, targeted mutagenesis, and chemical protein synthesis (Sambrook et al. (2000)).

In certain aspects of the invention the method of modifying the SPI may comprise one or more additional steps. In certain embodiments, one or more of the additional steps may be initial additional steps, meaning that these steps take place before other steps of the method of modification.

In one aspect, the method of the invention may comprise the additional step of analysing the structure of the SPI to determine the modification to be made to the SPI. The analysis may involve analysis of the amino acid sequence of the SPI and/or computational modelling of the structure of the SPI. Additionally or alternatively, the method may involve analysis of the structure of the SP or of the SPI bound to the SP. Such a structure may be in the form of a crystal structure, an infra-red spectrum, circular dichroism data, an ultra-violet spectrum, NMR spectroscopy, computational methods including but not limited to molecular mechanics, molecular dynamics and docking or hydrogen/deuterium exchange and mass spectroscopy.

The analysis may involve determination of the region of the SPI which is responsible for the interaction between the SP and the SPI which will be altered according to the method of modification of the SPI. For example, the method of modifying a SPI to enhance inhibition of a target SP described above may comprise the initial step of identifying residues in the SPI that interact with the catalytic triad of the target SP. The amino acid residues that interact with the catalytic triad may then be modified to displace one or more residues of the catalytic triad, or one or more atoms thereof, e.g. by the introduction of an MHKT sequence at this location.

The invention may comprise the additional step of analysing the structure of the target SP to determine the modification to be made to the SPI. The analysis may involve determination of the region and/or the residues of the target SP which is responsible for the interaction between the target SP and the SPI which will be altered according to the method of modification of the SPI. The analysis may involve structural analysis of the SP in the form of a crystal structure, an infra-red spectrum, circular dichroism data, an ultra-violet spectrum, an NMR spectrum or data from a computational method.
The analysis described above may involve comparing the structure of the SPI with the structure of another SPI, whose structure and/or function has previously been analysed. Such analysis may be performed on any data produced in relation to the SPI to be modified and another SP. In particular, such data may be derived from a crystal structure, an infra-red spectrum, circular dichroism data, or an ultra-violet spectrum, and NMR spectrum or data from a computational method.
In a further aspect of the invention, the SPI whose structure and/or function has previously been analysed may be a thrombin inhibitor. In yet a further aspect of the invention, the SPI whose structure and/or function has previously been analysed may be variegin.

In one aspect of the invention, the SPI which is to be modified by the method of the invention may be a thrombin inhibitor. According to another aspect of the invention, the SPI which is to be modified by the method of the invention may be selected from the group consisting of hirulog (SEQ ID NO: 14), Kunitz/BPTI-type inhibitors (e.g. bovine pancreatic trypsin inhibitor, shown in SEQ ID NO: 776), hirudin-related thrombin inhibitors, serpins, heparin cofactors, α1-antitrypsin-like serpins, kazal type direct inhibitors, and kunitz type/STI (soybean trypsin inhibitor) inhibitors. In another aspect, the SPI which is to be modified by the method of the invention may be any one of SEQ ID NOs: 17-153. Modified SPIs

The invention also includes modified SPIs obtainable or obtained by the methods of the invention.

In another aspect, the invention relates to modified SPIs which are obtained by any means. For example, the modified SPIs obtainable by the methods of the invention may also be produced by any methodology known in the art. Exemplary techniques useful for producing the modified SPIs described herein include chemical peptide synthesis, solid-phase or solution-phase peptide synthesis, in vitro translation from a nucleic acid molecule encoding a modified SPI, or cell-based production methods employing prokaryotic or eukaryotic recombinant expression systems. In an exemplary embodiment, a modified SPI is a polypeptide comprising a sequence set forth in any of SEQ ID NOs: 158-770. Such modified SPI compositions may be used in the methods of the invention, including methods of inhibiting a SP, as described below. In one aspect of the invention, the modified SPI obtainable or obtained by the methods of the invention may be a modified thrombin inhibitor.

In one aspect of the invention, the modified SPI obtainable or obtained by the methods of the invention may be a modified version of hirulog (SEQ ID NO: 14), Kunitz/BPTI-type inhibitors (e.g. bovine pancreatic trypsin inhibitor, shown in SEQ ID NO: 776), hirudin-related thrombin inhibitors, serpins, heparin cofactors, α1-antitrypsin-like serpins, kazal type direct inhibitors, and kunitz type/STI (sybean trypsin inhibitor) inhibitors. In another aspect, the SPI which is modified by the method of the invention may be any one of SEQ ID NOs: 17-153.

Modified versions of hirulog obtainable or obtained by methods of the invention may have the following consensus sequence:

(N-terminal peptide) —X₁—H—X₂-(G)ₙ- (exosite I binding peptide) (SEQ ID NO: 771)

In one aspect, the N-terminal peptide may comprise the sequence phenylalanine, phenylalanine-proline, phenylalanine-proline-arginine, or phenylalanine-proline, lysine.

In another aspect, the amino-terminal phenylalanine residue may be a modified phenylalanine residue. In one example this modified residue may be a _{D}-phenylalanine residue.

In one aspect, X₁ may be any amino acid. In another aspect, X₁ may be a methionine residue.

In one aspect, X₂ may be any amino acids. In another aspect, X₂ may be lysine or arginine residue.

In one aspect n may be one or more glycine amino acid residues. In another aspect n may be two, three, four, five or more glycine amino acid residues.

In one aspect the modified SPI may include one or more sulphated amino acid residues. In another aspect, the SPI may include one or more sulphated tyrosine residues.

In one aspect, the exosite I binding peptide may comprise one of the following sequences:

| | |
|---|---|
| FEEIPEEYL; | (SEQ ID NO: 772) |
| YEPIPEEA; | (SEQ ID NO: 773) |
| NGDFEEIPEEYL; | (SEQ ID NO: 774) |
| or | |
| APPFDFEAIPEEYL. | (SEQ ID NO: 775) |

The exosite I binding peptide may further comprise an area of ionic charge comprising one or more acidic residues. The one or more acidic residues may comprise one, two, three, four, five or more acidic residues. The term “acidic residue” may comprise aspartate and glutamate. The one or more acidic residues may comprise a glutamine residue and/or an aspartate residue. The area of ionic charge may comprise two glutatmate amino acids residues and two aspartate amino acid residues. The area of ionic charge that may comprise the sequence glu-glu-X-X-asp-asp, where X is any amino acid residue. In a still further example, a region of ionic charge may comprise the sequence glu-glu-tyr-lys-asp-asp.

In one aspect, the modified SPI may comprise a sequence selected from SEQ ID NOs: 158 to 770. In another aspect the modified SPI consists of one or more of SEQ ID NOs: 158 to 770.

Modified SPIs of the invention may be produced by chemical peptide synthesis, by recombinant peptide synthesis or using a host cell system.

The invention also includes functional equivalents of modified SPIs according to the invention, which retain the enhanced ability to inhibit SPs, as described previously. In one aspect, the term “functional equivalent” is intended to encompass peptide molecules having at least 50% sequence identity to a modified SPI produced according to the method of the invention. In another aspect, a functional equivalent may have 60%, 70%, 85%, 90%, 95%, 98%, 99% or more sequence identity to a modified SPI produced according to the method of the invention. Such functional equivalents preferably retain the enhanced ability to inhibit the target SP, as described previously.

The term “functional equivalents” also encompasses any polypeptide which comprises one or more conservative substitutions when compared to a modified SPI of the invention. In one aspect, the polypeptide comprises one or more conserved substitution. In another aspect, the polypeptide comprises two or more, three or more, four or more, or five or more conservative substitutions when compared to a modified SPI of the invention. A conserved substitution is an amino acid substitution wherein the characteristics of the substituted amino acid do not differ substantially from the amino acid which is normally found at that position. Conservative substitutions include the substitution of an acid amino acid for another acidic amino acid, a basic amino acid for another basic amino acid, an uncharged amino acid for another uncharged amino acid, a non-polar amino acid for another non-polar amino acid, a small amino acid for another small amino acid, or a bulky amino acid for another bulky amino acid. The acidic amino acids are aspartate and glutamate. The basic amino acids are arginine, histidine and lysine. The uncharged amino acids are asparagine, glutamine, serine, threonine, and tyrosine. The non-polar side chains are alanine, valine, leucine, isoleucine, proline, phenylalanine, methionine, tryptophan, glycine, and cysteine. Within the category of non-polar amino acids, alanine, valine, leucine, isoleucine, and glycine are considered to be small amino acids, and praline, phenylalanine, methionine, and tryptophan are considered to be bulky amino acids.

In a further aspect, the invention includes a fragment of a SPI produced according to the method of the invention. In another aspect, the fragment may comprise 2 or more amino acids. In another aspect, the fragment may comprise 3, 4, 5, 6, 7, 8, 9, 10, 11, 12, 13, 14, 15, 16, 17, 18, 19, 20, 25, 30, 35, 40, 45, or 50 amino acids. In another aspect, the fragment may consist of 2 or more amino acids. In another aspect, the fragment may consist of 2, 3, 4, 5, 6, 7, 8, 9, 10, 11, 12, 1, 14, 15, 16, 17, 18, 19, 20, 25, 30, 35, 40, 45, or 50 amino acids. Such fragments retain the enhanced ability to inhibit the target SP, as described previously.

In another aspect, a functional equivalent may be a fusion protein, obtained, for example, by cloning a polynucleotide encoding a modified SPI of the invention or variant or fragment thereof in frame to the coding sequences for a heterologous protein sequence. The term “heterologous”, when used herein, is intended to designate any polypeptide other than the modified SPI or its functional equivalent. Examples of heterologous sequences, comprising the fusion proteins, either at N- or at C-terminus, are the following: extracellular domains of membrane-bound protein, immunoglobulin constant regions (Fc region), multimerization domains, domains of extracellular proteins, signal sequences, export sequences, or sequences allowing purification by affinity chromatography. Many of these heterologous sequences are commercially available in expression plasmids since these sequences are commonly included in the fusion proteins in order to provide additional properties without significantly impairing the specific biological activity of the protein fused to them (Terpe (2003)). Examples of such additional properties are a longer lasting half-life in body fluids, the extracellular localization, or an easier purification procedure as allowed by a tag such as a histidine or HA tag.

The heterologous protein may also be a marker domain. In one aspect, the marker domain may be a fluorescent tag, an epitope tag that allows purification by affinity binding, an enzyme tag that allows histochemical or fluorescent labelling, or a radiochemical tag. In another embodiment, the marker domain may be a radiochemical tag. Such fusion proteins will be useful as diagnostic tools.

Methods for the generation of fusion proteins are standard in the art and will be known to the skilled reader. For example, most general molecular biology, microbiology, recombinant DNA technology and immunological techniques can be found in Sambrook et al. (2000). Generally, fusion proteins may be most conveniently generated recombinantly from nucleic acid molecules in which two nucleic acid sequences are fused together in frame. These fusion proteins will be encoded by nucleic acid molecules that contain the relevant coding sequence of the fusion protein in question.

In one aspect, a functional equivalent of a modified SP according to the invention which may include any molecule which comprises a portion suitable for displacing one of the residues of the catalytic triad of the target SP. In one aspect, this molecule may be a protein molecule, and the portion suitable for displacing one of the residues of the catalytic triad may be an amino acid residue. It will be apparent to a person skilled in the art that this definition cannot encompass any residue individually, since the residue will require additional residues to be present in order to position the residue suitable for displacing one of the residues of the catalytic triad of the target SP in an orientation and location in which it is suitable for displacing one of the residues of the catalytic triad. In one aspect, the functional equivalent may include a histidine residue within a protein molecule, which is positioned and orientated in a manner suitable for displacing one of the residues of the catalytic triad of the target SP. The invention also includes synthetic analogs of the modified SPIs described above.

The fragment or functional equivalent of the modified SPI produced according to the method of the invention is capable of functioning as a SPI. By “capable of function as a SPI” is meant that the fragment or functional equivalent can inhibit the SP activity of a SP. In a further aspect, the fragment or functional equivalent may be capable of inhibiting the SP activity of the target SP.

It will be apparent to a person skilled in the art that a variety of assays may be used to assess whether the fragment or functional equivalent is capable of functioning as a SPI. By way of example, but not limitation, such an assay may be a SP amidolytic assay, as described above, wherein the formation of p-nitroaniline following incubation of the target SP with the modified SPI in the presence of S2238 is detected. The modified SPIs of the invention may have an IC₅₀ of less than 30 nM, less than 25 nM, less than 20 nM, less than 15 nM, less than 14 nM, less than 13 nM, less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM when assessed in such a SP amidolytic assay. SPIs produced according to the method of the invention may have a Ki of less than less than 15 nM, less than 10 nM, less than 5 nM, less than 1 nM, less than 750 pM, less than 500 pM, less than 400 pM, less than 300 pM, less than 250 pM, less than 200 pM, less than 150 pM, less than 100 pM, less than 50 pM, less than 30 pM, less than 25 pM, less than 20 pM, less than 15 pM, less than 10 pM, less than 5 pM, less than 1 pM, or less than 100 pM when assessed in such a SP amidolytic assay.

In one aspect, the invention includes a nucleic acid molecule encoding a modified SPI produced according to the method of the invention. In another aspect, the invention includes a nucleic acid molecule having at least 50% sequence identity to a nucleic acid molecule encoding a modified SPI produced according to the method of the invention. In another aspect, the invention includes nucleic acid molecules having at least 60%, at least 70%, at least 80%, at least 85%, at least 90%, at least 95%, at least 98%, at least 99% or more sequence identity to a nucleic acid molecule encoding a modified SPI produced according to the method of the invention. The invention also includes a fragment of a nucleic acid molecule encoding a modified SPI produced according to the method of the invention. In one aspect, the fragment may comprise 10 or more nucleotides. In another aspect, the fragment may comprise 12 or more, 14 or more, 16 or more, 18 or more, 10 or more, 25 or more, 30 or more, 40 or more, 50 or more, 60 or more, 70 or more, 80 or more, 90 or more, or 100 or more nucleotides. Nucleic acid molecules according to the invention may be in any form, including double-stranded and single-stranded RNA, DNA, and cDNA.

In a further aspect, the invention includes an antisense nucleic acid molecule which hybridises under high stringency hybridisation conditions to a nucleic acid molecule according to the invention. High stringency hybridisation conditions are defined herein as overnight incubation at 42° C. in a solution comprising 50% formamide, 5×SSC (150 mM N NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C.

The invention also includes cloning and expression vectors comprising the nucleic acid molecules of the invention. Such expression vectors may comprise the appropriate transcriptional and translational control sequences, including but not limited to enhancer elements, promoter-operator regions, termination stop sequences, mRNA stability sequences, start and stop codons or ribosomal binding sites, linked in frame with the nucleic acid molecule(s) of the invention. Additionally, it may be convenient to cause the modified SPIs of the invention to be secreted from certain hosts. Accordingly, further components of such vectors may include nucleic acid sequences encoding secretion, signalling and processing sequences.

Vectors according to the invention include plasmids and viruses (including both bacteriophage and eukaryotic viruses), as well as other linear or circular DNA carriers, such as those employing transposable elements or homologous recombination technology. Many such vectors and expression systems will be apparent to a person skilled in the art. Particularly suitable viral vectors include baculovirus-, adenovirus- and vaccinia virus-based vectors.

Suitable hosts for recombinant expression include commonly used prokaryotic species, such as E. coli, or eukaryotic yeasts that can be made to express high levels of recombinant proteins and that can easily be grown in large quantities. Mammalian cell lines grown in vitro are also suitable, particularly when using virus-driven expression systems. Another suitable expression system is the baculovirus expression system that involves the use of insect cells as hosts. An expression system may also constitute host cells that have the DNA incorporated into their genome. Proteins, or protein fragments may also be expressed in vivo, for example in insect larvae or in mammalian tissues. A variety of techniques may be used to introduce vectors into prokaryotic or eukaryotic cells. Suitable transformation or transfection techniques are well described in the literature (Sambrook et al. (2000)). In eukaryotic cells, expression systems may either be transient (e.g. episomal) or permanent (chromosomal integration) according to the needs of the system.

### Methods of Treatment

The invention further includes the use of modified SPIs obtainable or obtained according to methods of the invention in therapy.

The uses and methods may also be performed using a modified SPI that is obtained by any means.

The invention includes a method of inhibiting a SP comprising administering to a subject a molecule of the invention.

By “molecule of the invention” is meant a modified SPI obtainable or obtained by a method of the invention, a nucleic acid encoding a modified SPI obtainable or obtained by a method of the invention, a vector comprising a nucleic acid encoding a modified SPI obtainable or obtained by a method of the invention, and a host cell containing a vector comprising a nucleic acid encoding a modified SPI obtainable or obtained by a method of the invention. A “molecule of the invention” also encompasses a modified SPI that is obtainable by the methods of the invention, but which is produced by any means. Accordingly, modified SPI molecules of the invention may be produced using any methodology known in the art, e.g., chemical peptide synthesis, solid-phase or solution-phase peptide synthesis, in vitro translation from a nucleic acid molecule encoding a modified SPI, or cell-based production methods employing prokaryotic or eukaryotic recombinant expression systems. In an exemplary embodiment, a “molecule of the invention” includes a polypeptide comprising a sequence set forth in any of SEQ ID NOs: 158-770. Such modified SPI molecules may be used in the methods of the invention, including any methods of treatment set forth herein.

The subject is generally an animal. The term “animal” encompasses any organism classified as a member of the animal kingdom. In general the animal is a mammal such as humans, cows, sheep, pigs, camels, horses, dogs, cats, monkeys, mice, rats, hamsters, and rabbits.

The method may involve administering the molecule of the invention in a therapeutically effective amount. The term “therapeutically effective amount” refers to the amount of compound needed to treat or ameliorate a targeted disease or condition. The term “prophylactically effective amount” used herein refers to the amount of compound needed to prevent a targeted disease or condition. The exact dosage will generally be dependent on the subject's status as the time of administration. Factors that may be taken into consideration when determining dosage include the severity of the disease state in the subject, the general health of the subject, the age, weight, gender, diet, time and frequency of administration, drug combinations, reaction sensitivities and the subject's tolerance or response to therapy. The precise amount can be determined by routine experimentation, but may ultimately lie with the judgement of the clinician or veterinarian. Generally, an effective dose will be from 0.01 mg/kg (mass of drug compared to mass of subject) to 50 mg/kg, preferably 0.05 mg/kg to 10 mg/kg. The molecule of the invention may be supplied in the form of a pharmaceutical composition in conjunction with a pharmaceutically acceptable carrier.

The term “pharmaceutically acceptable carrier”, as used herein, includes genes, polypeptides, antibodies, liposomes, polysaccharides, polylactic acids, polyglycolic acids and inactive virus particles or indeed any other agent provided that the excipient does not itself induce toxicity effects or cause the production of antibodies that are harmful to the individual receiving the pharmaceutical composition. Pharmaceutically acceptable carriers may additionally contain liquids such as water, saline, glycerol, ethanol or auxiliary substances such as wetting or emulsifying agents, pH buffering substances and the like. Excipients may enable the pharmaceutical compositions to be formulated into tablets, pills, dragees, capsules, liquids, gels, syrups, slurries, suspensions to aid intake by the subject. A thorough discussion of pharmaceutically acceptable carriers is available in Remington's Pharmaceutical Sciences (Mack Pub. Co., N.J. 1991).

In one embodiment, the invention provides methods of treatment involving modified thrombin inhibitors obtainable or obtained by the methods of the invention.

The invention includes a method of treating a subject suffering from a coagulopathy or preventing a subject developing a coagulopathy comprising administering a modified thrombin inhibitor obtainable or obtained by a method of the invention.

The invention also includes a modified thrombin inhibitor obtainable or obtained by a method of the invention for use in the treatment of a subject suffering from a coagulopathy or the prevention of a subject developing a coagulopathy.

By “coagulopathy” is meant any disorder of blood coagulation.

Treatment when anticoagulation is desirable includes procedures involving percutaneous, transvascular or transorgan catheterisation for diagnostic or therapeutic reasons. Such procedures may include but are not confined to: coronary angioplasty; endovascular stent procedures; direct administration of thrombolytic agents via an arterial or venous catheter such as following stroke or coronary thrombosis; electrical cardioversion; placement of cardiac pacemaker leads; intravascular and intracardiac monitoring of pressure, gaseous saturation or other diagnostic parameters; radiological and other procedures involving percutaneous or transorgan catheterisation; to ensure the patency of long-term, indwelling, intravascular parentral nutritional catheters; to ensure the patency of vascular access ports whether long or short term.

Additional in vivo applications of the methods of the invention include emergency anticoagulation after a thromboembolic event including but not limited to: acute myocardial infarction; thrombotic stroke; deep venous thrombosis; thrombophlebitis; pulmonary embolism; embolic and micro-embolic episodes where the source may be the heart, atherosclerotic plaque, valvular or vascular prostheses or an unknown source; disseminated intravascular coagulation (DIC).

The methods of the invention may also be used to prevent coagulation during organ perfusion procedures such as during cardiopulmonary bypass, hepatic bypass and as an adjunct to organ transplantation. The massive thrombotic reaction precipitated by cardiac pulmonary bypass cannot fully be antagonised by indirect thrombin inhibitors such as heparin and its analogues (Edmunds & Colman (2006)).

Further instances when anticoagulation is desirable include during haemodialysis, haemofiltration or plasma exchange procedures. Anticoagulation may also be desirable during surgical procedures involving cross clamping of blood vessels in order to minimise the risk of coagulation in the distal circulation. Such procedures may include but are not confined to endarterectomy, insertion of vascular prostheses, repair of aortic and other arterial aneurysms.

Additionally, the methods and the modified thrombin inhibitors obtainable or obtained of the invention may be useful to induce anticoagulation in heparin-resistant subjects.

The methods and modified thrombin inhibitors obtainable or obtained by the methods of the invention may also be useful in the treatment or prevention of heparin-induced thrombocytopaenia. Such treatment may be administered to a subject with or at risk from HIT and with or without active thrombosis and may be administered until platelet counts have recovered to within the range of normal or until the risk of thrombosis has passed (Girolami & Girolami (2006), Lewis & Hursting (2007)). The molecules of the invention may be administered by any suitable route. Preferred routes of administration include intravenous, intramuscular or subcutaneous injection, oral administration, subligual administration and transdermal administration. The treatment may be continuously administered by intravenous infusion or as a single or repeated bolus injection. The molecules of the invention may be administered individually to a subject or may be administered in combination with other agents, drugs or hormones. For example, the molecules of the invention may be administered with oral anticoagulants such as coumarin derivatives until such time as the subject has become stabilised, following which the subject may be treated with the coumarin derivatives alone.

The invention further provides that the modified SPIs produced by the method of the invention may be used in diagnosis. Since these methods involve inhibiting SP activity specifically by interaction with the target SP, they can be used to detect the presence of the target SP and hence to diagnose conditions caused by SP accumulation, such as a fibrin or platelet thrombus, caused by an accumulation of thrombin. The invention therefore provides methods of diagnosing a condition caused by SP accumulation by administering a modified SPI of the invention as described above to a subject or to tissue isolated from a subject, and detecting the presence of said SPI or fragment or functional equivalent thereof, wherein the detection of said modified SPI or fragment or functional equivalent bound to the target SP is indicative of said disease or condition. The modified SPI or functional equivalent may be in the form of a fusion protein comprising a marker domain, as described in more detail above, to facilitate detection. In one aspect, the marker domain may be a radiochemical tag so that detection can be carried out using known imaging methods.

According to a further aspect of the invention, the in vivo method of the invention may be used to treat a malignant disease or a condition associated with malignant disease.

It has been recognised for decades that malignant disease is often associated with an increased tendency to thromboembolic episodes, caused by an increase in levels of the SP thrombin. Trousseau's syndrome, for example, is characterised by fleeting thrombophlebitis and underlying malignancy and thrombin inhibitors such as heparin have been used in its management (Varki (2007)). More recently it has become apparent that the generation of procoagulant factors including thrombin may be a cause rather than a result of certain aspects of malignant disease (Nierodzik & Karpatkin (2006)). There are many instances wherein it may be desirable to inhibit a SP and then neutralise such inhibition. By way of example, but not limitation, such inhibition and neuralisation may be advantageous during surgery, wherein target SP inhibition is required to prevent thrombin-induced coagulation whilst the surgery is taking place, and reversal of the inhibition is advantageous upon completion of the surgery in order to allow wound healing.

Where the SPI is a thrombin inhibitor, thrombin activity may be neutralised by the administration of a cationic peptide, e.g. protamine sulphate. Any of the methods of treatment relating to thrombin inhibition described herein may therefore describe the additional step of administering to the subject an amount of a cationic peptide to result in neutralisation of the thrombin inhibition. In one aspect, the amount of cationic peptide which is administered may be between 0.01 mg/ml and 1 mg/ml. In another aspect, the amount of cationic peptide which is administered may be 0.01 mg/ml or more, 0.02 mg/ml or more, 0.03 mg·ml or more, 0.04 mg/ml or more, 0.05 mg/ml or more, 0.06 mg/ml or more, 0.07 mg/ml or more, 0.08 mg/ml or more, 0.09 mg/ml or more, 0.1 mg/ml or more, 0.11 mg/ml or more, 0.12 mg/ml or more, 0.13 mg·ml or more, 0.14 mg/ml or more, 0.15 mg·ml or more, 0.16 mg/ml or more, 0.18 mg/ml or more, 0.19 mg/ml or more, 0.2 mg/ml or more, 0.3 mg·ml or more, 0.4 mg·ml or more, 0.5 mg·ml or more, or 1 mg/ml.

Various aspects and embodiments of the present invention will now be described in more detail by way of example. It will be appreciated that modification of detail may be made without departing from the scope of the invention.

### BRIEF DESCRIPTION OF FIGURES

FIG. 1 shows the catalytic reaction scheme of a typical SP. The polypeptide substrate binds to the SP such that the scissile bond is inserted into the active site of the enzyme, and its carbonyl carbon is located near the nucleophilic serine of the SP. The serine —OH attacks the carbonyl carbon, and the nitrogen of the SP's histidine accepts the hydrogen from the —OH of the serine, generating a tetrahedral intermediate. Next, the nitrogen-carbon in the peptide bond is broken, generating an acyl-enzyme intermediate, to which water is added, generating another tetrahedral intermediate. In a final reaction, the C-terminus of the peptide is ejected, and the SP is returned to its original state.

FIG. 2 shows the structure of the thrombin-s-variegin complex compared to other thrombin inhibitor structures.

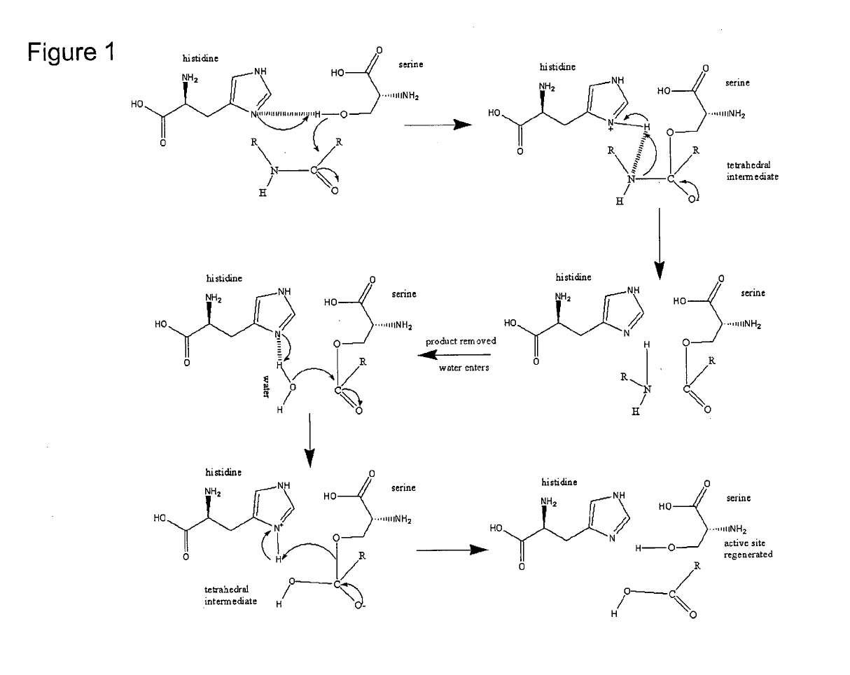

(A) Thrombin-s-variegin complex structure. Thrombin A-chain backbone is coloured as light blue ribbon, B-chain backbone is coloured as white ribbon and s-variegin backbone and side chain atoms are showed as pink sticks.
(B) Thrombin-hirulog-1 complex structure (PDB: 2HGT). Hirulog-1 is coloured as red sticks.
(C) Thrombin-hirulog-3 complex structure (PDB: 1ABI). Hirulog-3 is coloured as yellow sticks.
(D) Thrombin-hirugen complex structure (PDB: 1HGT). Hirugen is coloured as green sticks.
(E) Thrombin-PPACK complex structure (PDB: 1PPB). PPACK is coloured as orange sticks.
(F) Wild-type, inhibitor- and Na+-free thrombin (PDB: 2AFQ). Structure represents ‘slow’ form thrombin and is without an inhibitor.

FIG. 3 shows analysis of the cleavage of s-variegin by thrombin at 37° C. and 24° C. The relative percentage of uncleaved s-variegin (▪), cleavage product of mass 1045 (representing N-terminal fragment SDQGDVAEPK; SEQ ID NO: 2) ( [symbol] ) and cleavage product of mass 2582 (representing C-terminal fragment MHKTAPPFDFEAIPEEYLDDES; MH22; SEQ ID NO: 3) (▪) was calculated by integrating the area under the peaks in RP-HPLC analysis. Cleavage proceeded faster at 37° C. than at room temperature (24° C.) (n=2, error bars represent S.D.).

(A) Results obtained by incubating s-variegin and thrombin at 37° C. Only 180 min was needed for complete cleavage.

(B) Results obtained by incubating s-variegin and thrombin at 24° C. 360 min was needed for ˜90% of cleavage.

FIG. 4 shows that s-variegin and EP25 retained their activities after being cleaved by thrombin.

(A) S-variegin was incubated with thrombin (3.33 nM) for up to 24 hours at room temperature and at various time points assayed for the ability to inhibit thrombin amidolytic activity on 100 μM S2238 (n=3, error bars represent S.D.).

(B) Similar experiments were carried out replacing s-variegin with EP25 (n=3, error bars represent S.D.).

FIG. 5 shows the inhibition of human plasma thrombin by MH22, s-variegin and hirulog-1. The ability of MH22, s-variegin and hirulog-1 to inhibit amidolytic activity of human plasma derived thrombin were assayed using active site directed substrate S2238 (100 μM). Dose response curves of thrombin (1.65 nM) inhibited by MH22 (◯) s-variegin (▴) and hirulog-1 (▪) all showed inhibition when they are present in similar molar concentrations with thrombin.

Concentrations used for MH22 (▪) and s-variegin were 0.03 nM, 0.1 nM, 0.3 nM, 1 nM, 3 nM, 10 nM, 30 nM, 100 nM, 300 nM and 1000 nM. IC₅₀ of inhibition are 11.46±0.71 nM and 8.25±0.45 nM, respectively (n=3, error bars represent S.D.). Concentrations used for hirulog-1 (▴) were 0.3 nM, 1 nM, 3 nM, 10 nM, 30 nM, 100 nM, 300 nM, 1000 nM, 3000 nM and 10000 nM. IC₅₀ of inhibition is 72.6±3.9 nM (n=3, error bars represent S.D).

FIG. 6 shows the apparent inhibitory constant (Ki′) of MH22. Since MH22 behaved as a tight-binding inhibitor, inhibition of thrombin (1.65 nM) by MH22 at different concentrations (0.195 nM, 0.391 nM, 0.781 nM, 1.56 nM, 3.12 nM, 6.25 nM, 12.5 nM, 25 nM, 50 nM, 100 nM) was examined using different concentrations of S2238 as the substrate. Reactions were started with the addition of thrombin. Shown in the figure are experiments performed with 100 μM S2238. Data obtained were fitted to equations to derive an apparent inhibitory constant (Ki′) (mean±S.D.) of 14.31±0.26 nM (n=3, error bars represent S.D.).

FIG. 7 shows the inhibitory constant Ki of MH22. The apparent inhibitory constant (Ki′) of MH22 was determined with six different concentrations of substrate S2238 (12.5 μM, 25 μM, 50 μM, 75 μM, 100 μM and 150 μM). A plot of Ki′ against substrate concentration remained constant throughout, indicating that MH22 non-competitively (Kᵢ′=(S+Kₘ)/[(Kₘ/Kᵢ)+(S/αKᵢ)] and Kᵢ′=KO inhibits thrombin amidolytic activity on S2238. Fitting the Ki′ values by linear regression derived the inhibitory constant Ki as 14.11±0.29 nM (n=3 for each S2238 concentration, error bars represent S.D.).

FIG. 8 shows the equilibrium scheme for variegin inhibition of thrombin. In the absence of variegin, S2238 binds to thrombin (Ks is the equilibrium constant for thrombin-S2238 dissociation, shown as blue arrows) and hydrolyzed by thrombin to release colored product pNA (Kp is the forward rate constant for pNA formation, green arrow).

In the presence of variegin, thrombin binds to variegin (Ki-v is the inhibitory constant of variegin, shown as brown arrows) thus S2238 hydrolysis is inhibited competitively. Upon binding, thrombin cleaves variegin into MH22 (kc is the forward rate constant for cleavage, shown as a violet arrow).

MH22 remained bound to thrombin, acting as a classical non-competitive inhibitor of thrombin (Ki-m is the inhibitory constant of MH22). MH22 binds to free thrombin or S2238 bound thrombin with the same affinity, α=1, thus Ki-m=αKi−m (shown as red arrows). Similarly, Ks=αKs, binding of S2238 to thrombin is unaffected by MH22.

FIG. 9 shows the thrombin catalytic triad in s-variegin bound and hirugen bound structures.

(A) Thrombin catalytic triad ^{T}His57, ^{T}Asp102 and ^{T}Ser195 when unoccupied in the thrombin-hirugen structure (green) have the intact charge relay hydrogen bonding system. In the thrombin-s-variegin structure (pink), ^{T}Ser195 Oγ is displaced by 1.10 Å (cyan arrow). The distance between ^{T}His57 Nε and ^{T}Ser195 Oγ is 3.77 Å, thus a hydrogen bond is not formed and the charge relay system is broken.

(B) The displacement of ^{T}Ser195 Oγ is due to an interaction between s-variegin (shown in gray) and the catalytic triad of thrombin. The ^{v}His12 backbone N (donor) engaged ^{T}Ser195 Oγ (acceptor) through a hydrogen bond (2.77 Å) while the ^{v}His12 side chain Nδ (acceptor) could only contribute a weak hydrogen bond with ^{T}Ser195 Oγ (donor) (3.68 Å). The ^{v}His12 backbone N also forms a hydrogen bond with ^{T}Gly193 backbone N and ^{T}Cys42 Sγ via a water molecule (light blue). Thus, ^{T}Ser195 Oγ is rendered a weak nucleophile, and incapable of attacking the backbone carbon of the substrate. Oxyanion hole formation is also disturbed due to the involvement of ^{T}Gly193 backbone N in this hydrogen bond network.

FIG. 10 shows prime subsite interactions between thrombin and s-variegin. For s-variegin, only residues P2′ to P5′ (^{v}His12 to ^{v}Ala15) are shown. Density for s-variegin P1′ ^{v}Met11 cannot be traced in the structure. Thrombin S2′ subsite (red) (formed by ^{T}Cys42, ^{T}His57, ^{T}Trp60D, ^{T}Lys60F, ^{T}Glu192 and ^{T}Ser195) partially overlaps with the S1′ subsite observed in hirulog-3. The s-variegin P3′ ^{v}Lys13 side chain runs close and parallel with the ^{T}Glu192 side chain, and its backbone is in contact with ^{T}Leu41, forming the S3′ subsite (cyan). S-variegin P4′ ^{v}Thr14 side chain is directed towards the bottom of the autolysis loop, occupying a small pocket formed by ^{T}Gly142, ^{T}Asn143, ^{T}Glu192, ^{T}Gly193 and ^{T}Glu151, forming the S4′ subsite (pink). The thrombin S5′ subsite (green) is lined by ^{T}Leu40 at the bottom, which allows s-variegin P5′ ^{v}Ala15 to burry its side chain in the interface.

FIG. 11 shows s-variegin fitted firmly into the canyon-like cleft of thrombin.

(A) Thrombin has a deep canyon-like cleft (boxed) starting from active site, and extending to exosite-I.

(B) On the whole s-variegin (pink CPK model) fitted firmly at the bottom of the canyon-like cleft in an extended conformation, covering the catalytic pocket, prime subsites and exosite-I. The bottom of the cleft is composed of mainly apolar residues. The walls of the cleft are formed by the 60- and autolysis loops near the thrombin active site, along with the 34- and 70-loops at exosite-I.

(C) Thrombin residues that interfaced with s-variegin are coloured according to their positions: catalytic pocket—blue; 60-loop—red; autolysis loop—cyan; 34-loop—yellow; 70-loop—green; bottom of the cleft—orange. A ball and stick model of s-variegin is shown in pink.

(D) s-variegin interacts with thrombin through specific side-chain contacts. All but five residues (^{v}Phe18, ^{v}Asp19, ^{v}Ala22, ^{v}Glu26 and ^{v}Tyr27, all coloured white) on s-variegin have their side chains buried in the interface with thrombin.

FIG. 12 shows the design of new variegin variants. New variegin variants were designed to improve thrombin-variegin interactions. The approach was to first optimise the length of vareign before optimising several key positions on variegin.

FIG. 13 shows thrombin inhibition by variegin variant EP21; a slow, tight-binding, competitive inhibitor.

(A) EP21 (0.3 nM, 1 nM, 3 nM, 10 nM, 30 nM, 100 nM, 300 nM, 1000 nM, 3000 nM and 10000 nM) inhibition of thrombin (1.65 nM) amidolytic activity with S2238 (100 μM) showed a pre-incubation time-dependent shift due to slow binding. The IC₅₀ values were 176.9±6.8 nM without pre-incubation (solid line) and 16.2±2.9 nM with 20 min pre-incubation (dotted line) (n=3, error bars represent S.D.).

(B) Progression curves (not shown) of thrombin (1.65 nM) inhibition by different concentrations of EP21 (18.8 nM, 25 nM, 37.5 nM, 50 nM, 75 nM, 100 nM and 150 nM) at 100 μM S2238 were fitted to the equation P=V_{f}t+(Vᵢ−V_{f})(1−e^{-kt})/k+Pₒ describing a slow binding inhibitor to obtain a k value for each concentration of EP21 used. A plot of k against EP21 concentration (solid line) is a hyperbolic curve described by the equation k=K₄+K₃Iₜ/[Iₜ+Kᵢ′(1+S/Kₘ)] and hence was fitted to the equation to obtain a Ki′ of 1.66±0.36 nM, representing the dissociation constant of initial collision complex EI. The overall inhibitory constant (Ki) was calculated from the equation Kₜ=Kᵢ′[K₄/(K₃+K₄)] as 0.315±0.024 nM (n=3, error bars represent S.D.).

FIG. 14 shows thrombin inhibition by variegin variant MH18; a fast, tight-binding, non-competitive inhibitor.

(A) The ability of MH18 (0.1 nM, 0.3 nM, 1 nM, 3 nM, 10 nM, 30 nM, 100 nM, 300 nM, 1000 nM, 3000 nM and 10000 nM) to inhibit amidolytic activity of thrombin (1.65 nM) was assayed in 100 μM S2238. Dose-response curves are independent of pre-incubation time. IC₅₀ values were 10.9±1.2 nM without pre-incubation (solid line) and 11.7±1.9 nM after 20 min pre-incubation (dotted line) (n=3, error bars represent S.D.). (B) Since MH18 behaved as a fast and tight-binding inhibitor, thrombin (1.65 nM) inhibition was tested with 0.39 nM, 0.78 nM, 1.56 nM, 3.13 nM, 6.25 nM, 12.5 nM, 25 nM, 50 nM, 100 nM and 200 nM of MH18 at 100 μM of S2238 (solid line). The apparent inhibitory constant (Ki′) obtained by fitting data to the equation Vₛ=(Vₒ/2Eₜ) {[(Kᵢ′+Iₜ−Eₜ)²+4Kᵢ′Eₜ]^{1/2}−(Kᵢ′+Iₜ−Eₜ)} was 14.9±3.5 nM. The inhibitory constant (Ki) was calculated to be 14.9±3.5 nM based on equations Kᵢ′=(S+Kₘ)/[(Kₘ/Kₜ)+(S/αKᵢ)] and Kᵢ′=Kᵢ (n=3, error bars represent S.D.).

FIG. 15 shows thrombin inhibition by variegin variant DV24; a fast, tight-binding, competitive inhibitor.

(A) Dose-response curves of thrombin (1.65 nM) inhibition by DV24 (0.1 nM, 0.3 nM, 1 nM, 3 nM, 10 nM, 30 nM, 100 nM, 300 nM, 1000 nM and 3000 nM) in 100 μM S2238 showed a right shift with increased pre-incubation time due to cleavage. IC₅₀ values were 7.49±0.28 nM without pre-incubation (solid line) and 10.07±0.60 nM with 20 min pre-incubation (dotted line) (n=3, error bars represent S.D.).

(B) Since DV24 behaved as a fast and tight-binding inhibitor, thrombin (1.65 nM) inhibition was tested with 0.39 nM, 0.78 nM, 1.56 nM, 3.13 nM, 6.25 nM, 12.5 nM, 25 nM, 50 nM, 100 nM and 200 nM of DV24 at 100 μM of S2238 (solid line). The apparent inhibitory constant (Ki′) obtained by fitting data to the equation Vₛ=(Vₒ/2Eₜ) {[(Kᵢ′+Iₜ−Eₜ)²+4Kᵢ′Eₜ]^{1/2}−(Kᵢ′−Eₜ)} was 9.74±0.91 nM. The inhibitory constant (Ki) was calculated to be 0.306±0.029 nM based on the equation Kᵢ′=Kᵢ(1+S/Kₘ) (n=3, error bars represent S.D.).

FIG. 16 shows thrombin inhibition by variegin variant DV24K10R; a fast, tight-binding, competitive inhibitor.

(A) Dose-response curves of thrombin (1.65 nM) inhibition by DV24K10R (0.1 nM, 0.3 nM, 1 nM, 3 nM, 10 nM, 30 nM, 100 nM, 300 nM, 1000 nM and 3000 nM) in 100 μM S2238 showed a right shift with increased pre-incubation time due to cleavage. IC₅₀ values were 6.98±0.76 nM without pre-incubation (solid line) and 12.01±0.41 nM after 20 min pre-incubation (dotted line) (n=3, error bars represent S.D.).

(B) Since DV24K10R behaved as a fast and tight-binding inhibitor, thrombin (1.65 nM) inhibition was tested with 0.39 nM, 0.78 nM, 1.56 nM, 3.13 nM, 6.25 nM, 12.5 nM, 25 nM, 50 nM, 100 nM and 200 nM of DV24K10R at 100 μM of S2238 (solid line). The apparent inhibitory constant (Ki′) obtained by fitting data to the equation Vₛ=(Vₒ/2Eₜ) {[(Kᵢ′+Iₜ−Eₜ)²+4Kᵢ′Eₜ]^{1/2}−(Kᵢ′+Iₜ−Eₜ)} was 8.27±0.85 nM. The inhibitory constant (Ki) is calculated to be 0.259±0.015 nM based on equation (4) (n=3, error bars represent S.D.).

FIG. 17 shows the presence of a ^{v}Pro16-^{v}Pro17 (yellow) dipeptide sequence in s-variegin resulted in a kink in its backbone. Overlaying s-variegin (pink, only Cα positions traced) and hirulog-3 (green, only Cα positions traced) based on their thrombin structures revealed displacement of ^{v}Phe18 and ^{v}Asp19 from their corresponding residues Gly10 and Asp11 of hirulog-3 by 3.16 Å and 1.70 Å (measured by Cα positions) (cyan double headed arrow). Consequently, the ^{v}Asp19 side chain points in the opposite direction to the analogous Asp11 side chain in hirulog-3. This Asp11 in hirulog-3 makes a strong ion-pair with ^{T}Arg73 (white). Due to the displacement of ^{v}Asp19, the nearest possible distance between ^{T}Arg73 NH2 and ^{v}Asp19 OD1 is 9.22 Å, which rendered this interaction in the thrombin-s-variegin structure impossible.

FIG. 18 shows thrombin inhibition by variegin variant DV23; a fast, tight-binding, competitive inhibitor.

(A) Dose-response curves of thrombin (1.65 nM) inhibition by DV23 (0.1 nM, 0.3 nM, 1 nM, 3 nM, 10 nM, 30 nM, 100 nM, 300 nM, 1000 nM and 3000 nM) in 100 μM S2238 showed a right shift with increased pre-incubation time due to cleavage. IC₅₀ were 45.4±1.6 nM without pre-incubation (solid line) and 77.8±6.1 nM after 20 min pre-incubation (dotted line) (n=3, error bars represent S.D.).

(B) Thrombin (1.65 nM) inhibition was tested with 3.91 nM, 7.81 nM, 15.6 nM, 31.3 nM, 62.5 nM, 125 nM, 250 nM and 500 nM of DV23 at 100 μM of S2238. The apparent inhibitory constant (Ki′) obtained by fitting data to the equation Vₛ=(Vₒ/2Eₜ) {[(Kᵢ′+Iₜ−Eₜ)²+4Kᵢ′Eₜ]^{1/2}−(Kᵢ′+Iₜ−Eₜ)} was 69.6±7.8 nM. The inhibitory constant (Ki) was calculated to be 2.19±0.23 nM based on the equation Kᵢ′=Kᵢ′=(1+S/Kₘ) (n=3, error bars represent S.D.).

FIG. 19 shows thrombin inhibition by variegin variant DV23K10R; a fast, tight-binding, competitive inhibitor.

(A) DV23K10R (0.1 nM, 0.3 nM, 1 nM, 3 nM, 10 nM, 30 nM, 100 nM, 300 nM, 1000 nM and 3000 nM) inhibited thrombin (1.65 nM) in the presence of 100 μM S2238. Loss of activity after cleavage was rapid, indicated by the strong right shift of dose-response curve. IC₅₀ values were 12.9±1.0 nM without pre-incubation (solid line) and 101.9±1.2 nM after 20 min pre-incubation (dotted line) (n=3, error bars represent S.D.).

(B) Thrombin (1.65 nM) inhibition was tested with 3.91 nM, 7.81 nM, 15.6 nM, 31.3 nM, 62.5 nM, 125 nM, 250 nM and 500 nM of DV23K10R at 100 μM of S2238 (solid line). The apparent inhibitory constant (Ki′) obtained by fitting data to the equation Vₛ=(Vₒ/2Eₜ) {[(Kᵢ′+Iₜ−Eₜ)²+4Kᵢ′Eₜ]^{1/2}−(Kᵢ′+Iₜ−Eₜ)} was 19.1±1.9 nM. The inhibitory constant (Ki) was calculated to be 0.600±0.010 nM based on the equation Kᵢ′=Kᵢ(1+S/Kₘ) (n=3, error bar represents S.D.).

FIG. 20 shows the delay time-to-occlusion (TTO) for zebrafish larvae injected with different peptides. Zebrafish 4 dpf (days post fertilisation) larvae were injected with 10 nl of different peptides at 500 μM or 10 nl of PBS as a control. The larvae caudal vein was injured by laser ablation 20 minutes after injection of the peptides or PBS. TTO after laser ablation were recorded up to 150 seconds for comparison of the antithrombotic effects of different peptides. TTO of PBS, hirulog-1, s-variegin, EP25 and MH22 were 19.0±3.2 seconds, 45.0±5.5 seconds, 120.8±7.4 seconds, 22.5±6.2 seconds and 33.3±2.9 seconds, respectively. Within 150 seconds, no thrombi were formed in larvae injected with DV24K10RY^{sulf}. With the exception of the slow binding inhibitor EP25, the abilities of the peptides to prolong TTO generally correlated with their Ki (n=4, error bars represent S.D.).

FIG. 21 shows the ability of protamine sulphate to neutralise the inhibition of thrombin amidolytic activity by the peptides, which was assayed using the chromogenic substrate S2238. Protamine sulphate (3 mg/ml, 1 mg/ml, 0.3 mg/ml, 0.1 mg/ml, 0.03 mg/ml, 0.01 mg/ml, 0.003 mg/ml and 0.001 mg/ml) was incubated with peptides at their IC₅₀ concentrations (solid lines)—8.25 nM s-variegin (▪), 11.5 nM MH22 () and 1.4 nM DV24K10RY^{sulf} (▴)—for 10 min before addition of thrombin (1.65 nM). Amidolytic activity of thrombin was assayed with 100 μM S2238. Percentages of inhibition in the presence and absence of protamine sulphate were compared for calculation of percentages of reversal. s-variegin and MH22 can be reversed to similar extent but higher concentrations of protamine sulphate are needed for effective reversal of DV24K10RYsulf.

Similar experiments were conducted with the peptides at their IC90 concentrations (dotted lines): 167 nM for s-variegin (□), 224 nM for MH22 (◯) and 13.6 nM for DV24K10RY^{sulf} (Δ). Higher concentrations of protamine sulphate are needed for reversal of all three peptides. s-variegin and MH22 are again neutralized to the same extent, while it is more difficult to neutralize DV24K10RY^{sulf}. Therefore, the peptides acidic C-terminal residues are most likely responsible for protamine sulphate binding. This invention is further illustrated by the following examples which should not be construed as limiting. The contents of all references, patents and published patent applications cited throughout this application are incorporated herein by reference.

### EXAMPLES

The following examples and definitions of parameters are used throughout the examples:

V=(VₘₐₓS)/(S+Kₘ)

where V is the initial rate of reaction, S is the concentration of substrate S2238 and Kₘ is the Michaelis-Menten constant of substrate for the enzyme (thrombin).

y=A₂+(A₁−A₂)/[1+(x/x₀)^{H}]

where y is percentage of inhibition, A₂ is right horizontal asymptote, A₁ is left horizontal asymptote, x is log 10 of inhibitor concentration, x₀ is point of inflection and H is the slope of the curve. IC₅₀ was calculated by substituting ‘50’ into y.

Vₛ=(Vₒ/2Eₜ){[(Kᵢ′+Iₜ−Eₜ)²+4Kᵢ′Eₜ]^{1/2}−(Kᵢ′+Iₜ−Eₜ)}

where Vₛ is steady state velocity in the presence of inhibitor, Vₒ is velocity observed in the absence of inhibitor, Eₜ is total enzyme concentration, Iₜ is total inhibitor concentration and Kᵢ′ is apparent inhibitory constant.

Kᵢ′=Kᵢ(1+S/Kₘ)

where Kᵢ′ increases linearly with S, K; is the inhibitory constant, S is the concentration of substrate and Kₘ is the Michaelis-Menten constant for S2238.

Kᵢ′=(S+Kₘ)/[(Kₘ/Kᵢ)+(S/αKᵢ)]

where α is the modifying constant of the inhibitor on the affinity of the enzyme for its substrate, and likewise the effect of the substrate on the affinity of the enzyme for the inhibitor. α<1 when binding of one supported the other, α>1 when binding of one impedes the other and when α=1, binding of one has no effect on the other. For a mixed-type non-competitive inhibitor, α is either <1 or >1.

Kᵢ′=Kᵢ

where Kᵢ′ remained constant with increasing S, K; is the inhibitory constant, S is the concentration of substrate S2238 and Kₘ is the Michaelis-Menten constant for S2238

P=V_{f}t+(Vᵢ−V_{f})(1−e^{-kt})k+Pₒ

k=K₄+K₃Iₜ/[Iₜ+Kᵢ′(1+S/Kₘ)]

wherein Kᵢ is the overall inhibitory constant.

Kᵢ=Kᵢ′[K₄/(K₃+K₄)]

where P is the amount of product formed, P. the is initial amount of product, V_{f} is final steady state velocity, Vᵢ is initial velocity, t is time, and k is apparent first-order rate constant.

### Example 1

### Determination of the Crystal Structure of S-Variegin Bound to Thrombin Materials

4-(2-Hydroxyethyl)piperazine-1-ethanesulfonic acid (HEPES), HEPES sodium salt and polyethylene glycol (PEG) 8000 were from Sigma Aldrich (St. Louis, Mo., USA). Crystallization trays and grease were purchased from Hampton Research (Aliso Viejo, Calif., USA).

### Synthesis, Purification and Mass Spectrometry of Peptides

All peptides used in the studies were synthesized using solid phase peptide synthesis methods on an Applied Biosystems Pioneer Model 433A Peptide Synthesizer (Foster City, Calif., USA). The synthesized peptides were assembled on support resins pre-loaded with respective C-terminal amino acids, which cleaves to release peptides with free carboxylic acid at the C-terminus. Fmoc groups of amino acids were removed by 20% v/v piperidine in DMF and coupled using HATU/DIPEA in situ neutralization chemistry. Cleavage of synthesized peptides from resins and side chain protection groups were typically carried out using a cocktail of TFA/1,2-ethanedithiol/thioanisole/water (90:4:4:2% v/v) at room temperature for 2 h. Cleaved peptides were precipitated with cold diethyl ether. Precipitated peptides were dissolved in either water or 0.1% TFA and lyophilized before purification.

Synthetic crude peptides were purified to homogeneity by RP-HPLC on ÄKTA™ purifier system (GE Healthcare, Uppsala, Sweden) with SunFire™ C18 (100 Å, 5 μm; 250 mm×10 mm) (Waters, Milford, Mass.) column. Typically peptides were eluted using an optimized linear elution gradient created by a combination of two solvents (solvent A: 0.1% TFA in water and solvent B: 0.1% TFA and 80% acetonitrile in water).

Of special note are peptides containing sulphotyrosine (DV24Y^{sulf}, DV24K10RY^{sulf} and MH18Y^{sulf}), of which the sulphate groups are acid labile. Cleavage of these peptides was carried out with 90% aqueous TFA on ice for 5 h as previously described (Kitagawa et al., 2001). Purification of the peptides containing sulphotyrosine (DV24Y^{sulf}, DV24K10R Y^{sulf} and MH18Y^{sulf} and phosphotyrosine (DV24Y^{phos} and DV24K10R Y^{phos}) were performed with solvent containing 0.1% FA as ion pairing agent instead of TFA. The sulphate moiety on Tyr27 is unstable during ionization in mass spectrometry analysis, thus non-sulphated masses were observed. Identification of sulphated peptides was on the basis of: (1) non-sulphated masses of the peptides; (2) as opposed to tyrosine residue that absorbs UV at 280 nm, sulphotyrosine residue does not; and (3) sulphated and non-sulphated peptides do not co-elute in RP-HPLC.

### Thrombin

Two different sources of thrombin—recombinant α-thrombin (based on human α-thrombin sequence) and human plasma derived thrombin, both were generous gifts from the Chemo-Sero-Therapeutic Research Institute (KAKETSUKEN, Japan). Recombinant α-thrombin was desalted with the HiTrap™ Desalting Column (GE Healthcare, Uppsala, Sweden) in 20 mM ammonium bicarbonate (NH₄HCO₃) and lyophilized before being used for crystallization. Human plasma derived thrombin was used to assay thrombin inhibitory activities of the peptides.

### Crystallization of Thrombin-S-Variegin Complex

The reported crystallization conditions that were used to crystallized thrombin-hirugen and thrombin-hirulog-1 complexes of human α-thrombin (Skrzypczak-Jankun et al., 1991) were modified and optimized. Lyophilized s-variegin was dissolved in 50 mM HEPES buffer (pH 7.4) containing 375 mM NaCl to a concentration of 8.34 μM (3 mg/ml). Desalted, lyophilized recombinant α-thrombin was subsequently dissolved in the s-variegin solution to a final concentration of 5.56 μM (20 mg/ml). The amount of s-variegin in this mixture was 1.5-fold in excess of thrombin. Crystallization of the thrombin-s-variegin complex was achieved using the hanging drop vapor diffusion method. Typically, 1 μl of protein solution was mixed with 1 μl of precipitant buffer (100 mM HEPES buffer pH 7.4, containing 20 to 25% (w/v) PEG 8000) and were equilibrated against 1 ml of precipitant buffer at 4° C. Crystals appeared after approximately four weeks and were harvested for data collection two weeks later. The entire process for setting up, growing and harvesting of crystals were performed in cold room (4° C.) as the crystals are unstable at room temperature.

### Data Collection

Prior to data collection, crystals were briefly soaked in a cryoprotectant solution containing the mother liquor, supplemented with 25% (v/v) glycerol, and flash cooled at 100 K in nitrogen (gas) cold stream (Cryostream cooler, Oxford Cryosystem, Oxford, United Kingdom). Synchrotron data were measured at the Beamline X29 (National Synchrotron Light Source, Brookhaven, USA). Data sets were collected (Table 1) using the Quantum 4-CCD detector. The diffraction data were processed using the program HKL2000 (Otwinowski and Minor, 1997). The crystal belonged to the monoclinic space group C2 and diffracted up to 2.7 Å resolution with a=124.66 Å, b=50.83 Å, c=61.54 Å and V=385390.59 A3 Da-1 and corresponded to a solvent content of 59.09%.

### Structure Solution and Refinement

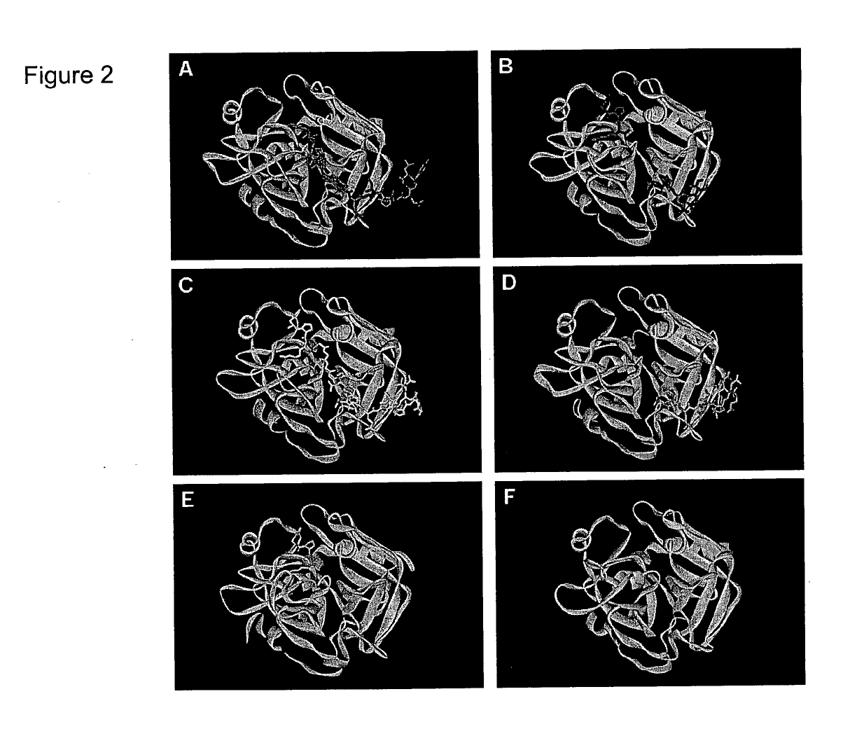

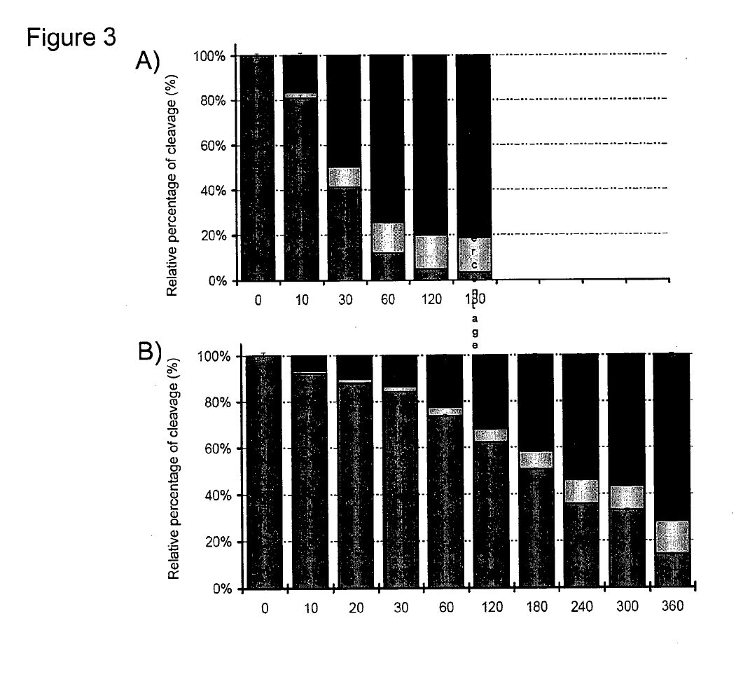

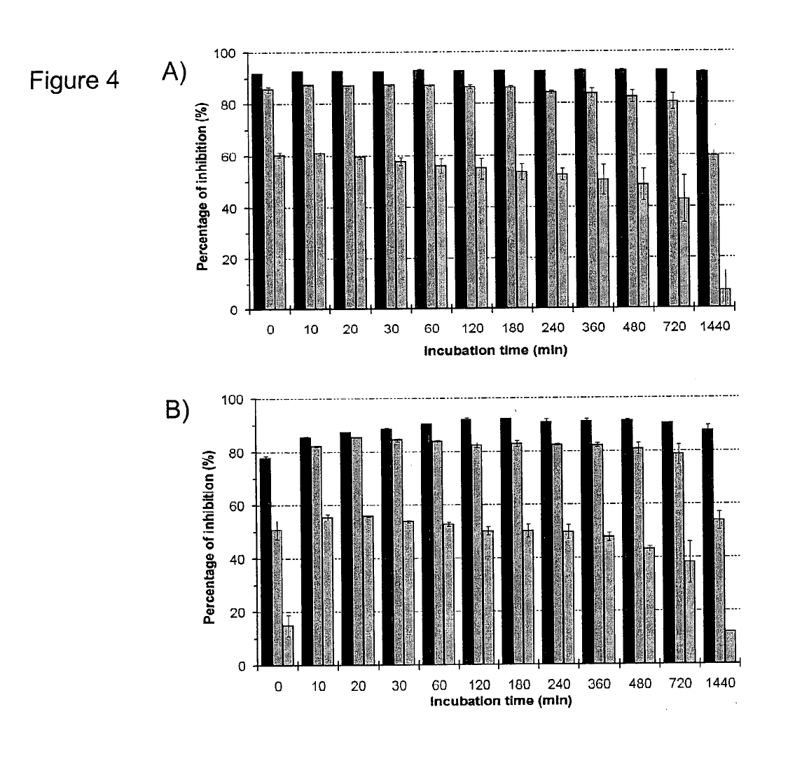

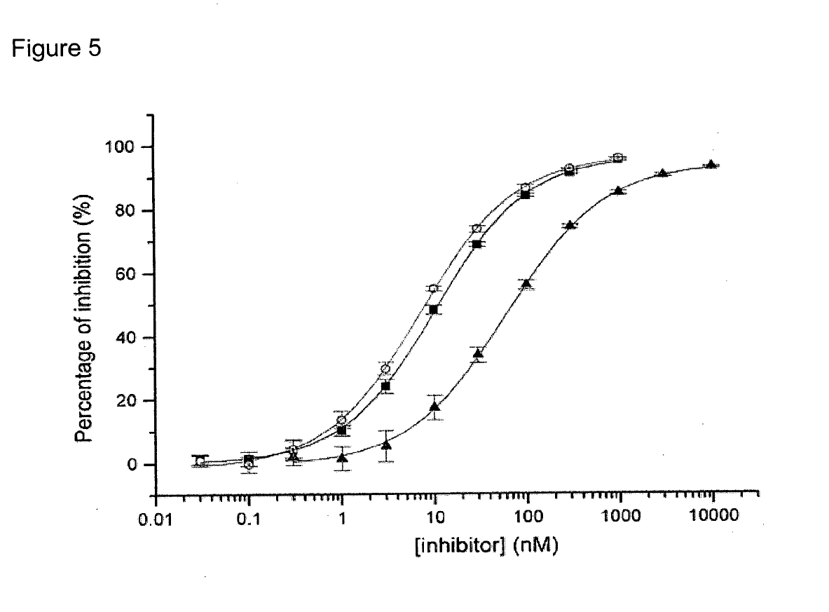

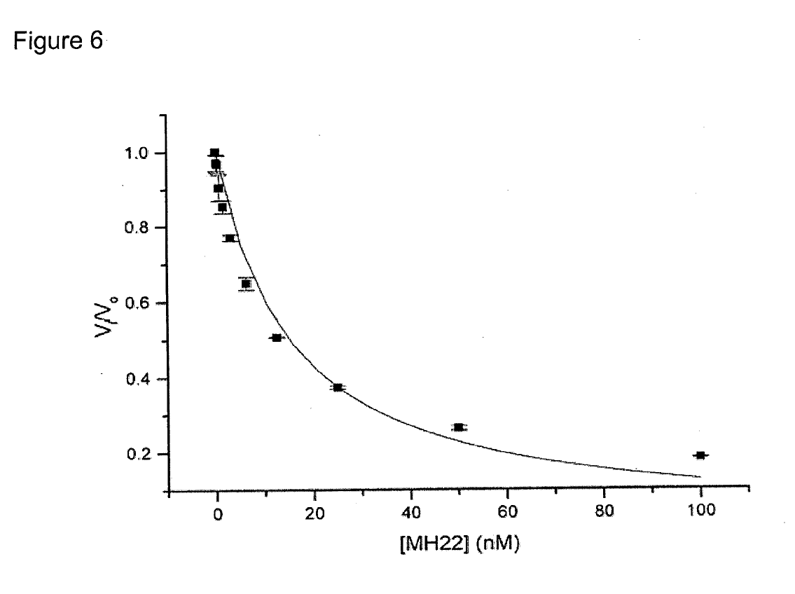

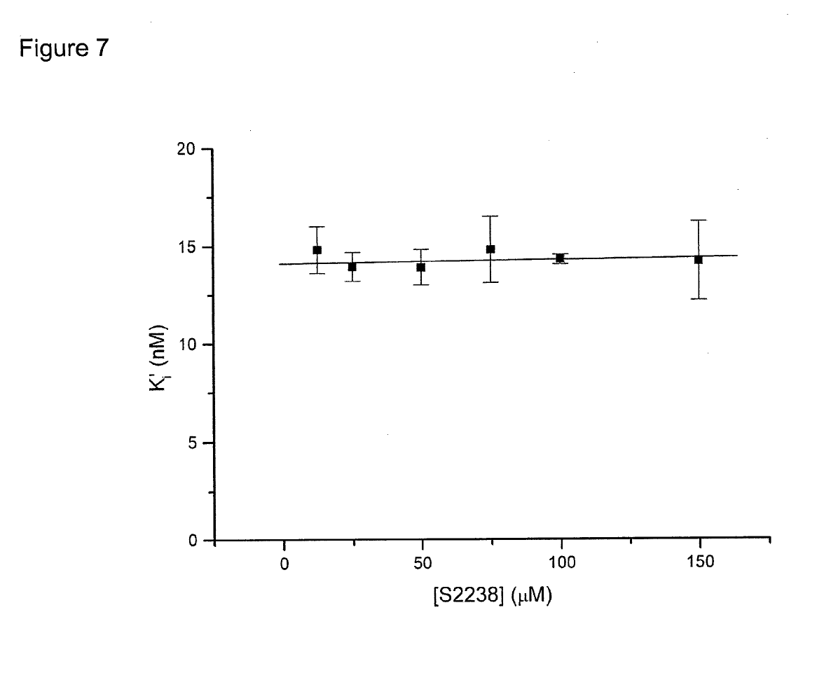

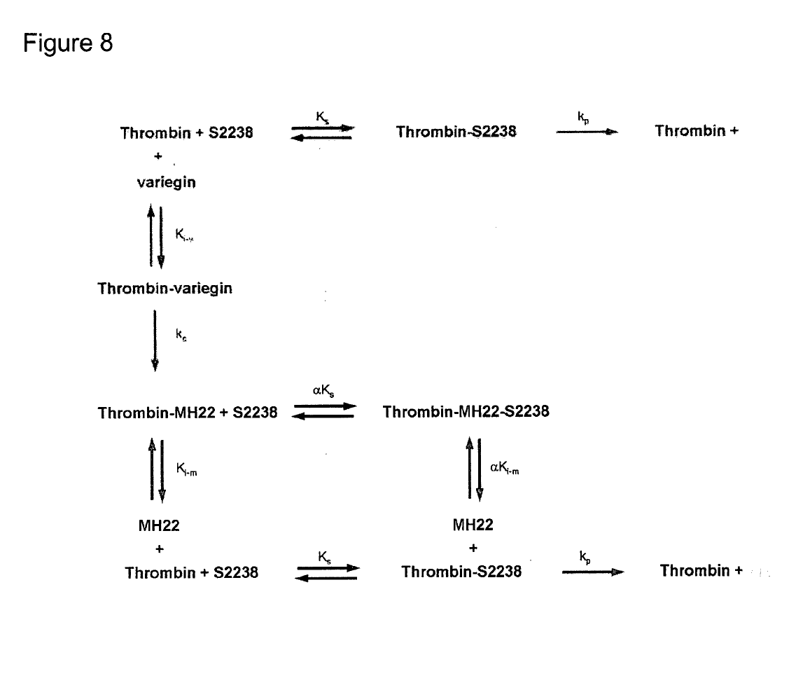

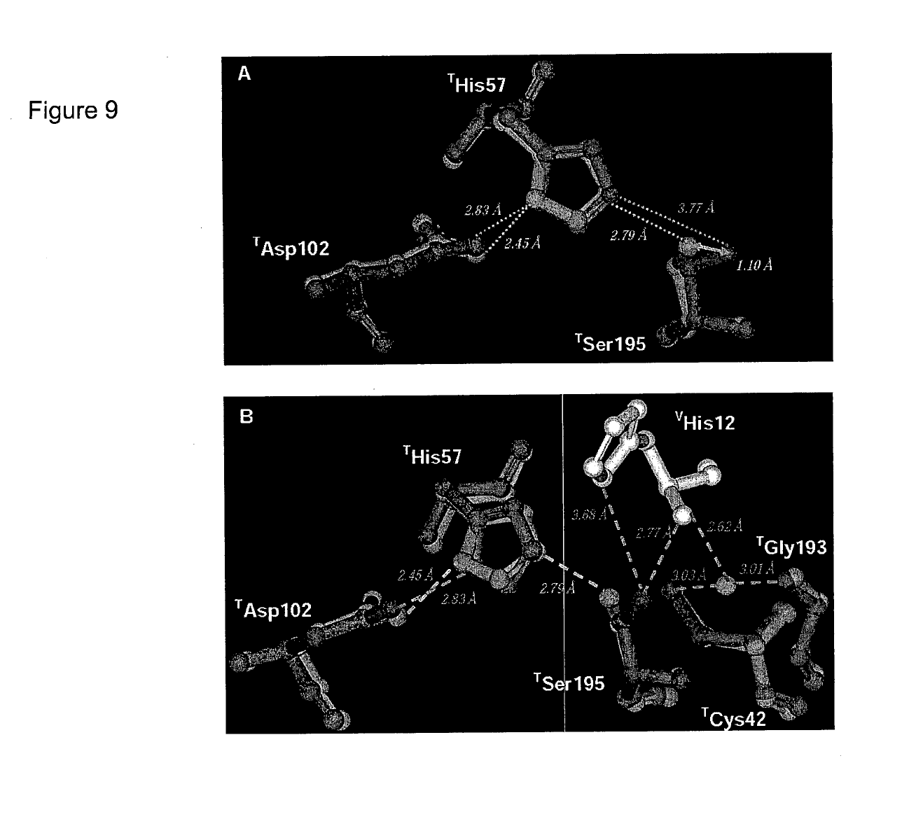

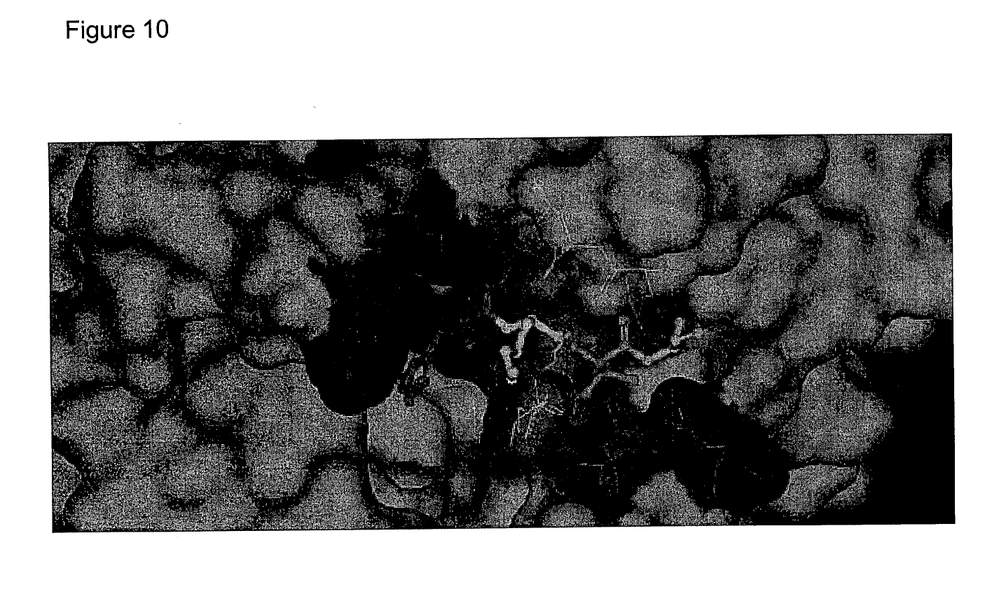

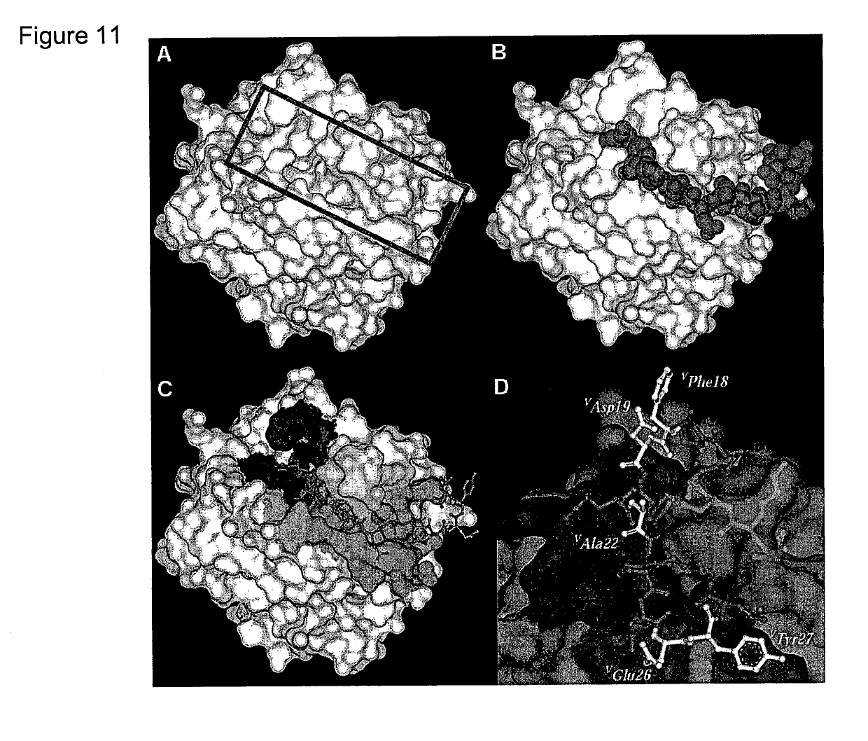

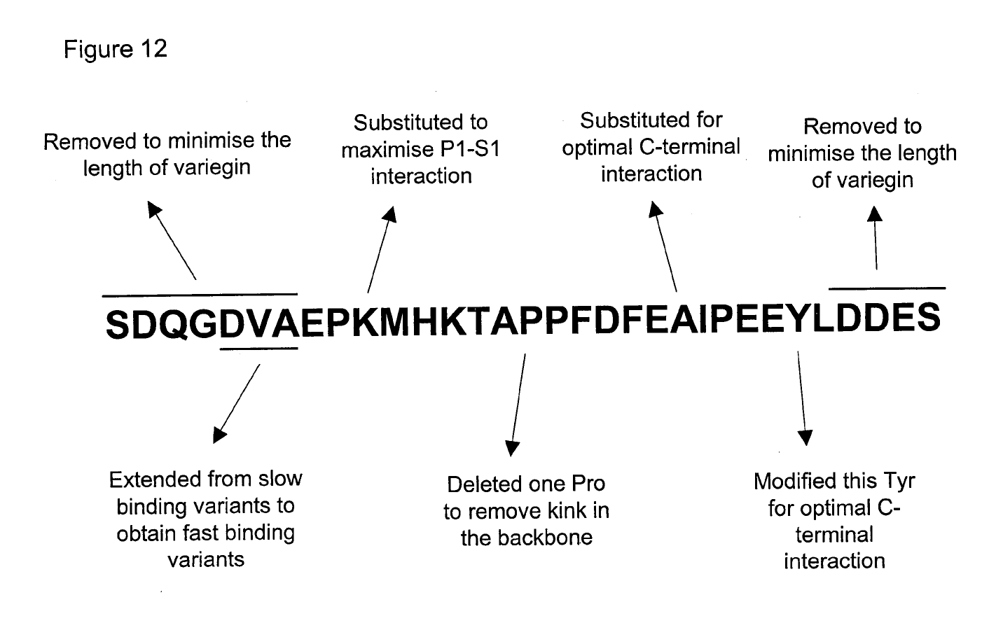

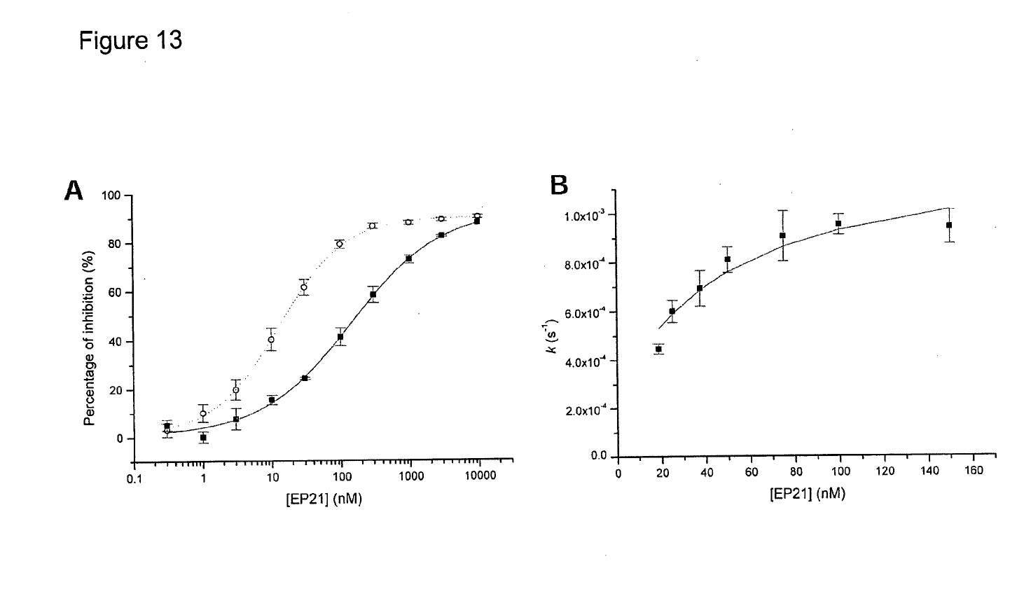

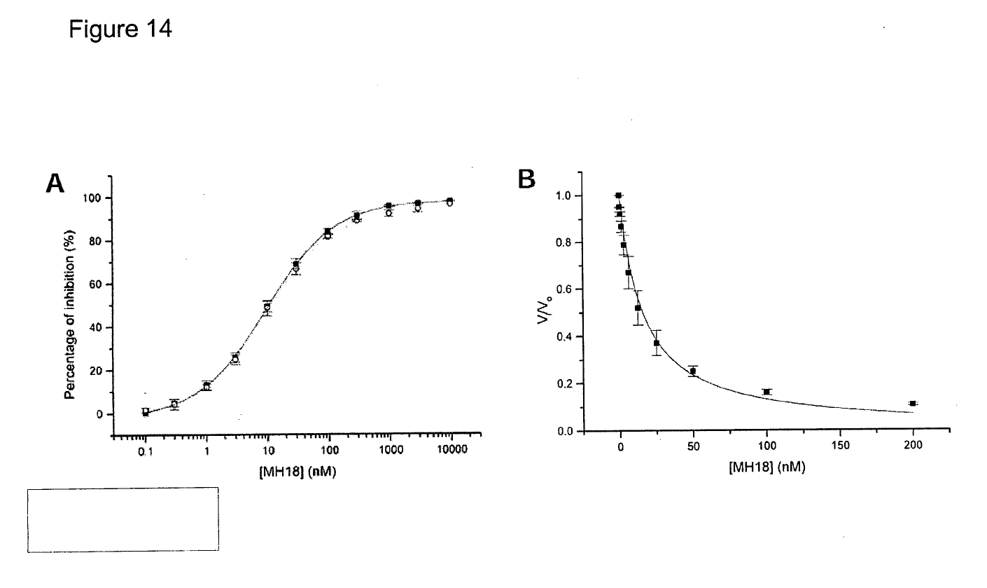

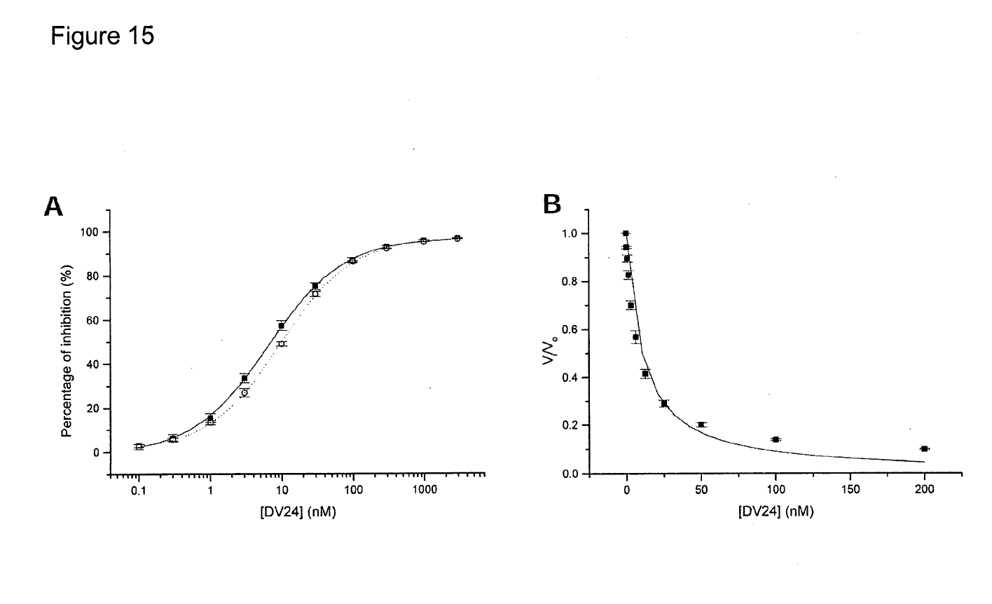

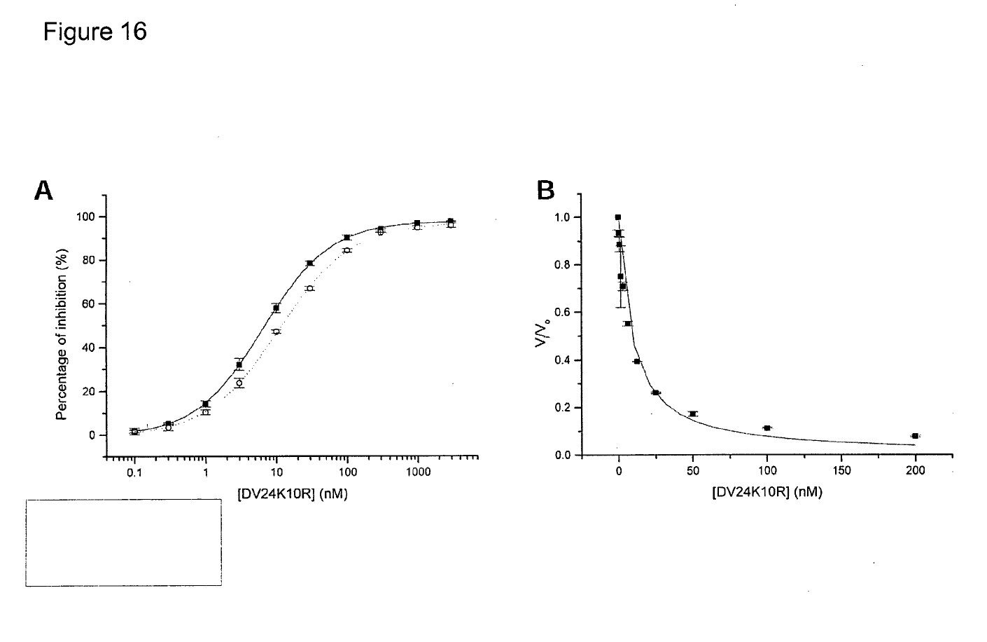

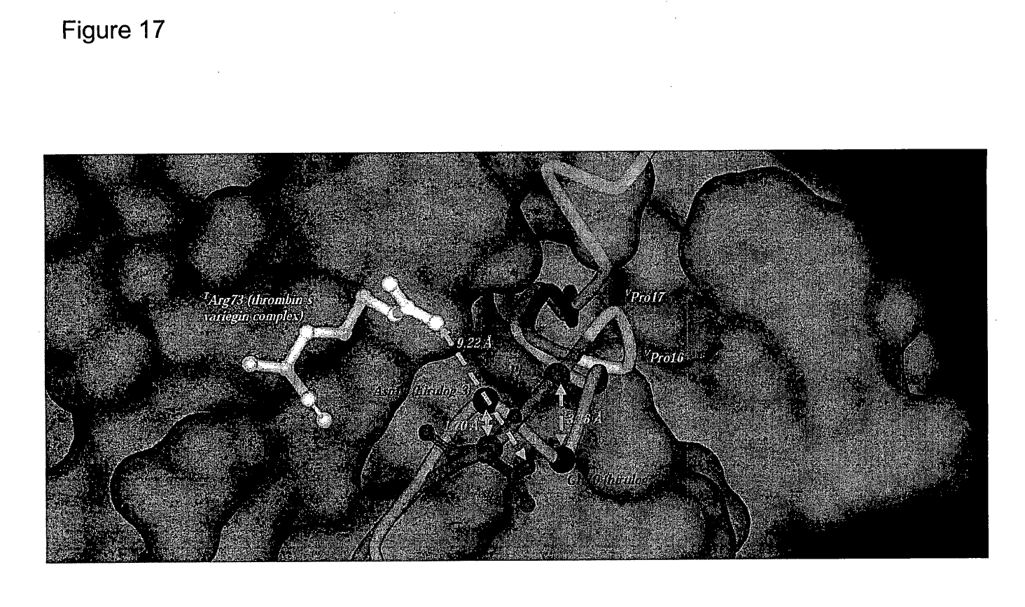

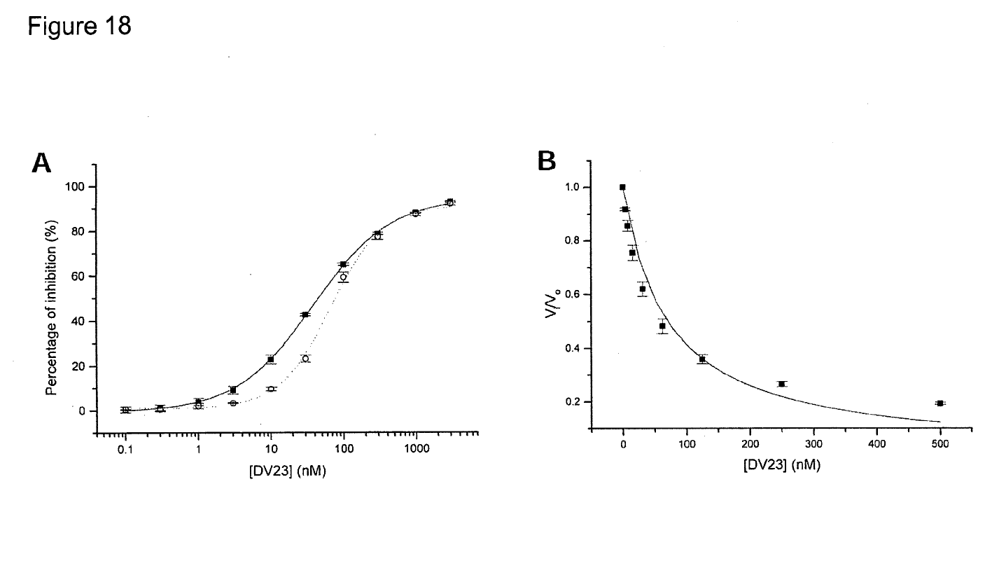

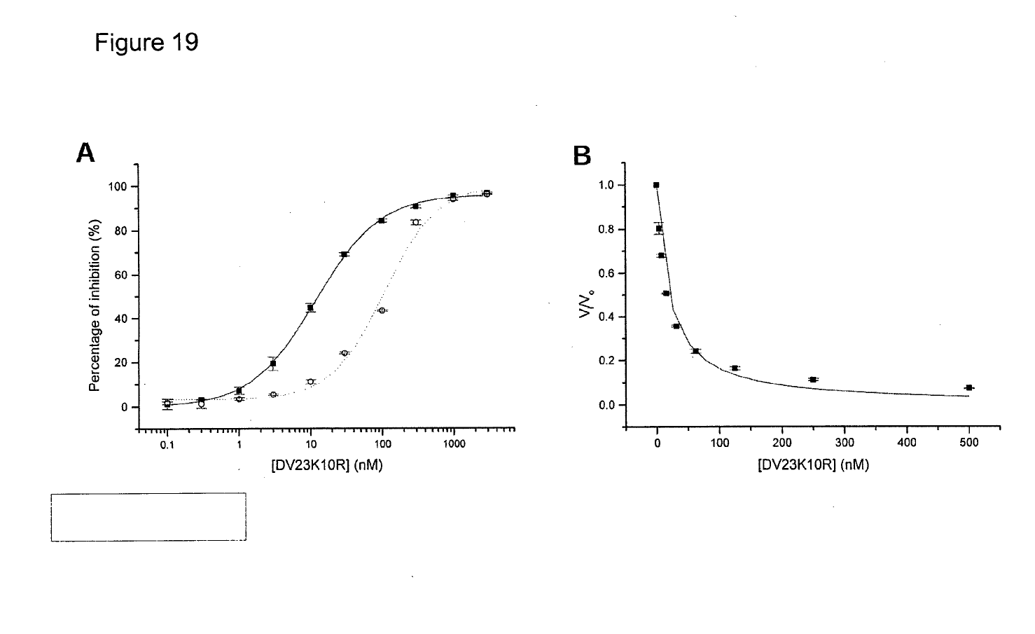

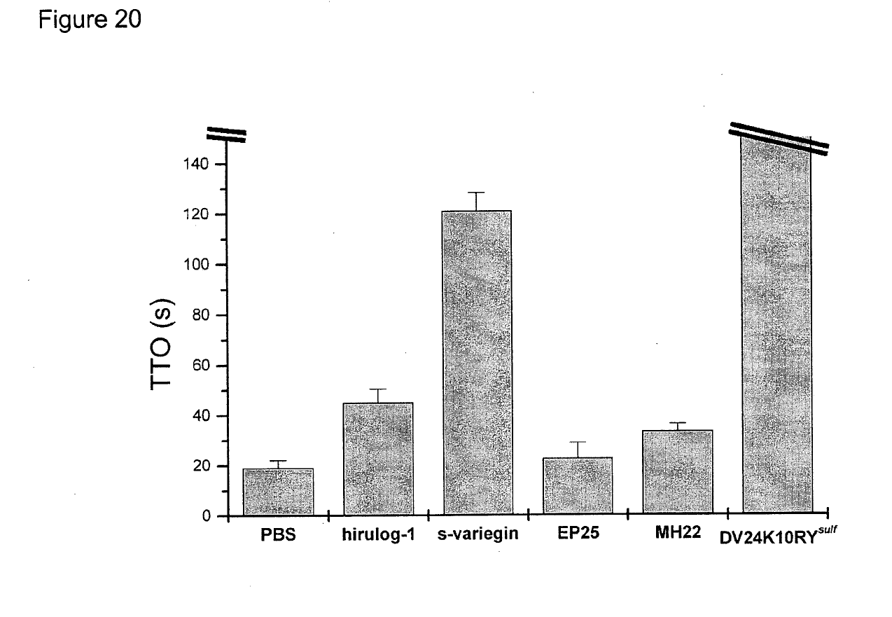

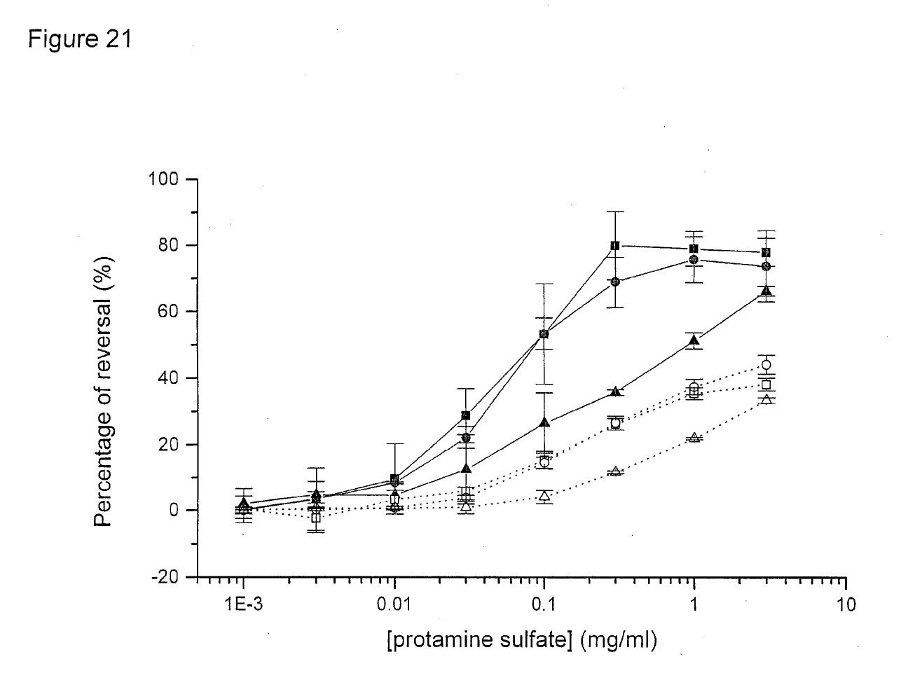

The structure of thrombin-s-variegin complex was solved by the molecular replacement method using the MolRep program (Vagin and Teplyakov, 2000). The coordinates of thrombin-hirulog-3 structure (PDB code 1ABI) (Qiu et al., 1992) were used as a search model. The rotation search located one thrombin-peptide complex molecule in the asymmetric unit. The rigid body refinement after determining the translation components gave a correlation coefficient of 0.60 and R=0.48. The resultant electron density map was of good quality. The Fourier and difference Fourier maps clearly showed electron density for s-variegin. Several cycles of map fitting using the program O version 7.0 (Jones et al., 1991) and refinement using the program CNS version 1.1 (Brunger et al., 1998) led to convergence of R-values. The crystallographic and refinement statistics are given in Table 1. The correctness of stereochemistry of the model was verified using PROCHECK (Laskowsi et al., 1993). Online server PISA (Krissinel and Henrick, 2007) was used to analyze the protein (FIG. 2).

TABLE 1
Crystallographic data and refinement statistics from the current model
Crystallographic data and refinement statistics*

| Data set | Thrombin-s-variegin complex | | |
|---|---|---|---|
| Crystal | | | |
| Space Group | C2 | | |
| Unit Cell Parameter | a = 124.66 Å | b = 50.83 Å | c = 61.54 Å |
| | α = 90.0° | β = 98.7° | γ = 90.0° |

| | |
|---|---|
| Data collection | |
| Resolution range (Å) | 50-2.7 |
| Wavelength (Å) | 0.9795 |
| Observed reflections | 261706 |
| Unique reflections | 15123 |
| Completeness (%) | 96.3 |
| Overall (I/σI) | 19.4 |
| Redundancy | 17.3 |
| R_{sym}^{a} (%) | 5.2 |
| Refinement and quality | |
| Resolution range (Å) I > σ(I) | 20-2.7 |
| R_{work}^{b} | 0.2598 |
| R_{free}^{c} | 0.3301 |
| RMSD bond lengths (Å) | 0.007 |
| RMSD bond angles(°) | 1.5 |
| Average B-factors (Å²) | |
| Protein atoms (2404 atoms) | 66.709 |
| Water molecules (203 atoms) | 68.160 |
| Ramachandran plot | |
| Most favored regions (%) | 76.2 |
| Additional allowed | 22.5 |
| regions (%) | |
| Generously allowed | 1.3 |
| regions (%) | |
| Disallowed regions (%) | 0 |

*Statistics from the current model.
^{a}R_{sym} = Σₕₖₗ Σₗ [|Iᵢ (hkl) − <I(hkl)>|]/Σₕₖₗ I(hkl)
^{b}R_{work} = Σ|F_{obs} − F_{calc}|/Σ|F_{obs}| where F_{calc} and F_{obs} are the calculated and observed structure factor amplitudes, respectively.
^{c}R_{free} = as for R_{work}, but for 8.0% of the total reflections chosen at random and omitted from refinement.

TABLE 2
Root-mean-square deviations (RMSD) between the thrombin-
s-variegin complex and other structures

| | 2HGT | 1ABI | 1HGT | 1PPB | 2AFQ |
|---|---|---|---|---|---|
| | RMSD (Å) | | | | |
| A-chain | | | | | |
| Cα | 0.34 | 0.35 | 0.35 | 0.63 | 0.61 |
| Backbone atoms | 0.56 | 0.60 | 0.67 | 0.59 | 0.75 |
| Side chain atoms | 1.68 | 1.59 | 2.27 | 1.75 | 1.61 |
| B-chain | | | | | |
| Cα | 0.59 | 0.55 | 0.52 | 0.70 | 1.35 |
| Backbone atoms | 0.65 | 0.59 | 0.59 | 0.73 | 1.33 |
| Side chain atoms | 1.33 | 1.21 | 1.19 | 1.29 | 2.30 |
| DFEA(E)IPEEYL | | | | | |
| Cα | NP | 3.62 | 3.87 | NP | NP |
| Backbone atoms | NP | 3.40 | 3.63 | NP | NP |
| Side chain atoms | NP | 7.00 | 7.06 | NP | NP |
| DFEA(E)I | | | | | |
| Cα | 1.46 | 1.22 | 1.26 | NP | NP |
| Backbone atoms | 1.46 | 1.24 | 1.34 | NP | NP |
| Side chain atoms | 3.05 | 2.96 | 2.88 | NP | NP |
| PEEYL | | | | | |
| Cα | NP | 4.98 | 5.33 | NP | NP |
| Backbone atoms | NP | 4.64 | 4.96 | NP | NP |
| Side chain atoms | NP | 9.01 | 9.11 | NP | NP |

2HGT and 1ABI represent thrombin inhibited at both active site and exosite-I, similar to the thrombin-s-variegin complex. 1HGT represents thrombin inhibited at exosite-I only. 1PPB represents thrombin inhibited at active site only. 2AFQ represents inhibitor and Na⁺-free thrombin. Highest differences were found in comparison with 2AFQ mainly due to the extensive changes in surface loops in ‘slow’ form thrombin. RMSD were calculated from Cα, backbone and side chain atoms for thrombin A-chain and B-chain as well as a C-terminal segment (DFEA(E)IPEEYL) which is common in s-variegin, hirulog-1, hirulog-3 and hirugen.
NP: relevant atoms are not present.

### Example 2

### GENERATION of a Non-Competitive Inhibitor of Variegin Following Cleavage by Thrombin

Materials, thrombin, methods of synthesis, purification and mass spectrometry analysis of peptides are as described for Example 1.

### RP-HPLC Analysis of the Cleavage

Peptides were incubated with recombinant α-thrombin at both room temperature in 50 mM Tris buffer (pH 7.4) containing 100 mM NaCl and 1 mg/ml BSA. Reaction mixtures without thrombin were set up as control. After various incubation times, the reactions were quenched with 0.1% TFA buffer (pH 1.8) and loaded onto a SunFire™ C18 column attached to an ÄKTA™ purifier. New peaks other than those present in the chromatogram of both control reaction mixture and 0 min incubation were identified as cleavage products and subjected to ESI-MS to verify their masses. The peaks were integrated to calculate the area under the peaks and the relative percentage of each peak to determine the extent of cleavage.

### Cleavage of Peptides by Thrombin

Variegin is hypothesized to canonically bind thrombin active site, and it is therefore thought that it may be cleaved by thrombin which is similar to other serine protease inhibitors (Witting et al., 1992; Bode and Huber, 1992). Therefore we examined the cleavage of s-variegin by thrombin and its effects on peptides activities. RP-HPLC analysis showed that s-variegin was indeed cleaved by thrombin at both 37° C. and room temperature (˜25° C.). At 0 min of incubation, only peaks corresponding to full-length s-variegin and thrombin were present. Two new peaks of cleavage products appeared and increased with longer incubation times (FIG. 3). These new peaks had masses of 1045 Da (SDQGDVAEPK; SEQ ID NO: 2) and 2582 Da (MHKTAPPFDFEAIPEEYLDDES; MH22; SEQ ID NO: 3) respectively, and corresponded to cleavage at the Lys10-Met11 peptide bond. Cleavage proceeded faster at 37° C. (FIG. 3A) than at 25° C. (FIG. 3B).

### Inhibition of Thrombin Amidolytic Activity by Cleavage Product, MH22

To test if variegin cleavage product is indeed responsible for its prolonged activity, the C-terminal fragment of cleavage (MHKTAPPFDFEAIPEEYLDDES; MH22; SEQ ID NO: 3) was synthesized. This fragment was selected primarily because preliminary data from thrombin-variegin structure obtained by X-ray diffraction suggested that MH22 binds to thrombin after cleavage.

The inhibitory Constant Ki of MH22

The inhibitory constant, Ki of MH22 was determined using S2238 as substrate. MH22 is a fast and tight binding inhibitor. Apparent inhibitory constants, Ki′ were determined in the presence of different concentrations of S2238 using the equation Vₛ=(Vₒ/2Eₜ) {[(Kᵢ′+Iₜ−Eₜ)²+4Kᵢ′Eₜ]^{1/2}−(Kᵢ′+Iₜ−Eₜ)}. Unlike s-variegin which has Ki′ increases linearly with increasing concentrations of S2238 (as shown by the equation Kᵢ′=Kᵢ(1+S/Kₘ)), MH22 Ki′ values remained constant with changes in S2238 concentrations. This behaviour of the curve fits an equation that describes non-competitive inhibition where α=1 (using the equation Kᵢ′=(S+Kₘ)/[(Kₘ/Kᵢ)+(S/αKᵢ)]) and hence, Ki′=Ki (as shown by the equation Kᵢ′=Kᵢ). Fitting the Ki′ values by linear regression derived a value of 14.11±0.29 nM for Ki (FIG. 7). s-Variegin showed a Ki of 0.318±0.020 nM when assayed with human plasma derived thrombin (Ki for recombinant α-thrombin was 0.146±0.014 nM). Thus, the full-length peptide s-variegin is a competitive inhibitor, its cleavage product MH22 is a non-competitive inhibitor of thrombin active site function.

MH22 inhibited thrombin amidolytic activity at equimolar concentration (˜15%) and progress curves of inhibition showed that steady state equilibrium was achieved upon mixing. Thus, similar to s-variegin, MH22 is a fast and tight-binding inhibitor. Dose-response curve showed IC₅₀ value of 11.46±0.71 nM (FIG. 5). s-Variegin inhibition of human plasma derived thrombin has an IC₅₀ value of 8.25±0.45 nM (FIG. 5), slightly higher than that of the recombinant α-thrombin (5.40±0.95 nM) (data not shown).

MH22 was shown to non-competitively inhibit thrombin. Typically, a non-competitive inhibitor binds at a site away from the enzyme active site and allosterically inhibits the active site function. However, the MHKT tetrapeptide is immediately after the scissile bond. Intuitively, binding of this segment to thrombin is likely to be within the active site. The substrate used in the experiments, S2238, has a chemical structure of D-Phe-Pipecolyl-Arg-pNA, with its Arg side chain inserted into thrombin S1 subsite and cleavage occurs between Arg-pNA. With the pNA moiety also occupying the S1′ in the immediate proximity of scissile bond during amidolysis, its presence should theoretically interfere with binding of MH22 to the same site. In such a case, a should be >1 (using the equation Kᵢ′=(S+Kₘ)/[(Kₘ/Kᵢ)+(S/αKᵢ)]) and the plot of Ki′ against substrate concentrations should increase in a hyperbolic manner with increasing concentration of substrate (Copeland, 2000). This type of non-competitive inhibition is usually termed ‘mixed inhibition’. However, under our experimental conditions, the apparent inhibitory constant, Ki′, remained strictly constant with changes in substrate concentrations. Thus, MH22 act as a classical non-competitive inhibitor—binding to both free thrombin and thrombin-substrate complex with the same affinity (FIG. 8). On the same note, the assumption that pNA interferes with MH22 binding does not hold. Therefore, binding sites of MH22 and pNA on thrombin are not overlapping, indicating that residue immediately after the scissile bond (Met11) may not bind to thrombin or binds at a different site rather than the usually observed S1′.

### Example 3

### Design and Characterisation of Variegin Variants

Thirteen new variegin variants were designed based on the thrombin-s-variegin structure as well as background information available on thrombin interactions. The general approach was to first optimize the length of variegin before optimizing several key positions on variegin to obtain maximum interaction with thrombin (FIG. 12).

### Example 4

### Thrombin Inhibitory Activity

Activities of s-variegin (SEQ ID NO: 1), EP25 (SEQ ID NO: 6), MH22 (SEQ ID NO: 3) and hirulog-1 (SEQ ID NO: 14) were assayed by their abilities to inhibit thrombin amidolytic activity on S2238.

The activity of each peptide was determined by the inhibition of recombinant α-thrombin amidolytic activity assayed using the chromogenic substrate S2238. All assays were performed in 96-well microtiter plates in 50 mM Tris buffer (pH 7.4) containing 100 mM NaCl and 1 mg/ml BSA at room temperature. Typically, 100 μl of peptide and 100 μl of recombinant α-thrombin were pre-incubated for different durations before the addition of 100 μl of S2238. Details of each experiment are described along with the graphs representing the results obtained. The rates of formation of colored product pNA were followed at 405 nm for 10 min with a SPECTRAMax Plus microplate spectrophotometer. Percentage inhibition was calculated by taking the rate of increase in absorbance in the absence of inhibitor as 0%. Dose-response curves were fitted using Origin software to calculate IC₅₀ values with the following logistic sigmoidal equation: y=A₂+(A₁−A₂)/[1+(x/x₀)^{H}], where y is percentage of inhibition, A₂ is right horizontal asymptote, A₁ is left horizontal asymptote, x is log 10 of inhibitor concentration, x₀ is point of inflection and H is the slope of the curve. IC₅₀ was calculated by substituting ‘50’ into y.

Effects of pre-incubation times (hence cleavage) on thrombin inhibitory activities of peptides were performed with the same assay, varying pre-incubation times and/or concentrations of BSA. For experiments to ascertain integrity of thrombin exosite-I during extended incubation time, parallel sets of assay were performed with or without addition of freshly prepared inhibitors after 28 h of pre-incubation. All data obtained were fitted using Origin software to the y=A₂+(A₁−A₂)/[1+(x/x₀)^{H}] equation for calculation of IC₅₀ values and to the equations:

Vₛ=(Vₒ/2Eₜ){[(Kᵢ′+Iₜ−Eₜ)²+4Kᵢ′Eₜ]^{1/2}−(Kᵢ′+Iₜ−Eₜ)} for tight binding inhibition;

Kᵢ′=Kᵢ(1+S/Kₘ) for competitive inhibition;

Kᵢ′ (S+Kₘ)/[(Kₘ/Kᵢ)+(S/αKₜ)] for non-competitive inhibition;

Kᵢ′=Kₜ for classical non-competitive inhibition;

P=V_{f}t+(Vᵢ−V_{f})(1−e^{-kt})/k+Pₒ for slow-binding inhibition;

k=K₄+K₃Iₜ/[Iₜ+Kᵢ′(1+S/Kₘ)] for calculation of the dissociation constant; and

Kᵢ=Kᵢ′[K₄/(K₃+K₄)] for calculation of the overall inhibitory constant.

### Inhibition of Thrombin Amidolytic Activity by EP21 And MH18

The lack of electron densities for the four s-variegin C-terminal residues [^{v}(29DDES32)] in the complex structure indicated that these four residues are unlikely to interact strongly with thrombin. Further, these residues were not present in hirulogs or hirugen. Considering the vast differences between C-terminal conformations of s-variegin and hirulog/hirugen, it would be interesting to examine the role of these residues. Two truncation variants, EP21 (SEQ ID NO: 8) and MH18 (SEQ ID NO: 9), corresponded to EP25 and MH22 respectively but lacking those four C-terminal residues, were designed and characterized. Since EP25 and s-variegin binds to thrombin with the same affinity, C-terminal truncation variant of s-variegin was not synthesized. Progress curves of thrombin inhibition by EP21 (SEQ ID NO: 8) showed two-phase equilibria in the absence of pre-incubation, typical of a slow binding inhibitor. EP21 activity was dependent on pre-incubation time. IC₅₀ decreased from 176.9±6.8 nM (without pre-incubation) to 16.2±2.9 nM (after 20 min pre-incubation) (FIG. 13A). By fitting data to the equations P=V_{f}t+(Vᵢ−V_{f})(1−e^{-kt})/k+Pₒ, k=K₄+K₃Iₜ/[Iₜ+Kᵢ′(1+S/Kₘ)], and Kᵢ=Kᵢ′[K₄/(K₃+K₄)], the inhibitory constant, Ki, calculated for EP21 inhibition of thrombin is 0.315±0.024 nM (FIG. 13 B). All the values are similar to that of EP25 (SEQ ID NO: 6) indicates that truncation of four C-terminal residues does not significantly alter the peptide activity.

MH18 (SEQ ID NO: 9) inhibited thrombin amidolytic activity at equimolar concentration (˜15%) and steady state equilibrium was achieved upon mixing. Thus, MH18 is a fast, tight-binding inhibitor for thrombin. Dose-response curves showed IC₅₀ values of 10.9±1.2 nM (without pre-incubation) and 11.7±1.9 nM (after 20 min pre-incubation) (FIG. 14A). These values are essentially identical with data obtained with MH22 (SEQ ID NO: 3). Apparent inhibitory constant, Ki′ of MH18 were obtained with 100 μM of S2238, fitting data with the equation Vₛ=(Vₒ/2Eₜ) {[(Kᵢ′+Iₜ−Eₜ)²+4Kᵢ′Eₜ]^{1/2}−(Kᵢ′+Iₜ−Eₜ)} (FIG. 14B). Assuming that MH18 is a non-competitive inhibitor like MH22, equations Kᵢ′=(S+Kₘ)/[(Kₘ/Kᵢ)+(S/αKᵢ)] and Kᵢ′=Kᵢ were solved to derive the inhibitory constant Ki of 14.9±3.5 nM which is consistent with Ki of MH22. This set of results ascertained the above conclusion that the four C-terminal residues are not involved in binding to thrombin.

Inhibition of Thrombin Amidolytic Activity by DV24

Our earlier data showed that the seven N-terminal residues of variegin are responsible for its fast binding kinetics; when removed, the binding characteristics changed from fast to slow without loss in affinity (data not shown). We then postulated that the highly basic thrombin exosite-II could help to steer variegin N-terminus residues (which contains two negatively charged residues in its sequence 1 SDQGDVA7) into an optimal orientation close to the active site allowing rapid formation of short-range interactions. Since thrombin exosite-II is located about 10 Å away from the active site (Page et al., 2005), this distance could theoretically be covered by at least three N-terminal residues in an extended conformation. In order to produce a peptide that retained the fast-binding property, we designed and characterized a peptide extending EP21 by three residues at the N-terminus. One out of the two acidic N-terminal residues, ^{v}Asp5, is present in this variant, which is named DV24 (SEQ ID NO: 10).

Instead of the two-phase equilibria usually observed for slow binding inhibitor, DV24 (SEQ ID NO: 10) progress curves of thrombin inhibition were similar to s-variegin—reaching steady state equilibrium upon mixing. Thus, DV24 is a fast and tight-binding inhibitor. Activity of DV24 decreased with increasing pre-incubation time due to cleavage by thrombin. Dose-response curves showed IC₅₀ values of 7.49±0.28 nM (without pre-incubation) and 10.07±0.60 nM (after 20 min pre-incubation) (FIG. 15A). Assuming competitive inhibition, the equations Vₛ=(Vₒ/2Eₜ) {[(Kᵢ′+Iₜ−Eₜ)²+4Kᵢ′Eₜ]^{1/2}−(Kᵢ′+Iₜ−Eₜ)} and Kᵢ′=Kᵢ(1+S/Kₘ) were used to derive the inhibitory constant Ki of 0.306±0.029 nM, consistent with Ki of s-variegin (FIG. 15B). Therefore, we managed to design a peptide that is eight residues shorter than s-variegin but retained the fast-binding kinetic with the same Ki.

### Inhibition of Thrombin Amidolytic Activity by DV24K10R

One difference between variegin and other thrombin substrates/inhibitors is the presence of Lys in the P1 position of the scissile bond. Typically, Arg is found in this position for thrombin substrates. The electrostatic interaction between the side chain guanidinium group of Arg and the side chain carboxylate group of ^{T}Asp 189 in the S1 subsite is usually preferred. In contrast, P1 Lys usually interacts with Asp 189 through a water molecule (Perona and Craik, 1995), resulting in reduced affinity and specificity (Vindigni et al., 1997). The absence of electron density for residues before the scissile bond [^{v}(1SDQGDVAEPK10)] in the thrombin-s-variegin structure probably implies the lack of strong affinity for thrombin within this segment. Therefore, using DV24 as template sequence, the P1 residue Lys10 was replaced by Arg in a new variant named DV24K10R (SEQ ID NO: 11).

IC₅₀ obtained for DV24K10R is 6.98±0.76 nM without pre-incubation, which is similar to IC₅₀ of DV24 (7.49±0.28 nM). However, IC₅₀ for DV24K10R is 12.01±0.41 nM after 20 min pre-incubation, slightly higher than that of DV24 (10.07±0.60 nM). It is likely that cleavage of the peptide proceeds faster with the presence of P1 Arg (FIG. 16A). Affinity to thrombin has increased slightly, indicated by a small drop in Ki value 30 to 0.259±0.015 nM (compared to 0.306±0.029 nM for DV24) (FIG. 16B). Thus, substitution of Lys10 by Arg only minimally improved thrombin affinity of variegin despite previous observations that P1 Lys generally binds 10-fold weaker than P1 Arg (Page et al., 2005). With this observation in mind, subsequent designs of new variants are typically performed with both Lys and Arg at P1 position.

### Inhibition of Thrombin Amidolytic Activity by DV23 and DV23K10R

The phenyl group of VPhe20 is inserted into an apolar cavity in thrombin and interacts with ^{T}Phe34 by π-π stacking. This interaction is also present in hirulog, hirugen and hirudin complex structures and marks the start of the C-terminal segment—DFEA(E)IPEEYL—where s-variegin and hirulog/hirugen are almost identical. In s-variegin, there are nine residues present in between the P1 Lys residue and the Phe [V(11 MHKTAPPFD19)]. However, in hirulog-⅓, the same distance is spanned by only eight residues (4PGGGGNGD11). Analysis of the thrombin-s-variegin structure showed that ^{v}Pro16 and ^{v}Pro17 induced a kink in its backbone, causing a slight bend upwards, away from thrombin. This in turn caused a displacement of ^{v}Phe18 and ^{v}Asp19 by about 3.16 Å and 1.70 Å from their corresponding residues in hirulog-3—Gly10 and Asp11—as measured by distances between their Cα atoms (FIG. 17). Crucially, Asp11 of hirulog-3 make an ion pair with ^{T}Arg73 which is absent between the analogous ^{v}Asp19 and ^{T}Arg73. In fact ^{v}Asp19 side chain points to the opposite direction into the solvent, creating a 9.22 Å distance between the ^{v}Asp190S and ^{T}Arg73 NH2 (FIG. 17). Therefore, Prol 6 was deleted from s-variegin sequence to remove the kink in the backbone for the repositioning of ^{v}Asp19 so as to restore the ionic interaction. Using DV24 and DV24K10R as template sequences, variants DV23 (SEQ ID NO: 13) and DV23K10R (SEQ ID NO: 12) were designed, synthesized and characterized.

Both DV23 and DV23K10R showed decrease in activities compared to their templates. DV23 IC₅₀ values are 45.4±1.6 nM (without pre-incubation) and 77.8±6.1 nM (after 20 min pre-incubation) (FIG. 18A). DV23 Ki is 2.19±0.23 nM (FIG. 18B). All values showed an average of ˜7-fold reduction in activity compared to DV24. The other variant, DV23K10R is also less active compared to its template, DV24K10R. The peptide IC₅₀ values for the peptides are 12.9±1.0 nM (without pre-incubation) and 101.9±1.2 nM (20 min pre-incubation) (FIG. 19A). DV23K10R Ki is 0.600±0.010 nM (FIG. 19B). Its affinity to thrombin is about 2-fold weaker than DV24K10R. While DV23K10R is more active than DV23 without pre-incubation with thrombin, the trend is reversed after 20 min of pre-incubation. This is in agreement with the observation that peptide with Arg at P1 (DV24K10R) is hydrolyzed by thrombin at a faster rate than peptide with Lys at P1 (DV24). Moreover, the rapid loss of activity also implies that the cleavage product no longer inhibits thrombin potently. Thus, the deletion of ^{v}Pro16 appears to have an adverse effect on the activities of both the intact peptide and cleavage product. Considering the proximity of variegin P′ residues to ^{v}Pro16, removal of this residue probably compromised the interactions within the prime subsites.

### Example 5

### Interactions of Thrombin Inhibitors with the Thrombin Active Site & Displacement of Residues of Catalytic Triad

Of the 17 observed s-variegin residues, selected few N-terminal residues are of special interest. The thrombin-s-variegin structure was compared with thrombin-hirugen structure (PDB: 1HGT) as they shared one common characteristic—both occupy the exosite-I but not the non-prime subsites of active site (since N-terminal cleavage fragment of s-variegin is not present). Of the three catalytic residues (^{T}His57, ^{T}Asp102 and ^{T}Ser195), the most striking difference was with the Oγ atom of ^{T}Ser195. In the thrombin-s-variegin structure, ^{T}Ser195 Oγ is displaced by 1.1 Å. As a result, the hydrogen bond with NE of ^{T}His57 (which should be part of the catalytic charge relay system) is absent in the thrombin-s-variegin structure. The distance between the two atoms increased to 3.77 Å (FIG. 9A). The corresponding distance in the thrombin-hirugen structure is 2.79 Å (FIG. 9A). The displacement of ^{T}Ser195 Oγ is due to interaction with s-variegin. Particularly, a new and extensive network of hydrogen bonds between ^{v}His12, ^{T}Ser195, ^{T}Gly193 and ^{T}Cys42 as well as a water molecule perturbs the catalytic charge relay network. Crucially, ^{v}His12 backbone N (donor) is engaged with Oγ of ^{T}Ser195 (acceptor) through hydrogen bond (2.77 Å) while ^{v}His12 side chain No (acceptor) could contribute a weak hydrogen bond with ^{T}Ser195 Oγ (donor) (3.68 Å). In addition, the ^{v}His12 backbone N also hydrogen bonds to backbone N of ^{T}Gly193 and Sγ of ^{T}Cys42 via a water molecule. Effectively, the electrons on ^{T}Ser195 Oγ get delocalized into this hydrogen bonding network, rendering it a weak nucleophile and incapable of attacking the backbone C of the substrate efficiently. In addition, involvement of main chain N of ^{T}Gly193 in this hydrogen network prevents the formation of the oxyanion hole, further reducing the catalytic capability of this complex (FIG. 9B).

No other major structural changes are observed around the active site cleft, including the non-prime subsites (occupied by substrates residues N-terminal to the scissile bond) and Na⁺binding loop. This indicates that C-terminal cleavage fragment of s-variegin (MH22; SEQ ID NO: 3) does not affect binding affinities of small peptidyl substrates, such as S2238. This observation supports the classical non-competitive inhibition observed for MH22. As shown in FIG. 8, substrate (S2238) binding (Ks) to thrombin was not affected by MH22, but rate of product formation (kp) of the reaction decreased due to inefficient catalysis. The condition for the observed classical non-competitive inhibition precludes binding of ^{v}Met11 in the same site occupied by pNA moiety of S2238. This assumption is supported by the absence of ^{v}Met11 density in the structure. Thus, ^{v}Met11 might not be in direct contacts with thrombin and was disordered in the complex. The lack of interactions between ^{v}Met11 and thrombin is consistent with the thinking that thrombin. S1′ prefer residues with small side chain such as Gly, Ala or Ser (Bode et al., 1992).

### Example 6

### Interaction of Variegin with Exosites of Thrombin & Smooth Fitting of Variegin into the Cannon-Like Cleft of Thrombin

In addition to the extensive network of hydrogen bonds, other interactions between s-variegin P2′ to P5′ (^{v}His12, ^{v}Lys13, ^{v}Thr14 and ^{v}Ala15) with thrombin further anchored this moiety in the prime subsites of thrombin. Extensive interface contacts between ^{v}His12 to ^{v}Ala15 of s-variegin and thrombin surface formed by residues in 60-loop, autolysis loop and 34-loop was observed (FIG. 10). Comparing residues to residues, P2′ of s-variegin (^{v}His12) is displaced by 2.77 Å towards the active site, compared to P2′ of hirulog-3 (Gly5) (measured by the displacement of Cα atoms). This discrepancy can be partially explained with the use of β-homo-Arg as P1 in hirulog-3 which displaced the backbone atoms of P1′ by about a bond length. It could also be due to the involvement of ^{v}His12 in the extensive hydrogen bonding network with thrombin active site, which draws the residues closer towards ^{T}Ser195 (FIG. 9B). Consequently, the ^{v}His12 side chain occupied part of the small S1′ subsite observed in hirulog-3. ^{v}His12 backbone O is hydrogen bonded to ^{T}Lys60F Nζ ((2.74 Å). P2′ ^{v}His12 in s-variegin structure is surrounded by and in contact with ^{T}Cys42, ^{T}His57, ^{T}Trp60D, ^{T}Lys60F, ^{T}Glu192 and ^{T}Ser195. Partial occupation by ^{v}His12 in S1′ limits the space available to accommodate the bulky side chain of P1′ ^{v}Met. Thus, it is possible for P1′ ^{v}Met to point out into the solvent. P3′ VLys side chain runs close and parallel with ^{T}Glu192 side chain, allowing hydrophobic interactions between the aliphatic side chains of both residues. However, strong ionic pairing between ^{v}Lys13 and ^{T}Glu21 is not likely as the distance between the two charges is more than 5.0 Å. The backbone of ^{v}Lys13 is also in contact with ^{T}Leu41. P4′ ^{v}Thr side chain appeared to be directed towards the base of the autolysis loop, which made the analysis less certain due to the flexible nature of the loop. Nonetheless, the side chain occupies a small cavity lined by ^{T}Gly142, ^{T}Asn143, ^{T}Glu192, ^{T}Gly193 and ^{T}Glu151. Hydrogen bonds are observed between ^{v}Thr140y and ^{T}Glu192 backbone O (2.53 Å) and ^{T}Asn143 Oδ (3.29 Å). P5′ Ala side chain was buried deep into the bottom of the canyon-like cleft lined by Leu40 providing a hydrophobic contact.

Thrombin-s-variegin binding in exosite-I is mainly driven by hydrophobic interactions. On the whole s-variegin fitta firmly into the canyon-like cleft extending from the thrombin active site to exosite-I (FIGS. 11A & B). Many apolar residues in between these loops lined the bottom of the cleft. The walls of the cleft are formed by the 60- and autolysis loop near thrombin active site as well as 34- and 70-loops at around exosite-I (Rydel et al., 1991; Bode et al., 1992; Huntington, 2005). The binding of s-variegin with thrombin is driven mainly by hydrophobic contacts at the apolar bottom and the wall of the cleft. The thrombin residues that are involved in binding are: (i) at the bottom of these surface loops: ^{T}Met32, ^{T}Leu40, ^{T}Leu41, ^{T}Cys42, ^{T}Leu65, ^{T}Arg67, ^{T}Lys81, ^{T}Ile82 and ^{T}Met84; (ii) in 60-loop: ^{T}Trp60D and ^{T}Lys60F; (iii) in autolysis loop: ^{T}Gly142, ^{T}Asn143 and ^{T}Gln151; (iv) in 34-loop: ^{T}Phe34, ^{T}Lys36, ^{T}Pro37, ^{T}Gln38 and ^{T}Glu39; (iv) in 70-loop: ^{T}Arg73, ^{T}Thr74, ^{T}Arg75, ^{T}Tyr76 and ^{T}Arg77A (FIG. 11C). Specific side chains interactions are important, as all but five residues on the entire s-variegin polypeptide chain (^{v}Phe18, ^{v}Asp19, ^{v}Ala22, ^{v}Glu26 and ^{v}Tyr27) have their side chains buried in the interfaces with thrombin (FIG. 11D). Also, some of the strongest hydrophobic contacts observed include: (1) ^{v}Phe20 with ^{T}Met32, ^{T}Phe34 (π-π stacking) and ^{T}Leu40; (2) ^{v}Ile23 with ^{T}Phe34, ^{T}Leu65, ^{T}Tyr76 and ^{T}Ile82; (3) ^{v}Pro24 with ^{T}Tyr76.

In contrast to the apolar nature of the bottom of canyon-like cleft, the top surfaces of these loops, especially the 70-loop, are dominated by positively-charged residues. These basic residues include ^{T}Arg35, ^{T}Lys36, ^{T}Arg73, ^{T}Arg75, ^{T}Lys81, ^{T}Arg77A, ^{T}Lys 109, ^{T}Lys110 and ^{T}Lys149E, forming a positively-charged entrance over the apolar canyon-like cleft. Despite the presence of multiple acidic residues in s-variegin C-terminus (^{v}Asp19, ^{v}Glu21, ^{v}Glu25 and ^{v}Glu26), only one ion pair is formed between ^{v}Glu21 and ^{T}Arg75. Interestingly, in hirulog, hirugen and hirudin structures the analogous Glu makes an ion pair with ^{T}Arg75 of a 2-fold symmetry-related molecule (Rydel et al., 1990; Skrzypczak-Jankun et al., 1991; Qiu et al., 1992).

This interaction was suggested to happen within the same thrombin-inhibitor pair in solution although structurally it was not observed (Rydel et al., 1990). In our structure, the ^{T}Arg75 side chain is rotated by 96.8° about Cγ compared to ^{T}Arg75 of hirulog-3 to facilitate the electrostatic interaction with ^{v}Glu21, providing structural evidence for the existent of the predicted interaction. Similarly, in hirulog-⅓ and hirugen structures, only one ion pair can be observed. However, this interaction is between ^{T}Arg73 and an Asp corresponds to ^{v}Asp19. Formation of an equivalent ion pair in the thrombin-s-variegin structure was not possible as ^{v}Asp19 side chain points in an opposite direction into the solvent. This change in side chain orientation is most likely due to the kink in s-variegin backbone which is induced by Pro16-Pro17.

At the end of the canyon-like cleft is a relatively flatter surface formed by thrombin residues ^{T}Asp63-^{T}Ile68 and ^{T}Lys81-^{T}Leu85. Despite being present in close proximity, s-variegin C-terminal residues ^{v}Pro24 to ^{v}Leu28 stacked loosely on top of this surface with two of the side chains (^{v}Glu26 and ^{v}Tyr27) pointing into the solvent (FIG. 11D). This s-variegin segment is in a different conformation when compared to hirulog-3/hirugen despite sharing similar sequences. Compared to the constricted canyon-like cleft, the relatively open surface around this region could be responsible for the hirudin/hirulog/hirugen/s-variegin C-terminal conformational heterogeneity discussed above.

### Example 7

### An Animal Model of Venous Thrombosis

### Materials

Adult zebrafish and zebrafish larvae were maintained in Department of Biological Sciences, University of North Texas, Denton, Tex., USA.

### Synthesis, Purification and Mass Spectrometry of Peptides

Synthesis, purification and mass spectrometry analysis of all peptides followed the procedures described in Example 1.

### Breeding of Zebrafish

The zebrafish breeding tank was assembled with two 1 L tanks. The bottom of one tank was cut off and placed onto a sterilized mesh. This tank was subsequently inserted into a second tank with intact bottom. A pair of zebrafish was then placed into the breeding tank at the end of a light cycle. The mesh served to isolate the pair of zebrafish in the top tank. Within the first 2 hours of the next light cycle, the fish begin to spawn and eggs collect at the bottom of the breeding tank under the protection of the mesh. After removal of fish, water in the breeding tank was filtered through a brine shrimp net which retains the eggs. The net was immediately inverted over a Petri dish containing E3 media (5 mM NaCl, 0.17 mM KCl, 0.33 mM CaCl₂, 0.33 mM MgSO₄ and 10-5% methylene blue), releasing the eggs and other contaminating materials such as feces. The eggs were subsequently transferred into fresh E3 media with a plastic Pasteur pipette. This cleaning step was repeated twice before the eggs were transferred into a new tank and maintained at 28.5° C. for hatching.

### Microinjection

Larvae at 4 days-post-fertilization (dpf) were used to determine in vivo activities of peptides in venous thrombosis model. Intravenous microinjections of peptides were performed using Nanoject II (Drummond, Broomall, Pa., USA) with glass injection needles (3.5-in. capillaries) pulled on a vertical pipette puller (Knopf, Tujunga, Calif.). The tips of the pulled needles were clipped using small scissors and filled with 500 μM of peptides dissolved in phosphate buffered solution (PBS). 10 nl of peptides or PBS were injected into the larvae circulation through the posterior (caudal) cardinal vein.

### Mounting of Zebrafish Larvae in Agarose

Each larvae injected with peptides were placed in 0.5 ml of distilled water added with 6 μl of 10 mM Tricaine solution for anesthetization. To this water containing larvae equal volume of 1% low-melt agarose solution (maintained at 35° C. in a water bath) was added. The mixture (with anesthetized larvae) was poured onto a glass microscopic slide within a rectangular rubber gasket to mount the larvae flat on their side in agarose.

### Laser Ablation

Laser ablation of larvae veins were performed with pulsed nitrogen laser light pumped through coumarin 440 dye (445 nm) (MicroPoint Laser system, Photonic Instrument, St Charles, Ill., USA) at 10 pulses/second with laser intensity setting at 10. Accuracy of the laser was tested before ablations. Laser ablation of each larva was carried out 20 min after microinjection of the peptide. Glass slides were placed under Optipnot phase-contrast fluorescence microscope (Nikon, Melville, N.Y., USA). The larvae were viewed with 20× lens (10× eyepiece) to locate the site for laser ablations, which was five somites towards the caudal end from the anal pore (data not shown). Laser beam aimed at the caudal vein within the ablation site was triggered for 3 s. The process was recorded using a digital camera attached to a video home system (VHS) recorder and a monitor. Thrombus formation following vein injury due to laser ablation was monitored and the time taken for complete occlusion of injured vein was recorded.

Five inhibitors were selected as representative peptides to test for their antithrombotic effects in vivo using venous thrombosis model of zebrafish larvae. They are: (1) s-variegin (the full-length sequence of native variegin; a fast, tight-binding competitive inhibitor; Ki=0.318±0.020 nM; SEQ ID NO: 1); (2) EP25 (without seven N-terminal residues, has similar affinity to thrombin, but it is a slow binding inhibitor; Ki=0.370±0.11 nM; SEQ ID NO: 6) (3) MH22 (the cleavage product that is a fast and tight-binding, non-competitive inhibitor; Ki=14.11±0.29 nM; SEQ ID NO: 3); (4) DV24K10RYsulf (a peptide with potent in vitro activity; Ki=0.0420±0.0061 nM; SEQ ID NO: 16); and (5) hirulog-1 (a fast, tight-binding, competitive inhibitor currently in the market; Ki=2.94±0.12 nM; SEQ ID NO: 14). Hirulog-1 was used as positive control of the experiments.

All five peptides were injected into the zebrafish larvae circulation through the posterior (caudal) cardinal vein at a single dose (500 μM, 10 nl). Antithrombotic effects of the peptides are measured by the abilities of all peptides to delay time-to-occlusion (TTO) of caudal vein after laser ablation. After laser ablation, control TTO of a wild-type 4 dpf larva is about 21 s (Jagadeeswaran et al., 2006). Typically, if thrombus formation is inhibited (due to either an antithrombotic agent or genetic defect), TTO can be delayed up to 150 s, beyond which complete occlusion will not occur (Seongcheol Kim, personal communication). Therefore, the dose for injection (500 μM, 10 nl) was carefully selected based on a few preliminary experiments such that a definite TTO can be obtained for most, if not all, of the peptides (data not shown).

Zebrafish larvae injected with the same volume of PBS have a TTO (mean±S.D.) of 19.0±3.2 s (FIG. 20). s-variegin (Ki=0.318±0.020 nM) is strongly antithrombotic, with TTO of 120.8±7.4 s. In contrast, EP25, despite having similar Ki as s-variegin, does not show any activity. With a TTO of 22.5±6.2 s, EP25 did not show any significant antithrombotic effect compared with same batch of larvae injected with PBS. MH22 (Ki=14.11±0.29 nM) showed good activity with TTO of 33.3±2.9 s. DV24K10RYsulf (Ki=0.0420±0.0061 nM) is the most potent inhibitor and it completely inhibited of thrombus formation (data not shown). Hirulog-1/bivalirudin (Ki=2.94±0.12 nM), as reference drug, prolonged the TTO to 45.0±5.5 s. Overall, other than EP25, the antithrombotic effects of the peptides correlated well with their affinities for thrombin. Thus, slow binding inhibition mode (EP25) is not desirable for in vivo efficacy while both fast, competitive (s-variegin, DV24K10RYsulf and hirulog-1) and fast, non-competitive (MH22) inhibition are effective (FIG. 20). Our results are consistent with similar observations reported earlier about the importance of rapid thrombin inhibition for efficacious antithrombotic agents (Stone and Tapparelli, 1995).

### Example 8

### Neutralization of Thrombin Inhibitory Activity of Peptides

The ability of protamine sulphate to neutralise inhibition of thrombin amidolytic activity by the peptides was assayed using the chromogenic substrate S2238. Protamine is a mixture of highly cationic peptides originally extracted from fish sperm nuclei. Protamine sulphate is clinically used for the reversal of anticoagulant effect of heparin by binding to the anionic heparin molecules (Schulman and Bijsterveld, 2007). Variegin has several acidic residues at its C-terminus which could be the target for protamine sulphate. This option was first explored since there are ample clinical experiences for protamine sulphate administration.

All assays were performed in 96-well microtiter plates in 50 mM Tris buffer (pH 7.4) containing 100 mM NaCl and 1 mg/ml BSA at room temperature. Typically, 100 μl of peptide and 100 μl of protamine sulphate were pre-incubated for 10 min before the addition of 50 μl of human plasma derived thrombin. 50 μl of S2238 were added to initiate the reaction. The rates of formation of coloured product pNA were followed at 405 nm for 10 min with SPECTRAMax Plus microplate spectrophotometer (Molecular Devices, Sunnyvale, Calif., USA). Percentages of inhibition in the presence and absence of protamine sulphate were compared for calculation of percentages of reversal. Fixed concentrations of s-variegin, DV24K10RY^{sulf} and MH22 (at their respective IC₅₀ and IC₉₀) were incubated with various concentrations of protamine sulfate before assaying their residual thrombin inhibitory activities. Protamine sulfate reversed the effects of all three peptides dose-dependently (FIG. 21). Activities of s-variegin and MH22 were reversed to similar extent. At IC₅₀ of s-variegin (8.25 nM) and MH22 (11.5 nM), approximately 50% of reversal were achieved with 0.1 mg/ml of protamine sulfate. Percentage of reversal saturated at around 75% despite high concentrations of protamine sulphate. In contrast, for DV24K10RY^{sulf} its IC₅₀ concentration (1.4 nM), 1 mg/ml of protamine sulphate was needed for ˜50% of reversal. As expected, concentrations of protamine sulphate needed to neutralize effects of peptides at their IC₉₀s were higher than at IC₅₀s (FIG. 21). Therefore, protamine sulphate can neutralize most of the effect of variegin peptides. s-variegin and MH22 has identical C-termini (represented by MH22 sequence) but DV24K10RY^{sulf} C-terminus (represented by MH18Y^{sulf} sequence) is sulfated and has stronger affinity for thrombin. S-variegin and MH22 were neutralized to the similar extent. Higher concentrations of protamine sulphate are needed for DV24K10RY^{sulf} reversal. Therefore, the binding between protamine sulphate and the peptides are likely to be mediated through the acidic C-termini of variegin peptides.

### REFERENCES

Huntington J A. (2005) Molecular recognition mechanisms of thrombin Journal of Thrombosis and Haemostasis 3:1861-1872.
Davie E W, Fujikawa K, Kisiel W (1991) The coagulation cascade: initiation, maintenance, and regulation Biochemistry 30:10363-10370.
Davie E W. (2003) A brief historical review of the waterfall/cascade of blood coagulation Journal of Biological Chemistry 278:50819-50832.
Lane D A, Philippou H, Huntington J A. (2005) Directing thrombin Blood 106:2605-2612.
Hirsh J, O'Donnell M, Weitz J I (2005) New anticoagulants Blood 105:453-463.
Gurm H S, Bhatt D L (2005) Thrombin, an ideal target for pharmacological inhibition: a review of direct thrombin inhibitor American Heart Journal 149:S43-S53.
Bates S M, Weitz J I (2006) The status of new anticoagulant British Journal of Haematology 134:3-19.
WO2003091284 Anticoagulants Evolutec Limited
Maraganore J M, Bourdon P, Jablonski J, Ramachandran K L, Fenton J W (1990) Design and characterization of hirulogs: a novel class of bivalent peptide inhibitors of thrombin Biochemistry 29:7095-7101.
Skrzypczak-Jankun E, Carperos V E, Ravichandran K G et al. (1991) Structure of the hirugen and hirulog 1 complexes of alpha-thrombin Journal of Molecular Biology 221:1379-1393.
Sambrook et al. (2000) Molecular Cloning: A Laboratory Manual
Edmunds and Colman (2006) The Annals of Thoracic Surgery 82: 2315-22.
Girolami and Girolami (2006) Seminars in thrombosis and hemostatsis 32: 803-9
Lewis, B. E., and M. J. Hursting (2007) Expert Review of Cardiovascular Therapy 5: 57-68.
Varki A. (2007) Trousseau's Syndrome: multiple definitions and multiple mechanisms. Blood.
Nierodzik & Karpatkin (2006) Thrombin induces tumor growth, metastasis, and angiogenesis: Evidence for a thrombin-regulated dormant tumor phenotype Cancer Cell 10(5):355-62.
Terpe K, (2003) Applied Microbiology and Biotechnology 60: 523-33, 2003.
Eriksson, B. I., Smith, H., Yasothan, U., and Kirkpatrick, P. (2008). Dabigatran etexilate. Nat. Rev. Drug Discov. 7, 557-558.
Greinacher, A. and Warkentin, T. E. (2008). The direct thrombin inhibitor hirudin. Thromb. Haemost. 99, 819-829.
Warkentin, T. E., Greinacher, A., and Koster, A. (2008). Bivalirudin. Thromb. Haemost. 99, 830-839.
Yeh, R. W. and Jang, I. K. (2006). Argatroban: update. Am. Heart J. 151, 1131-1138.
Bode, W., Turk, D., and Karshikov, A. (1992). The refined 1.9-A X-ray crystal structure of D-Phe-Pro-Arg chloromethylketone-inhibited human alpha-thrombin: structure analysis, overall structure, electrostatic properties, detailed active-site geometry, and structure-function relationships. Protein Sci. 1, 426-471.
Brunger, A. T., Adams, P. D., Clore, G. M., DeLano, W. L., Gros, P., Grosse [symbol] Kunstleve, R. W., Jiang, J. S., Kuszewski, J., Nilges, M., Pannu, N. S., Read, R. J., Rice, L. M., Simonson, T., and Warren, G. L. (1998). Crystallography & NMR system: A new software suite for macromolecular structure determination. Acta Crystallogr. D. Biol. Crystallogr. 54, 905-921.
Jones, T. A., Zou, J. Y., Cowan, S. W., and Kjeldgaard, M. (1991). Improved methods for building protein models in electron density maps and the location of errors in these models. Acta Crystallogr. A 47 (Pt 2), 110-119.
Kitagawa, K., Aida, C., Fujiwara, H., Yagami, T., Futaki, S., Kogire, M., and Inoue, K. (2001). Facile solid-phase synthesis of sulphated tyrosine-containing peptides: total synthesis of human big gastrin-II and cholecystokinin (CCK)-39. J. Org. Chem. 66, 1-10.
Krissinel, E. and Henrick, K. (2007). Inference of macromolecular assemblies from crystalline state. J. Mol. Biol. 372, 774-797.
Laskowsi, R. A., MacArthur, M. W., Moss, D. S., and Thornton, J. M. (1993). PROCHECK: a program to check the stereochemical quality of protein structures. J. Appl. Crystallogr. 26, 283-291.
Otlewski, J., Jelen, F., Zakrzewska, M., and Oleksy, A. (2005). The many faces of protease-protein inhibitor interaction. EMBO J. 24, 1303-1310.
Page, M. J., Macgillivray, R. T., and Di Cera, E. (2005). Determinants of specificity in coagulation proteases. J. Thromb. Haemost. 3, 2401-2408.
Perona, J. J. and Craik, C. S. (1995). Structural basis of substrate specificity in the serine proteases. Protein Sci. 4, 337-360.
Qiu, X., Padmanabhan, K. P., Carperos, V. E., Tulinsky, A., Kline, T., Maraganore, J. M., and Fenton, J. W. (1992). Structure of the hirulog 3-thrombin complex and nature of the S′ subsites of substrates and inhibitors. Biochemistry 31, 11689-11697.
Rydel, T. J., Ravichandran, K. G., Tulinsky, A., Bode, W., Huber, R., Roitsch, C., and Fenton, J. W. (1990). The structure of a complex of recombinant hirudin and human alpha-thrombin. Science 249, 277-280.
Rydel, T. J., Tulinsky, A., Bode, W., and Huber, R. (1991). Refined structure of the hirudin-thrombin complex. J. Mol. Biol. 221, 583-601.
Schulman, S, and Bijsterveld, N. R. (2007). Anticoagulants and their reversal. Transfus. Med. Rev. 21, 37-48.
Skrzypczak-Jankun, E., Carperos, V. E., Ravichandran, K. G., Tulinsky, A., Westbrook, M., and Maraganore, J. M. (1991). Structure of the hirugen and hirulog 1 complexes of alpha-thrombin. J. Mol. Biol. 221, 1379-1393.
Stone, S. R. and Tapparelli, C. (1995). Thrombin inhibitors as antithrombotic agents: the importance of rapid inhibition. J. Enzyme Inhib. 9, 3-15.
Vagin, A. and Teplyakov, A. (2000). An approach to multi-copy search in molecular replacement. Acta Crystallogr. D. Biol. Crystallogr. 56, 1622-1624.
Vindigni, A., Dang, Q. D., and Di Cera, E. (1997). Site-specific dissection of substrate recognition by thrombin. Nat. Biotechnol. 15, 891-895.
Witting, J. I., Bourdon, P., Brezniak, D. V., Maraganore, J. M., and Fenton, J. W. (1992). Thrombin-specific inhibition by and slow cleavage of hirulog-1. Biochem. J. 283 (Pt 3), 737-743.
Cho Yeow Koh, Maria Kazimirova, Adama Trimnell, Peter Takac, Milan Labuda, Patricia A. Nuttall, and R. Manjunatha Kini (2007) J. Biol. Chem., 282(40) 29101-29113.

Sequence listing

SEQ ID NO: 1 (s-variegin)
SDQGDVAEPKMHKTAPPFDFEAIPEEYLDDES
SEQ ID NO: 2 (varigein cleavage product)
SDQGDVAEPK
SEQ ID NO: 3 (MH22)
MHKTAPPFDFEAIPEEYLDDES
SEQ ID NO: 4 (variegin E31H)
SDQGDVAEPKMHKTAPPFDFEAIPEEYLDDHS
SEQ ID NO: 5 (MH22A22E)
MHKTAPPFDFEEIPEEYLDDES
SEQ ID NO: 6 (EP25)
EPKMHKTAPPFDFEAIPEEYLDDES
SEQ ID NO: 7 (EP25A22E)
EPKMHKTAPPFDFEEIPEEYLDDES
SEQ ID NO: 8 (EP21)
EPKMHKTAPPFDFEAIPEEYL
SEQ ID NO: 9 (MH18)
MHKTAPPFDFEAIPEEYL
SEQ ID NO: 10 (DV24)
DVAEPKMHKTAPPFDFEAIPEEYL
SEQ ID NO: 11 (DV24K10R)
DVAEPRMHKTAPPFDFEAIPEEYL
SEQ ID NO: 12 (DV23K10R)
DVAEPRMHKTAPFDFEAIPEEYL
SEQ ID NO: 13 (DV23)
DVAEPKMHKTAPFDFEAIPEEYL
SEQ ID NO: 14 (Hirulog)
_{D}FPRPGGGGNGDFEEIPEEYL
SEQ ID NO: 15 (MH22A22E)
MHKTAPPFDFEEIPEEYLDDES
SEQ ID NO: 16 (DV24K10RYsulf)
DVAEPRMHKTAPPFDFEAIPEEY*L (*The Y is sulphated)
SEQ ID NO: 17 (Hirullin-P18)
VSYTDCTSGQNYCLCGGNFCGDGKHCEMDGSENKCVDGEGTPKRQTSGP
SDFEEFSLDDIEQ
SEQ ID NO: 18 (Hirudin variant-1)
VVYTDCTESGQNLCLCEGSNVCGQGNKCILGSDGEKNQCVTGEGTPKPQ
SHNDGDFEEIPEEYLQ
SEQ ID NO: 19 (Hirudin variant-2)
AICVSQAITYTDCTESGQNLCLCEGSNVCGKGNKCILGSNGKGNQCVTGE
GTPNPESHNNGDFEEIPEEYLQ
SEQ ID NO: 20 (Hirudin-3B′)
VVYTDCTESGQNLCLCQGSNVCGQGNKCILGSNGEKNQCVTGEGTPKP
QSHNDGDFEEIPEEYLQ
SEQ ID NO: 21 (Hirudin-3B)
VVYTDCTESGQNLCLCQDSNVCGQGNKCILGSNGEKNQCVTGEGTPKP
QSHNDGDFEEIPEEYLQ
SEQ ID NO: 22 (Hirudin-3A′)
VVYTDCTESGEDLCLCEGSNVCGEGNKCILGSDGEKNECVTGEGTPKP
QSHNDGDFEEIPEEYLQ
SEQ ID NO: 23 (Hirudin-3A)
VVYTDCTESGQNLCLCEDSNVCGEGNKCILGSNGEKNQCVTGEGTPKP
QSHNDGDFEEIPEEYLQ
SEQ ID NO: 24 (Hirudin)
VVYTDCTESGQNLCLCEGSNVCGQGNKCILGSDGEKNQCVTGEGTPGP
QSHNDGDFEEPEEYL
SEQ ID NO: 25 (Hirudin-HM2)
MFSLKLFVVFLAVCICVSQAVSYTDCTESGQNYCLCVGSNVCGEGKNCQ
LSSSGNQCVHGEGTPKPKSQTEGDFEEIPDEDILN
SEQ ID NO: 26 (Hirudin-HM1)
MFSLKLFVVFLAVCICVSQAVSYTDCTESGQNYCLCVGGNLCGGGKHC
EMDGSGNKCVDGEGTPKPKSQTEGDFEEIPDEDILN
SEQ ID NO: 27 (Hirudin-3)
VVYTDCTESGQNLCLCEDSNVCGQGNKCILGSNGEKNQCVTGEGTPKP
QSHNDGDFEEIPEEYLQ
SEQ ID NO: 28 (Hirudin-2B)
ITYTDCTESGQNLCLCEGSNVCGKGNKCILGSNGEENQCVTGEGTPKPQ
SHNDGDFEEIPEEYLQ
SEQ ID NO: 29 (Hirudin-2′)
ITYTDCTESGQDLCLCEGSDVCGKGNKCILGSNGEENQCVTGEGTPKPQ
SHNDGDFEEIPEEYLQ
SEQ ID NO: 30 (Hirudin-2)
ITYTDCTESGQDLCLCEGSNVCGKGNKCILGSNGEENQCVTGEGTPKPQ
SHNDGDFEEIPEEYLQ
SEQ ID NO: 31 (Hirudin-2A)
ITYTDCTESGQNLCLCEGSNVCGNGNKCKLGSDGEENQCVTGEGTPKP
QSHNDGDFEEIPEEYLQ
SEQ ID NO: 32 (Hirudin-P6)
MRYTACTESGQNQCICEGNDVCGQGRNCQFDSSGKKCVEGEGTRKPQ
NEGQHDFDPIPEEYLS
SEQ ID NO: 33 (Hirudin-PA)
ITYTDCTESGQNLCLCEGSNVCGKGNKCILGSQGKDNQCVTGEGTPKPQ
SHNQGDFEPIPEDAYDE
SEQ ID NO: 34 (Sequence 25 from patent U.S. Pat.
No. 5,985,833)
NGDFEEIPEEYL
SEQ ID NO: 35 (Sequence 24 from patent U.S. Pat.
No. 5,985,833)
VEYEALYPEDD
SEQ ID NO: 36 (Sequence 23 from patent U.S. Pat.
No. 5,985,833)
EVEYEALYPEDD
SEQ ID NO: 37 (Sequence 22 from patent U.S. Pat.
No. 5,985,833)
AEVEYEALYPE
SEQ ID NO: 38 (Sequence 21 from patent U.S. Pat.
No. 5,985,833)
AEVEYEALYPED
SEQ ID NO: 39 (Sequence 20 from patent U.S. Pat.
No. 5,985,833)
AEVEYEALYPEDD
SEQ ID NO: 40 (Sequence 19 from patent U.S. Pat.
No. 5,985,833)
EALYPEDDL
SEQ ID NO: 41 (Sequence 3 from patent U.S. Pat.
No. 5,985,833)
VSVEHEVDVEYP
SEQ ID NO: 42 (Sequence 4 from patent U.S. Pat.
No. 5,985,833)
VRVEHHVEIEYD
SEQ ID NO: 43 (Sequence 5 from patent U.S. Pat.
No. 5,985,833)
NGDFEEIPEEYLQ
SEQ ID NO: 44 (Sequence 6 from patent U.S. Pat.
No. 5,985,833)
FPRFPRP
SEQ ID NO: 45 (Sequence 7 from patent U.S. Pat.
No. 5,985,833)
QSHNDG
SEQ ID NO: 46 (Sequence 9 from patent U.S. Pat.
No. 5,985,833)
AVRPEHPAETEYESLYPEDDL
SEQ ID NO: 47 (Sequence 10 from patent U.S. Pat.
No. 5,985,833)
PEHPAETEY
SEQ ID NO: 48 (Sequence 11 from patent U.S. Pat.
No. 5,985,833)
EHPAETEYESLYPEDDL
SEQ ID NO: 49 (Sequence 12 from patent U.S. Pat.
No. 5,985,833)
EHPAETEFESLYPEDDL
SEQ ID NO: 50 (Sequence 13 from patent U.S. Pat.
No. 5,985,833)
AETEYESLYPEDDL
SEQ ID NO: 51 (Sequence 14 from patent U.S. Pat.
No. 5,985,833)
VRPEHPAEVEYEALYPEDDL
SEQ ID NO: 52 (Sequence 15 from patent U.S. Pat.
No. 5,985,833)
PEHPAEVEY
SEQ ID NO: 53 (Sequence 16 from patent U.S. Pat.
No. 5,985,833)
EHPAEVEYEALYPEDDL
SEQ ID NO: 54 (Sequence 17 from patent U.S. Pat.
No. 5,985,833)
AEVEYEALYPEDDL
SEQ ID NO: 55 (Sequence 18 from patent U.S. Pat.
No. 5,985,833)
EYEALYPEDDL
SEQ ID NO: 56 (Sequence 1 from patent U.S. Pat.
No. 5,985,833)
AGDV
SEQ ID NO: 57 (Sequence 2 from patent U.S. Pat.
No. 5,985,833)
VRPEHPAETEYESLYPEDDL
SEQ ID NO: 58 (thrombin inhibitor, putative
[Ixodes scapularis])
MHQEGDFKMGHCSDLKVSALEIPYKGNKMSMVILLPEDVEGLSDLEEHL
TAPKLLALLGGMYVTSDVNLHFPKFKLEQSMGLKDVLMAMGVKDFFTFL
ADLSGISATGNLCASDVIHKAFVEVNEEGTEAAAATAILMDCIPQVVNF
FVDHPFMFLICSHDPDAVLFMGSIREL
SEQ ID NO: 59 (inhibitor, putative
[Ixodes scapularis])
MHQKGDFKMGHCSDLKVTALEIPYKGNKMSMIILLPEDVEGLSVLEEHL
TAPKLSALLGGMYVTPDVNLRLPKFKLEQSIGLKDVLMAMGVKDFFTSL
ADLSGISAAGNLCASDVIHKAFVEVNEEGTEAAAATAIPMMLMCARFPQ
VVNFFVDHPFMFLIHSHDPDVVLFMGSIREL
SEQ ID NO: 60 (thrombin inhibitor, putative
[Ixodes scapularis])
MASDFSNSLISFSVDLYKKLKSESDGASNFICSPFSIAAALSMTLAGAK
HDTAKQISNALHMQDTTVHENFAYFFSKLPGYAPDVILHVANRLYAEET
YNTLDEFTHLLEKSYSTTVEKVDFKRNAEKTRLQVNTWVEEVTQSKIKD
LLAEGTIDDFTSLIIINAVYFKGLWHDQFDPKRTSQQEFHLTADRTKMV
DMMHHKQRFRMCRHPNFKVSALEIPYKGQKMSMVILLPEEIDGLADLEE
TLTSSKIREIIQELSYQGDIELSLPRFKLEHTVGLKNVLAAMGIEDMFD
ALKCDLSGISPDNALVVSDVVHKAFIEVNEEGTEAAAATAMVMLCCMSF
PTRFTVDHPFLFLIRCHDPDVILFIGSVAQI
SEQ ID NO: 61 (serine protease inhibitor
[Echinococcus multilocularis])
MFAKTSFIPFTQALYAQLQPSEGRSNFFMSPLSVYSALSLALAGSESET
REELVSVLGLAPGKDIDTIVKSLGENLQAVADGDAKKTLVEANGVFIQA
GSQIRETYTSAVSKHLKADMKQLDFGGDSEGSRISINRWIAEKTREKVK
DLLAQGSITPMTHVVLANAVYFKGVWKCKFEKSKTDRSGVFHSLDSGDV
RVSMMTQKASYPMADFVDLEVRALKVPFETHEMLIVLPEKNDGLPNLLK
QLSANAKHLEEMLTSDQYFDTEVVLKLPKFSLGGHNMKLKEPLHKMGLK
SAFDAERADFSGITNDRSLAVSDVYHQAVIDVDEEGAEAAAATAMPMMV
RCMPAPPVDFFVDHPFIFFIVTKTGIPVFMGHVVHPESK
SEQ ID NO: 62 (thrombostasin precursor
[Stomoxys calcitrans])
MKYFVFIGIIALSAVSQAQNGRWQGDLHGLSGHRGSGLPGLSGHRGSGQ
PGLSGHRGSEQSGEAGAPSYDYFSQPGLSGSRRHGQRDLEVEDSRPARS
LDPLNSVPEWNENDEDDHEFGPYRKPQDNQRDRRRPQRSFDSFYSVPEW
NEDNEADQGYRKRQDNQQNRHRAQPQRPLHPHYSHPEWEEEDEGDQQFG
RYRKPHDNQQDRRRGKAQPQMRLHPFNSHPEWEDEDEGDQQFGPYRRPQ
DNQQNRRGESRPIRSNGDELSSDDQLASFFGFPAGDVPEDLKNLVRLFS
GPNNDFGGNNQFEED
SEQ ID NO: 63 (Antithrombin-III)
MISNGIGTVTAGKRSICLLPLLLIGLWGCVTCHRSPVEDVCTAKPRDIP
VNPMCIYRSSEKKATEGQGSEQKIPGATNRRVWELSKANSHFATAFYQH
LADSKNNNDNIFLSPLSISTAFAMTKLGACNNTLKQLMEVFKFDTISEK
TSDQIHFFFAKLNCRLYRKANKSSELVSANRLFGGKSITFNETYQDISE
VVYGAKLQPLDFKGNAEQSRLTINQWISNKTEGRITDVIPPQAINEFTV
LVLVNTIYFKGLWKSKFSPENTRKELFYKADGESCSVLMMYQESKFRYR
RVAESTQVLELPFKGDDITMVLILPKLEKTLAKVEQELTPDMLQEWLDE
LTETLLVVHMPRFRIEDSFSVKEQLQDMGLEDLFSPEKSRLPGIVAEGR
SDLYVSDAFHKAFLEVNEEGSEAAASTVISIAGRSLNSDRVTFKANRPI
LVLIREVALNTIIFMGRVANPCVD
SEQ ID NO: 64 (Alpha-1-antitrypsin-like protein
GS55-MS)
MPSSISWGLLLLAGLSCLVAGSLAEDAQETGASKHDQEHPASHRIAPNL
AEFALSLYRVLAHESNTTNIFFSPVSIAMALASLSLGTKADTHTQIMEG
LGFNLTETAESDIHQGFQHLLQTLNKPNSQLQLTTGNGLFIDHNLKLLD
KFLQDVKNLYHSEAFSTDFTNTEEAKKQINTYVEKGTQGKIVDLVKDLN
RDSVLALVNYIFFKGKWEKPFEVDHTKEEDFHVDQVTTVRVPMMNRMGM
FEVHYCSTLASWVLQMDYLGNATAIFLLPDEGKLQHLEDTITKEILAKF
LKNRESSSVNLHFPKLNISGTMDLKPVLTRLGITNVFSYKADLSGITED
DPLRVSQALHKAVLTIDERGTEAAGATFLEMMPMSLPPEVKFDKPFLVV
IIEHSTKSPLFVGKVVNPTLH
SEQ ID NO: 65 (unnamed protein product
[Homo sapiens])
MLKKPLSAVTWLCIFIVAFVSHPAWLQKLSKHKTPAQPQLKAANCCEEV
KELKAQVANLSSLLSELNKKQERDWVSVVMQVMELESNSKRMESRLTDA
ESKYSEMNNQIDIMQLQAAQTVTQTSADAIYDCSSLYQKNYRISGVYKL
PPDDFLGSPELEVFCDMETSGGGWTIIQRRKSGLVSFYRDWKQYKQGFG
SIRGDFWLGNEHIHRLSRQPTRLRVEMEDWEGNLRYAEYSHFVLGNELN
SYRLFLGNYTGNVGNDALQYHNNTAFSTKDKDNDNCLDKCAQLRKGGYW
YNCCTDSNLNGVYYRLGEHNKHLDGITWYGWHGSTYSLKRVEMKIRPED
FKP
SEQ ID NO: 66 (neuroserpin
[Mus musculus])
MTYLELLALLALQSVVTGATFPDETITEWSVNMYNHLRGTGEDENILFS
PLSIALAMGMMELGAQGSTRKEIRHSMGYEGLKGGEEFSFLRDFSNMAS
AEENQYVMKLANSLFVQNGFHVNEEFLQMLKMYFNAEVNHVDFSQNVAV
ANSINKWVENYTNSLLKDLVSPEDFDGVTNLALINAVYFKGNWKSQFRP
ENTRTFSFTKDDESEVQIPMMYQQGEFYYGEFSDGSNEAGGIYQVLEIP
YEGDEISMMLALSRQEVPLATLEPLLKAQLIEEWANSVKKQKVEVYLPR
FTVEQEIDLKDILKALGVTEIFIKDANLTAMSDKKELFLSKAVHKSCIE
VNEEGSEAAAASGMIAISRMAVLYPQVIVDHPFLYLIRNRKSGIILFMG
RVMNPETMNTSGHDFEEL
SEQ ID NO: 67 (Glia-derived nexin)
MNWHLPLFLLASVTLPSICSHFNPLSLEELGSNTGIQVFNQIVKSRPHD
NIVISPHGIASVLGMLQLGADGRTKKQLAMVMRYGVNGVGKILKKINKA
IVSKKNKDIVTVANAVFVKNASEIEVPFVTRNKDVFQCEVRNVNFEDPA
SACDSINAWVKNETRDMIDNLLSPDLIDGVLTRLVLVNAVYFKGLWKSR
FQPENTKKRTFVAADGKSYQVPMLAQLSVFRCGSTSAPNDLWYNFIELP
YHGESISMLIALPTESSTPLSAIIPHISTKTIDSWMSIMVPKRVQVILP
KFTAVAQTDLKEPLKVLGITDMFDSSKANFAKITTGSENLHVSHILQKA
KIEVSEDGTKASAATTAILIARSSPPWFIVDRPFLFFIRHNPTGAVLFM
GQINKP
SEQ ID NO: 68 (Glia-derived nexin)
MNWHFPFFILTTVTLYSVHSQFNSLSLEELGSNTGIQVFNQIIKSRPHE
NVVVSPHGIASILGMLQLGADGKTKKQLSTVMRYNVNGVGKVLKKINKA
IVSKKNKDIVTVANAVFLRNGFKMEVPFAVRNKDVFQCEVQNVNFQDPA
SASESINFWVKNETRGMIDNLLSPNLIDGALTRLVLVNAVYFKGLWKSR
FQPESTKKRTFVAGDGKSYQVPMLAQLSVFRSGSTRTPNGLWYNFIELP
YHGESISMLIALPTESSTPLSAIIPHITTKTIDSWMNTMVPKRMQLVLP
KFTAVAQTDLKEPLKALGITEMFEPSKANFTKITRSESLHVSHILQKAK
IEVSEDGTKASAATTAILIARSSPPWFIVDRPFLFSIRHNPTGAILFLG
QVNKP
SEQ ID NO: 69 (Neuroserpin)
MAYLGLLSLVALQSLVTGAAFPDETIAEWSVNVYNHLRATGEDENILFS
PLSIALAMGVMELGAQGSTLKEIRHSMGYESLKSGEEFSFLRDFSSMVS
AEEGQYVMKIANSLFVQNGFHINEEFLQMMKMYFNAEVNHVDFSENVAV
ANYINKWVENYTNSLLKDLVSPGDFDAVTHLALINAVYFKGNWKSQFRP
ENTRTFSFTKDDESEVQIPMMYQQGEFYYGEFSDGSNEAGGIYQVLEIP
YEGDEISMMLVLSRQEVPLATLEPLLKPQLIEEWANSVKKQKVEVYLPR
FTVEQEIDLKDILKALGVTEIFIKDANLTAMSDKKELFLSKAVHKSFIE
VNEEGSEAAVASGMIAISRMAVLFPQVIVDHPFLFLIKNRKTGTILFMG
RVMHPETMNTSGHDFEEL
SEQ ID NO: 70 (serpin peptidase inhibitor,
clade C, member 1 [Homo sapiens])
MYSNVIGTVTSGKRKVYLLSLLLIGFWDCVTCHGSPVDICTAKPRDIPM
NPMCIYRSPEKKATEDEGSEQKIPEATNRRVWELSKANSRFATTFYQHL
ADSKNDNDNIFLSPLSISTAFAMTKLGACNDTLQQLMEVFKFDTISEKT
SDQIHFFFAKLNCRLYRKANKSSKLVSANRLFGDKSLTFNETYQDISEL
VYGAKLQPLDFKENAEQSRAAINKWVSNKTEGRITDVIPSEAINELTVL
VLVNTIYFKGLWKSKFSPENTRKELFYKADGESCSASMMYQEGKFRYRR
VAEGTQVLELPFKGDDITMVLILPKPEKSLAKVEKELTPEVLQEWLDEL
EEMMLVVHMPRFRIEDGFSLKEQLQDMGLVDLFSPEKSKLPGIVAEGRD
DLYVSDAFHKAFLEVNEEGSEAAASTAVVIAGRSLNPNRVTFKANRPFL
VFIREVPLNTIIFMGRVANPCVK
SEQ ID NO: 71 (heparin cofactor II
[Xenopus laevis])
MKLLHLATIFLLIHATLGGVKDLQEHFEDTSTGINPRGSQTQAVENLLD
DTVTNDLSTEGEDEEDYLDFDKIFGEDEDYIDIIDAAPEIKNSETQQGN
IFELFHGKTRVQRLNIINANFGFNLYRAIKNNTDASENILLAPVGISTA
MATISLGTKGQTLEQVLLTLGFKDFLNASSKYEILTLHNVFRKLTHRLF
RRNFGYTLRSVNDIYVKRDFLIREPFKNNLKNYYFAEAQTVDFGYKDFL
TKANKRIQQLTKGLIEEALTNVDPALLMLLVNCIYFKGTWENKFPVEYT
QNMNFRLNEKELVKVPMMKTKGNFLVAADPELDCAVLQLPYVGNISMLI
VLPHKLSGMKLLEKQISPQVVERWQNIMTNRTREVFLPRFKLEKSYDLQ
KVLSNMGATDLFTHGDFSGVSDKDINIGLFQHQGTITVNEEGTEAAAVT
VVGFMPLSTQARFVADRPFLFLIYEHRTNCLVFMGRVANPTKS
SEQ ID NO: 72 (heparin cofactor II
[Gallus gallus])
GTFCGIKDFSDHFESLKDAHTHENGTYNMPDLPLEFHRENTITNDLIPE
EEEEEDYLDLDKILGEDDYSDIIDAAPHIVSEIQQGNILELFQGKTRIQ
RLNILNANFGFNLYRSVADKANSSDNILMAPVGISTAMAMISLGLKGQT
QQEVLSVLGFEDFINASAKYELMTVHNLFRKLTHRLFRRNFGYTLRSVN
DLYIRKDFSILNDFRNNMKTYYFADAQPADFSDPNFITKTNERILKLTK
GLIKEALVNVNPTTLMMILNCLYFKGTWENKFPVEMTTKRSFRLNEKQT
IKVPMMQTKGNFLAAADPELDCGVIQLPFVGNISMLIVLPHKLSGMKAL
EKQITPQVVEKWQKSMTNRTREVVLPKFKLEKNYNLIGFLRSMGIEELF
SEKGNYCGVSEEKVSIDRFNHQGTITVNEEGTEAGAITNVGFMPLSTQI
RFIVDRPFLFLIYEHRTNCLLFMGRVVNPAKP
SEQ ID NO: 73 (Heparin cofactor 2)
MQHRPHLLLISLTIMSVCGGSNGLTDQLNNKNLTMPLLPIEFHKENTVT
NDWIPEGEEDDDYLDLEKLLSEDDDYIDIIDAVSPTDSEASAGNILQLF
QGKSRIQRLNILNAKFAFSLYRALKDQANAFDNIFIAPVGISTAMGMIS
LGLKGETHEQVHSVLHFRDFVNASSKYEILTIHNLFRKLTHRLFRRNFG
YTLRSVNDLYVQKQFPIREDFKAKVREYYFAEAQAADFSDPAFISKANN
HILKVTKGLIKEALENVDPATQMMILNCIYFKGTWVNKFPVEMTHNHNF
RLNEREVVKVSMMQTKGNFLAANDQELACDVLQLEYVGGISMLIVVPHK
LSGMKTLEAQLTPQVVERWQKSMTNRTREVLLPKFKLEKNYNLVEALKS
MGVTELFDKNGNMSGISDQGITMDLFKHQGTITVNEEGTQAAAVTTVGF
MPLSTQVRFTVDRPFLFLVYEHRTSCLLFMGKVANPVRS
SEQ ID NO: 74 (serine (or cysteine) proteinase
inhibitor, clade D, member 1 precursor
[Mus musculus])
MKHPLCTLLSLITFMCIGSKGLAEQLTNENLTTSFLPANFHKENTVTND
WIPEGEEDEDYLDLEKLLGEDDDYIYIIDAVSPTDSESSAGNILQLFQG
KSRIQRLNILNAKFAFNLYRVLKDQATTSDNLFIAPVGISTAMGMISLG
LRGETHEEVHSVLHFRDFVNASSKYEVTTIHNLFRKLTHRLFRRNFGYT
LRSVNGLYIQKQFPIREDFKAAMREFYFAEAQEANFPDPAFISKANNHI
LKLTKGLIKEALENIDPATQMLILNCIYFKGTWVNKFPVEMTHNHNFRL
NEREVVKVSMMQTKGNFLAANDQELDCDILQLEYVGGISMLIVVPRKLS
GMKTLEAQLTPQVVERWQKSMTNRTREVLLPKFKLEKNYNLVEVLKSMG
ITKLFNKNGNMSGISDQRIAIDLFKHQSTITVNEEGTQAAAVTTVGFMP
LSTQVRFTVDRPFLFLVYEHRTSCLLFMGKVTNPAKS
SEQ ID NO: 75 (Heparin cofactor 2)
MKHPAYTLLLSLIMSMCAGSKGLAEQLTKENLTVSLLPPNFHKENTVTN
DWIPEGEEDDDYLDLEKLLSEDDDYIYVVDAVSPTDSESSAGNILQLFQ
GKSRIQRLNILNAKFAFNLYRVLKDQATSSDNIFIAPVGISTAMGMISL
GLRGETHEEVHSVLHFKDFVNASSKYEVTTIHNLFRKLTHRLFRRNFGY
TLQSVNDLYIQKQFPIREDFKAAMREFYFAEAQEADFSDPAFISKANSH
ILKLTKGLIKEALENTDSATQMMILNCIYFKGAWMNKFPVEMTHNHNFR
LNEREVVKVSMMQTKGNFLAANDQELDCDILQLEYVGGISMLIVIPRKL
SGMKTLEAQLTPQVVERWQKSMTNRTREVLLPKFKLEKNYNLVEVLKSM
GITKLFNKNGNMSGISDQRIIIDLFKHQSTITVNEEGTQAAAVTTVGFM
PLSTQVRFTVDRPFLFLVYEHRTSCLLFMGRVANPAKS
SEQ ID NO: 76 (serine (or cysteine) proteinase
inhibitor, clade D (heparin cofactor),
member 1 [Danio rerio])
MWLVPVIVVACLLNSPALAGVKDLSSHFSTLEKEKTVDARGLSPGGENT
DMESIPLDFHRENTVTNDLPEGQDDEDYVDFDKILGEDDYSEGDHIDEI
STPAPDLDLFYEPSDPKIRRARLLRLFHGQTRLQRINVVNARFGFRLYR
KLRNRLNQTDNILLAPVGISIAMGMMGLGVGPNTQEQLFQTVGFAEFVN
ASNHYDNSTVHKLFRKLTHRLFRRNFGYTLRSVNDLYVKRNVQIQDSFR
ADAKTYYFAEPQSVDFADPAFLVKANQRIQKITKGLIKEPLKSVDPNMA
VMLLNYLYFKGTWEQKFPKELTHHRQFRVNEKKQVRVLMMQNKGSYLAA
ADHELNCDILQLPYAGNISMLIAVPQKLSGMRSLEQEISPTLVNKWLSN
MTNRTREVVFPRFKLEQNYDLIEHLKEMGMTDIFTEKGDFSPMTSEKVI
INWFKHQGSITVNEEGTEAAAMTHIGFMPLSTQTRFIVDRPFLFLIYEH
RTGCVVFMGRVVDPSQT
SEQ ID NO: 77 (serine (or cysteine) proteinase
inhibitor, clade D (heparin cofactor),
member 1 [Danio rerio])
MWLVPVIVVACLLNSPALAGVKDLSSHFSTLEKEKTVDARGLSPGGENT
DMESIPLDFHRENTVTNDLPEGQDDEDYVDFDKILGEDDYSEGDHIDEI
STPAPDLDLFYEPSDPKIRRARLLRLFHGQTRLQRINVVNARFGFRLYR
KLRNRLNQTDNILLAPVGISIAMGMMGLGVGPNTQEQLFQTVGFAEFVN
ASNHYDNSTVHKLFRKLTHRLFRRNFGYTLRSVNDLYVKRNVQIQDSFR
ADAKTYYFAEPQSVDFADPAFLVKANQRIQKITKGLIKEPLKSVDPNMA
VMLLNYLYFKGTWEQKFPKELTHHRQFRVNEKKQVRVLMMQNKGSYLAA
ADHELNCDILQLPYAGNISMLIAVPQKLSGMRSLEQEISPTLVNKWLSN
MTNRTREVVFPRFKLEQNYDLIEHLKEMGMTDIFTEKGDFSPMTSEKVI
INWFKHQGSITVNEEGTEAAAMTHIGFMPLSTQTRFIVDRPFLFLIYEH
RTGCVVFMGRVVDPSQS
SEQ ID NO: 78 (serpin peptidase inhibitor,
clade D (heparin cofactor), member 1
[synthetic construct])
MWMLQRGVDQPGRLSLCSVFPPSFSSAKMKHSLNALLIFLIITSAWGGS
KGPLDQLEKGGETAQSADPQWEQLNNKNLSMPLLPADFHKENTVTNDWI
PEGEEDDDYLDLEKIFSEDDDYIDIVDSLSVSPTDSDVSAGNILQLFHG
KSRIQRLNILNAKFAFNLYRVLKDQVNTFDNIFIAPVGISTAMGMISLG
LKGETHEQVHSILHFKDFVNASSKYEITTIHNLFRKLTHRLFRRNFGYT
LRSVNDLYIQKQFPILLDFKTKVREYYFAEAQIADFSDPAFISKTNNHI
MKLTKGLIKDALENIDPATQMMILNCIYFKGSWVNKFPVEMTHNHNFRL
NEREVVKVSMMQTKGNFLAANDQELDCDILQLEYVGGISMLIVVPHKMS
GMKTLEAQLTPRVVERWQKSMTNRTREVLLPKFKLEKNYNLVESLKLMG
IRMLFDKNGNMAGISDQRIAIDLFKHQGTITVNEEGTQATTVTTVGFMP
LSTQVRFTVDRPFLFLIYEHRTSCLLFMGRVANPSRS
SEQ ID NO: 79 (Serine (or cysteine) proteinase
inhibitor, clade D (heparin cofactor),
member 1 [Danio rerio])
MWLVPVIVVACLLNSPALAGVKDLSSHFSTLEKEKTVDARGLSPGGENT
DMESIPLDFHRENTVTNDLPEGQDDEDYVDFDKILGEDDYSEGDHIDEI
STPAPDLDLFYEPSDPKIRRARLLRLFHGQTRLQRINVVNARFGFRLYR
KLRNRLNQTDNILLAPVGISIAMGMMGLGVGPNTQEQLFQTVGFAEFVN
ASNHYDNSTVHKLFRKLTHRLFRRNFGYTLRSVNDLYVKRNVQIQDSFR
ADAKTYYFAEPQSVDFADPAFLVKANQRIQKITKGLIKEPLKSVDPNMA
VMLLNYLYFKGTWEQKFPKELTHHRQFRVNEKKQVRVLMMQNKGSYLAA
ADHELNCDILQLPYAGNISMLIAVPQKLSGMRSLEQEISPTLVNKWLSN
MTNRTREVVFPRFKLEQNYDLIEHLKEMGMTDIFTEKGDFSPMTSEKVI
INWFKHQGSITVNEEGTEAAAMTHIGFMPLSTQTRFIVDRPFPFLIYEH
RTGCVVFMGRVVDPSQT
SEQ ID NO: 80 (Serpin B6)
MDVLAEANGTFALNLLKTLGKDNSKNVFFSPMSMSCALAMVYMGAKGNT
AAQMAQILSFNKSGGGGDIHQGFQSLLTEVNKTGTQYLLRMANRLFGEK
SCDFLSSFRDSCQKFYQAEMEELDFISAVEKSRKHINTWVAEKTEGKIA
ELLSPGSVDPLTRLVLVNAVYFRGNWDEQFDKENTEERLFKVSKNEEKP
VQMMFKQSTFKKTYIGEIFTQILVLPYVGKELNMIIMLPDETTDLRTVE
KELTYEKFVEWTRLDMMDEEEVEVSLPRFKLEESYDMESVLRNLGMTDA
FELGKADFSGMSQTDLSLSKVVHKSFVEVNEEGTEAAAATAAIMMMRCA
RFVPRFCADHPFLFFIQHSKTNGILFCGRFSSP
SEQ ID NO: 81 (thrombin inhibitor
[Homo sapiens])
MDVLAEANGTFALNLLKTLGKDNSKNVFFSPMSMSCALAMVYMGAKGNTA
AQMAQILSFNKSGGGGDIHQGFQSLLTEVNKTGTQYLLRVANRLFGEKS
CDFLSSFRDSCQKFYQAEMEELDFISAVEKSRKHINTWVAEKTEGKIAE
LLSPGSVDPLTRLVLVNAVYFRGNWDGQFDKENTEERLFKVSKNEEKPV
QMMFKQSTFKKTYIGEIFTQILVLPYVGKELNMIIMLPDETTDLRTVEK
ELTYEKFVEWTRLDMMDEEEVEVSLPRFKLEESYDMESVLRNLGMTDAF
ELGKADFSGMSQTDLSLSKVVHKSFVEVNEEGTEAAAATAAIMMMRCARF
VPRFCADHPFLFFIQHRKTNGILFCGRFSSP
SEQ ID NO: 82 (Serpin B6)
MDVLAEANGTFALNLLKTLGKDNSKNVFFSPMSMSCALAMVYMGAKGNT
AAQMAQVLSFNKSGGGGDIHQGFQSLLTEVNKTGTQYLLRTANRLFGEK
SCDFLSSFRDSCQKFYQAEMEELDFISAVEKSRKHINSWVAEKTEGKIA
ELLSPGSVDPLTRLVLVNAVYFKGNWNEQFDKENTEERRFKVSKNEEKP
VQMMFMQSTFRKTYIGEIFTQILVLPYVGKELNMIIMLPDETTDLRTVE
KELTYEKFVEWTRLDMMDEEKVEVSLPRFKLEESYDMESVLCSLGMTDA
FELGKADFSGMSKADLCLSKVVHKSFVEVNEEGTEAAAATAAIMMMRCA
RFVPRFCADHPFLFFIQHSKTNGVLFCGRFSSP
SEQ ID NO: 83 (similar to Placental thrombin
inhibitor (Cytoplasmic antiproteinase) (CAP)
(Protease inhibitor 6) (PI-6) (Serpin B6)
isoform 2 [Canis familiaris])
MDTLSEANGTFAISLLKKLGEDGSKNVFFSPMSISSALSMVFMGAKGNT
AAQMSQTLSLSKSGGGGDVHQGFQALLNEVNSAEARYLLRTANRLFGEK
TCGFLSSFKDSCRTFYQAEMEELDFLSACEQSREHITAGVTEGRKVKTR
GKIVDLLSPGSVDPGTNLILVNAIYFKGNWDKQFNKEQTTERPFKVSKN
EKKPVQMMFKKSTFQMTYIGEIFTKILVLPYVGRELNMIIMLPDENVSL
ETVETELTYEKFTEWTRPDMLDEEEVEVFLPRFKLEEEYDMKAVLCSLG
MTDAFEQSKADFSGMSSRGDLYLSKVVHKSFVEVNEEGTEAAAASAAVM
MLRCARIVPRFCADRPFLFFIQHSKSRSVLFCGRFSSP
SEQ ID NO: 84 (similar to Placental thrombin
inhibitor (Cytoplasmic antiproteinase) (CAP)
(Protease inhibitor 6) (PI-6) (Serpin B6)
isoform 3 [Canis familiaris])
MDTLSEANGTFAISLLKKLGEDGSKNVFFSPMSISSALSMVFMGAKGNT
AAQMSQTLSLSKSGGGGDVHQGFQALLNEVNSAEARYLLRTANRLFGEK
TCGFLSVSPLSPARKIVDLLSPGSVDPGTNLILVNAIYFKGNWDKQFNK
EQTTERPFKVSKNEKKPVQMMFKKSTFQMTYIGEIFTKILVLPYVGREL
NMIIMLPDENVSLETVETELTYEKFTEWTRPDMLDEEEVEVFLPRFKLE
EEYDMKAVLCSLGMTDAFEQSKADFSGMSSRGDLYLSKVVHKSFVEVNE
EGTEAAAASAAVMMLRCARIVPRFCADRPFLFFIQHSKSRSVLFCGRFS
SP
SEQ ID NO: 85 (similar to Serpin B6 (Placental
thrombin inhibitor) (Cytoplasmic antiproteinase)
(CAP) (Proteinase inhibitor 6) (PI-6) isoform 1
[Equus caballus])
MDTLSEANGTFALNLLKKLGEDNSKNVFFSPMSISSALAMVFMGAKGNT
AAQMSQVLSLSKSGGEVGDVHQGFQSLLSEINRPGTQYLLRTANRLFGE
KSYDFLSSFKDSCHKFYQAEMEQLDFISATEESRKHINTWVAKKTEGKI
TELLSSDSVDLLTKLILVNAIYFKGNWDDQFDKQQTKERPFKVSKNEEK
PVQMMFKKSTFKRTYIGEIFTQILMLPYVGEELNMIIMLPDENTDLKTV
EKELTYEKFVEWTRPDMMDETEMEVFLPRFKLEEDYDMEAVLRSLGMTD
AFEQARADFSGMSSRADLFLSKVVHKSFVEVNEEGTEAAAATAAVMMMR
CVRIIPRFCADHPFLFFIQHSKTNSILFCGRFSSP
SEQ ID NO: 86 (Serpin B6)
MDPLQEANGTFALNLLKILGEDSSKNVFLSPMSISSALAMVFMGAKGTT
ASQMAQALALDKCSGNGGGDVHQGFQSLLTEVNKTGTQYLLRTANRLFG
DKTCDLLASFKDSCLKFYEAELEELDFQGATEESRQHINTWVAKKTEDK
IKEVLSPGTVNSDTSLVLVNAIYFKGNWEKQFNKEHTREMPFKVSKNEE
KPVQMMFKKSTFKMTYIGEIFTKILLLPYVSSELNMIIMLPDEHVELST
VEKEVTYEKFIEWTRLDKMDEEEVEVFLPKFKLEENYNMNDALYKLGMT
DAFGGRADFSGMSSKQGLFLSKVVHKAFVEVNEEGTEAAAATAGMMTVR
CMRFTPRFCADHPFLFFIHHVKTNGILFCGRFSSP
SEQ ID NO: 87 (similar to thrombin inhibitor
isoform 2 [Bos taurus])
MEELDFLSATEESRKHINTWVAEKTEGKIRDLLPANSVNAMTRLVLVNA
IYFKGNWDTQFNKEHTKERPFRVSKNMEKPVQMMFKKSTFKSTYIGEIS
TQILVLPYVGKELNMVILLPSESTDLNTVEKALTYEKFVTWTKLDVMDE
DEVEVFLPRFTLEESYDMECVLRDLGVTDAFEAAQADFSGMSCQQDLHL
SKIVHKSFVEVTEEGTEAAAATVARITPRILRIVPRFCADHPFLFFIQH
SRTGAILFCGRFCSP
SEQ ID NO: 88 (thrombin inhibitor
[Bos taurus])
MDALSEANGTFALTLLKKLGEGNSKNVFISPLSISSALAMVLLGAKGNT
AAQMCQTLSLNKSSGGGEDVHQGFQNLLSEVNRRDTQYLLRTANRLFGE
KTYDFLSSFKDSCHKFYQAEMEELDFVSATEQSRKHINTWVAEKTEGKI
RDLLPANSVNPMTRLVLVNAIYFKGNWDTQFNKEHTEERPFRVSKNVEK
PVQMMFKKSTCKITYIGEISTQILVLPYVGQELNMVILLPSESTDLNTV
EKALTYEKFIAWTKPDVMDEEEVEVFLPRFTLEESYDMEEFLQELGMTD
AFEETRADFSGMSSGRGLHLSKVMHKSFVEVTEEGTEAAAATGAVVMMR
CLMVVPRFNANHPFLFFIQHSKTGAILFCGRFCSP
SEQ ID NO: 89 (similar to thrombin inhibitor
[Monodelphis domestica])
MVMNFPITFEPNILLSGSQVYKRWCVRMPALSEANNTFALNFFKKIGEE
ESEENVFYSPLSLYYALTMVLEGATGETAEQIQQVLSLSKNTDVHQSFQ
SFLAEVNKTGAPPLLRVANALFGEKTCGFLSPFKESCQKFYFSNVEELD
FAHMPEAARKHINDWVEEKTEGKISELLANDSVDVMTNLVLVNAIYFNG
KWEKPFDKAKTAEKMFNISEKKQKPVQMMYQRSTFSMTFIEDIPTQILV
LPYAGGHMDMVILLPVENKHLKMLKKRLTSENLVDWINPEMMNEIEVEV
FLPKFILAEHLDVEVILQKLGMLDAFDKTKADFSKMSARNDLCLSKVIH
KAYVEVNEEGTVAVSSTAAVMMTRSERMSLEFKADHPFIFYLIEKQTNK
IVFIGEVTSP
SEQ ID NO: 90 (similar to Placental thrombin
inhibitor (Protease inhibitor 6) (PI-6)
(Serpin B6) [Rattus norvegicus])
METLNVRRQILTRSSPDDFGMLSIKITSGEMLKDLESLRLAFWDSKKVS
ILLSVSDEIVQLTIMDPLLKANGNFAIKLFKVLGEDISKNVFFSLPSIS
SALSMILMGANGTTASQICQAMSLDKCNSIGGGDVHQHFLSLLTKVNKT
DTRCMLRKANSVFIEDSFEILASFKDACHKLYEAEIEELDFKGAPEQSR
QHINTWVAKKTEDIIRELLPPCTVNSNTCLFLVNVIYFKGSLEKPFNKA
DTREMPFKVSMVTTVHLLNERKSVYMFYKISTFKMTYVEEVSTKILLLL
YVSIELTMIMLFSPRGWKHEIVGGCANCLDARDRNKAQIEEWACALRHL
PAASGVKWDSACPEVEIMDSFHMKFSSELHFGLDFSLLCGWTESFVVLS
KRYASEKQVSKPLFS
SEQ ID NO: 91 (similar to Serpin B6 (Placental
thrombin inhibitor) (Cytoplasmic antiproteinase)
(CAP) (Protease inhibitor 6) (PI-6)
[Macaca mulatta])
MAAGKASGESEEASPSLTAEEREALGGLDRWRQDEGPTGQGKAAASRSA
DTVSPAGAALAPGPAAGSALIVAADDGRGASGKLFPASALPPPTVNGRV
SGAGPFLEPGQHSVSLEAPETRRWGPELVKAKASGQGGVDPRVPPRLSQ
CSGPGTPPASPRVFEVRGGSGVSTRRSTRSPPWVGKAGGASVCGGRGSG
AGQRAAGPGTRVGQRRAGTREPREWGVAARGCDPRRPSRAGARASVRLG
FPSGASQLRRTATRPVGRAWPRARPVTPLTRGPRAHRPSAHGRGGRRGM
GAAQSLPGHRSAIMDVLAEANGTFALNLLKTLGKDNSKNVFFSPMSMSC
ALAMVYMGAKGNTAAQMAQVLSFNKSGGGGDIHQGFQSLLTEVNKTGTQ
YLLRTANRLFGEKSCDFLSSFRDSCQKFYQAEMEELDFISAVEKSRKHI
NSWVAEKTEGENVLLSTRNSI
SEQ ID NO: 92 (similar to Serpin B6 (Placental
thrombin inhibitor) (Cytoplasmic antiproteinase)
(CAP) (Protease inhibitor 6) (PI-6)
[Macaca mulatta])
MAKAHYRFLTENSQAVAVFTRIEIGRFAHIRKSRGLRDPPRPPAQAPAG
LTVMDALSEGNGTFALNLLKKLGENNSPNLKILFGNWQGPNKEKPVQMM
FKKSTFQMTYAKEILNKILVLSYVGKELNMLPDENTDLKMLMSVEKELS
YERLIEWTKPDNMHEREMEVFLPRFKLEETYNMEDVLRSMDMVDALEQD
RADLKDLYLSKVMHKSFVEVNEEGTEAAAATTEEIVLCCASYSLRFCAD
HPFLFFIQHNKTNGILFCCRFSSP
SEQ ID NO: 93 (similar to thrombin inhibitor
[Bos taurus])
MDALSEANGTFALTLLKKLGEGNSKNVLIAPLSISSALAMVLLGARGNT
AAQMCQTLSLNKSSGGGEDVHQGFQNLLCEVNRTDTRYLLRTANRLFGE
KTYNFLSSFKDSCRKFYQAEMEELDFVCATEESRKHINTWVAEKTEGKI
RDLLSANSVYPMTCLVLVNAIYFKGNWDKQFYKVHTKERPFQVSKVNKT
VQMMFRKSTFKMTYIAEICTQILVLPYVGQELNMVILLPRERTDLNTVE
KALTYEKFVVWTKPDMLAEEEVEVFLPRFTLEESYDMECVLRDLGMTDG
FNMARADFIGLSCQPGLHLSKVVHKPFVEVTEEGTEAVAASEARIRGLS
LRTVPRFCANRPFLFFIQHSSTGAILFCGRFCSP
SEQ ID NO: 94 (similar to thrombin inhibitor
[Bos taurus])
MDALSEANGTFALTLLKKLGEGSSKNVLIAPLSISSALAMVLLGARGNT
AAQMCQTLSLNKSSGGGEDVHQDFQNLLSEVNRTDTQYLLRTANRLFGE
KPYDFLSSFKNACHIFYQAEMEELDFVSATEEPTKHINTWVAEKTEGKI
RDLLPANSVNPMTRLVLVSAVYFKGNWAKPFLKGRTMEGIFNVCKNVQK
RALMIYNWSTFKTACIAEICSQILVLPYVGQELNMVILLPFESTDLITV
EKALTYEKFVTWTKPDVLAEVEVEVFLPCFTLEESYDMECVLRDLGMTD
AFNAARADFSGMSCQPGLHLSKVMHKSFLEVTEEGTEAVAASEARIRRS
LGVVHHFYANRPFLFFIQHSRTGAILFCGRFCSP
SEQ ID NO: 95 (Alpha-1-antitrypsin 1-2)
MTPSISWGLLLLAGLCCMVPSFLAEDVQETDTSQKDQSPASHEIATNLG
DFAISLYRELVHQSNTSNIFFSPVSIATAFAMLSLGSKGDTHTQILEGL
QFNLTQTSEADIHKSFQHLLQTLNRPDSELQLSTGNGLFVNNDLKLVEK
FLEEAKNHYQAEVFSVNFAESEEAKKVINDFVEKGTQGKIVEAVKELDQ
DTVFALANYILFKGKWKKPFDPENTEEAEFHVDKSTTVKVPMMMLSGML
DVHHCSILSSWVLLMDYAGNASAVFLLPEDGKMQHLEQTLNKELISKIL
LNRRRRLVQIHIPRLSISGDYNLKTLMSPLGITRIFNNGADLSGITEEN
APLKLSKAVHKAVLTIDETGTEAAAATVFEAVPMSMPPILRFDHPFLFI
IFEEHTQSPIFVGKVVDPTHK
SEQ ID NO: 96 (Alpha-1-antitrypsin 1-1)
MTPSISWGLLLLAGLCCLVPSFLAEDVQETDTSQKDQSPASHEIATNLG
DFAISLYRELVHQSNTSNIFFSPVSIATAFAMLSLGSKGDTHTQILEGL
QFNLTQTSEADIHKSFQHLLQTLNRPDSELQLSTGNGLFVNNDLKLVEK
FLEEAKNHYQAEVFSVNFAESEEAKKVINDFVEKGTQGKIAEAVKKLDQ
DTVFALANYILFKGKWKKPFDPENTEEAEFHVDESTTVKVPMMTLSGML
HVHHCSTLSSWVLLMDYAGNATAVFLLPDDGKMQHLEQTLSKELISKFL
LNRRRRLAQIHFPRLSISGEYNLKTLMSPLGITRIFNNGADLSGITEEN
APLKLSQAVHKAVLTIDETGTEAAAVTVLQMVPMSMPPILRFDHPFLFI
IFEEHTQSPIFLGKVVDPTHK
SEQ ID NO: 97 (Alpha-1-antiproteinase)
MTPSISWGLLLLAGLFCLVPSFLAEDVQETDTSRRDSVPASHDTPYNLE
LSISLYRELGHKSTTSNIFFSQVSIATAFAMLSLGEKGDTHTQILEGLQ
FNLTQTSEADIHKAFQHLLQTLNRPDSELQLSTGNGSLLNNDLKLVEKF
LEEAKNNYHSEVFSVNFAESEEAKKVINDFVEKGTQGKIAEAVKDPDED
TVFALANYILFKGKWKKPFDPKHTEEAEFHVDTVTTVKVPMMTLTGMLD
VHHCSTLSSWVLLMDYLGNRTAVFLLPDDGKMQHLEQTLNKELISKFLL
NRHRRLAQVHLPRLSLSGNYTLNTLMSHLGITRIFNNGADLSGITEENA
PLKLSKAADKAVLTMDETGTEAAAATVLQAVPMSMPPILNFNKPFIFII
VEEHTQSPLFVGKVVDPTRK
SEQ ID NO: 98 (Alpha-1-antiproteinase)
MTPSISWRLLLLAGLCCLVPSYLAEDVQETDTSQKDQSPASHEMATNLG
DFAFSLYRELVHQSNTSNIFFSPVSIATAFALLSLGSKGDTQTQILEGL
QFNLTQTSEADIHKVFQHLLQTLNRPDSELQLSTGNGLFVNNDLKLVEK
FLEEAKNHYQSEVFSVNFAKSEEARKMINDFVEKGTQGKIVDAVKDLDE
DTVFALANYIFFQGKWKTPFDPEHTTEADFHVNESTTVRVPMMNLMRML
DVHYCSTLSSWVLMMDYLGNATAVFLLPDDGKMQHLEQTLNKELISKFL
LNRHRSLAEIHFPRLSISGSYNLKALMAPLGITRVFNNGADLSGITEEN
APLRLSKAVHKAVLTIDERGTEAAATTIVEAVFMSLPPILHFNHPFVFT
IVETHTQTPLFVGKVVDPTRK
SEQ ID NO: 99 (Alpha-1-antiproteinase)
MAPSISRGLLLLAALCCLAPSFLAEDAQETDTSQQDQSPTYRKISSNLA
DFAFSLYRELVHQSNTSNIFFSPMSITTAFAMLSLGSKGDTRKQILEGL
EFNLTQIPEADIHKAFHHLLQTLNRPDSELQLNTGNGLFVNKNLKLVEK
FLEEVKNNYHSEAFSVNFADSEEAKKVINDYVEKGTQGKIVDLMKQLDE
DTVFALVNYIFFKGKWKRPFNPEHTRDADFHVDKSTTVKVPMMNRLGMF
DMHYCSTLSSWVLMMDYLGNATAIFLLPDDGKMQHLEQTLTKDLISRFL
LNRQTRSAILYFPKLSISGTYNLKTLLSSLGITRVFNNDADLSGITEDA
PLKLSQAVHKAVLTLDERGTEAAGATVVEAVPMSLPPQVKFDHPFIFMI
VESETQSPLFVGKVIDPTR
SEQ ID NO: 100 (Alpha-1-antitrypsin)
MKPSISWGILLLAGLCCLVPSFLAEDAQETDASKQDQEHQACCKIAPNL
ADFSFNLYRELVHQSNTTNIFFSPVSIATAFAMLSLGTKGVTHTQILEG
LGFNLTEIAEAEVHKGFHNLLQTFNRPDNELQLTTGNGLFIHNNLKLVD
KFLEEVKNDYHSEAFSVNFTDSEEAKKVINGFVEKGTQGKIVDLVKDLD
KDTVLALVNYIFFKGKWKKPFDADNTEEADFHVDKTTTVKVPMMSRLGM
FDVHYVSTLSSWVLLMDYLGNATAIFILPDDGKMQHLEQTLNKEIIGKF
LKDRHTRSANVHFPKLSISGTYNLKTALDPLGITQVFSNGADLSGITED
VPLKLGKAVHKAVLTIDERGTEAAGATFMEIIPMSVPPEVNFNSPFIAI
IYDRQTAKSPLFVGKVVDPTR
SEQ ID NO: 101 (Alpha-1-antitrypsin)
HVEDPQGDAAQKTDTSHHDQEHSTFNKITPSLAEFAFSLYRQLAHQSNS
TNIFFSPVSIATAFAMLSLGTKADTHSEILEGLNFNLTEIPEAQIHEGF
QELLHTLNKPDSQLQLTTGNGLFLNKSVKVVDKFLEDVKKLYHSEAFSV
NFEDTEEAKKQINNYVEKGTQGKIVDLVKELDRDTVFALVNYIFFKGKW
ERPFEVEATKEEDFHVDQATTVKVPMMRRLGMFNIYHCEKLSSWVLLMK
YLGNATAIFFLPDEGKLQHLENELTHDIITKFLENENRRSANLHLPKLA
ITGTYDLKTVLGHLGITKVFSNGADLSGVTEDAPLKLSKAVHKAVLTID
EKGTEAAGAMFLEAIPMSIPPEVKFNKPFVFLMIEQNTKSPLFMGKVVN
PTQK
SEQ ID NO: 102 (Alpha-1-antitrypsin)
LLLAGLCCLLPGSLAEDPQGDAAQKTDTPPHDQNHPTLNKITPSLAEFA
FSLYRQLAHQSNSTNIFFSPVSIATAFAMLSLGTKADTHSEILEGLNFN
LTEIPEAQVHEGFQELLRTLNKPDSQLQLTTGNGLFLNKSLKVVDKFLE
DVKNLYHSEAFSVNFEDTEEAKKQINNYVEKGTQGKVVDLVKELDRDTV
FALVNYIFFKGKWERPFEVEATEEEDFHVDQATTVKVPMMRRLGMFNIY
HCEKLSSWVLLMKYLGNATAIFFLPDEGKLQHLENELTHDIITKFLENE
NRRSANLHLPKLAITGTYDLKTVLGHLGITKVFSNGADLSGVTEDAPLK
LSKAVHKAVLTIDEKGTEAAGAMFLEAIPMSIPPEVKFNKPFVFLMIEQ
NTKSPLFIGKVVNPTQK
SEQ ID NO: 103 (Alpha-1-antitrypsin)
MPSSVSWGILLLAGLCCLVPVSLAEDPQGDAAQKTDTSHHDQDHPTFNK
ITPNLAEFAFSLYRQLAHQSNSTNIFFSPVSIATAFAMLSLGTKADTHS
EILEGLHFNLTEIPEAQVHEGFQELLRTLNQPDSQLQLTTGNGLFLNES
LKLVDKFLEDVKKLYHSDAFTVNFGDTEEAKKQINDYVEKGTQGKIVDL
VKELDRDTVFALVNYIFFKGKWERPFEVKDTKEEDFHVDEVTTVKVPMM
RRLGMFNIHYCEKLSSWVLLMKYLGNATAIFFLPDEGKLQHLENELTHD
IITKFLENENRRSASLHLPKLSITGTYDLKRVLGQLGITKVFSNGADLS
GVTEEAPLKLSKAVHKAVLTIDEKGTEAAGAMFLEAIPMSIPPEVKFNK
PFVFLMIEQNTKSPLFVGKVVNPTQK
SEQ ID NO: 104 (Alpha-1-antiproteinase)
MALSITRGLLLLAALCCLAPISLAGVLQGHAVQETDDTSHQEAACHKIA
PNLANFAFSIYHHLAHQSNTSNIFFSPVSIASAFAMLSLGAKGNTHTEI
LKGLGFNLTELAEAEIHKGFQHLLHTLNQPNHQLQLTTGNGLFINESAK
LVDTFLEDVKNLYHSEAFSINFRDAEEAKKKINDYVEKGSHGKIVELVK
VLDPNTVFALVNYISFKGKWEKPFEMKHTTERDFHVDEQTTVKVPMMNR
LGMFDLHYCDKLASWVLLLDYVGNVTACFILPDLGKLQQLEDKLNNELL
AKFLEKKYASSANLHLPKLSISETYDLKSVLGDVGITEVFSDRADLSGI
TKEQPLKVSKALHKAALTIDEKGTEAVGSTFLEAIPMSLPPDVEFNRPF
LCILYDRNTKSPLFVGKVVNPTQA
SEQ ID NO: 105 (Alpha-1-antitrypsin)
MASSSTWGLLLLAGLCCLVPISLAEGLQGHAVQETDVPRHDHEQHQEAA
CHRIAPNLADFAFSLYRQVARQSNTSNIFLSPVTIARAFAMLSLGTKGA
THAEILEGLQFNLTEKAEAEIHEGFQHLLHTLNQPDNQLQLTTGNGLFI
DEKAKLVPKFLEDVKNLYHSEAFSINFRDTEEAKKCINDYVEKGSQGKI
VDLVDELDKDTVFALVNYIFFKGKWEKPFEVEQTTEEDFHVDEETTVKV
PMMNRLGMFDLHHCDKLSSWVLLMDYVATATAFFILPDQGKLHQLEDML
TKEIRAKFLEKRYPSSANLHLPKLTISGTYDLKSLLGNLGITKVFSDEA
DLSGVTEEQPLKLSKALHRAVLTIDEKGTEATGATILEAIPMSIPPNVK
FNKPFLFLIYDTKTKAVLFMGKVMNPTQK
SEQ ID NO: 106 (Alpha-1-antiproteinase F)
MPPSVSRALLLLAGLGCLLPGFLADEAQETAVSSHEQDHPACHRIAPSL
AEFALSLYREVAHESNTTNIFFSPVSIALAFAMLSLGAKGDTHTQVLEG
LKFNLTETAEAQIHDGFRHLLHTVNRPDSELQLAARNALVVHENLKLQH
KFLEDAKNLYQSEAFLVDFRDPEQAKTKINSHVEKGTRGKIVDLVQELD
ARTLLALVNYVFFKGKWEKPFEPENTKEEDFHVNATTTVRVPMMSRLGR
YDLFHCSTLASTVLRMDYKGNATALFLLPDEGKLQHLEDTLTTELITKF
LAKSSLRSVTVHFPKLSISGTYDLKPLLGKLGITQVFSDNADLSGITEQ
EPLKASQALHKAVLTIDERGTEAAGATYMEIIPMSLPDSITLDRPFLFV
IYSHEIKSPLFVGKVVDPTQH
SEQ ID NO: 107 (Alpha-1-antiproteinase)
MMPSTLSLCLMLAGLCSLVTSHLTEEIQASNDTENEYSSTRRISPYMTD
FSIDFYRLLVSKSNTTNIFFSPISIYTAFTLLALGAKSATRDQILTGLR
FNRTEISEEHIFEGFQQLLNTFNLPENELQLTTSNGLFIDKNLKLVAKF
LEDSKRLYASDTFSTNFEDNMAAKKQINDYVEKETQGKIVDLIQNLDSN
VVFVLVNCIFFKGKWEKPFMTELTTECPFHVDSKTTVPVQTMRRLGMFN
VFYDQDLSCWVLKMKYMGNATALFILPDTGKIEKVENALNKMLFHKWTR
NLKRRAISLYFPKVSISGNYDLKILRELGITDVFGSNADLSGITEETNL
KLSQAVHKAVVNIDEKGTEASGATFAEGIPMSIPPTVEFLRPFIFIILE
ENTKSVLFMGKVMNPTGN
SEQ ID NO: 108 (serine (or cysteine) proteinase
inhibitor, clade A (alpha-1 antiproteinase,
antitrypsin), member 5 [Homo sapiens])
MQLFLLLCLVLLSPQGASLHRHHPREMKKRVEDLHVGATVAPSSRRDFT
FDLYRALASAAPSQSIFFSPVSISMSLAMLSLGAGSSTKMQILEGLGLN
LQKSSEKELHRGFQQLLQELNQPRDGFQLSLGNALFTDLVVDLQDTFVS
AMKTLYLADTFPTNFRDSAGAMKQINDYVAKQTKGKIVDLLKNLDSNAV
VIMVNYIFFKAKWETSFNHKGTQEQDFYVTSETVVRVPMMSREDQYHYL
LDRNLSCRVVGVPYQGNATALFILPSEGKMQQVENGLSEKTLRKWLKMF
KKRQLELYLPKFSIEGSYQLEKVLPSLGISNVFTSHADLSGISNHSNIQ
VSEMVHKAVVEVDESGTRAAAATGTIFTFRSARLNSQRLVFNRPFLMFI
VDNNILFLGKVNRP
SEQ ID NO: 109 (serine (or cysteine) proteinase
inhibitor, clade A, member 5
[Mus musculus])
MRFFPILCLVLFISHGVASRRHSHSKKKKAKESSVGAVGPPSSKDFAFR
LYRALVSESPGQNVFFSPLSVSMSLGMLSLGAGLKTKTQILDGLGLSLQ
QGQEDKLHKGFQQLLQRFRQPSDGLQLSLGSALFKDPAVHIRDDFLSAM
KTLYMSDTFSTNFGNPEIAKKQINNYVAKQTKGKIVDFIKDLDSTHVMI
VVNYIFFRAKWQTAFSETNTHKMDFHVTPKRTTQVPMMNREDGYSYYLD
QNISCTVVGIPYQGNAIALFILPSEGKMKQVEDGLDERTLRNWLKMFTK
RRLDLYLPKFSIEATYKLENVLPKLGIQDVFTTHADLSGITDHTNIKLS
EMVHKSMMEVEESGTTAAAITGAIFTFRSARPSSLKIEFTRPFLLTLME
DSHILFVGKVTRP
SEQ ID NO: 110 (serine (or cysteine) peptidase
inhibitor, clade C (antithrombin), member 1
[Rattus norvegicus])
MYSPGIGSAVAGERKLCLLSLLLIGALGCAVCHGNPVDDICIAKPRDIP
VNPMCIYRSPAKKATEEDVLEQKVPEATNRRVWELSKANSRFATNFYQH
LADSKNDNDNIFLSPLSISTAFAMTKLGACNNTLKQLMEVFKFDTISEK
TSDQIHFFFAKLNCRLYRKANKSSNLVSANRLFGDKSLTFNESYQDVSE
IVYGAKLQPLDFKENPEQSRVTINNWVANKTEGRIKDVIPQGAIDELTA
LVLVNTIYFKGLWKSKFSPENTRKEPFHKVDGQSCLVPMMYQEGKFKYR
RVGEGTQVLEMPFKGDDITMVLILPKPEKSLAKVEQELTPELLQEWLDE
LSEVMLVVHVPRFRIEDSFSLKEQLQDMGLVDLFSPEKSQLPGIIAEGR
DDLFVSDAFHKAFLEVNEEGSEAAASTSVVITGRSLNPSRVTFKANRPF
LVLIREVALNTIIFMGRVSNPCVN
SEQ ID NO: 111 (Antithrombin-III)
MYSPGAGSGAAGERKLCLLSLLLIGALGCAICHGNPVDDICIAKPRDIP
VNPLCIYRSPGKKATEEDGSEQKVPEATNRRVWELSKANSRFATNFYQH
LADSKNDNDNIFLSPLSISTAFAMTKLGACNDTLKQLMEVFKFDTISEK
TSDQIHFFFAKLNCRLYRKANKSSDLVSANRLFGDKSLTFNESYQDVSE
VVYGAKLQPLDFKENPEQSRVTINNWVANKTEGRIKDVIPQGAINELTA
LVLVNTIYFKGLWKSKFSPENTRKEPFYKVDGQSCPVPMMYQEGKFKYR
RVAEGTQVLELPFKGDDITMVLILPKPEKSLAKVEQELTPELLQEWLDE
LSETMLVVHMPRFRTEDGFSLKEQLQDMGLIDLFSPEKSQLPGIVAGGR
DDLYVSDAFHKAFLEVNEEGSEAAASTSVVITGRSLNPNRVTFKANRPF
LVLIREVALNTIIFMGRVANPCVN
SEQ ID NO: 112 (Antithrombin-III)
MYSNVIGTITSGKRKVYLLSLLLIGFWDCVTCHGSPVDICIAKPRDIPM
NPMCIYRSPEKKATEDEGSEQKIPEATNRRVWELSKANSRFATTFYQHL
ADSKNDNDNIFLSPLSISTAFAMTKLGACNDTLQQLMEVFKFDTISEKT
SDQIHFFFAKLNCRLYRKANKSSKLVSANRLFGDKSLTFNETYQDISEL
VYGAKLQPLDFKENAEQSRAAINKWVSNKTEGRITDVIPPEAINELTVL
VLVNTIYFKGLWKSKFSPENTRKELFYKADGESCSASMMYQEGKFRYRR
VAEGTQVLELPFKGDDITMVLILPKPEKSLAKVEKELTPEVLQEWLDEL
EEMMLVVHMPRFRIEDGFSLKEQLQDMGLVDLFSPEKSKLPGIVAEGRD
DLYVSDAFHKAFLEVNEEGSEAAASTAVVIAGRSLNPNRVTFKANRPFL
VFIREVPLNTIIFMGRVANPCVK
SEQ ID NO: 113 (Antithrombin-III)
MISNGIGTVTTGKRSMCLFPLLLIGLWGCVTCHRSPVEDICTAKPRDIP
VNPMCIYRSPEKKATEGEGSEQKIPGATNRRVWELSKANSHFATAFYQH
LADSKNNNDNIFLSPLSISTAFAMTKLGACNNTLKQLMEVFKFDTISEK
TSDQIHFFFAKLNCRLYRKANKSSELVSANRLFGDKSITFNETYQDISE
VVYGAKLQPLDFKGNAEQSRLTINQWISNKTEGRITDVIPPQAIDEFTV
LVLVNTIYFKGLWKSKFSPENTKKELFYKADGESCSVPMMYQEGKFRYR
RVAEGTQVLELPFKGDDITMVLILPKLEKPLAKVERELTPDMLQEWLDE
LTETLLVVHMPHFRIEDSFSVKEQLQDMGLEDLFSPEKSRLPGIVAEGR
NDLYVSDAFHKAFLEVNEEGSEAAASTVISIAGRSLNLNRVTFQANRPF
LVLIREVALNTIIFMGRVANPCVN
SEQ ID NO: 114 (thrombin inhibitor infestin
precursor [Triatoma infestans])
LEENDCACPRVLHRVCGSDGNTYSNPCTLDCAKHEGKPDLVQVHEGPCD
PNDHDFEDPCECDNKFEPVCGTDHITYSNLCHLECAAFTTSPGVEVKYE
GECHAEIMEQHQILKSCICTKMYKPVCGTDGHTYPNLCVLKCRISSKPG
LKLAHVGKCGIGLLAVETKEVRNPCACFRNYVPVCGSDGKTYGNPCMLN
CAAQTKVPGLKLVHEGRCQRSNVEQF
SEQ ID NO: 115 (brasiliensin precursor
[Triatoma brasiliensis])
MRYLLLLGLAAFSAVSAEEKDPPCACPLIWKPVCGNDGQTYPNECLLNC
IKYVLKKDIEVAYQGICKHFVHAAAEAEELVESKPPCACPLIWKPVCGN
DGQTYPNECMLNCMKYILKKDIEVAYQGMCKHFVHAAAEAEELVESKNP
CECPRALHRVCGSDGNTYSNPCTLNCAKHEGKPDLVQVHEGPCSPDEHD
FEDPCECDNKFDPVCGTDKVTYRNLCXLECAMFTTSPGVEVDYEGECLA
ETVLLEENHCACPRVLHRVCGSDGNTYSNPCTLDCAKHEGKPDLVQVHE
GPCDPNDHDFEDPCECDNKFEPVCGTDHITYSNLCHLECAAFTTSPGVE
VKYEGECHAEIMEQHQILKSCICTKIYSPVCGTDGHTYPNLCILECHIS
FNPGLKLAHVGKCGTDLQDIETKQVRNPCACFRNYLPVCGSDGKTYGNP
CMLNCAAHTKVPGLKLAHKGRCQRSDVEQF
SEQ ID NO: 116 (Serine protease inhibitor
dipetalogastin)
LIKELVNMVIQHAEEEEVKELKNPCECPRALHRVCGSDGNTYSNPCMLN
CAKHEGNPDLVQVHKGPCDEHDHDFEDPCKCDNKFEPVCGDDQITYLNL
CHLECATFTTSPGVEVAYEGECHAETTNAMEVLFQGNPCECPRALHRVC
GSDGNTYSNPCMLTCAKHEGNPDLVQVHEGPCDEHDHDFEDTCQCDDTF
QPVCGDDEITYRNLCHLECATFTTSPGVEVKHEGECHPETKVNQLILKS
CMCPKIYKPVCGTDGRTYPNICVLKCHISSNPGLGLAHLGECKVAVLAK
ETGEVRNPCNCFRNFNPVCGTDGKTYGNLCMLGCAAETKVPGLKLLHNG
RCLPKEQL
SEQ ID NO: 117 (thrombin inhibitor protein
[Rhodnius prolixus])
RLLLLLGLAALVAAEGGEPCACPHALHRVCGSDGETYSNPCTLNCAKFN
GKPELVKVHDGPCEPDEDEDVCQECDGDEYKPVCGSDDITYDNNCRLEC
ASISSSPGVELKHEGPCRTEEKKILKRSDEFEMYRCACPKIYYPVCGTD
GETYPNLCVLECHMRMNPGLQLHHYGHCQHHHHHHPPPHHHHHHHPHHT
TEKPVEPCACPHALHRVCGSDGETYSNPCTLNCAKHNGKPGLVKVHDGP
CEPDEDEDVCQECDDVDYEPVCGTDDKTYDNNCRLECASISSSPGVELK
HEGICRKEEKKLPKRSVGLEHTCVCPYNYFPVCGTDGETYPNLCALQCR
MREVPGLELKHTGKCLPHLDFPDPV
SEQ ID NO: 118 (thrombin inhibitor protein
[Rhodnius prolixus])
MKRLLLLLGLAALVAAEGGEPCACPHALHRVCGSDGETYSNPCTLNCAK
FNGKPELVKVHDGPCEPDEDEDVCQECDGDEYKPVCGSDGITYDNNCRL
ECASISSSPGVELKHEGICRKEEKKLPKRSVGLEHTCVCPYNYFPVCGT
DGETYPNLCALQCRMREVPGLELKHTGKCLPHLDFPDPV
SEQ ID NO: 119 (thrombin inhibitor haemalin
[Haemaphysalis longicornis])
MKLFVFLALFGAAFAQRNGFCRLPAEPGICRAFMPRYYFDVEKGQCEQF
IYGGCKGNENNFETLKECQDACGEPERASDFEKADFETGCKAAPETGLC
KASFERWFFNAASGECEEFIYGGCGGNDNNYENKEECEFACKY
SEQ ID NO: 120 (boophilin [Boophilus
microplus])
MKCIILLAVLGTAFAQRNGFCRLPADEGICKALIPRFYFNTETGKCTMF
SYGGCGGNENNFETIEECQKACGAPERVNDFESADFKTGCEPAADSGSC
AGQLERWFYNVQSGECETFVYGGCGGNDNNYESEEECELVCKNM
SEQ ID NO: 121 (boophilin [Boophilus
microplus])
MKYLILLAVLGTAFAQRNGFCRLPADEGICKALIPRFYFNTETGKCTMF
SYGGCGGNENNFETIEDCQKACGAPERVSDFEGADFKTGCEPAADSGSC
AGQLERWFYNVRSGECETFVYGGCGGNDNNYESEEECELVCKNM
SEQ ID NO: 122 (Ornithodorin)
LNVLCNNPHTADCNNDAQVDRYFREGTTCLMSPACTSEGYASQHECQQA
CFVGGEDHSSEMHSSCLGDPPTSCAEGTDITYYDSDSKTCKVLAASCPS
GENTFESEVECQVACGAPIEG
SEQ ID NO: 123 (thrombin inhibitor
[Ornithodoros moubata])
LNVLSNNPHTADCNNDAQVDRYFREGTTCLMSPACTSEGYASQHECLRP
ALLAGKTTAVKCTAHALVTRPLPARKARTSPTTILIAKHVRY
SEQ ID NO: 124 (savignin [Ornithodoros
savignyi])
MLFYVVITLVAGTVSGLNVRCNNPHTANCENGAKLESYFREGETCVGSP
ACPGEGYATKEDCQKACFPGGGDHSTNVDSSCFGQPPTSCETGAEVTYY
DSGSRTCKVLQHGCPSSENAFDSEIECQVACGVSME
SEQ ID NO: 125 (thrombin inhibitor
[Amblyomma hebraeum])
MGFLVASAVLVCVTSQRVPGYCKKKPAVGPCKALIEKWYFDYSTQSCKT
FYYGGCGGNGNKFSSRKKCREACLPKRPSVPVCKQMPDPGFCRAYMPHW
FFNSKSGYCEGFVYGGCQGNDNRFKSCWQCMKKCRTAREANRLCWKLTK
EFNKKFLRNVPTAKPLPPK
SEQ ID NO: 126 (thrombin inhibitor
[Amblyomma americanum])
MRPQAFIGAFVFTLVLRQAAGIKWSRCFRPKAVGNCQNKVPAWYYDFWS
FRCKGFLYSGCGGNSNRFPTEEECQKSCLRKSKRKEVCSLKPKTGKCKA
AIPLWYYDPELDECRGLIYGGCKGNANRFETCLKCMKRCSGNNNARKIC
KKQTKKFLEENNLGSNRHHKKPSWPQLSIRIPFIEK
SEQ ID NO: 127 (thrombin inhibitor [Ixodes
scapularis])
MHQEGDFKMGHCSDLKVSALEIPYKGNKMSMVILLPEDVEGLSDLEEHL
TAPKLLALLGGMYVTSDVNLHFPKFKLEQSMGLKDVLMAMGVKDFFTFL
ADLSGISATGNLCASDVIHKAFVEVNEEGTEAAAATAILMDCIPQVVNF
FVDHPFMFLICSHDPDAVLFMGSIREL
SEQ ID NO: 128 (thrombin inhibitor [Ixodes
scapularis])
MHQKGDFKMGHCSDLKVTALEIPYKGNKMSMIILLPEDVEGLSVLEEHL
TAPKLSALLGGMYVTPDVNLRLPKFKLEQSIGLKDVLMAMGVKDFFTSL
ADLSGISAAGNLCASDVIHKAFVEVNEEGTEAAAATAIPMMLMCARFPQ
VVNFFVDHPFMFLIHSHDPDVVLFMGSIREL
SEQ ID NO: 129 (thrombin inhibitor [Ixodes
scapularis])
MASDFSNSLISFSVDLYKKLKSESDGASNFICSPFSIAAALSMTLAGAK
HDTAKQISNALHMQDTTVHENFAYFFSKLPGYAPDVILHVANRLYAEET
YNTLDEFTHLLEKSYSTTVEKVDFKRNAEKTRLQVNTWVEEVTQSKIKD
LLAEGTIDDFTSLIIINAVYFKGLWHDQFDPKRTSQQEFHLTADRTKMV
DMMHHKQRFRMCRHPNFKVSALEIPYKGQKMSMVILLPEEIDGLADLEE
TLTSSKIREIIQELSYQGDIELSLPRFKLEHTVGLKNVLAAMGIEDMFD
ALKCDLSGISPDNALVVSDVVHKAFIEVNEEGTEAAAATAMVMLCCMSF
PTRFTVDHPFLFLIRCHDPDVILFIGSVAQI
SEQ ID NO: 130 (tsetse thrombin inhibitor
precursor [Glossina morsitans
morsitans])
MKFFTVLFFLLSIIYLIVAAPGEPGAPIDYDEYGDSSEEVGGTPLHEIP
GIRL
SEQ ID NO: 131 (thrombin inhibitor madanin 2
[Haemaphysalis longicornis])
MKHFVILILAVVASAVVMAYPERDSAKDGNQEKERALLVKVQERYQGNQG
DYDEYDQDETTPPPDPTAQTARPRLRQNQD
SEQ ID NO: 132 (thrombin inhibitor madanin 1
[Haemaphysalis longicornis])
MKHFAILILAVVASAVVMAYPERDSAKEGNQEQERALHVKVQKRTDGDA
DYDEYEEDGTTPTPDPTAPTAKPRLRGNKP
SEQ ID NO: 133(Antithrombin-III)
MHLFIGVSLRPLGHGIPAPYAVEDICTAKPRDIPVNPICIYRNPEKKPQ
ERRGAGAGEGQDPGVHKPPASGSCPGPTRAFGRRSFLQAPGPTPRTMRR
TSSCRPS
SEQ ID NO: 134 (thrombin inhibitor
[Amblyomma hebraeum])
MGFLVASAVLVCVTSQRVPGYCKKKPAVGPCKALIEKWYFDYSTQSCKT
FYYGGCGGNGNKFSSRKKCREACLPKRPSVPVCKQMPDPGFCRAYMPHW
FFNSKSGYCEGFVYGGCQGNDNRFKSCWQCMKKCRTAREANRLCWKLTK
EFNKKFLRNVPTAKPLPPK
SEQ ID NO: 135 (thrombin inhibitor
[Amblyomma americanum])
MRPQAFIGAFVFTLVLRQAAGIKWSRCFRPKAVGNCQNKVPAWYYDFWS
FRCKGFLYSGCGGNSNRFPTEEECQKSCLRKSKRKEVCSLKPKTGKCKA
AIPLWYYDPELDECRGLIYGGCKGNANRFETCLKCMKRCSGNNNARKIC
KKQTKKFLEENNLGSNRHHKKPSWPQLSIRIPFIEK
SEQ ID NO: 136 (thrombin inhibitor, TTI =
antithrombotic peptide [Glossina
morsitans])
GEPGAPIDYDEYGDSSEEIG
SEQ ID NO: 137 (thrombin inhibitor
[Amblyomma americanum])
MEIKRCVHVLLTLATVYGTSVSISSICSLPQVKGNCRGIFEMWHYNSTK
DTCSLFTYGGCGGNENRFENCTQCMESCSTNENRTEICQLLEKEADEDY
YSGWDDNTGGDGYTAPPREAYDEDYDEE
SEQ ID NO: 138 (Theromin)
ECENTECPRACPGEYEFDEDGCNTCVCKGCDDAQCRCSSDANGCESFCT
CNTRCSAADECNPRCTCK
SEQ ID NO: 139 (monobin [Argas
monolakensis])
MRLLALFAFAVAVVSAQRNQMCQQPRTQGSCDASNQITKFFYTGSGCTS
APVCSDTDGGYGTEDECIQACTVQGGHHNEGAGEEGCSGDPPRGDCGGQ
VEERYYFDSTTRTCQTFEYRGCSSGNPDNSYETEIECEIACPSASS
SEQ ID NO: 140 (similar to thrombin inhibitor
[Hydra magnipapillata])
MCLRCRPCEPDLCPPRPLCNGGYVKGICGCCDTCAKVDGEECGGLWNMY
GKCDVGFVCAQVKRHTKGVCVSVKKKSNNKENTCALKPETGPCRAAIKA
WYYDYKTDTCKRFVYGGCGGNSNRFREKKMCQKTCYA
SEQ ID NO: 141 (Thrombin inhibitor subunit 1)
EKFPAVNQKPQAAXL
SEQ ID NO: 142 (similar to thrombin inhibitor
[Bos taurus])
MDALSEANGTFALTLLKKLGEGSSKNVFISPLSISSALAMVLMGARGNT
AAQMCQTLSLNKSSGGGEDVHQGFQNLLSEVNRTDPRLLAQNRQRGFSG
DKTYDFLSSFKDSCRXILPSRDGRAGLYQRYGAVQEAHKHLGS
SEQ ID NO: 143 (Trypsin inhibitor 1)
MATTMAKLITLVVLAILAFVEVSVSGYKTSISTITIEDNGRCTKSIPPI
CFPDGRP
SEQ ID NO: 144 (Phosphatidylethanolamine-binding
protein 1)
MPVDLGKWSGPLSLQEVEERPQHALHVKYTGTEVDELGKVLTPTQVKNR
PTSIAWDGLDPGKLYTLVLTDPDAPSRKDPKYREWHHFLVVNMKGNDIS
SGTVLSDYVGSGPPKGTGLHRYVWLVYEQSGPLKCDEPILSNRSGDHRG
KFKVASFRKKYELGPPVAGTCYQAEWDDYVPKLCEQLSGK
SEQ ID NO: 145 (bothrojaracin = thrombin-
induced platelet aggregation inhibitor 15 kda
polypeptide {N-terminal} [Bothrops
jararaca])
DCPSDWSPYGQGCYXF
SEQ ID NO: 146 (thrombostasin [Haematobia
irritans])
NINIMKHFVVIGILALSAVCQAQNVLSGRRQHGAQGLSGYSGDNDWGYY
GEAGAPGSDYSGSSGQWAPLDFDYNSLPGLSGYNHEQQDYEEDSYRHVR
SAGPITLQLDDDDDDDSGIPIFEMDDEDEDSNDNQKFPLSFERFPENEK
NQVGLRARFNKFMAKFTSLFGRRRGVNVPNAA
SEQ ID NO: 147 (hemadin [Haemadipsa
sylvestris])
CDCGEKICLYGQSCNDGQCSGDPKPSSEFEEFEIDEEEK
SEQ ID NO: 148 ([Anopheles
gambiae str. PEST])
MASKLFVLAFLCLALVVVVQSAPQYARGDVPTYDEEDFDEESLKPHSSS
SSDDGEEEFDPSLLEEHADAPTARDPGRNPEFLRNSNTDEQASAPAASS
SESDE
SEQ ID NO: 149 (anophelin-like funestolin
[Anopheles funestus])
MATKLIVIAFLCAALIAVVQSAPQYAQGEEPTYDEDDDEPVKPHSSADP
DASYEEFDPSQLTEYANTAQDPGRRPHFLEQANSNNGDQLPSQSDSSSE
STEH
SEQ ID NO: 150 (salivary anti-thrombin anophelin
[Anopheles stephensi])
MASKVIVIALLCIALAAFVQGAPQYTHGEEPEYDEDDGADEPVQPHSSS
NHADTEDDFDLSLLDKPYANAPENADPGRRPEFLKQHNNENQSDSSSGS
TEN
SEQ ID NO: 151 (salivary anti-thrombin peptide
anophelin [Anopheles darlingi])
MANKLFLISLLCVALVAKIAQAAPQYAPGEEPSYDEDTDDKLIENDTSI
TDEDYAEIEASLSQAFGTAADPGRRLGEGKKP
SEQ ID NO: 152 (salivary anti-thrombin peptide
anophelin [Anopheles albimanus])
MANKLVLISLLCVVLVAKITQAAPQYAPGDEPSYDEDTDDSDKLVENDT
SITDEDYAAIEASLSETFNTAADPGRRLGEGSKP
SEQ ID NO: 153 (hirudin, * Y is sulphated)
VYTDCTESGQNLCLCEGSNVCGQGNKCILGSDGEKNQCVTGEGTPKPQS
HNDGDFEEIPEEY*LQ
SEQ ID NO: 154 (synthetic peptide)
FEEIPEEYL
SEQ ID NO: 155 (synthetic peptide)
YEPIPEEA
SEQ ID NO: 156 (synthetic peptide)
NGDFEEIPEEYL
SEQ ID NO: 157 (synthetic peptide)
APPFDFEAIPEEYL
SEQ ID NO: 158 (modified hirulog)
FPRMHKGGGNGDFEEIPEEYL
SEQ ID NO: 159 (modified hirulog)
FPRMHKGGNGDFEEIPEEYL
SEQ ID NO: 160 (modified hirulog)
FPRMHKGGGGNGDFEEIPEEYL
SEQ ID NO: 161 (modified hirulog)
FPRMHKTGGNGDFEEIPEEYL
SEQ ID NO: 162 (modified hirulog)
FPRMHKTGNGDFEEIPEEYL
SEQ ID NO: 163 (modified hirulog)
FPRMHKTGGGNGDFEEIPEEYL
SEQ ID NO: 164 (modified hirulog)
FPRMHKTAGNGDFEEIPEEYL
SEQ ID NO: 165 (modified hirulog)
FPRMHKTANGDFEEIPEEYL
SEQ ID NO: 166 (modified hirulog)
FPRMHKTAGGNGDFEEIPEEYL
SEQ ID NO: 167 (modified hirulog)
F_{D}PRMHKGGGNGDFEEIPEEYL
SEQ ID NO: 168 (modified hirulog)
F_{D}PRMHKGGNGDFEEIPEEYL
SEQ ID NO: 169 (modified hirulog)
F_{D}PRMHKGGGGNGDFEEIPEEYL
SEQ ID NO: 170 (modified hirulog)
F_{D}PRMHKTGGNGDFEEIPEEYL
SEQ ID NO: 171 (modified hirulog)
F_{D}PRMHKTGNGDFEEIPEEYL
SEQ ID NO: 172 (modified hirulog)
F_{D}PRMHKTGGGNGDFEEIPEEYL
SEQ ID NO: 173 (modified hirulog)
F_{D}PRMHKTAGNGDFEEIPEEYL
SEQ ID NO: 174 (modified hirulog)
F_{D}PRMHKTANGDFEEIPEEYL
SEQ ID NO: 175 (modified hirulog)
F_{D}PRMHKTAGGNGDFEEIPEEYL
SEQ ID NO: 176 (modified hirulog)
FPRXHKGGGNGDFEEIPEEYL
SEQ ID NO: 177 (modified hirulog)
FPRXHKGGNGDFEEIPEEYL
SEQ ID NO: 178 (modified hirulog)
FPRXHKGGGGNGDFEEIPEEYL
SEQ ID NO: 179 (modified hirulog)
FPRXHKTGGNGDFEEIPEEYL
SEQ ID NO: 180 (modified hirulog)
FPRXHKTGNGDFEEIPEEYL
SEQ ID NO: 181 (modified hirulog)
FPRXHKTGGGNGDFEEIPEEYL
SEQ ID NO: 182 (modified hirulog)
FPRXHKTAGNGDFEEIPEEYL
SEQ ID NO: 183 (modified hirulog)
FPRXHKTANGDFEEIPEEYL
SEQ ID NO: 184 (modified hirulog)
FPRXHKTAGGNGDFEEIPEEYL
SEQ ID NO: 185 (modified hirulog)
F_{D}PRXHKGGGNGDFEEIPEEYL
SEQ ID NO: 186 (modified hirulog)
F_{D}PRXHKGGNGDFEEIPEEYL
SEQ ID NO: 187 (modified hirulog)
F_{D}PRXHKGGGGNGDFEEIPEEYL
SEQ ID NO: 188 (modified hirulog)
F_{D}PRXHKTGGNGDFEEIPEEYL
SEQ ID NO: 189 (modified hirulog)
F_{D}PRXHKTGNGDFEEIPEEYL
SEQ ID NO: 190 (modified hirulog)
F_{D}PRXHKTGGGNGDFEEIPEEYL
SEQ ID NO: 191 (modified hirulog)
F_{D}PRXHKTAGNGDFEEIPEEYL
SEQ ID NO: 192 (modified hirulog)
F_{D}PRXHKTANGDFEEIPEEYL
SEQ ID NO: 193 (modified hirulog)
F_{D}PRXHKTAGGNGDFEEIPEEYL
SEQ ID NO: 194 (modified hirulog)
FPRMHXGGGNGDFEEIPEEYL
SEQ ID NO: 195 (modified hirulog)
FPRMHXGGNGDFEEIPEEYL
SEQ ID NO: 196 (modified hirulog)
FPRMHXGGGGNGDFEEIPEEYL
SEQ ID NO: 197 (modified hirulog)
FPRMHXTGGNGDFEEIPEEYL
SEQ ID NO: 198 (modified hirulog)
FPRMHXTGNGDFEEIPEEYL
SEQ ID NO: 199 (modified hirulog)
FPRMHXTGGGNGDFEEIPEEYL
SEQ ID NO: 200 (modified hirulog)
FPRMHXTAGNGDFEEIPEEYL
SEQ ID NO: 201 (modified hirulog)
FPRMHXTANGDFEEIPEEYL
SEQ ID NO: 202 (modified hirulog)
FPRMHXTAGGNGDFEEIPEEYL
SEQ ID NO: 203 (modified hirulog)
F_{D}PRMHXGGGNGDFEEIPEEYL
SEQ ID NO: 204 (modified hirulog)
F_{D}PRMHXGGNGDFEEIPEEYL
SEQ ID NO: 205 (modified hirulog)
F_{D}PRMHXGGGGNGDFEEIPEEYL
SEQ ID NO: 206 (modified hirulog)
F_{D}PRMHXTGGNGDFEEIPEEYL
SEQ ID NO: 207 (modified hirulog)
F_{D}PRMHXTGNGDFEEIPEEYL
SEQ ID NO: 208 (modified hirulog)
F_{D}PRMHXTGGGNGDFEEIPEEYL
SEQ ID NO: 209 (modified hirulog)
F_{D}PRMHXTAGNGDFEEIPEEYL
SEQ ID NO: 210 (modified hirulog)
F_{D}PRMHXTANGDFEEIPEEYL
SEQ ID NO: 211 (modified hirulog)
F_{D}PRMHXTAGGNGDFEEIPEEYL
SEQ ID NO: 212 (modified hirulog)
F_{D}PRXHXGGGNGDFEEIPEEYL
SEQ ID NO: 213 (modified hirulog)
F_{D}PRXHXGGNGDFEEIPEEYL
SEQ ID NO: 214 (modified hirulog)
F_{D}PRXHXGGGGNGDFEEIPEEYL
SEQ ID NO: 215 (modified hirulog)
F_{D}PRXHXTGGNGDFEEIPEEYL
SEQ ID NO: 216 (modified hirulog)
F_{D}PRXHXTGNGDFEEIPEEYL
SEQ ID NO: 217 (modified hirulog)
F_{D}PRXHXTGGGNGDFEEIPEEYL
SEQ ID NO: 218 (modified hirulog)
F_{D}PRXHXTAGNGDFEEIPEEYL
SEQ ID NO: 219 (modified hirulog)
F_{D}PRXHXTANGDFEEIPEEYL
SEQ ID NO: 220 (modified hirulog)
F_{D}PRXHXTAGGNGDFEEIPEEYL
SEQ ID NO: 221 (modified hirulog)
FPRMHRGGGNGDFEEIPEEYL
SEQ ID NO: 222 (modified hirulog)
FPRMHRGGNGDFEEIPEEYL
SEQ ID NO: 223 (modified hirulog)
FPRMHRGGGGNGDFEEIPEEYL
SEQ ID NO: 224 (modified hirulog)
FPRMHRTGGNGDFEEIPEEYL
SEQ ID NO: 225 (modified hirulog)
FPRMHRTGNGDFEEIPEEYL
SEQ ID NO: 226 (modified hirulog)
FPRMHRTGGGNGDFEEIPEEYL
SEQ ID NO: 227 (modified hirulog)
FPRMHRTAGNGDFEEIPEEYL
SEQ ID NO: 228 (modified hirulog)
FRMHRTANGDFEEIPEEYL
SEQ ID NO: 229 (modified hirulog)
FPRMHRTAGGNGDFEEIPEEYL
SEQ ID NO: 230 (modified hirulog)
F_{D}PRMHRGGGNGDFEEIPEEYL
SEQ ID NO: 231 (modified hirulog)
F_{D}PRMHRGGNGDFEEIPEEYL
SEQ ID NO: 232 (modified hirulog)
F_{D}PRMHRGGGGNGDFEEIPEEYL
SEQ ID NO: 233 (modified hirulog)
F_{D}PRMHRTGGNGDFEEIPEEYL
SEQ ID NO: 234 (modified hirulog)
F_{D}PRMHRTGNGDFEEIPEEYL
SEQ ID NO: 235 (modified hirulog)
F_{D}PRMHRTGGGNGDFEEIPEEYL
SEQ ID NO: 236 (modified hirulog)
F_{D}PRMHRTAGNGDFEEIPEEYL
SEQ ID NO: 237 (modified hirulog)
F_{D}PRMHRTANGDFEEIPEEYL
SEQ ID NO: 238 (modified hirulog)
F_{D}PRMHRTAGGNGDFEEIPEEYL
SEQ ID NO: 239 (modified hirulog)
F_{D}PRXHXGGGNGDFEEIPEEYL
SEQ ID NO: 240 (modified hirulog)
F_{D}PRXHXGGNGDFEEIPEEYL
SEQ ID NO: 241 (modified hirulog)
F_{D}PRXHXGGGGNGDFEEIPEEYL
SEQ ID NO: 242 (modified hirulog)
F_{D}PRXHXTGGNGDFEEIPEEYL
SEQ ID NO: 243 (modified hirulog)
F_{D}PRXHXTGNGDFEEIPEEYL
SEQ ID NO: 244 (modified hirulog)
F_{D}PRXHXTGGGNGDFEEIPEEYL
SEQ ID NO: 245 (modified hirulog)
F_{D}PRXHXTAGNGDFEEIPEEYL
SEQ ID NO: 246 (modified hirulog)
F_{D}PRXHXTANGDFEEIPEEYL
SEQ ID NO: 247 (modified hirulog)
F_{D}PRXHXTAGGNGDFEEIPEEYL
SEQ ID NO: 248 (modified hirulog)
FPKMHKGGGNGDFEEIPEEYL
SEQ ID NO: 249 (modified hirulog)
FPKMHKGGNGDFEEIPEEYL
SEQ ID NO: 250 (modified hirulog)
FPKMHKGGGGNGDFEEIPEEYL
SEQ ID NO: 252 (modified hirulog)
FPKMHKTGGNGDFEEIPEEYL
SEQ ID NO: 253 (modified hirulog)
FPKMHKTGNGDFEEIPEEYL
SEQ ID NO: 254 (modified hirulog)
FPKMHKTGGGNGDFEEIPEEYL
SEQ ID NO: 255 (modified hirulog)
FPKMHKTAGNGDFEEIPEEYL
SEQ ID NO: 256 (modified hirulog)
FPKMHKTANGDFEEIPEEYL
SEQ ID NO: 257 (modified hirulog)
FPKMHKTAGGNGDFEEIPEEYL
SEQ ID NO: 258 (modified hirulog)
F_{D}PKMHKGGGNGDFEEIPEEYL
SEQ ID NO: 259 (modified hirulog)
F_{D}PKMHKGGNGDFEEIPEEYL
SEQ ID NO: 260 (modified hirulog)
F_{D}PKMHKGGGGNGDFEEIPEEYL
SEQ ID NO: 261 (modified hirulog)
F_{D}PKMHKTGGNGDFEEIPEEYL
SEQ ID NO: 262 (modified hirulog)
F_{D}PKMHKTGNGDFEEIPEEYL
SEQ ID NO: 263 (modified hirulog)
F_{D}PKMHKTGGGNGDFEEIPEEYL
SEQ ID NO: 264 (modified hirulog)
F_{D}PKMHKTAGNGDFEEIPEEYL
SEQ ID NO: 265 (modified hirulog)
F_{D}PKMHKTANGDFEEIPEEYL
SEQ ID NO: 266 (modified hirulog)
F_{D}PKMHKTAGGNGDFEEIPEEYL
SEQ ID NO: 267 (modified hirulog)
F_{D}PKXHKGGGNGDFEEIPEEYL
SEQ ID NO: 268 (modified hirulog)
F_{D}PKXHKGGNGDFEEIPEEYL
SEQ ID NO: 269 (modified hirulog)
F_{D}PKXHKGGGGNGDFEEIPEEYL
SEQ ID NO: 270 (modified hirulog)
F_{D}PKXHKTGGNGDFEEIPEEYL
SEQ ID NO: 271 (modified hirulog)
F_{D}PKXHKTGNGDFEEIPEEYL
SEQ ID NO: 272 (modified hirulog)
F_{D}PKXHKTGGGNGDFEEIPEEYL
SEQ ID NO: 273 (modified hirulog)
F_{D}PKXHKTAGNGDFEEIPEEYL
SEQ ID NO: 274 (modified hirulog)
F_{D}PKXHKTANGDFEEIPEEYL
SEQ ID NO: 275 (modified hirulog)
F_{D}PKXHKTAGGNGDFEEIPEEYL
SEQ ID NO: 276 (modified hirulog)
F_{D}PKXHXGGGNGDFEEIPEEYL
SEQ ID NO: 277 (modified hirulog)
F_{D}PKXHXGGNGDFEEIPEEYL
SEQ ID NO: 278 (modified hirulog)
F_{D}PKXHXGGGGNGDFEEIPEEYL
SEQ ID NO: 279 (modified hirulog)
F_{D}PKXHXTGGNGDFEEIPEEYL
SEQ ID NO: 280 (modified hirulog)
F_{D}PKXHXTGNGDFEEIPEEYL
SEQ ID NO: 281 (modified hirulog)
F_{D}PKXHXTGGGNGDFEEIPEEYL
SEQ ID NO: 282 (modified hirulog)
F_{D}PKXHXTAGNGDFEEIPEEYL
SEQ ID NO: 283 (modified hirulog)
F_{D}PKXHXTANGDFEEIPEEYL
SEQ ID NO: 284 (modified hirulog)
F_{D}PKXHXTAGGNGDFEEIPEEYL
SEQ ID NO: 285 (modified hirulog)
F_{D}PKXHXGGGNGDFEEIPEEYL
SEQ ID NO: 286 (modified hirulog)
F_{D}PKXHXGGNGDFEEIPEEYL
SEQ ID NO: 287 (modified hirulog)
F_{D}PKXHXGGGGNGDFEEIPEEYL
SEQ ID NO: 288 (modified hirulog)
F_{D}PKXHXTGGNGDFEEIPEEYL
SEQ ID NO: 289 (modified hirulog)
F_{D}PKXHXTGNGDFEEIPEEYL
SEQ ID NO: 290 (modified hirulog)
F_{D}PKXHXTGGGNGDFEEIPEEYL
SEQ ID NO: 291 (modified hirulog)
F_{D}PKXHXTAGNGDFEEIPEEYL
SEQ ID NO: 292 (modified hirulog)
F_{D}PKXHXTANGDFEEIPEEYL
SEQ ID NO: 293 (modified hirulog)
F_{D}PKXHXTAGGNGDFEEIPEEYL
SEQ ID NO: 294 (modified hirulog)
PRMHKGGGNGDFEEIPEEYL
SEQ ID NO: 295 (modified hirulog)
PRMHKGGNGDFEEIPEEYL
SEQ ID NO: 296 (modified hirulog)
PRMHKGGGGNGDFEEIPEEYL
SEQ ID NO: 297 (modified hirulog)
PRMHKTGGNGDFEEIPEEYL
SEQ ID NO: 298 (modified hirulog)
PRMHKTGNGDFEEIPEEYL
SEQ ID NO: 299 (modified hirulog)
PRMHKTGGGNGDFEEIPEEYL
SEQ ID NO: 300 (modified hirulog)
PRMHKTAGNGDFEEIPEEYL
SEQ ID NO: 301 (modified hirulog)
PRMHKTANGDFEEIPEEYL
SEQ ID NO: 302 (modified hirulog)
PRMHKTAGGNGDFEEIPEEYL
SEQ ID NO: 303 (modified hirulog)
PRXHKGGGNGDFEEIPEEYL
SEQ ID NO: 304 (modified hirulog)
PRXHKGGNGDFEEIPEEYL
SEQ ID NO: 305 (modified hirulog)
PRXHKGGGGNGDFEEIPEEYL
SEQ ID NO: 306 (modified hirulog)
PRXHKTGGNGDFEEIPEEYL
SEQ ID NO: 307 (modified hirulog)
PRXHKTGNGDFEEIPEEYL
SEQ ID NO: 308 (modified hirulog)
PRXHKTGGGNGDFEEIPEEYL
SEQ ID NO: 309 (modified hirulog)
PRXHKTAGNGDFEEIPEEYL
SEQ ID NO: 310 (modified hirulog)
PRXHKTANGDFEEIPEEYL
SEQ ID NO: 311 (modified hirulog)
PRXHKTAGGNGDFEEIPEEYL
SEQ ID NO: 312 (modified hirulog)
PRXHXGGGNGDFEEIPEEYL
SEQ ID NO: 313 (modified hirulog)
PRXHXGGNGDFEEIPEEYL
SEQ ID NO: 314 (modified hirulog)
PRXHXGGGGNGDFEEIPEEYL
SEQ ID NO: 315 (modified hirulog)
PRXHXTGGNGDFEEIPEEYL
SEQ ID NO: 316 (modified hirulog)
PRXHXTGNGDFEEIPEEYL
SEQ ID NO: 317 (modified hirulog)
PRXHXTGGGNGDFEEIPEEYL
SEQ ID NO: 318 (modified hirulog)
PRXHXTAGNGDFEEIPEEYL
SEQ ID NO: 319 (modified hirulog)
PRXHXTANGDFEEIPEEYL
SEQ ID NO: 320 (modified hirulog)
PRXHXTAGGNGDFEEIPEEYL
SEQ ID NO: 321 (modified hirulog)
PRXHXGGGNGDFEEIPEEYL
SEQ ID NO: 322 (modified hirulog)
PRXHXGGNGDFEEIPEEYL
SEQ ID NO: 323 (modified hirulog)
PRXHXGGGGNGDFEEIPEEYL
SEQ ID NO: 324 (modified hirulog)
PRXHXTGGNGDFEEIPEEYL
SEQ ID NO: 325 (modified hirulog)
PRXHXTGNGDFEEIPEEYL
SEQ ID NO: 326 (modified hirulog)
PRXHXTGGGNGDFEEIPEEYL
SEQ ID NO: 327 (modified hirulog)
PRXHXTAGNGDFEEIPEEYL
SEQ ID NO: 328 (modified hirulog)
PRXHXTANGDFEEIPEEYL
SEQ ID NO: 329 (modified hirulog)
PRXHXTAGGNGDFEEIPEEYL
SEQ ID NO: 330 (modified hirulog)
PKXHXGGGNGDFEEIPEEYL
SEQ ID NO: 331 (modified hirulog)
PKXHXGGNGDFEEIPEEYL
SEQ ID NO: 332 (modified hirulog)
PKXHXGGGGNGDFEEIPEEYL
SEQ ID NO: 333 (modified hirulog)
PKXHXTGGNGDFEEIPEEYL
SEQ ID NO: 334 (modified hirulog)
PKXHXTGNGDFEEIPEEYL
SEQ ID NO: 335 (modified hirulog)
PKXHXTGGGNGDFEEIPEEYL
SEQ ID NO: 336 (modified hirulog)
PKXHXTAGNGDFEEIPEEYL
SEQ ID NO: 337 (modified hirulog)
PKXHXTANGDFEEIPEEYL
SEQ ID NO: 338 (modified hirulog)
PKXHXTAGGNGDFEEIPEEYL
SEQ ID NO: 339 (modified hirulog)
PKXHXGGGNGDFEEIPEEYL
SEQ ID NO: 340 (modified hirulog)
PKXHXGGNGDFEEIPEEYL
SEQ ID NO: 341 (modified hirulog)
PKXHXGGGGNGDFEEIPEEYL
SEQ ID NO: 342 (modified hirulog)
PKXHXTGGNGDFEEIPEEYL
SEQ ID NO: 343 (modified hirulog)
PKXHXTGNGDFEEIPEEYL
SEQ ID NO: 344 (modified hirulog)
PKXHXTGGGNGDFEEIPEEYL
SEQ ID NO: 345 (modified hirulog)
PKXHXTAGNGDFEEIPEEYL
SEQ ID NO: 346 (modified hirulog)
PKXHXTANGDFEEIPEEYL
SEQ ID NO: 347 (modified hirulog)
PKXHXTAGGNGDFEEIPEEYL
SEQ ID NO: 348 (modified hirulog)
RMHKGGGNGDFEEIPEEYL
SEQ ID NO: 349 (modified hirulog)
RMHKGGNGDFEEIPEEYL
SEQ ID NO: 350 (modified hirulog)
RMHKGGGGNGDFEEIPEEYL
SEQ ID NO: 351 (modified hirulog)
RMHKTGGNGDFEEIPEEYL
SEQ ID NO: 352 (modified hirulog)
RMHKTGNGDFEEIPEEYL
SEQ ID NO: 353 (modified hirulog)
RMHKTGGGNGDFEEIPEEYL
SEQ ID NO: 354 (modified hirulog)
RMHKTAGNGDFEEIPEEYL
SEQ ID NO: 355 (modified hirulog)
RMHKTANGDFEEIPEEYL
SEQ ID NO: 356 (modified hirulog)
RMHKTAGGNGDFEEIPEEYL
SEQ ID NO: 357 (modified hirulog)
RXHKGGGNGDFEEIPEEYL
SEQ ID NO: 358 (modified hirulog)
RXHKGGNGDFEEIPEEYL
SEQ ID NO: 359 (modified hirulog)
RXHKGGGGNGDFEEIPEEYL
SEQ ID NO: 360 (modified hirulog)
RXHKTGGNGDFEEIPEEYL
SEQ ID NO: 361 (modified hirulog)
RXHKTGNGDFEEIPEEYL
SEQ ID NO: 362 (modified hirulog)
RXHKTGGGNGDFEEIPEEYL
SEQ ID NO: 363 (modified hirulog)
RXHKTAGNGDFEEIPEEYL
SEQ ID NO: 364 (modified hirulog)
RXHKTANGDFEEIPEEYL
SEQ ID NO: 365 (modified hirulog)
RXHKTAGGNGDFEEIPEEYL
SEQ ID NO: 366 (modified hirulog)
RXHXGGGNGDFEEIPEEYL
SEQ ID NO: 367 (modified hirulog)
RXHXGGNGDFEEIPEEYL
SEQ ID NO: 368 (modified hirulog)
RXHXGGGGNGDFEEIPEEYL
SEQ ID NO: 369 (modified hirulog)
RXHXTGGNGDFEEIPEEYL
SEQ ID NO: 370 (modified hirulog)
RXHXTGNGDFEEIPEEYL
SEQ ID NO: 371 (modified hirulog)
RXHXTGGGNGDFEEIPEEYL
SEQ ID NO: 372 (modified hirulog)
RXHXTAGNGDFEEIPEEYL
SEQ ID NO: 373 (modified hirulog)
RXHXTANGDFEEIPEEYL
SEQ ID NO: 374 (modified hirulog)
RXHXTAGGNGDFEEIPEEYL
SEQ ID NO: 375 (modified hirulog)
RXHXGGGNGDFEEIPEEYL
SEQ ID NO: 376 (modified hirulog)
RXHXGGNGDFEEIPEEYL
SEQ ID NO: 377 (modified hirulog)
RXHXGGGGNGDFEEIPEEYL
SEQ ID NO: 378 (modified hirulog)
RXHXTGGNGDFEEIPEEYL
SEQ ID NO: 379 (modified hirulog)
RXHXTGNGDFEEIPEEYL
SEQ ID NO: 380 (modified hirulog)
RXHXTGGGNGDFEEIPEEYL
SEQ ID NO: 381 (modified hirulog)
RXHXTAGNGDFEEIPEEYL
SEQ ID NO: 382 (modified hirulog)
RXHXTANGDFEEIPEEYL
SEQ ID NO: 383 (modified hirulog)
RXHXTAGGNGDFEEIPEEYL
SEQ ID NO: 384 (modified hirulog)
KXHXGGGNGDFEEIPEEYL
SEQ ID NO: 385 (modified hirulog)
KXHXGGNGDFEEIPEEYL
SEQ ID NO: 386 (modified hirulog)
KXHXGGGGNGDFEEIPEEYL
SEQ ID NO: 387 (modified hirulog)
KXHXTGGNGDFEEIPEEYL
SEQ ID NO: 388 (modified hirulog)
KXHXTGNGDFEEIPEEYL
SEQ ID NO: 389 (modified hirulog)
KXHXTGGGNGDFEEIPEEYL
SEQ ID NO: 390 (modified hirulog)
KXHXTAGNGDFEEIPEEYL
SEQ ID NO: 391 (modified hirulog)
KXHXTANGDFEEIPEEYL
SEQ ID NO: 392 (modified hirulog)
KXHXTAGGNGDFEEIPEEYL
SEQ ID NO: 393 (modified hirulog)
KXHXGGGNGDFEEIPEEYL
SEQ ID NO: 394 (modified hirulog)
KXHXGGNGDFEEIPEEYL
SEQ ID NO: 395 (modified hirulog)
KXHXGGGGNGDFEEIPEEYL
SEQ ID NO: 396 (modified hirulog)
KXHXTGGNGDFEEIPEEYL
SEQ ID NO: 397 (modified hirulog)
KXHXTGNGDFEEIPEEYL
SEQ ID NO: 398 (modified hirulog)
KXHXTGGGNGDFEEIPEEYL
SEQ ID NO: 399 (modified hirulog)
KXHXTAGNGDFEEIPEEYL
SEQ ID NO: 400 (modified hirulog)
KXHXTANGDFEEIPEEYL
SEQ ID NO: 401 (modified hirulog)
KXHXTAGGNGDFEEIPEEYL
SEQ ID NO: 402 (modified hirulog)
MHKGGGNGDFEEIPEEYL
SEQ ID NO: 403 (modified hirulog)
MHKGGNGDFEEIPEEYL
SEQ ID NO: 404 (modified hirulog)
MHKGGGGNGDFEEIPEEYL
SEQ ID NO: 405 (modified hirulog)
MHKTGGNGDFEEIPEEYL
SEQ ID NO: 406 (modified hirulog)
MHKTGNGDFEEIPEEYL
SEQ ID NO: 407 (modified hirulog)
MHKTGGGNGDFEEIPEEYL
SEQ ID NO: 408 (modified hirulog)
MHKTAGNGDFEEIPEEYL
SEQ ID NO: 409 (modified hirulog)
MHKTANGDFEEIPEEYL
SEQ ID NO: 410 (modified hirulog)
MHKTAGGNGDFEEIPEEYL
SEQ ID NO: 411 (modified hirulog)
XHKGGGNGDFEEIPEEYL
SEQ ID NO: 412 (modified hirulog)
XHKGGNGDFEEIPEEYL
SEQ ID NO: 413 (modified hirulog)
XHKGGGGNGDFEEIPEEYL
SEQ ID NO: 414 (modified hirulog)
XHKTGGNGDFEEIPEEYL
SEQ ID NO: 415 (modified hirulog)
XHKTGNGDFEEIPEEYL
SEQ ID NO: 416 (modified hirulog)
XHKTGGGNGDFEEIPEEYL
SEQ ID NO: 417 (modified hirulog)
XHKTAGNGDFEEIPEEYL
SEQ ID NO: 418 (modified hirulog)
XHKTANGDFEEIPEEYL
SEQ ID NO: 419 (modified hirulog)
XHKTAGGNGDFEEIPEEYL
SEQ ID NO: 420 (modified hirulog)
XHXGGGNGDFEEIPEEYL
SEQ ID NO: 421 (modified hirulog)
XHXGGNGDFEEIPEEYL
SEQ ID NO: 422 (modified hirulog)
XHXGGGGNGDFEEIPEEYL
SEQ ID NO: 423 (modified hirulog)
XHXTGGNGDFEEIPEEYL
SEQ ID NO: 424 (modified hirulog)
XHXTGNGDFEEIPEEYL
SEQ ID NO: 425 (modified hirulog)
XHXTGGGNGDFEEIPEEYL
SEQ ID NO: 426 (modified hirulog)
XHXTAGNGDFEEIPEEYL
SEQ ID NO: 427 (modified hirulog)
XHXTANGDFEEIPEEYL
SEQ ID NO: 428 (modified hirulog)
XHXTAGGNGDFEEIPEEYL
SEQ ID NO: 429 (modified hirulog)
XHXGGGNGDFEEIPEEYL
SEQ ID NO: 430 (modified hirulog)
XHXGGNGDFEEIPEEYL
SEQ ID NO: 431 (modified hirulog)
XHXGGGGNGDFEEIPEEYL
SEQ ID NO: 432 (modified hirulog)
XHXTGGNGDFEEIPEEYL
SEQ ID NO: 433 (modified hirulog)
XHXTGNGDFEEIPEEYL
SEQ ID NO: 434 (modified hirulog)
XHXTGGGNGDFEEIPEEYL
SEQ ID NO: 435 (modified hirulog)
XHXTAGNGDFEEIPEEYL
SEQ ID NO: 436 (modified hirulog)
XHXTANGDFEEIPEEYL
SEQ ID NO: 437 (modified hirulog)
XHXTAGGNGDFEEIPEEYL
SEQ ID NO: 438 (modified hirulog)
XHXGGGNGDFEEIPEEYL
SEQ ID NO: 439 (modified hirulog)
XHXGGNGDFEEIPEEYL
SEQ ID NO: 440 (modified hirulog)
XHXGGGGNGDFEEIPEEYL
SEQ ID NO: 441 (modified hirulog)
XHXTGGNGDFEEIPEEYL
SEQ ID NO: 442 (modified hirulog)
XHXTGNGDFEEIPEEYL
SEQ ID NO: 443 (modified hirulog)
XHXTGGGNGDFEEIPEEYL
SEQ ID NO: 444 (modified hirulog)
XHXTAGNGDFEEIPEEYL
SEQ ID NO: 445 (modified hirulog)
XHXTANGDFEEIPEEYL
SEQ ID NO: 446 (modified hirulog)
XHXTAGGNGDFEEIPEEYL
SEQ ID NO: 447 (modified hirulog)
XHXGGGNGDFEEIPEEYL
SEQ ID NO: 448 (modified hirulog)
XHXGGNGDFEEIPEEYL
SEQ ID NO: 449 (modified hirulog)
XHXGGGGNGDFEEIPEEYL
SEQ ID NO: 450 (modified hirulog)
XHXTGGNGDFEEIPEEYL
SEQ ID NO: 451 (modified hirulog)
XHXTGNGDFEEIPEEYL
SEQ ID NO: 452 (modified hirulog)
XHXTGGGNGDFEEIPEEYL
SEQ ID NO: 453 (modified hirulog)
XHXTAGNGDFEEIPEEYL
SEQ ID NO: 454 (modified hirulog)
XHXTANGDFEEIPEEYL
SEQ ID NO: 455 (modified hirulog)
XHXTAGGNGDFEEIPEEYL
SEQ ID NO: 456 (modified hirulog)
FPRMHKGGGNGDYEPIPEEA
SEQ ID NO: 457 (modified hirulog)
FPRMHKGGNGDYEPIPEEA
SEQ ID NO: 458 (modified hirulog)
FPRMHKGGGGNGDYEPIPEEA
SEQ ID NO: 459 (modified hirulog)
FPRMHKTGGNGDYEPIPEEA
SEQ ID NO: 460 (modified hirulog)
FPRMHKTGNGDYEPIPEEA
SEQ ID NO: 461 (modified hirulog)
FPRMHKTGGGNGDYEPIPEEA
SEQ ID NO: 462 (modified hirulog)
FPRMHKTAGNGDYEPIPEEA
SEQ ID NO: 463 (modified hirulog)
FPRMHKTANGDYEPIPEEA
SEQ ID NO: 464 (modified hirulog)
FPRMHKTAGGNGDYEPIPEEA
SEQ ID NO: 465 (modified hirulog)
F_{D}PRMHKGGGNGDYEPIPEEA
SEQ ID NO: 466 (modified hirulog)
F_{D}PRMHKGGNGDYEPIPEEA
SEQ ID NO: 467 (modified hirulog)
F_{D}PRMHKGGGGNGDYEPIPEEA
SEQ ID NO: 468 (modified hirulog)
F_{D}PRMHKTGGNGDYEPIPEEA
SEQ ID NO: 469 (modified hirulog)
F_{D}PRMHKTGNGDYEPIPEEA
SEQ ID NO: 470 (modified hirulog)
F_{D}PRMHKTGGGNGDYEPIPEEA
SEQ ID NO: 471 (modified hirulog)
F_{D}PRMHKTAGNGDYEPIPEEA
SEQ ID NO: 472 (modified hirulog)
F_{D}PRMHKTANGDYEPIPEEA
SEQ ID NO: 473 (modified hirulog)
F_{D}PRMHKTAGGNGDYEPIPEEA
SEQ ID NO: 474 (modified hirulog)
F_{D}PRXHKGGGNGDYEPIPEEA
SEQ ID NO: 475 (modified hirulog)
F_{D}PRXHKGGNGDYEPIPEE
SEQ ID NO: 476 (modified hirulog)
F_{D}PRXHKGGGGNGDYEPIPEEA
SEQ ID NO: 477 (modified hirulog)
F_{D}PRXHKTGGNGDYEPIPEEA
SEQ ID NO: 478 (modified hirulog)
F_{D}PRXHKTGNGDYEPIPEEA
SEQ ID NO: 479 (modified hirulog)
F_{D}PRXHKTGGGNGDFYEPIPEEA
SEQ ID NO: 480 (modified hirulog)
F_{D}PRXHKTAGNGDYEPIPEEA
SEQ ID NO: 481 (modified hirulog)
F_{D}PRXHKTANGDYEPIPEEA
SEQ ID NO: 482 (modified hirulog)
F_{D}PRXHKTAGGNGDYEPIPEEA
SEQ ID NO: 483 (modified hirulog)
F_{D}PRXHXGGGNGDYEPIPEEA
SEQ ID NO: 484 (modified hirulog)
F_{D}PRXHXGGNGDYEPIPEEA
SEQ ID NO: 485 (modified hirulog)
F_{D}PRXHXGGGGNGDYEPIPEEA
SEQ ID NO: 486 (modified hirulog)
F_{D}PRXHXTGGNGDYEPIPEEA
SEQ ID NO: 487 (modified hirulog)
F_{D}PRXHXTGNGDYEPIPEEA
SEQ ID NO: 488 (modified hirulog)
F_{D}PRXHXTGGGNGDYEPIPEEA
SEQ ID NO: 489 (modified hirulog)
F_{D}PRXHXTAGNGDYEPIPEEA
SEQ ID NO: 490 (modified hirulog)
F_{D}PRXHXTANGDYEPIPEEA
SEQ ID NO: 491 (modified hirulog)
F_{D}PRXHXTAGGNGDYEPIPEEA
SEQ ID NO: 492 (modified hirulog)
F_{D}PRXHXGGGNGDYEPIPEEA
SEQ ID NO: 493 (modified hirulog)
F_{D}PRXHXGGNGDYEPIPEEA
SEQ ID NO: 494 (modified hirulog)
F_{D}PRXHXGGGGNGDYEPIPEEA
SEQ ID NO: 495 (modified hirulog)
F_{D}PRXHXTGGNGDYEPIPEEA
SEQ ID NO: 496 (modified hirulog)
F_{D}PRXHXTGNGDYEPIPEEA
SEQ ID NO: 497 (modified hirulog)
F_{D}PRXHXTGGGNGDYEPIPEEA
SEQ ID NO: 498 (modified hirulog)
F_{D}PRXHXTAGNGDYEPIPEEA
SEQ ID NO: 499 (modified hirulog)
F_{D}PRXHXTANGDYEPIPEEA
SEQ ID NO: 500 (modified hirulog)
F_{D}PRXHXTAGGNGDYEPIPEEA
SEQ ID NO: 501 (modified hirulog)
F_{D}PKXHXGGGNGDYEPIPEEA
SEQ ID NO: 502 (modified hirulog)
F_{D}PKXHXGGNGDYEPIPEEA
SEQ ID NO: 503 (modified hirulog)
F_{D}PKXHXGGGGNGDYEPIPEEA
SEQ ID NO: 504 (modified hirulog)
F_{D}PKXHXTGGNGDYEPIPEEA
SEQ ID NO: 505 (modified hirulog)
F_{D}PKXHXTGNGDYEPIPEEA
SEQ ID NO: 506 (modified hirulog)
F_{D}PKXHXTGGGNGDYEPIPEEA
SEQ ID NO: 507 (modified hirulog)
F_{D}PKXHXTAGNGDYEPIPEEA
SEQ ID NO: 508 (modified hirulog)
F_{D}PKXHXTANGDYEPIPEEA
SEQ ID NO: 509 (modified hirulog)
F_{D}PKXHXTAGGNGDYEPIPEEA
SEQ ID NO: 510 (modified hirulog)
F_{D}PKXHXGGGNGDYEPIPEEA
SEQ ID NO: 511 (modified hirulog)
F_{D}PKXHXGGNGDYEPIPEEA
SEQ ID NO: 512 (modified hirulog)
F_{D}PKXHXGGGGNGDYEPIPEEA
SEQ ID NO: 513 (modified hirulog)
F_{D}PKXHXTGGNGDYEPIPEEA
SEQ ID NO: 514 (modified hirulog)
F_{D}PKXHXTGNGDYEPIPEEA
SEQ ID NO: 515 (modified hirulog)
F_{D}PKXHXTGGGNGDYEPIPEEA
SEQ ID NO: 516 (modified hirulog)
F_{D}PKXHXTAGNGDYEPIPEEA
SEQ ID NO: 517 (modified hirulog)
F_{D}PKXHXTANGDYEPIPEEA
SEQ ID NO: 518 (modified hirulog)
F_{D}PKXHXTAGGNGDYEPIPEEA
SEQ ID NO: 519 (modified hirulog)
PKXHXGGGNGDYEPIPEEA
SEQ ID NO: 520 (modified hirulog)
PKXHXGGNGDYEPIPEEA
SEQ ID NO: 521 (modified hirulog)
PKXHXGGGGNGDYEPIPEEA
SEQ ID NO: 522 (modified hirulog)
PKXHXTGGNGDYEPIPEEA
SEQ ID NO: 523 (modified hirulog)
PKXHXTGNGDYEPIPEEA
SEQ ID NO: 524 (modified hirulog)
PKXHXTGGGNGDYEPIPEEA
SEQ ID NO: 525 (modified hirulog)
PKXHXTAGNGDYEPIPEEA
SEQ ID NO: 526 (modified hirulog)
PKXHXTANGDYEPIPEEA
SEQ ID NO: 527 (modified hirulog)
PKXHXTAGGNGDYEPIPEEA
SEQ ID NO: 528 (modified hirulog)
PKXHXGGGNGDYEPIPEEA
SEQ ID NO: 529 (modified hirulog)
PKXHXGGNGDYEPIPEEA
SEQ ID NO: 530 (modified hirulog)
PKXHXGGGGNGDYEPIPEEA
SEQ ID NO: 531 (modified hirulog)
PKXHXTGGNGDYEPIPEEA
SEQ ID NO: 532 (modified hirulog)
PKXHXTGNGDYEPIPEEA
SEQ ID NO: 533 (modified hirulog)
PKXHXTGGGNGDYEPIPEEA
SEQ ID NO: 534 (modified hirulog)
PKXHXTAGNGDYEPIPEEA
SEQ ID NO: 536 (modified hirulog)
PKXHXTANGDYEPIPEEA
SEQ ID NO: 537 (modified hirulog)
PKXHXTAGGNGDYEPIPEEA
SEQ ID NO: 538 (modified hirulog)
KXHXGGGNGDYEPIPEEA
SEQ ID NO: 539 (modified hirulog)
KXHXGGNGDYEPIPEEA
SEQ ID NO: 540 (modified hirulog)
KXHXGGGGNGDYEPIPEEA
SEQ ID NO: 541 (modified hirulog)
KXHXTGGNGDYEPIPEEA
SEQ ID NO: 542 (modified hirulog)
KXHXTGNGDYEPIPEEA
SEQ ID NO: 543 (modified hirulog)
KXHXTGGGNGDYEPIPEEA
SEQ ID NO: 544 (modified hirulog)
KXHXTAGNGDYEPIPEEA
SEQ ID NO: 545 (modified hirulog)
KXHXTANGDYEPIPEEA
SEQ ID NO: 546 (modified hirulog)
KXHXTAGGNGDYEPIPEEA
SEQ ID NO: 547 (modified hirulog)
KXHXGGGNGDYEPIPEEA
SEQ ID NO: 548 (modified hirulog)
KXHXGGNGDYEPIPEEA
SEQ ID NO: 549 (modified hirulog)
KXHXGGGGNGDYEPIPEEA
SEQ ID NO: 550 (modified hirulog)
KXHXTGGNGDYEPIPEEA
SEQ ID NO: 551 (modified hirulog)
KXHXTGNGDYEPIPEEA
SEQ ID NO: 552 (modified hirulog)
KXHXTGGGNGDYEPIPEEA
SEQ ID NO: 553 (modified hirulog)
KXHXTAGNGDYEPIPEEA
SEQ ID NO: 554 (modified hirulog)
KXHXTANGDYEPIPEEA
SEQ ID NO: 555 (modified hirulog)
KXHXTAGGNGDYEPIPEEA
SEQ ID NO: 556 (modified hirulog)
FPRMHKGGGAPPFDFEAIPEEYL
SEQ ID NO: 557 (modified hirulog)
FPRMHKGGAPPFDFEAIPEEYL
SEQ ID NO: 558 (modified hirulog)
FPRMHKGGGGAPPFDFEAIPEEYL
SEQ ID NO: 559 (modified hirulog)
FPRMHKTGGAPPFDFEAIPEEYL
SEQ ID NO: 560 (modified hirulog)
FPRMHKTGAPPFDFEAIPEEYL
SEQ ID NO: 561 (modified hirulog)
FPRMHKTGGGAPPFDFEAIPEEYL
SEQ ID NO: 562 (modified hirulog)
FPRMHKTAGAPPFDFEAIPEEYL
SEQ ID NO: 563 (modified hirulog)
FPRMHKTAAPPFDFEAIPEEYL
SEQ ID NO: 564 (modified hirulog)
FPRMHKTAGGAPPFDFEAIPEEYL
SEQ ID NO: 565 (modified hirulog)
F_{D}PRMHKGGGAPPFDFEAIPEEYL
SEQ ID NO: 566 (modified hirulog)
F_{D}PRMHKGGAPPFDFEAIPEEYL
SEQ ID NO: 567 (modified hirulog)
F_{D}PRMHKGGGGAPPFDFEAIPEEYL
SEQ ID NO: 568 (modified hirulog)
F_{D}PRMHKTGGAPPFDFEAIPEEYL
SEQ ID NO: 569 (modified hirulog)
F_{D}PRMHKTGAPPFDFEAIPEEYL
SEQ ID NO: 570 (modified hirulog)
F_{D}PRMHKTGGGAPPFDFEAIPEEYL
SEQ ID NO: 571 (modified hirulog)
F_{D}PRMHKTAGAPPFDFEAIPEEYL
SEQ ID NO: 572 (modified hirulog)
F_{D}PRMHKTAAPPFDFEAIPEEYL
SEQ ID NO: 573 (modified hirulog)
F_{D}PRMHKTAGGAPPFDFEAIPEEYL
SEQ ID NO: 574 (modified hirulog)
F_{D}PRXHKGGGAPPFDFEAIPEEYL
SEQ ID NO: 575 (modified hirulog)
F_{D}PRXHKGGAPPFDFEAIPEEYL
SEQ ID NO: 576 (modified hirulog)
F_{D}PRXHKGGGGAPPFDFEAIPEEYL
SEQ ID NO: 577 (modified hirulog)
F_{D}PRXHKTGGAPPFDFEAIPEEYL
SEQ ID NO: 578 (modified hirulog)
F_{D}PRXHKTGAPPFDFEAIPEEYL
SEQ ID NO: 579 (modified hirulog)
F_{D}PRXHKTGGGAPPFDFEAIPEEYL
SEQ ID NO: 580 (modified hirulog)
F_{D}PRXHKTAGAPPFDFEAIPEEYL
SEQ ID NO: 581 (modified hirulog)
F_{D}PRXHKTAAPPFDFEAIPEEYL
SEQ ID NO: 582 (modified hirulog)
F_{D}PRXHKTAGAPPFDFEAIPEEYL
SEQ ID NO: 583 (modified hirulog)
F_{D}PRXHXGGGAPPFDFEAIPEEYLL
SEQ ID NO: 584 (modified hirulog)
F_{D}PRXHXGGAPPFDFEAIPEEYL
SEQ ID NO: 585 (modified hirulog)
F_{D}PRXHXGGGGAPPFDFEAIPEEYL
SEQ ID NO: 586 (modified hirulog)
F_{D}PRXHXTGGAPPFDFEAIPEEYL
SEQ ID NO: 587 (modified hirulog)
F_{D}PRXHXTGAPPFDFEAIPEEYL
SEQ ID NO: 588 (modified hirulog)
F_{D}PRXHXTGGGAPPFDFEAIPEEYL
SEQ ID NO: 589 (modified hirulog)
F_{D}PRXHXTAGAPPFDFEAIPEEYL
SEQ ID NO: 590 (modified hirulog)
F_{D}PRXHXTAAPPFDFEAIPEEYL
SEQ ID NO: 591 (modified hirulog)
F_{D}PRXHXTAGGAPPFDFEAIPEEYL
F_{D}PRXHXGGGAPPFDFEAIPEEYL
SEQ ID NO: 592 (modified hirulog)
F_{D}PRXHXGGAPPFDFEAIPEEYL
SEQ ID NO: 593 (modified hirulog)
F_{D}PRXHXGGGGAPPFDFEAIPEEYL
SEQ ID NO: 594 (modified hirulog)
F_{D}PRXHXTGGAPPFDFEAIPEEYL
SEQ ID NO: 595 (modified hirulog)
F_{D}PRXHXTGAPPFDFEAIPEEYL
SEQ ID NO: 596 (modified hirulog)
F_{D}PRXHXTGGGAPPFDFEAIPEEYL
SEQ ID NO: 597 (modified hirulog)
F_{D}PRXHXTAGAPPFDFEAIPEEYL
SEQ ID NO: 598 (modified hirulog)
F_{D}PRXHXTAAPPFDFEAIPEEYL
SEQ ID NO: 599 (modified hirulog)
F_{D}PRXHXTAGGAPPFDFEAIPEEYL
SEQ ID NO: 600 (modified hirulog)
F_{D}PKXHXGGGAPPFDFEAIPEEYL
SEQ ID NO: 601 (modified hirulog)
F_{D}PKXHXGGAPPFDFEAIPEEYL
SEQ ID NO: 602 (modified hirulog)
F_{D}PKXHXGGGGAPPFDFEAIPEEYL
SEQ ID NO: 603 (modified hirulog)
F_{D}PKXHXTGGAPPFDFEAIPEEYL
SEQ ID NO: 604 (modified hirulog)
F_{D}PKXHXTGAPPFDFEAIPEEYL
SEQ ID NO: 605 (modified hirulog)
F_{D}PKXHXTGGGAPPFDFEAIPEEYL
SEQ ID NO: 606 (modified hirulog)
F_{D}PKXHXTAGAPPFDFEAIPEEYL
SEQ ID NO: 607 (modified hirulog)
F_{D}PKXHXTAAPPFDFEAIPEEYL
SEQ ID NO: 608 (modified hirulog)
F_{D}PKXHXTAGGAPPFDFEAIPEEYL
SEQ ID NO: 609 (modified hirulog)
F_{D}PKXHXGGGAPPFDFEAIPEEYL
SEQ ID NO: 610 (modified hirulog)
F_{D}PKXHXGGAPPFDFEAIPEEYL
SEQ ID NO: 611 (modified hirulog)
F_{D}PKXHXGGGGAPPFDFEAIPEEYL
SEQ ID NO: 612 (modified hirulog)
F_{D}PKXHXTGGAPPFDFEAIPEEYL
SEQ ID NO: 613 (modified hirulog)
F_{D}PKXHXTGAPPFDFEAIPEEYL
SEQ ID NO: 614 (modified hirulog)
F_{D}PKXHXTGGGAPPFDFEAIPEEYL
SEQ ID NO: 615 (modified hirulog)
F_{D}PKXHXTAGAPPFDFEAIPEEYL
SEQ ID NO: 616 (modified hirulog)
F_{D}PKXHXTAAPPFDFEAIPEEYL
SEQ ID NO: 617 (modified hirulog)
F_{D}PKXHXTAGGAPPFDFEAIPEEYL
SEQ ID NO: 618 (modified hirulog)
PKXHXGGGAPPFDFEAIPEEYL
SEQ ID NO: 619 (modified hirulog)
PKXHXGGAPPFDFEAIPEEYL
SEQ ID NO: 620 (modified hirulog)
PKXHXGGGGAPPFDFEAIPEEYL
SEQ ID NO: 621 (modified hirulog)
PKXHXTGGAPPFDFEAIPEEYL
SEQ ID NO: 622 (modified hirulog)
PKXHXTGAPPFDFEAIPEEYL
SEQ ID NO: 623 (modified hirulog)
PKXHXTGGGAPPFDFEAIPEEYL
SEQ ID NO: 624 (modified hirulog)
PKXHXTAGAPPFDFEAIPEEYL
SEQ ID NO: 625 (modified hirulog)
PKXHXTAAPPFDFEAIPEEYL
SEQ ID NO: 626 (modified hirulog)
PKXHXTAGGAPPFDFEAIPEEYL
SEQ ID NO: 627 (modified hirulog)
PKXHXGGGAPPFDFEAIPEEYL
SEQ ID NO: 628 (modified hirulog)
PKXHXGGAPPFDFEAIPEEYL
SEQ ID NO: 629 (modified hirulog)
PKXHXGGGGAPPFDFEAIPEEYL
SEQ ID NO: 630 (modified hirulog)
PKXHXTGGAPPFDFEAIPEEYL
SEQ ID NO: 631 (modified hirulog)
PKXHXTGAPPFDFEAIPEEYL
SEQ ID NO: 632 (modified hirulog)
PKXHXTGGGAPPFDFEAIPEEYL
SEQ ID NO: 633 (modified hirulog)
PKXHXTAGAPPFDFEAIPEEYL
SEQ ID NO: 634 (modified hirulog)
PKXHXTAAPPFDFEAIPEEYL
SEQ ID NO: 635 (modified hirulog)
PKXHXTAGGAPPFDFEAIPEEYL
SEQ ID NO: 636 (modified hirulog)
PKXHXGGGAPPFDFEAIPEEYL
SEQ ID NO: 637 (modified hirulog)
PKXHXGGAPPFDFEAIPEEYL
SEQ ID NO: 638 (modified hirulog)
PKXHXGGGGAPPFDFEAIPEEYL
SEQ ID NO: 639 (modified hirulog)
PKXHXTGGAPPFDFEAIPEEYL
SEQ ID NO: 640 (modified hirulog)
PKXHXTGAPPFDFEAIPEEYL
SEQ ID NO: 641 (modified hirulog)
PKXHXTGGGAPPFDFEAIPEEYL
SEQ ID NO: 642 (modified hirulog)
PKXHXTAGAPPFDFEAIPEEYL
SEQ ID NO: 643 (modified hirulog)
PKXHXTAAPPFDFEAIPEEYL
SEQ ID NO: 644 (modified hirulog)
PKXHXTAGGAPPFDFEAIPEEYL
SEQ ID NO: 645 (modified hirulog)
PKXHXGGGAPPFDFEAIPEEYL
SEQ ID NO: 646 (modified hirulog)
PKXHXGGAPPFDFEAIPEEYL
SEQ ID NO: 647 (modified hirulog)
PKXHXGGGGAPPFDFEAIPEEYL
SEQ ID NO: 648 (modified hirulog)
PKXHXTGGAPPFDFEAIPEEYL
SEQ ID NO: 649 (modified hirulog)
PKXHXTGAPPFDFEAIPEEYL
SEQ ID NO: 650 (modified hirulog)
PKXHXTGGGAPPFDFEAIPEEYL
SEQ ID NO: 651 (modified hirulog)
PKXHXTAGAPPFDFEAIPEEYL
SEQ ID NO: 652 (modified hirulog)
PKXHXTAAPPFDFEAIPEEYL
SEQ ID NO: 653 (modified hirulog)
PKXHXTAGGAPPFDFEAIPEEYL
SEQ ID NO: 654 (modified hirulog)
KXHXGGGAPPFDFEAIPEEYL
SEQ ID NO: 655 (modified hirulog)
KXHXGGAPPFDFEAIPEEYL
SEQ ID NO: 656 (modified hirulog)
KXHXGGGGAPPFDFEAIPEEYL
SEQ ID NO: 657 (modified hirulog)
KXHXTGGAPPFDFEAIPEEYL
SEQ ID NO: 658 (modified hirulog)
KXHXTGAPPFDFEAIPEEYL
SEQ ID NO: 659 (modified hirulog)
KXHXTGGGAPPFDFEAIPEEYL
SEQ ID NO: 660 (modified hirulog)
KXHXTAGAPPFDFEAIPEEYL
SEQ ID NO: 661 (modified hirulog)
KXHXTAAPPFDFEAIPEEYL
SEQ ID NO: 662 (modified hirulog)
KXHXTAGGAPPFDFEAIPEEYL
SEQ ID NO: 663 (modified hirulog)
KXHXGGGAPPFDFEAIPEEYL
SEQ ID NO: 664 (modified hirulog)
KXHXGGAPPFDFEAIPEEYL
SEQ ID NO: 665 (modified hirulog)
KXHXGGGGAPPFDFEAIPEEYL
SEQ ID NO: 666 (modified hirulog)
KXHXTGGAPPFDFEAIPEEYL
SEQ ID NO: 667 (modified hirulog)
KXHXTGAPPFDFEAIPEEYL
SEQ ID NO: 668 (modified hirulog)
KXHXTGGGAPPFDFEAIPEEYL
SEQ ID NO: 669 (modified hirulog)
KXHXTAGAPPFDFEAIPEEYL
SEQ ID NO: 670 (modified hirulog)
KXHXTAAPPFDFEAIPEEYL
SEQ ID NO: 671 (modified hirulog)
KXHXTAGGAPPFDFEAIPEEYL
SEQ ID NO: 672 (modified hirulog)
KXHXGGGAPPFDFEAIPEEYL
SEQ ID NO: 673 (modified hirulog)
KXHXGGAPPFDFEAIPEEYL
SEQ ID NO: 674 (modified hirulog)
KXHXGGGGAPPFDFEAIPEEYL
SEQ ID NO: 675 (modified hirulog)
KXHXTGGAPPFDFEAIPEEYL
SEQ ID NO: 676 (modified hirulog)
KXHXTGAPPFDFEAIPEEYL
SEQ ID NO: 677 (modified hirulog)
KXHXTGGGAPPFDFEAIPEEYL
SEQ ID NO: 678 (modified hirulog)
KXHXTAGAPPFDFEAIPEEYL
SEQ ID NO: 679 (modified hirulog)
KXHXTAAPPFDFEAIPEEYL
SEQ ID NO: 680 (modified hirulog)
KXHXTAGGAPPFDFEAIPEEYL
SEQ ID NO: 681 (modified hirulog)
KXHXGGGAPPFDFEAIPEEYL
SEQ ID NO: 682 (modified hirulog)
KXHXGGAPPFDFEAIPEEYL
SEQ ID NO: 683 (modified hirulog)
KXHXGGGGAPPFDFEAIPEEYL
SEQ ID NO: 684 (modified hirulog)
KXHXTGGAPPFDFEAIPEEYL
SEQ ID NO: 685 (modified hirulog)
KXHXTGAPPFDFEAIPEEYL
SEQ ID NO: 686 (modified hirulog)
KXHXTGGGAPPFDFEAIPEEYL
SEQ ID NO: 687 (modified hirulog)
KXHXTAGAPPFDFEAIPEEYL
SEQ ID NO: 688 (modified hirulog)
KXHXTAAPPFDFEAIPEEYL
SEQ ID NO: 689 (modified hirulog)
KXHXTAGGAPPFDFEAIPEEYL
SEQ ID NO: 690 (modified hirudin)
FPRMHKGNGDFEEIPEEDILN
SEQ ID NO: 691 (modified hirudin)
FPRMHKGGNGDFEEIPEEDILN
SEQ ID NO: 692 (modified hirudin)
FPRMHKGGGNGDFEEIPEEDILN
SEQ ID NO: 693 (modified hirudin)
FPRMHKGNGDFEEIPDEDILN
SEQ ID NO: 694 (modified hirudin)
FPRMHKGGNGDFEEIPDEDILN
SEQ ID NO: 695 (modified hirudin)
FPRMHKGGGNGDFEEIPDEDILN
SEQ ID NO: 696 (modified hirudin)
FPRMHKGNGDFEEIPEAYDE
SEQ ID NO: 697 (modified hirudin)
FPRMHKGGNGDFEEIPEAYDE
SEQ ID NO: 698 (modified hirudin)
FPRMHKGGGNGDFEEIPEAYDE
SEQ ID NO: 699 (modified hirudin)
FPRMHKGNGDFEAIPEAYDE
SEQ ID NO: 700 (modified hirudin)
FPRMHKGGNGDFEAIPEAYDE
SEQ ID NO: 701 (modified hirudin)
FPRMHKGGGNGDFEAIPEAYDE
SEQ ID NO: 702 (modified hirudin)
FPRMHKGNGDFEPIPEAYDE
SEQ ID NO: 703 (modified hirudin)
FPRMHKGGNGDFEPIPEAYDE
SEQ ID NO: 704 (modified hirudin)
FPRMHKGGGNGDFEPIPEAYDE
SEQ ID NO: 705 (modified hirudin)
FPRMHKGNGDFEEFPEAYDE
SEQ ID NO: 706 (modified hirudin)
FPRMHKGGNGDFEEFPEAYDE
SEQ ID NO: 707 (modified hirudin)
FPRMHKGGGNGDFEEFPEAYDE
SEQ ID NO: 708 (modified hirudin)
FPRMHKGNGDFEAFPEAYDE
SEQ ID NO: 709 (modified hirudin)
FPRMHKGGNGDFEAFPEAYDE
SEQ ID NO: 710 (modified hirudin)
FPRMHKGGGNGDFEAFPEAYDE
SEQ ID NO: 711 (modified hirudin)
FPRMHKGNGDFEPFPEAYDE
SEQ ID NO: 712 (modified hirudin)
FPRMHKGGNGDFEPFPEAYDE
SEQ ID NO: 713 (modified hirudin)
FPRMHKGGGNGDFEPFPEAYDE
SEQ ID NO: 714 (modified hirudin)
FPRMHKGNGDFEEIPDAYDE
SEQ ID NO: 715 (modified hirudin)
FPRMHKGGNGDFEEIPDAYDE
SEQ ID NO: 716 (modified hirudin)
FPRMHKGGGNGDFEEIPDAYDE
SEQ ID NO: 717 (modified hirudin)
FPRMHKGNGDFEAIPDAYDE
SEQ ID NO: 718 (modified hirudin)
FPRMHKGGNGDFEAIPDAYDE
SEQ ID NO: 719 (modified hirudin)
FPRMHKGGGNGDFEAIPDAYDE
SEQ ID NO: 720 (modified hirudin)
FPRMHKGNGDFEPIPDAYDE
SEQ ID NO: 721 (modified hirudin)
FPRMHKGGNGDFEPIPDAYDE
SEQ ID NO: 722 (modified hirudin)
FPRMHKGGGNGDFEPIPDAYDE
SEQ ID NO: 723 (modified hirudin)
FPRMHKGNGDFEEFPDAYDE
SEQ ID NO: 724 (modified hirudin)
FPRMHKGGNGDFEEFPDAYDE
SEQ ID NO: 725 (modified hirudin)
FPRMHKGGGNGDFEEFPDAYDE
SEQ ID NO: 726 (modified hirudin)
FPRMHKGGNGDFEAFPDAYDE
SEQ ID NO: 727 (modified hirudin)
FPRMHKGNGDFEAFPDAYDE
SEQ ID NO: 728 (modified hirudin)
FPRMHKGGGNGDFEAFPDAYDE
SEQ ID NO: 729 (modified hirudin)
FPRMHKGNGDFEPFPDAYDE
SEQ ID NO: 730 (modified hirudin)
FPRMHKGGNGDFEPFPDAYDE
SEQ ID NO: 731 (modified hirudin)
FPRMHKGGGNGDFEPFPDAYDE
SEQ ID NO: 732 (modified haemadin)
FPRMHKGNGDFEEIEIDEE
SEQ ID NO: 733 (modified haemadin)
FPRMHKGGNGDFEEIEIDEE
SEQ ID NO: 734 (modified haemadin)
FPRMHKGGGNGDFEEIEIDEE
SEQ ID NO: 735 (modified haemadin)
FPRMHKGNGDFEEFEIDEE
SEQ ID NO: 736 (modified haemadin)
FPRMHKGGNGDFEEFEIDEE
SEQ ID NO: 737 (modified haemadin)
FPRMHKGGGNGDFEEFEIDEE
SEQ ID NO: 738 (modified boophilin)
FPRMHKGVNDFEEIPEEYL
SEQ ID NO: 739 (modified boophilin)
FPRMHKGGVNDFEEIPEEYL
SEQ ID NO: 740 (modified boophilin)
FPRMHKGGGVNDFEEIPEEYL
SEQ ID NO: 741 (modified boophilin)
FPRMHKGVNDFEEIEIDEE
SEQ ID NO: 742 (modified boophilin)
FPRMHKGGVNDFEEIEIDEE
SEQ ID NO: 743 (modified boophilin)
FPRMHKGGGVNDFEEIEIDEE
SEQ ID NO: 744 (modified boophilin)
FPRMHKGVNDFEEFEIDEE
SEQ ID NO: 745 (modified boophilin)
FPRMHKGGVNDFEEFEIDEE
SEQ ID NO: 746 (modified boophilin)
FPRMHKGGGVNDFEEFEIDEE
SEQ ID NO: 747 (modified heparin co-factor Ii)
FPRMHKGNGDFDYLDLEYL
SEQ ID NO: 748 (modified heparin co-factor Ii)
FPRMHKGGNGDFDYLDLEYL
SEQ ID NO: 749 (modified heparin co-factor Ii)
FPRMHKGGGNGDFDYLDLEYL
SEQ ID NO: 750 (modified heparin co-factor Ii)
FPRMHKGNGDFDYVDLEYL
SEQ ID NO: 751 (modified heparin co-factor Ii)
FPRMHKGGNGDFDYVDLEYL
SEQ ID NO: 752 (modified heparin co-factor Ii)
FPRMHKGGGNGDFDYVDLEYL
SEQ ID NO: 753 (modified heparin co-factor Ii)
FPRMHKGNGDFDYLDFEYL
SEQ ID NO: 754 (modified heparin co-factor Ii)
FPRMHKGGNGDFDYLDFEYL
SEQ ID NO: 755 (modified heparin co-factor Ii)
FPRMHKGGGNGDFDYLDFEYL
SEQ ID NO: 756 (modified heparin co-factor Ii)
FPRMHKGNGDFDYVDFEYL
SEQ ID NO: 757 (modified heparin co-factor Ii)
FPRMHKGGNGDFDYVDFEYL
SEQ ID NO: 758 (modified heparin co-factor Ii)
FPRMHKGGGNGDFDYVDFEYL
SEQ ID NO: 759 (modified heparin co-factor Ii)
FPRMHKGNGDFDYLDLDYL
SEQ ID NO: 760 (modified heparin co-factor Ii)
FPRMHKGGNGDFDYLDLDYL
SEQ ID NO: 761 (modified heparin co-factor Ii)
FPRMHKGGGNGDFDYLDLDYL
SEQ ID NO: 762 (modified heparin co-factor Ii)
FPRMHKGNGDFDYVDLDYL
SEQ ID NO: 763 (modified heparin co-factor Ii)
FPRMHKGGNGDFDYVDLDYL
SEQ ID NO: 764 (modified heparin co-factor Ii)
FPRMHKGGGNGDFDYVDLDYL
SEQ ID NO: 765 (modified heparin co-factor Ii)
FPRMHKGNGDFDYLDFDYL
SEQ ID NO: 766 (modified heparin co-factor Ii)
FPRMHKGGNGDFDYLDFDYL
SEQ ID NO: 767 (modified heparin co-factor Ii)
FPRMHKGGGNGDFDYLDFDYL
SEQ ID NO: 768 (modified heparin co-factor Ii)
FPRMHKGNGDFDYVDFDYL
SEQ ID NO: 769 (modified heparin co-factor Ii)
FPRMHKGGNGDFDYVDFDYL
SEQ ID NO: 770 (modified heparin co-factor Ii)
FPRMHKGGGNGDFDYVDFDYL
SEQ ID NO: 771 (consensus sequence)
(N-terminal peptide)-X₁-H-X₂-(G)ₙ-(exosite I
binding peptide)
SEQ ID NO: 772 (synthetic peptide)
FEEIPEEYL
SEQ ID NO: 773 (synthetic peptide)
YEPIPEEA
SEQ ID NO: 774 (synthetic peptide)
NGDFEEIPEEYL
SEQ ID NO: 775 (synthetic peptide)
APPFDFEAIPEEYL
SEQ ID NO: 776 (BPTI—bovine pancreatic trypsin
inhibitor)
MKMSRLCLSVALLVLLGTLAASTPGCDTSNQAKAQRPDFCLEPPYTGP
CKARIIRYFYNAKAGLCQTFVYGGCRAKRNDFKSAEDCMRTCGGAIGP
WENL

1-50. (canceled)

51. A method of producing a modified serine protease inhibitor (SPI) displaying enhanced inhibition of a target serine protease (SP) comprising modifying the SPI such that binding of the SPI to its target SP displaces one or more of the amino acid residues in the catalytic triad of the target SP, or one or more atoms of said amino acid residues.

52. The method of claim 51, wherein:
(a) the method comprises the introduction of one or more amino acid residues into the SPI which are capable of displacing one or more of the amino acid residues of the catalytic triad of the target SP, or one or more atoms of said amino acid residues; or
(b) said method produces a modified SPI which displays a prolonged duration of inhibition; or
(c) the one or more of the residues in the catalytic triad of the target serine protease which is displaced comprises the catalytic serine residue; or
(d) the SPI is a thrombin inhibitor; or
(e) said method further comprises the step of modifying the SPI so that it is capable of being neutralised, comprising the introduction of an area of ionic charge into the SPI, wherein the area of ionic charge is capable of interacting with an area of opposite ionic charge on a neutralising agent.

53. The method of claim 52(a), wherein
(i) said one or more introduced amino acid residues are introduced by substitution or insertion; or
(ii) said one or more amino acid residues capable of displacing one or more of the residues of the catalytic triad of the target SP, or one or more atoms thereof comprises a histidine residue; or
(iii) said one or more introduced amino acids comprises a methionine-histidine sequence;
(iv) said one or more introduced amino acids comprises a methionine-histidine-lysine sequence; or
(v) said one or more introduced amino acids comprises a methionine-histidine-lysine-threonine sequence.

54. The method of claim 52(e), wherein
(i) said introduced area of ionic charge is introduced towards the carboxy-terminus of the SPI; or
(ii) said introduced area of ionic charge is an area of anionic charge; or
(iii) said introduced area of ionic charge comprises one or more acidic residues;
optionally wherein said one or more acidic residues comprises one or more glutamine residues; or
(iv) said neutralising agent is protamine sulphate.

55. The method of modifying an SPI according to claim 52(d), wherein the SPI is selected from the group consisting of any one of SEQ ID NOs: 14 and 17-153.

56. A composition of matter selected from the group consisting of:
(i) a modified SPI obtainable or obtained by the method of claim 51, or a fragment or functional equivalent thereof;
(ii) a modified SPI which displays enhanced inhibition of a target SPI, wherein the binding of the SPI to its target SP displaces one or more of the amino acid residues in the catalytic triad of the target SP, or one or more atoms of said amino acid residues;
(iii) a modified SPI comprising or consisting of a sequence selected from any one of SEQ ID NOs: 158-770, or a fragment or functional equivalent thereof;
(iv) a nucleic acid molecule encoding a modified SPI according to (i), (ii) or (iii), or an anti-sense nucleic acid molecule which hybridises under high stringency hybridisation conditions to said nucleic acid molecule encoding a modified SPI;
(v) a vector comprising a nucleic acid sequence of (iv); and
(vi) a host cell comprising the vector of (v) or the nucleic acid molecule of (iv).

57. The composition of matter according to claim 56(i) or (ii) wherein said modified SPI is a thrombin inhibitor.

58. The composition of matter according to claim 57, wherein said modified SPI:
(i) comprises the consensus sequence: N-terminal peptide) —X₁—H—X₂-(G)ₙ- (exosite I binding peptide) (SEQ ID NO: 771); or
(ii) comprises or consists of a sequence selected from any one of SEQ ID NOs: 158-770, or a fragment or functional equivalent thereof.

59. A method of inhibiting a target SP comprising administering to a subject a composition of matter according to claim 56(i), (ii) or (iii).

60. A method of treating a subject suffering from a coagulopathy or preventing a subject developing a coagulopathy comprising administering a composition of matter according to claim 57.

61. A method of neutralising thrombin inhibition in a subject comprising:
(a) administering a composition of matter according to claim 57; and
(b) subsequently administering to the subject an amount of protamine sulphate sufficient to result in neutralisation of the thrombin inhibition.

62. The composition of matter comprising a modified SPI according to claim 56(ii), wherein said modified SPI comprises one or more amino acid residues which are capable of displacing one or more of the amino acid residues of the catalytic triad of the target SP, or one or more atoms of said amino acid residues.

63. The composition of matter according to claim 62, wherein:
(a) said SPI displays a prolonged duration of inhibition; or
(b) said one or more amino acid residues capable of displacing one or more of the residues of the catalytic triad of the target SP, or one or more atoms thereof comprises a histidine residue; or
(c) said one or more amino acid residues capable of displacing one or more of the residues of the catalytic triad of the target SP comprises a methionine-histidine sequence; or
(d) said one or more amino acid residues capable of displacing one or more of the residues of the catalytic triad of the target SP comprises a methionine-histidine-lysine sequence; or
(e) wherein said one or more amino acid residues capable of displacing one or more of the residues of the catalytic triad of the target SP comprises a methionine-histidine-lysine-threonine sequence.

64. The composition of matter comprising a modified SPI according to claim 56(ii), wherein the one or more amino acid residues in the catalytic triad of the target serine protease which is displaced comprises the catalytic serine residue.

65. The composition of matter comprising a modified SPI according to claim 56(ii), wherein said modified SPI further comprises an area of ionic charge, wherein the area of ionic charge is capable of interacting with an area of opposite ionic charge on a neutralising agent.

66. The composition of matter according to claim 65, wherein said area of ionic charge is positioned towards the carboxy-terminus of the SPI.

67. The composition of matter according to claim 65, wherein said area of ionic charge is an area of anionic charge.

68. The composition of matter according to claim 65, wherein said area of ionic charge comprises one or more acidic residues.

69. The composition of matter according to claim 68, wherein said one or more acidic residues comprises one or more glutamine residues.

70. The composition of matter according to claim 65, wherein said neutralising agent is protamine sulphate.
